SEQUENCE LISTING (198 sequences)

SEQ ID NO: 1 - DNA, 201 bp, Homo sapiens
caaggggggtggagggagagcggggaaggagggggtgtcagctcaactgtaaaagctgcacagattttttttctttctctctctctgcctctccatagatcgtttctgtttcatgccctgtctcatttcgcatagctaaaaaagaatgctaattaagatcccttgtcttaacctgaaaaataatgactcggctgtaattag

SEQ ID NO: 2 - DNA, 201 bp, Homo sapiens
caaggggggtggagggagagcggggaaggagggggtgtcagctcaactgtaaaagctgcacagattttttttctttctctctctctgcctctccatagattgtttctgtttcatgccctgtctcatttcgcatagctaaaaaagaatgctaattaagatcccttgtcttaacctgaaaaataatgactcggctgtaattag

SEQ ID NO: 3 - DNA, 201 bp, Homo sapiens
ttatcatatcgcttccacttcctttatggcatcatctccaatgggcttcttctccctcttggatccaggttctcagattggggacatgcagagtccaaggaacattccattctcctccctggtctagaacaaggagggcttagatatatgagcaggtggctggggctggcgagctatgtagtctccaatggcttttccctg

SEQ ID NO: 4 - DNA, 201 bp, Homo sapiens
ttatcatatcgcttccacttcctttatggcatcatctccaatgggcttcttctccctcttggatccaggttctcagattggggacatgcagagtccaagggacattccattctcctccctggtctagaacaaggagggcttagatatatgagcaggtggctggggctggcgagctatgtagtctccaatggcttttccctg

SEQ ID NO: 5 - DNA, 201 bp, Homo sapiens
tcacatgctgtatttagacagtctgaatagcaaacactgagaaatgctaggccagaggatttgggccccaatccttttaaccagcaaatgggacagtccagtgaaacagacagccatgttagtggcttttgatttgattccactgacatacacacagttcgaattcaatgtcatatttattttaacattgcagtgaaaaat

SEQ ID NO: 6 - DNA, 201 bp, Homo sapiens
tcacatgctgtatttagacagtctgaatagcaaacactgagaaatgctaggccagaggatttgggccccaatccttttaaccagcaaatgggacagtccaatgaaacagacagccatgttagtggcttttgatttgattccactgacatacacacagttcgaattcaatgtcatatttattttaacattgcagtgaaaaat

SEQ ID NO: 7 - DNA, 201 bp, Homo sapiens
ttcacttagatgtgtctgatgctgttaatgtgatggtgtatgttgggattcccatcggggagggtgctcatgatgaaggtatgctttctagaatccactgttttaggatggtgaggctttgtcttgggaatacagttttgtgttttcatccttagggccagtggcttttccttttttttttctttttcttttttctgtttt

SEQ ID NO: 8 - DNA, 201 bp, Homo sapiens
ttcacttagatgtgtctgatgctgttaatgtgatggtgtatgttgggattcccatcggggagggtgctcatgatgaaggtatgctttctagaatccactgctttaggatggtgaggctttgtcttgggaatacagttttgtgttttcatccttagggccagtggcttttccttttttttttctttttcttttttctgtttt

SEQ ID NO: 9 - DNA, 201 bp, Homo sapiens
tctaaatgtaagagttcagactcacattctattaaaatttagccctaaaatgacaagccttcttaaagccttatttttcaaaagcgccccccccattcttattcagattaagagttgccaaaataccttctgaactacactgcattgttgtgccgagaacaccgagcactgaactttgcaaagaccttcgtctttgagaag

SEQ ID NO: 10 - DNA, 201 bp, Homo sapiens
tctaaatgtaagagttcagactcacattctattaaaatttagccctaaaatgacaagccttcttaaagccttatttttcaaaagcgccccccccattcttgttcagattaagagttgccaaaataccttctgaactacactgcattgttgtgccgagaacaccgagcactgaactttgcaaagaccttcgtctttgagaag

SEQ ID NO: 11 - DNA, 201 bp, Homo sapiens
gccttggctcagcagtgacccagaccttccccaggatgctgagagctgttaccttgtccacaccctgctggagttctacttgaaaactgttttcaaaaaccaccacaatagaacagttgaagtcaggactctgaagtcattctctactctggccaacaactttgttctcatcgtgtcacaactgcaacccagtgtgagtag

SEQ ID NO: 12 - DNA, 201 bp, Homo sapiens
gccttggctcagcagtgacccagaccttccccaggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtgtgagtag

SEQ ID NO: 13 - DNA, 201 bp, Homo sapiens
ggaggtatgcgtaaggcctcagccagaagccagtggctaagtttgggaatggagattgggagaaaggaaggcatggatttttacatatgagccttcaatgatgttgcctggttactattattaaagaatttctcagcttttataattcctgtggccagcctagttcaggtggtagaacaccagactcttagcaaatgaatg

SEQ ID NO: 14 - DNA, 201 bp, Homo sapiens
ggaggtatgcgtaaggcctcagccagaagccagtggctaagtttgggaatggagattgggagaaaggaaggcatggatttttacatatgagccttcaatggtgttgcctggttactattattaaagaatttctcagcttttataattcctgtggccagcctagttcaggtggtagaacaccagactcttagcaaatgaatg

SEQ ID NO: 15 - DNA, 201 bp, Homo sapiens
gtagagctggggcactctcaacccacacactgctgtcgaatgaatgaatgaatgaatgaatgaatggggagaacaaagtggctggaactcagacctccttccactggagtcatccctagaacagcgtttcccactgaaaacctcaaggcctcacttctccgtctccctcctcacctgccgagggctcacaatgatgctgtt

SEQ ID NO: 16 - DNA, 201 bp, Homo sapiens
gtagagctggggcactctcaacccacacactgctgtcgaatgaatgaatgaatgaatgaatgaatggggagaacaaagtggctggaactcagacctccttgcactggagtcatccctagaacagcgtttcccactgaaaacctcaaggcctcacttctccgtctccctcctcacctgccgagggctcacaatgatgctgtt

SEQ ID NO: 17 - DNA, 201 bp, Homo sapiens
atcagctggctctttctaaattgacacaccacatgtccctaaaattctctcttcaagtaataccaccatcaaagcaggacatttcccagagccttagagcgtggtgtctgctcagtgggactcaaccccagaagaagctgttaaatcacccactgtttcagtttacaaacttcttacgacttggcaacaagtgaaactaca

SEQ ID NO: 18 - DNA, 201 bp, Homo sapiens
atcagctggctctttctaaattgacacaccacatgtccctaaaattctctcttcaagtaataccaccatcaaagcaggacatttcccagagccttagagcctggtgtctgctcagtgggactcaaccccagaagaagctgttaaatcacccactgtttcagtttacaaacttcttacgacttggcaacaagtgaaactaca

SEQ ID NO: 19 - DNA, 201 bp, Homo sapiens
tctccctcttgcatgcagtgcctgtatacatatagatccaggcagcaacaaaaagtgggtaaatgtaaagaatgttatgtaaatttcatgaggaggccaagttcaagcttttttaaaggcagtttattcttggacaggtatggccagagatggtgccactgtggtgagattttaacaactgtcaaatgtttaaaactccca

SEQ ID NO: 20 - DNA, 201 bp, Homo sapiens
tctccctcttgcatgcagtgcctgtatacatatagatccaggcagcaacaaaaagtgggtaaatgtaaagaatgttatgtaaatttcatgaggaggccaacttcaagcttttttaaaggcagtttattcttggacaggtatggccagagatggtgccactgtggtgagattttaacaactgtcaaatgtttaaaactccca

SEQ ID NO: 21 - DNA, 201 bp, Homo sapiens
agcaggctgggagctctgcgttctctcccaagtaggtctgatggtcacgatgttctcatcagccccattctaggcgcctatgcaagttccaaagaagttctttcctagacctggggtaagcaggccacctcaggagctgcaatcacatcacggcccccaaagctctgtcagagttcttgcctggtccccctaagtccatgg

SEQ ID NO: 22 - DNA, 201 bp, Homo sapiens
agcaggctgggagctctgcgttctctcccaagtaggtctgatggtcacgatgttctcatcagccccattctaggcgcctatgcaagttccaaagaagttcattcctagacctggggtaagcaggccacctcaggagctgcaatcacatcacggcccccaaagctctgtcagagttcttgcctggtccccctaagtccatgg

SEQ ID NO: 23 - DNA, 202 bp, Homo sapiens
ccctacataaaagagattctgcaatgggcagcactaacatgaaacagtgttcagaagtacccattttccctcagattctaaactgacaaggtttccacttatcaggttatgaagttctaaagctgcaagacatccttgaggtcatcacaggatatttatttattttttcttcgggtgcatccaatagttatcaacttttcct

SEQ ID NO: 24 - DNA, 202 bp, Homo sapiens
ccctacataaaagagattctgcaatgggcagcactaacatgaaacagtgttcagaagtacccattttccctcagattctaaactgacaaggtttccacttaccaggttatgaagttctaaagctgcaagacatccttgaggtcatcacaggatatttatttattttttcttcgggtgcatccaatagttatcaacttttcct

SEQ ID NO: 25 - DNA, 201 bp, Homo sapiens
aagtttgaagagccattttggtaaacggtttttattaaagatgctatggaacataaagttgtattgcatgcaatttgaagtaacttatttgactatgaatgttatcggattactgaattgtatcaatttgtttgtgttcaatatcagctttgataattgtgtaccttaagatattgaaggagaaaatagataatttacaag

SEQ ID NO: 26 - DNA, 201 bp, Homo sapiens
aagtttgaagagccattttggtaaacggtttttattaaagatgctatggaacataaagttgtattgcatgcaatttgaagtaacttatttgactatgaatattatcggattactgaattgtatcaatttgtttgtgttcaatatcagctttgataattgtgtaccttaagatattgaaggagaaaatagataatttacaag

SEQ ID NO: 27 - DNA, 201 bp, Homo sapiens
aatcatccattgctcatcctaggaagacgggttgagaatccctaatttgagggtcagttcctgcagaagtgccctttgcctccactcaatgcctcaatttcttttctgcatgactgagagtctcagtgttggaacgggacagtatttatgtatgagtttttcctatttattttgagtctgtgaggtcttcttgtcatgtga

SEQ ID NO: 28 - DNA, 201 bp, Homo sapiens
aatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtga

SEQ ID NO: 29 - DNA, 201 bp, Homo sapiens
tggaggcttctgtttctgcattctgccacgagagctaggtccttgatcttttctttagattgaaagtctgtctctgaacacaattatttgtaaaagttagaagttcttttttaaatcattaaaagaggcttgctgaaggatacactggctgtgtgtatttatctcaattctgcttgatgggccagactaattgttagagga

SEQ ID NO: 30 - DNA, 201 bp, Homo sapiens
tggaggcttctgtttctgcattctgccacgagagctaggtccttgatcttttctttagattgaaagtctgtctctgaacacaattatttgtaaaagttagtagttcttttttaaatcattaaaagaggcttgctgaaggatacactggctgtgtgtatttatctcaattctgcttgatgggccagactaattgttagagga

SEQ ID NO: 31 - DNA, 201 bp, Homo sapiens
cgtcgctggggccccatagtgtgcatcatgtccaacctgtaactctctccccctcttcttccatgaggtcctgagaccaggattcctaaaacaaacaggacgagggacctttagacacgcaaggagacatgcctctagcaggatcagggggactggggtcgggagggtggggcaggaaggaagccagaatcagaagtatcc

SEQ ID NO: 32 - DNA, 201 bp, Homo sapiens
cgtcgctggggccccatagtgtgcatcatgtccaacctgtaactctctccccctcttcttccatgaggtcctgagaccaggattcctaaaacaaacaggatgagggacctttagacacgcaaggagacatgcctctagcaggatcagggggactggggtcgggagggtggggcaggaaggaagccagaatcagaagtatcc

SEQ ID NO: 33 - DNA, 201 bp, Homo sapiens
cttcataacctcataaatgtttatacggtggaagcccctttcccaatcagggccctggtccacctgctcccggaagagccgaagctctgcagaggagatcgcctcgttctcagggatgctgctgaggttaaagaggaaacgaaaagcagagttttcactggtccctgggatgttctccagatgttctaggcacagttagga

SEQ ID NO: 34 - DNA, 201 bp, Homo sapiens
cttcataacctcataaatgtttatacggtggaagcccctttcccaatcagggccctggtccacctgctcccggaagagccgaagctctgcagaggagatcacctcgttctcagggatgctgctgaggttaaagaggaaacgaaaagcagagttttcactggtccctgggatgttctccagatgttctaggcacagttagga

SEQ ID NO: 35 - DNA, 201 bp, Homo sapiens
agtctgaagggtctgtggcatcagggcaggggcctgccctttcaactccatccccagatgtctatcaggtaccaaaggctgcttccatagctagtctagccgaaccatttccgagctacaaggcagtgaatgaaagtaaaaacaaagaaacactggttaaattttaaaaatttattctttctcttttgttgctgttgattt

SEQ ID NO: 36 - DNA, 201 bp, Homo sapiens
agtctgaagggtctgtggcatcagggcaggggcctgccctttcaactccatccccagatgtctatcaggtaccaaaggctgcttccatagctagtctagctgaaccatttccgagctacaaggcagtgaatgaaagtaaaaacaaagaaacactggttaaattttaaaaatttattctttctcttttgttgctgttgattt

SEQ ID NO: 37 - DNA, 210 bp, Homo sapiens
gcctttcctatgaatttaaatatgcctttaaaataagtcactgttgacagggtcatgagtttccgagtatagttttctttttatcttatttttactcgtctgttgaaaagataatcaaggcctacattttagctgaggataatgaacttttttcctcattcggctgtataatacataaccacagcaagactgacatccacttaggatgat

SEQ ID NO: 38 - DNA, 210 bp, Homo sapiens
gcctttcctatgaatttaaatatgcctttaaaataagtcactgttgacagggtcatgagtttccgagtatagttttctttttatcttatttttactcgtccgttgaaaagataatcaaggcctacattttagctgaggataatgaacttttttcctcattcggctgtataatacataaccacagcaagactgacatccacttaggatgat

SEQ ID NO: 39 - DNA, 201 bp, Homo sapiens
ggagacagcccaccttgaaggggaaggagatggggaaggccccttgcagctgaaagtcccactggctggcctcaggctgtcttattccgcttgaaaataggcaaaaagtctactgtggtatttgtaataaactctatctgctgaaagggcctgcaggccatcctgggagtaaagggctgccttcccatctaatttattgta

SEQ ID NO: 40 - DNA, 201 bp, Homo sapiens
ggagacagcccaccttgaaggggaaggagatggggaaggccccttgcagctgaaagtcccactggctggcctcaggctgtcttattccgcttgaaaatagccaaaaagtctactgtggtatttgtaataaactctatctgctgaaagggcctgcaggccatcctgggagtaaagggctgccttcccatctaatttattgta

SEQ ID NO: 41 - DNA, 201 bp, Homo sapiens
gaggggaagctggagccccaactttgatcctccattggagtggcccaaatctttccatctagggcaagtcctgaaaggcccaaggccccctccccagtctagccttggcctccagcctggagaagggctaacatcagctcattgtcaaggccacccccaccccagaacagaaccgtgtctctgataaaggttttgaagtga

SEQ ID NO: 42 - DNA, 201 bp, Homo sapiens
gaggggaagctggagccccaactttgatcctccattggagtggcccaaatctttccatctagggcaagtcctgaaaggcccaaggccccctccccagtctggccttggcctccagcctggagaagggctaacatcagctcattgtcaaggccacccccaccccagaacagaaccgtgtctctgataaaggttttgaagtga

SEQ ID NO: 43 - DNA, 201 bp, Homo sapiens
ttgcaaaggcgacaataaattattgacgcttaacctttccagtttataagactggaatataatttcaaaccacacattagtacttatgttgcacaatgagcaaagaaattagtttcaaatttacctcagcgtttgtgtatcgggcaaaaatcgttttgcccgattccgtattggtatacttttgcttcagttgcatatctt

SEQ ID NO: 44 - DNA, 201 bp, Homo sapiens
ttgcaaaggcgacaataaattattgacgcttaacctttccagtttataagactggaatataatttcaaaccacacattagtacttatgttgcacaatgagaaaagaaattagtttcaaatttacctcagcgtttgtgtatcgggcaaaaatcgttttgcccgattccgtattggtatacttttgcttcagttgcatatctt

SEQ ID NO: 45 - DNA, 201 bp, Homo sapiens
ccttgaatagctaaagggtaagatgaaagtatttagctgtagggggaaaaagcattgacaggttggaaaagtaaaagtcagattctccttgctctgaaatcttgtacagggcaggttctactaggtatgttacaatgcagaaaaaacatgaaataattgagaggaatttggtgcaatattatcttcttggcttcttttgag

SEQ ID NO: 46 - DNA, 201 bp, Homo sapiens
ccttgaatagctaaagggtaagatgaaagtatttagctgtagggggaaaaagcattgacaggttggaaaagtaaaagtcagattctccttgctctgaaattttgtacagggcaggttctactaggtatgttacaatgcagaaaaaacatgaaataattgagaggaatttggtgcaatattatcttcttggcttcttttgag

SEQ ID NO: 47 - DNA, 201 bp, Homo sapiens
taatcaaaagaagtatcgggtttgtacataattttccaaaaattgtagttgttttcagttgtgtgtatttaagatgaaaagtctacatggaaggttactccggcaaagtgcttagcacgtttgcttttttgcagtgctactgttgagttcacaagttcaagtccagaaaaaaaaagtggataatccactctgctgactttc

SEQ ID NO: 48 - DNA, 201 bp, Homo sapiens
taatcaaaagaagtatcgggtttgtacataattttccaaaaattgtagttgttttcagttgtgtgtatttaagatgaaaagtctacatggaaggttactctggcaaagtgcttagcacgtttgcttttttgcagtgctactgttgagttcacaagttcaagtccagaaaaaaaaagtggataatccactctgctgactttc

SEQ ID NO: 49 - DNA, 201 bp, Homo sapiens
ttgagttcagctataccaaaagtctgaaccttctgcacttagaacaaggcaaccaccaagcttcacttgcactgaggccgtgtctccaatggaaatgaggtagctggcttgcaggagcttcccaactcagggaagtagaactcctgagtcacctccatatgcaaatgatttcacagtaatgctgttgaacttcacttccca

SEQ ID NO: 50 - DNA, 201 bp, Homo sapiens
ttgagttcagctataccaaaagtctgaaccttctgcacttagaacaaggcaaccaccaagcttcacttgcactgaggccgtgtctccaatggaaatgaggcagctggcttgcaggagcttcccaactcagggaagtagaactcctgagtcacctccatatgcaaatgatttcacagtaatgctgttgaacttcacttccca

SEQ ID NO: 51 - DNA, 201 bp, Homo sapiens
agacgtaatccagcattggaacttctgatcttcaagcagggattctcaacctgtggtttaggggttcatcggggctgagcgtgacaagaggaaggaatggacccgtgggatgcaggcaatgtgggacttaaaaggcccaagcactgaaaatggaacctggcgaaagcagaggaggagaatgaagaaagatggagtcaaaca

SEQ ID NO: 52 - DNA, 201 bp, Homo sapiens
agacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaaca

SEQ ID NO: 53 - DNA, 201 bp, Homo sapiens
atccctcccatccatccttcagtctgccacacacgcagtctacgttacacacatgtcacgtaaagcaggatgacatccatgtcacatacatagacatattgaccgaaatgtggcccttcggttgcatatattctcatacatgaatatatttatagaaatatatgcacatatttttgtatattggatatatttatgtaacta

SEQ ID NO: 54 - DNA, 201 bp, Homo sapiens
atccctcccatccatccttcagtctgccacacacgcagtctacgttacacacatgtcacgtaaagcaggatgacatccatgtcacatacatagacatattaaccgaaatgtggcccttcggttgcatatattctcatacatgaatatatttatagaaatatatgcacatatttttgtatattggatatatttatgtaacta

SEQ ID NO: 55 - DNA, 201 bp, Homo sapiens
gggagacagttgtgattaatgagatggcatgtgaagcgcccagcatcatgtctagaaaatatgtttcctgtgctctcacttgtcaccattcctagggacagaggtagaggaagaagagtgtatctgggcccctgcagatgaaagcagtgacaattgtacatgccagagaagaaaaacgcatatataacctatgcactggga

SEQ ID NO: 56 - DNA, 201 bp, Homo sapiens
gggagacagttgtgattaatgagatggcatgtgaagcgcccagcatcatgtctagaaaatatgtttcctgtgctctcacttgtcaccattcctagggacaaaggtagaggaagaagagtgtatctgggcccctgcagatgaaagcagtgacaattgtacatgccagagaagaaaaacgcatatataacctatgcactggga

SEQ ID NO: 57 - DNA, 201 bp, Homo sapiens
aaagatagtctgatagatgctgcagccccattttagcaaatgtgacctcacaggcctgaatgccatcgctattccacatctacaggatagacggaaaggagagaaataaaaaaataggtacctaacactggcaagaggatgatgactcatgttatttcacttaacctttttatcttttaacatgaaggactcatacaggtt

SEQ ID NO: 58 - DNA, 201 bp, Homo sapiens
aaagatagtctgatagatgctgcagccccattttagcaaatgtgacctcacaggcctgaatgccatcgctattccacatctacaggatagacggaaaggaaagaaataaaaaaataggtacctaacactggcaagaggatgatgactcatgttatttcacttaacctttttatcttttaacatgaaggactcatacaggtt

SEQ ID NO: 59 - DNA, 201 bp, Homo sapiens
ctgagaataaggaatatttgtatttgcaaggtattggacacatattaccaaatggccttgcaattaagagtcctgccagcactgtgtgaaaatcgtactagctaattaggtaacatttttaggtgggctacatttccagatcagaagaactcaacttgtttctcagatcacttgttgaatcctgatgaaggagaagaaatc

SEQ ID NO: 60 - DNA, 201 bp, Homo sapiens
ctgagaataaggaatatttgtatttgcaaggtattggacacatattaccaaatggccttgcaattaagagtcctgccagcactgtgtgaaaatcgtactacctaattaggtaacatttttaggtgggctacatttccagatcagaagaactcaacttgtttctcagatcacttgttgaatcctgatgaaggagaagaaatc

SEQ ID NO: 61 - DNA, 201 bp, Homo sapiens
ttggcataaggcagcatggtgtggcagttaagagatgggctgtgcagcccatcctgagctccagtcctgagtttgctacttacttctgtggcctctggaaacttatccaacctcttggtgcttcagtttcctcatctgtgaaattagaatttataataattgcacctacctcccaggggtaactaaatgaataaatataat

SEQ ID NO: 62 - DNA, 201 bp, Homo sapiens
ttggcataaggcagcatggtgtggcagttaagagatgggctgtgcagcccatcctgagctccagtcctgagtttgctacttacttctgtggcctctggaaccttatccaacctcttggtgcttcagtttcctcatctgtgaaattagaatttataataattgcacctacctcccaggggtaactaaatgaataaatataat

SEQ ID NO: 63 - DNA, 201 bp, Homo sapiens
gaagtctggggtggcgccgcagagctcacctgaggaacagggcacaggtgctctggcccagcacatagtcgggaattacgtcgccaaattcccagggcacgttgcgcacgaacttcagtacgggattgcccctctggggagggacgaagggcagaagccatcaatagggatgacccttgataaccacagggccctcctcca

SEQ ID NO: 64 - DNA, 201 bp, Homo sapiens
gaagtctggggtggcgccgcagagctcacctgaggaacagggcacaggtgctctggcccagcacatagtcgggaattacgtcgccaaattcccagggcacattgcgcacgaacttcagtacgggattgcccctctggggagggacgaagggcagaagccatcaatagggatgacccttgataaccacagggccctcctcca

SEQ ID NO: 65 - DNA, 201 bp, Homo sapiens
ccggggtctcctggaccttgacagagcagctaactccgagagcagtgggcaggtggccgcccctgaggcttcacgccgggagaagccaccttcccaccccctcataccgcctcgtgccagcagcctcgcacaggccctagctttacgctcatcacctaaacttgtactttatttttctgatagaaatggtttcctctggat

SEQ ID NO: 66 - DNA, 201 bp, Homo sapiens
ccggggtctcctggaccttgacagagcagctaactccgagagcagtgggcaggtggccgcccctgaggcttcacgccgggagaagccaccttcccaccccttcataccgcctcgtgccagcagcctcgcacaggccctagctttacgctcatcacctaaacttgtactttatttttctgatagaaatggtttcctctggat

SEQ ID NO: 67 - DNA, 201 bp, Homo sapiens
gcttcttcctccagaagtgggcgctgggcagtcacgcagggtttgaaccggaagcgggagtaggtagctgcgtggctaacggagaaaagaagccgtggccacgggaggaggcgagaggagtcgggatctgcgctgcagccaccgccgcggttgatactactttgaccttccgagtgcagtggtaggggcgcggaggcaacg

SEQ ID NO: 68 - DNA, 201 bp, Homo sapiens
gcttcttcctccagaagtgggcgctgggcagtcacgcagggtttgaaccggaagcgggagtaggtagctgcgtggctaacggagaaaagaagccgtggccgcgggaggaggcgagaggagtcgggatctgcgctgcagccaccgccgcggttgatactactttgaccttccgagtgcagtggtaggggcgcggaggcaacg

SEQ ID NO: 69 - DNA, 201 bp, Homo sapiens
gagctggactggatgactctggaggccccttccaaacctcaagtgtccggcgctttgattgcctgagtttctgacacttcagggcccagaggtcctgcgaagggcagaactggacccccatgccagtgctgctgcaggagggcccatatactagggtctgctgagctgttgtcactgatcggtgggcgctgggggggtagg

SEQ ID NO: 70 - DNA, 201 bp, Homo sapiens
gagctggactggatgactctggaggccccttccaaacctcaagtgtccggcgctttgattgcctgagtttctgacacttcagggcccagaggtcctgcgaggggcagaactggacccccatgccagtgctgctgcaggagggcccatatactagggtctgctgagctgttgtcactgatcggtgggcgctgggggggtagg

SEQ ID NO: 71 - DNA, 201 bp, Homo sapiens
gctcgaattatactttgattgagggcgtcattcaggatgaattcgtatttgatgatcctcataaagttgtatttcacattgctcaggaagctaaaaggtgatgacctaggcttgatgatttctaaaaccatgatcacaagtgcagattaatgtctatgtacaaacacagatgatatacacagtctagtacaaacagggaaa

SEQ ID NO: 72 - DNA, 201 bp, Homo sapiens
gctcgaattatactttgattgagggcgtcattcaggatgaattcgtatttgatgatcctcataaagttgtatttcacattgctcaggaagctaaaaggtggtgacctaggcttgatgatttctaaaaccatgatcacaagtgcagattaatgtctatgtacaaacacagatgatatacacagtctagtacaaacagggaaa

SEQ ID NO: 73 - DNA, 201 bp, Homo sapiens
gcctgtaatcccaacactttgagaagctgaggtgggaggagtgcttgaggccaggagttcaagaccagcctgggcaacatagggagacccccatctttacgaagaaaaaaaaaaggggaaaagaaaatcttttaaatctttggatttgatcactacaagtattattttacaagtgaaataaacataccattttcttttaga

SEQ ID NO: 74 - DNA, 201 bp, Homo sapiens
gcctgtaatcccaacactttgagaagctgaggtgggaggagtgcttgaggccaggagttcaagaccagcctgggcaacatagggagacccccatctttacaaagaaaaaaaaaaggggaaaagaaaatcttttaaatctttggatttgatcactacaagtattattttacaagtgaaataaacataccattttcttttaga

SEQ ID NO: 75 - DNA, 201 bp, Homo sapiens
gttccctgtttctccagtaaaggactggcttttgcttgggcagaggtggtgctgctgggtgtgcagctgccacagactccaaaggcgtagaagtttgtgctaacacacggagtcattctggctctctgctgaggcccctgttttctggcaggtgccctccttggaaactggttttggctctgatcagcggttctttttgca

SEQ ID NO: 76 - DNA, 201 bp, Homo sapiens
gttccctgtttctccagtaaaggactggcttttgcttgggcagaggtggtgctgctgggtgtgcagctgccacagactccaaaggcgtagaagtttgtgccaacacacggagtcattctggctctctgctgaggcccctgttttctggcaggtgccctccttggaaactggttttggctctgatcagcggttctttttgca

SEQ ID NO: 77 - DNA, 201 bp, Homo sapiens
agggtttattccctggtgccaccagccttcctgtgggccccttagcaatgtcttaggaaaggagatcaacattttcaaattagatgtttcaactgtgctcctgttttgtcttgaaagtggcaccagaggtgcttctgcctgtgcagcgggtgctgctggtaacagtggctgcttctctctctctctctcttttttgggggc

SEQ ID NO: 78 - DNA, 201 bp, Homo sapiens
agggtttattccctggtgccaccagccttcctgtgggccccttagcaatgtcttaggaaaggagatcaacattttcaaattagatgtttcaactgtgctcttgttttgtcttgaaagtggcaccagaggtgcttctgcctgtgcagcgggtgctgctggtaacagtggctgcttctctctctctctctcttttttgggggc

SEQ ID NO: 79 - DNA, 201 bp, Homo sapiens
attaacttaaataaaaatcaatgctatcatataagtgacatcttgttatatttaaaatacataatatacctttcaatttgtggaggctgcttcttggactgaactgctttcaggaattcagtaaaatattctggctttacaattggacgtccacaaatgagtgcacatattgtctacaatgaagaaaacatgtgaatatat

SEQ ID NO: 80 - DNA, 201 bp, Homo sapiens
attaacttaaataaaaatcaatgctatcatataagtgacatcttgttatatttaaaatacataatatacctttcaatttgtggaggctgcttcttggactcaactgctttcaggaattcagtaaaatattctggctttacaattggacgtccacaaatgagtgcacatattgtctacaatgaagaaaacatgtgaatatat

SEQ ID NO: 81 - DNA, 240 bp, Homo sapiens
tccccctgctccaagcagagctggctatgctgggcctcaccagtgctctggatggtgccttctgtgtggatagcatcactgcgctgaaggccctggagcgagcaagcagcccctcagaacacggcccaaggaagagctatagcctaggcagcatctacactcgcctgtatgggcagtcccctccagactcgcacacggctgagggtgatgtcctggccctgctcagcatctgtcagtgga

SEQ ID NO: 82 - DNA, 240 bp, Homo sapiens
tccccctgctccaagcagagctggctatgctgggcctcaccagtgctctggatggtgccttctgtgtggatagcatcactgcgctgaaggccctggagcgagcaagcagcccctcagaacacggcccaaggaagagctacagcctaggcagcatctacactcgcctgtatgggcagtcccctccagactcgcacacggctgagggtgatgtcctggccctgctcagcatctgtcagtgga

SEQ ID NO: 83 - DNA, 201 bp, Homo sapiens
cataggcagagatgatacctaattctgcatttgattgtcactttttgtacctgcattaatttaataaaatattcttatttattttgttacttggtacaccggcatgtccattttcttgtttattttgtgtttaataaaatgttcagtttaacatcccagtggagaaagttacttggaatatttgcagccttgttcctagtt

SEQ ID NO: 84 - DNA, 201 bp, Homo sapiens
cataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatcccagtggagaaagttacttggaatatttgcagccttgttcctagtt

SEQ ID NO: 85 - DNA, 201 bp, Homo sapiens
aaatatccagtgtacttatgaactcatgtttggctcttttaaaaccttttctaaaagctagatcagcatttttctattttacaagttttttgtataaaaatgtgaacatatgagatattgtgagaaatcattttaagtttcatttaaatcatggtgcctcttttgatactttcttaaaattgtgcaagaagaaatcatttt

SEQ ID NO: 86 - DNA, 201 bp, Homo sapiens
aaatatccagtgtacttatgaactcatgtttggctcttttaaaaccttttctaaaagctagatcagcatttttctattttacaagttttttgtataaaaaggtgaacatatgagatattgtgagaaatcattttaagtttcatttaaatcatggtgcctcttttgatactttcttaaaattgtgcaagaagaaatcatttt

SEQ ID NO: 87 - DNA, 201 bp, Homo sapiens
aaacctacgcgattaatcatcagtgtacagtttaggactaacaatccatttattagtagcagaaagaagtttaaaatcttgctttctgatataatttgttvtgtaggccccaatatgggaggtaaatcaacatatattcgacaaactggggtgatagtactcatggcccaaattgggtgttttgtgccatgtgagtcagca

SEQ ID NO: 88 - DNA, 201 bp, Homo sapiens
aaacctacgcgattaatcatcagtgtacagtttaggactaacaatccatttattagtagcagaaagaagtttaaaatcttgctttctgatataatttgttttgtaggccccaatatgggaggtaaatcaacatatattcgacaaactggggtgatagtactcatggcccaaattgggtgttttgtgccatgtgagtcagca

SEQ ID NO: 89 - DNA, 201 bp, Homo sapiens
catggagacaagaccttattaactagaattatcactcgactttggaaattactttaaaactgaatgccattctcatggatagtaaaatatgacacccaagccggttctcaacactttgaaatcatttgagtttggattagacaaaactccataaaatatacacatttgcatgcacagagaaagatcaagattatgtgtaca

SEQ ID NO: 90 - DNA, 201 bp, Homo sapiens
catggagacaagaccttattaactagaattatcactcgactttggaaattactttaaaactgaatgccattctcatggatagtaaaatatgacacccaaggcggttctcaacactttgaaatcatttgagtttggattagacaaaactccataaaatatacacatttgcatgcacagagaaagatcaagattatgtgtaca

SEQ ID NO: 91 - DNA, 201 bp, Homo sapiens
tattttcatagcattgggaatggtaccacctcccgaaaatgtcagaatgaattctgttaatttcaagaacattctacagtgggagtcacctgcttttgccgaagggaacctgactttcacagctcagtacctaaggtgggtctggcctcactattggcaggaacgcaccggaggagccagccctgggctggtcactgggtt

SEQ ID NO: 92 - DNA, 201 bp, Homo sapiens
tattttcatagcattgggaatggtaccacctcccgaaaatgtcagaatgaattctgttaatttcaagaacattctacagtgggagtcacctgcttttgccaaagggaacctgactttcacagctcagtacctaaggtgggtctggcctcactattggcaggaacgcaccggaggagccagccctgggctggtcactgggtt

SEQ ID NO: 93 - DNA, 201 bp, Homo sapiens
tcctttgactccatgtctcacatccaggttacattgatgcaaggggtgggcttccaagtcctttggcagctcctgtggctctgcaactccaatgcccctgaggctctgcagtgtacagccccctttagctgcattcacaggctggcattgagtgcctgcagcttttccaggtgcacagtgcaagcagttggtgaatctacc

SEQ ID NO: 94 - DNA, 201 bp, Homo sapiens
tcctttgactccatgtctcacatccaggttacattgatgcaaggggtgggcttccaagtcctttggcagctcctgtggctctgcaactccaatgcccctgtggctctgcagtgtacagccccctttagctgcattcacaggctggcattgagtgcctgcagcttttccaggtgcacagtgcaagcagttggtgaatctacc

SEQ ID NO: 95 - DNA, 201 bp, Homo sapiens
gctgggccatattactgctgtcatttatggccaaaagtttgctcaaacattttccagttaccagagccacatctcaagggtctgacactgggaaaacaccgcgtgcggatcgggcacacgctgatgcttgccctgctcagggctatctagtgttccctgccagaacctatgcacgtgtggtgagagcttaaagcaatggat

SEQ ID NO: 96 - DNA, 201 bp, Homo sapiens
gctgggccatattactgctgtcatttatggccaaaagtttgctcaaacattttccagttaccagagccacatctcaagggtctgacactgggaaaacaccacgtgcggatcgggcacacgctgatgcttgccctgctcagggctatctagtgttccctgccagaacctatgcacgtgtggtgagagcttaaagcaatggat

SEQ ID NO: 97 - DNA, 201 bp, Homo sapiens
tgtgttctccgctggcaggccccagtctgactcccctccagattcctggcattgcccagcacaggataaggagcagggttggcgtgtgaggccttacctccgggagggcagccgccgacgcatgcggtgacagtccagcacccactccttacgcacgatgcggcctcccaggcctaggacctggctgtacttgggggtgtt

SEQ ID NO: 98 - DNA, 201 bp, Homo sapiens
tgtgttctccgctggcaggccccagtctgactcccctccagattcctggcattgcccagcacaggataaggagcagggttggcgtgtgaggccttacctctgggagggcagccgccgacgcatgcggtgacagtccagcacccactccttacgcacgatgcggcctcccaggcctaggacctggctgtacttgggggtgtt

SEQ ID NO: 99 - DNA, 201 bp, Homo sapiens
atatcttaaagaagaaagaaaacaattaaggaaatacaatactaagaaaacaaggggaaaaaacaatctccccaaggtggatccacccagcaaaccttgagagcatttcctcttatccacctgaataaaaatgaccagccctttccaaatggcagagagcactgagaggagacacaaggagcagcccgcaagcaccaagtg

SEQ ID NO: 100 - DNA, 201 bp, Homo sapiens
atatcttaaagaagaaagaaaacaattaaggaaatacaatactaagaaaacaaggggaaaaaacaatctccccaaggtggatccacccagcaaaccttgacagcatttcctcttatccacctgaataaaaatgaccagccctttccaaatggcagagagcactgagaggagacacaaggagcagcccgcaagcaccaagtg

SEQ ID NO: 101 - DNA, 201 bp, Homo sapiens
atgaaggttctgagaaggcattggggaggctgagatgaaggagttgggatggggtgatgaagttaaggatcaaatgggtgttacaaggaaaggttgggaacggtgcccagttgggggtgtattgacatactggggtacgttggggccagggaaggagatggggttgggttggcataaaggctggggaaggagttagagtaa

SEQ ID NO: 102 - DNA, 201 bp, Homo sapiens
atgaaggttctgagaaggcattggggaggctgagatgaaggagttgggatggggtgatgaagttaaggatcaaatgggtgttacaaggaaaggttgggaatggtgcccagttgggggtgtattgacatactggggtacgttggggccagggaaggagatggggttgggttggcataaaggctggggaaggagttagagtaa

SEQ ID NO: 103 - DNA, 201 bp, Homo sapiens
aaaaattataatattgggcttctttctaaatcgttcacagagaagctcagtaaataaatagaaatgggggttgaggtatcagaggtaataaatattctatgagagaggtacaataaggtttctcaaggggctgggtcagctatcccagagccccagatccgattttggagacctctaatttatgtcctagagtctatagag

SEQ ID NO: 104 - DNA, 201 bp, Homo sapiens
aaaaattataatattgggcttctttctaaatcgttcacagagaagctcagtaaataaatagaaatgggggttgaggtatcagaggtaataaatattctataagagaggtacaataaggtttctcaaggggctgggtcagctatcccagagccccagatccgattttggagacctctaatttatgtcctagagtctatagag

SEQ ID NO: 105 - DNA, 201 bp, Homo sapiens
aaaagttaaaattattgttttcctatatgtaatgatactttttaggcatagatttttaagatacaaaaataaaatgtagtttagtattttatcattaccacctgctgcaatgagtctgccagatcagaggctttcctcactaggaaacctagctttaaatcaaatactggttttcttcttgtaattcacacttgtctattg

SEQ ID NO: 106 - DNA, 201 bp, Homo sapiens
aaaagttaaaattattgttttcctatatgtaatgatactttttaggcatagatttttaagatacaaaaataaaatgtagtttagtattttatcattaccatctgctgcaatgagtctgccagatcagaggctttcctcactaggaaacctagctttaaatcaaatactggttttcttcttgtaattcacacttgtctattg

SEQ ID NO: 107 - DNA, 201 bp, Homo sapiens
ccaaagtaatactatgtctgaacataatacctgaaagatagcatttaggcaatggatattcactgtggtggttctagctagtgttcaaatgttttctggatcaaagcttgaagcaaataagaacgaaaggaaacataactatcagtatcggagttggtgcatgggccggctttaaccagtcatgtttcctagtctatagct

SEQ ID NO: 108 - DNA, 201 bp, Homo sapiens
ccaaagtaatactatgtctgaacataatacctgaaagatagcatttaggcaatggatattcactgtggtggttctagctagtgttcaaatgttttctggaacaaagcttgaagcaaataagaacgaaaggaaacataactatcagtatcggagttggtgcatgggccggctttaaccagtcatgtttcctagtctatagct

SEQ ID NO: 109 - DNA, 201 bp, Homo sapiens
cgttgtcatttttataaaactttgaaatgaggaaactttgataggatgtggattaagaactagggagggggaaagaaggatgggactattacatccacataatacctctgatcaagtatttttgacatttactgtggataaattgtttttaagttttcatgaatgaattgctaagaagggaaaatatccatcctgaaggtg

SEQ ID NO: 110 - DNA, 201 bp, Homo sapiens
cgttgtcatttttataaaactttgaaatgaggaaactttgataggatgtggattaagaactagggagggggaaagaaggatgggactattacatccacatgatacctctgatcaagtatttttgacatttactgtggataaattgtttttaagttttcatgaatgaattgctaagaagggaaaatatccatcctgaaggtg

SEQ ID NO: 111 - DNA, 201 bp, Homo sapiens
agattctagatttggatttaaaattagaactatgtgtagaatgtggctatttctgcaagctcaattttcaattaaaaaatatgaaaaataaaccagtagaccactgttacagttcacattatttaactacaatgtcctctcaagtgtatctttccaagggaagtaactttagaaaaaaatctggacaaacttattcacagt

SEQ ID NO: 112 - DNA, 201 bp, Homo sapiens
agattctagatttggatttaaaattagaactatgtgtagaatgtggctatttctgcaagctcaattttcaattaaaaaatatgaaaaataaaccagtagatcactgttacagttcacattatttaactacaatgtcctctcaagtgtatctttccaagggaagtaactttagaaaaaaatctggacaaacttattcacagt

SEQ ID NO: 113 - DNA, 201 bp, Homo sapiens; identical to SEQ ID NO: 67

SEQ ID NO: 114 - DNA, 201 bp, Homo sapiens; identical to SEQ ID NO: 68

SEQ ID NO: 115 - DNA, 201 bp, Homo sapiens
gctgaagcaggtgaagaaagtggcagaagccacgcggtggcaaaaaggagtcacacactccacctggagacgccttgaagtaactgcacgaaatttgagggtggccaggcagttctacaacagccgctcacagggagagccagaacacagaagaactcagatgactggtagtattaccttcttcataatcccaggcttggg

SEQ ID NO: 116 - DNA, 201 bp, Homo sapiens
gctgaagcaggtgaagaaagtggcagaagccacgcggtggcaaaaaggagtcacacactccacctggagacgccttgaagtaactgcacgaaatttgaggatggccaggcagttctacaacagccgctcacagggagagccagaacacagaagaactcagatgactggtagtattaccttcttcataatcccaggcttggg

SEQ ID NO: 117 - DNA, 201 bp, Homo sapiens; identical to SEQ ID NO: 9

SEQ ID NO: 118 - DNA, 201 bp, Homo sapiens; identical to SEQ ID NO: 10

SEQ ID NO: 119 - DNA, 201 bp, Homo sapiens
aggagggagaaaccatcatgctgaggtgccacagctggaaggacaagcctctggtcaaggtcacattcttccagaatggaaaatcccagaaattctcccastttggatcccaccttctccatcccacaagcaaaccacagtcacagtggtgattaccactgcacaggaaacataggctacacgctgttctcatccaagcct

SEQ ID NO: 120 - DNA, 201 bp, Homo sapiens
aggagggagaaaccatcatgctgaggtgccacagctggaaggacaagcctctggtcaaggtcacattcttccagaatggaaaatcccagaaattctcccaatttggatcccaccttctccatcccacaagcaaaccacagtcacagtggtgattaccactgcacaggaaacataggctacacgctgttctcatccaagcct

SEQ ID NO: 121 - DNA, 201 bp, Homo sapiens
gaaatagcaagggaaatcataaagtacgtgagtctgatagcattggctttagattggaggcatgttagcttttctgctgagagagaaggaaaagtgatgctaataggaaagtcttaagctggagaggaaggagtatgagggaattatatttgctattgtgtttttgctgagaaatagaaagcatgctcatttgctaagaat

SEQ ID NO: 122 - DNA, 201 bp, Homo sapiens
gaaatagcaagggaaatcataaagtacgtgagtctgatagcattggctttagattggaggcatgttagcttttctgctgagagagaaggaaaagtgatgccaataggaaagtcttaagctggagaggaaggagtatgagggaattatatttgctattgtgtttttgctgagaaatagaaagcatgctcatttgctaagaat

SEQ ID NO: 123 - DNA, 201 bp, Homo sapiens
aacacaataattgagaagaggaactcataagcaaagctaaagaactcatccttagagaatacaagttcaaactgagaaaggttttacttttaagacaaaaaaaatagcattaacacacatccaaatgccagtgcaggagttcactgggtttataaaagagaaaactaaactcaagtgtcaaataaaaactggagccgtcaa

SEQ ID NO: 124 - DNA, 201 bp, Homo sapiens
aacacaataattgagaagaggaactcataagcaaagctaaagaactcatccttagagaatacaagttcaaactgagaaaggttttacttttaagacaaaacaaatagcattaacacacatccaaatgccagtgcaggagttcactgggtttataaaagagaaaactaaactcaagtgtcaaataaaaactggagccgtcaa

SEQ ID NO: 125 - DNA, 201 bp, Homo sapiens
ctggctaaaaatacgagctcaggggtgcaaccctgccttggtgctcacctggttgatgcacagcacagggctctggaaggcactgctcagctcacgcagcatggcccccagggactgcagatgcctggccctgggggcggaggcctggctgtcaaattcacagcggaatggggctgccaccgagtcgatgaccaccaggcg

SEQ ID NO: 126 - DNA, 201 bp, Homo sapiens
ctggctaaaaatacgagctcaggggtgcaaccctgccttggtgctcacctggttgatgcacagcacagggctctggaaggcactgctcagctcacgcagcgtggcccccagggactgcagatgcctggccctgggggcggaggcctggctgtcaaattcacagcggaatggggctgccaccgagtcgatgaccaccaggcg

SEQ ID NO: 127 - DNA, 201 bp, Homo sapiens
ttttctggcatccacagccctaaccctgaatatccaaatgtactcgtgcatcaaggatctactcaagtctcacctcctctgggaaccttctttgacctatcacagctctcattgaggacctcccttctatttctgatacaaatagtttctactattctaaatagtccctagtttagtaatttattatatatatactgtctt

SEQ ID NO: 128 - DNA, 201 bp, Homo sapiens
ttttctggcatccacagccctaaccctgaatatccaaatgtactcgtgcatcaaggatctactcaagtctcacctcctctgggaaccttctttgacctattacagctctcattgaggacctcccttctatttctgatacaaatagtttctactattctaaatagtccctagtttagtaatttattatatatatactgtctt

SEQ ID NO: 129 - DNA, 201 bp, Homo sapiens
cactcaggaacttttatgcaggtgctagaaactttatgtcaaagtggccacaagattgtttaataggagacgaacgaatgtaactccatgtttactgctagaaaccaaagctttgtgtaaaatcttgaatttatggggcgggagggtaggaaagcctgtacctgtctgtttttttcctgatccttttccctcattcctgaa

SEQ ID NO: 130 - DNA, 201 bp, Homo sapiens
cactcaggaacttttatgcaggtgctagaaactttatgtcaaagtggccacaagattgtttaataggagacgaacgaatgtaactccatgtttactgctaaaaaccaaagctttgtgtaaaatcttgaatttatggggcgggagggtaggaaagcctgtacctgtctgtttttttcctgatccttttccctcattcctgaa

SEQ ID NO: 131 - DNA, 201 bp, Homo sapiens
taggtgcaaactgaagttttatttagaatgaaaaaatagacaacaaattacagattttaaagagctaaaaaattccacagatgtcacggagtccaggggastaacagcctggttggaggggcgctctccgcctcttctgtatttttggctctctctgagggtagtggttttaccagcatgctttatcaaaaactaaactat

SEQ ID NO: 132 - DNA, 201 bp, Homo sapiens
taggtgcaaactgaagttttatttagaatgaaaaaatagacaacaaattacagattttaaagagctaaaaaattccacagatgtcacggagtccaggggaataacagcctggttggaggggcgctctccgcctcttctgtatttttggctctctctgagggtagtggttttaccagcatgctttatcaaaaactaaactat

SEQ ID NO: 133 - DNA, 201 bp, Homo sapiens
ttgatcaaaataacgatctactttgagtcaagcaaaacctagaggaaatataccaaacaacttgaaaatcatttgtataaaaatcattgcacattattttactcgtttgaaaagtaaaaaagtgtatttacaaaatattttgcagtcaaaattataaagtgaatggatatctcgaagtaacacgtcttgatacagacagtt

SEQ ID NO: 134 - DNA, 201 bp, Homo sapiens
ttgatcaaaataacgatctactttgagtcaagcaaaacctagaggaaatataccaaacaacttgaaaatcatttgtataaaaatcattgcacattattttgctcgtttgaaaagtaaaaaagtgtatttacaaaatattttgcagtcaaaattataaagtgaatggatatctcgaagtaacacgtcttgatacagacagtt

SEQ ID NO: 135 - DNA, 201 bp, Homo sapiens
aataaaagaaaaaaagaaagtaaaggaagagtggttctgcttcttagcgctagcctcaatgacgacctaagctgcacttttccccctagttgtgtcttgcgatgctaaaggacgtcacattgcacaatcttaataaggtttccaatcagccccacccgctctggccccaccctcaccctccaacaaagatttatcaaatgt

SEQ ID NO: 136 - DNA, 201 bp, Homo sapiens
aataaaagaaaaaaagaaagtaaaggaagagtggttctgcttcttagcgctagcctcaatgacgacctaagctgcacttttccccctagttgtgtcttgccatgctaaaggacgtcacattgcacaatcttaataaggtttccaatcagccccacccgctctggccccaccctcaccctccaacaaagatttatcaaatgt

SEQ ID NO: 137 - DNA, 201 bp, Homo sapiens
catggctgtcctcccgctgcctgggcccctcagaggcagctcatgctgagctggagagccagcctgcctctcacattctcctccccgtcccaggccatcacttcccatgtcctctgcatctgccctgaacagctccaggtccatccctgctctgccccagccttagagcagcccctcagactgggctgcactcccctcatc

SEQ ID NO: 138 - DNA, 201 bp, Homo sapiens
catggctgtcctcccgctgcctgggcccctcagaggcagctcatgctgagctggagagccagcctgcctctcacattctcctccccgtcccaggccatcagttcccatgtcctctgcatctgccctgaacagctccaggtccatccctgctctgccccagccttagagcagcccctcagactgggctgcactcccctcatc

SEQ ID NO: 139 - DNA, 201 bp, Homo sapiens
aaaattataatattgggcttctttctaaatcgttcacagagaagctcagtaaataaatagaaatgggggttgaggtatcagaggtaataaatattctatasagaggtacaataaggtttctcaaggggctgggtcagctatcccagagccccagatccgattttggagacctctaatttatgtcctagagtctatagagtc

SEQ ID NO: 140 - DNA, 201 bp, Homo sapiens
aaaattataatattgggcttctttctaaatcgttcacagagaagctcagtaaataaatagaaatgggggttgaggtatcagaggtaataaatattctatawagaggtacaataaggtttctcaaggggctgggtcagctatcccagagccccagatccgattttggagacctctaatttatgtcctagagtctatagagtc

SEQ ID NO: 141 - DNA, 201 bp, Homo sapiens
caagagaatctgatcaatttgaagtccctgtttgggaatgaggcacttatcagcatgaagaattttttctcattctgtgccattttaaaaatagaatatttgtatattaactttataattgggttgtggtttttttgctcagctttttatatttttataagaagctaaatagaagaataattgtatctctgacaggttttt

SEQ ID NO: 142 - DNA, 202 bp, Homo sapiens
caagagaatctgatcaatttgaagtccctgtttgggaatgaggcacttatcagcatgaagaattttttctcattctgtgccattttaaaaatagaatattttgtatattaactttataattgggttgtggtttttttgctcagctttttatatttttataagaagctaaatagaagaataattgtatctctgacaggttttt

SEQ ID NO: 143 - DNA, 201 bp, Homo sapiens
gggcagctgaatacgggggtctggatctcagggcaaggaggcgagtccaggagtgtgatcatgcacggatccagcatggcaagtgacagagaggaggagacggggtctcttgagctggggcctgtagaagcttctctacccagcccccatcagagttcaccccaatttctggccctcaagcctggctcatgctacaccccc

SEQ ID NO: 144 - DNA, 201 bp, Homo sapiens
gggcagctgaatacgggggtctggatctcagggcaaggaggcgagtccaggagtgtgatcatgcacggatccagcatggcaagtgacagagaggaggagatggggtctcttgagctggggcctgtagaagcttctctacccagcccccatcagagttcaccccaatttctggccctcaagcctggctcatgctacaccccc

SEQ ID NO: 145 - DNA, 201 bp, Homo sapiens
gccctgcttcagcctgcagcacccttcaactcccaggagctgggcttgccactctgctcaccttgtggagctccatctgcctttcctccccaattccccccctccctgcactcgtctcttcccacaagagccctgtctccttttcctagctattcccatctgaggccatctttattcatttagtttttagagacagggttt

SEQ ID NO: 146 - DNA, 201 bp, Homo sapiens
gccctgcttcagcctgcagcacccttcaactcccaggagctgggcttgccactctgctcaccttgtggagctccatctgcctttcctccccaattcccccactccctgcactcgtctcttcccacaagagccctgtctccttttcctagctattcccatctgaggccatctttattcatttagtttttagagacagggttt

SEQ ID NO: 147 - DNA, 201 bp, Homo sapiens
ccttagtttccccaagtaaaaatgagggggtgggctaaatatcctcaaagttcccaagcagcccttccattttactttccagagactggcttcctacagtgcaggcggggtcacaggatgtgttccaggctcctttaccccgatttcattaggattctcaggcacatgtttccacctcttcagctgtcccccaccccaact

SEQ ID NO: 148 - DNA, 201 bp, Homo sapiens
ccttagtttccccaagtaaaaatgagggggtgggctaaatatcctcaaagttcccaagcagcccttccattttactttccagagactggcttcctacagttcaggcggggtcacaggatgtgttccaggctcctttaccccgatttcattaggattctcaggcacatgtttccacctcttcagctgtcccccaccccaact

SEQ ID NO: 149 - DNA, 201 bp, Homo sapiens
aaccggttcaatcctcctccagcaagtatgtggactggacttgtgtgatttctggtcctgacttcctttggtttgctcaggttcacagagtgtttccaaaggggctggcctcccaggaagggactattcagaagggcaatgaggttctttccatcaacggcaagtctctcaaggggaccacgcaccatgatgccttggcca

SEQ ID NO: 150 - DNA, 201 bp, Homo sapiens
aaccggttcaatcctcctccagcaagtatgtggactggacttgtgtgatttctggtcctgacttcctttggtttgctcaggttcacagagtgtttccaaatgggctggcctcccaggaagggactattcagaagggcaatgaggttctttccatcaacggcaagtctctcaaggggaccacgcaccatgatgccttggcca

SEQ ID NO: 151 - DNA, 201 bp, Homo sapiens
tcttttctgcaaggagagagactcttctgagaaactggagcgaggagctgaggaaattttcaactcaacggaatttcccctgagactgtaaagaaacacaaaataagtaagtataacttacaggtagaaacttgacaagagggaaacagaaaagaaggaaaaagaaaaagtcatcaaaaatgatggcaacctccaaagtca

SEQ ID NO: 152 - DNA, 201 bp, Homo sapiens
tcttttctgcaaggagagagactcttctgagaaactggagcgaggagctgaggaaattttcaactcaacggaatttcccctgagactgtaaagaaacacagaataagtaagtataacttacaggtagaaacttgacaagagggaaacagaaaagaaggaaaaagaaaaagtcatcaaaaatgatggcaacctccaaagtca

SEQ ID NO: 153 - DNA, 201 bp, Homo sapiens
ccctggaggggaaggtgcagtaggcgtcgtcctcccctgacagaggctgcaggggttggggggaagaccctgtgggtgccccggccacaccctcatcaggttcttcctctgagtaggggtcgtaagtaaagtacacctggcaggcctctatctccaaggcatccgggaggtggaagaagaagtaaccctggttggtgaagc

SEQ ID NO: 154 - DNA, 201 bp, Homo sapiens
ccctggaggggaaggtgcagtaggcgtcgtcctcccctgacagaggctgcaggggttggggggaagaccctgtgggtgccccggccacaccctcatcagggtcttcctctgagtaggggtcgtaagtaaagtacacctggcaggcctctatctccaaggcatccgggaggtggaagaagaagtaaccctggttggtgaagc

SEQ ID NO: 155 - DNA, 201 bp, Homo sapiens
caatcctgtctttctgagcacactttagtggccgatgatttttgtcatcagccaccatcctattgcaaaggttccaactgtatatattcccaatagcaacatagcttctaccatgaacagaaaacattctgatttggaaaaagagagggaggtatggactgggggccagagtcctttccaaggcttctccaattctgccca

SEQ ID NO: 156 - DNA, 201 bp, Homo sapiens
caatcctgtctttctgagcacactttagtggccgatgatttttgtcatcagccaccatcctattgcaaaggttccaactgtatatattcccaatagcaacgtagcttctaccatgaacagaaaacattctgatttggaaaaagagagggaggtatggactgggggccagagtcctttccaaggcttctccaattctgccca

SEQ ID NO: 157 - DNA, 201 bp, Homo sapiens
cccagtgccttgtgtggtgactggcatctggtaggcactcaataaatatttgctgaataaatgagttctgcaaaacaggtttatgaggccaaggtgggtgcatcacttgaggtcaggagttcgagaccagcctggccaacagggtgaaaccccgtctctcctaaaaatacaaaaattagttgagtgtagtggcacacgcct

SEQ ID NO: 158 - DNA, 201 bp, Homo sapiens
cccagtgccttgtgtggtgactggcatctggtaggcactcaataaatatttgctgaataaatgagttctgcaaaacaggtttatgaggccaaggtgggtgaatcacttgaggtcaggagttcgagaccagcctggccaacagggtgaaaccccgtctctcctaaaaatacaaaaattagttgagtgtagtggcacacgcct

SEQ ID NO: 159 - DNA, 200 bp, Homo sapiens
taaaatctaggaaaatcttttttgtgaatactatttatactggaatgcaaattcacataaaaatacttcctttaaagcatgttctatttcacacaagaactaaatgtgagctacttttaaaattgcaagctttatttttaatcaaaccaattcaattttaagtttttagatcattaagtctcatacaacttcctctaatt

SEQ ID NO: 160 - DNA, 200 bp, Homo sapiens
taaaatctaggaaaatcttttttgtgaatactatttatactggaatgcaaattcacataaaaatacttcctttaaagcatgttctatttcacacaagaattaaatgtgagctacttttaaaattgcaagctttatttttaatcaaaccaattcaattttaagtttttagatcattaagtctcatacaacttcctctaatt

SEQ ID NO: 161 - DNA, 201 bp, Homo sapiens
gatgttgggcccttcgtgagaattggcttcctgaagataaagggaaagataaaggggaaatctctgagaaagccaaactggaaaactcaacacaagctgaggagggctttgatgttcctgattgtaaaaaaactatagtgaatgattccagagagtcatgtgttgaggaaaatgatgataaaattacacaagcttcacaat

SEQ ID NO: 162 - DNA, 201 bp, Homo sapiens
gatgttgggcccttcgtgagaattggcttcctgaagataaagggaaagataaaggggaaatctctgagaaagccaaactggaaaactcaacacaagctgaagagggctttgatgttcctgattgtaaaaaaactatagtgaatgattccagagagtcatgtgttgaggaaaatgatgataaaattacacaagcttcacaat

SEQ ID NO: 163 - DNA, 201 bp, Homo sapiens
agaaaggaggcgactcctagatccagggatagccccgtctggtgccagtgtggaagctgcctcccatctcaactccctgagagccacaggtgcctggaggsgctgtgctgccggaaaaagccgggggcctgcatcaccacctcagagctgttcaggaagctggtcctgtccagacacgtcctgcagttcctcctgctctac

SEQ ID NO: 164 - DNA, 201 bp, Homo sapiens
agaaaggaggcgactcctagatccagggatagccccgtctggtgccagtgtggaagctgcctcccatctcaactccctgagagccacaggtgcctggaggagctgtgctgccggaaaaagccgggggcctgcatcaccacctcagagctgttcaggaagctggtcctgtccagacacgtcctgcagttcctcctgctctac

SEQ ID NO: 165 - DNA, 231 bp, Homo sapiens
ttactcacatttccacatgggaactggcacaggtggggagtgggtaaaggagtccagcaggctgaatgccttcaacaatcattttaccacatggtcctcatttactctcagctgcctcatatgtgtcacctcacaagtaatcaaataaaatgggcatgtggctaagctttgtaaatagtgaaaacatgaatgtcaattttttttacatatttctattacagatatagcttc

SEQ ID NO: 166 - DNA, 231 bp, Homo sapiens
ttactcacatttccacatgggaactggcacaggtggggagtgggtaaaggagtccagcaggctgaatgccttcaacaatcattttaccacatggtcctcacttactctcagctgcctcatatgtgtcacctcacaagtaatcaaataaaatgggcatgtggctaagctttgtaaatagtgaaaacatgaatgtcaattttttttacatatttctattacagatatagcttc

SEQ ID NO: 167 - DNA, 200 bp, Homo sapiens
tactttaaccgaagtgagtcaggtcttactgaaaaactgaccacagcctgtccgctcccagccttccccagaccagggctgcccaggcagagcgtgagggagactagtgtttactcagctcatttttttcctcctctctcctctaagtcacggggctttgagtagggcttccccagccaccgtgcccctcccagcagacc

SEQ ID NO: 168 - DNA, 200 bp, Homo sapiens
tactttaaccgaagtgagtcaggtcttactgaaaaactgaccacagcctgtccgctcccagccttccccagaccagggctgcccaggcagagcgtgagggggactagtgtttactcagctcatttttttcctcctctctcctctaagtcacggggctttgagtagggcttccccagccaccgtgcccctcccagcagacc

SEQ ID NO: 169 - DNA, 201 bp, Homo sapiens
tcccctttccttgactctacatctaatttatttggaaagcatgtcaagcttaccttcttaatccttctctggagtctatcctattcacattgcttctaccggctgcctgatctgtgttaccatgagtcctctcttgaacaatggtctgctaaattggtttcccatttccagtcttgactccctacaagtgaggcttcacat

SEQ ID NO: 170 - DNA, 201 bp, Homo sapiens
tcccctttccttgactctacatctaatttatttggaaagcatgtcaagcttaccttcttaatccttctctggagtctatcctattcacattgcttctaccagctgcctgatctgtgttaccatgagtcctctcttgaacaatggtctgctaaattggtttcccatttccagtcttgactccctacaagtgaggcttcacat

SEQ ID NO: 171 - DNA, 201 bp, Homo sapiens
catttccatggtatggttagagggatagctacgagccatgacatggactaccagtaaacttctgtgggagaaggagggaaaatgacaatctagcatagattaacagtctcctaaatgcaaatagaatagttgacagttatttttttttaaaaagttcttctgtcatgttaatgacacaaaacttctcttttttgaaggcaa

SEQ ID NO: 172 - DNA, 201 bp, Homo sapiens
catttccatggtatggttagagggatagctacgagccatgacatggactaccagtaaacttctgtgggagaaggagggaaaatgacaatctagcatagatgaacagtctcctaaatgcaaatagaatagttgacagttatttttttttaaaaagttcttctgtcatgttaatgacacaaaacttctcttttttgaaggcaa

SEQ ID NO: 173 - DNA, 201 bp, Homo sapiens
ccttctcacaaatgtaatgtcaagaacatttctattatagtctagttaaaatgttctaaaaatgtgttaattgtaggttttggattctaaaaatgtctccctgaagaatggcacaaagcactttttctgttatttaatacgttgtatgcactcagtaaatattatagcagtccatggtcagggtttattacattagaactg

SEQ ID NO: 174 - DNA, 201 bp, Homo sapiens
ccttctcacaaatgtaatgtcaagaacatttctattatagtctagttaaaatgttctaaaaatgtgttaattgtaggttttggattctaaaaatgtctccatgaagaatggcacaaagcactttttctgttatttaatacgttgtatgcactcagtaaatattatagcagtccatggtcagggtttattacattagaactg

SEQ ID NO: 175 - DNA, 201 bp, Homo sapiens
tttgggctctttttaatgatcactcacaaatgtatgtttcttttagctggccagtacttttgagtaaagcccctatagtttgacttgcactacaaatgcaatttttttttaataacatttgccctacttgtgctttgtgtttctttcattattatgacataagctacctgggtccacttgtcttttcttttttttgtttca

SEQ ID NO: 176 - DNA, 201 bp, Homo sapiens
tttgggctctttttaatgatcactcacaaatgtatgtttcttttagctggccagtacttttgagtaaagcccctatagtttgacttgcactacaaatgcattttttttttaataacatttgccctacttgtgctttgtgtttctttcattattatgacataagctacctgggtccacttgtcttttcttttttttgtttca

SEQ ID NO: 177 - DNA, 201 bp, Homo sapiens
tcaactatttatgagatgtatcttttgctctctcttgctctcttatttgtaccggtttttgtatataaaattcatgtttccaatctctctctccctgatcagtgacagtcactagcttatcttgaacagatatttaattttgctaacactcagctctgccctccccgatcccctggctccccagcacacattcctttgaaa

SEQ ID NO: 178 - DNA, 201 bp, Homo sapiens
tcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaa

SEQ ID NO: 179 - DNA, 201 bp, Homo sapiens; identical to SEQ ID NO: 71

SEQ ID NO: 180 - DNA, 201 bp, Homo sapiens
gctcgaattatactttgattgagggcgtcattcaggatgaattcgtatttgatgatcctcataaagttgtatttcacattgctcaggaagctaaaaggtgctgacctaggcttgatgatttctaaaaccatgatcacaagtgcagattaatgtctatgtacaaacacagatgatatacacagtctagtacaaacagggaaa

SEQ ID NO: 181 - DNA, 201 bp, Homo sapiens
aaggagagtcaggctgggaaacaaggtaggagaagagggtctgtcaacatgggggcctccggagggtgtcagtgggaggagggggcaacaggacacctgagggatggaagggtcaggaggcagacacctgtaagaattgctctcctttactgagcacctcccatgtggctaagcagcctcctgtcactcaacaccctgcga

SEQ ID NO: 182 - DNA, 201 bp, Homo sapiens
aaggagagtcaggctgggaaacaaggtaggagaagagggtctgtcaacatgggggcctccggagggtgtcagtgggaggagggggcaacaggacacctgaaggatggaagggtcaggaggcagacacctgtaagaattgctctcctttactgagcacctcccatgtggctaagcagcctcctgtcactcaacaccctgcga

SEQ ID NO: 183 - DNA, 201 bp, Homo sapiens
ccacggtacttactccccactgatggaccagtggtttccagtcatgagcgttagactgacttgtttgtcttccattccattgttttgaaactcagtatgccgcccctgtcttgctgtcatgaaatcagcaagagaggatgacacatcaaataataactcggattccagcccacattggattcatcagcatttggaccaata

SEQ ID NO: 184 - DNA, 201 bp, Homo sapiens
ccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaata

SEQ ID NO: 185 - DNA, 201 bp, Homo sapiens
cttttctctaggtctgaagcctcagctgcagtggagattgtcatttgcccacttattgccaggaaccagagtttatgtactgtttgttgctttcatgctggaagttaccctccaaagctgttttcttaacatttctagattctatataaatcaaagaattatgctttgaagcttgtgggctggtatgtgggtggccttgag

SEQ ID NO: 186 - DNA, 201 bp, Homo sapiens
cttttctctaggtctgaagcctcagctgcagtggagattgtcatttgcccacttattgccaggaaccagagtttatgtactgtttgttgctttcatgctgaaagttaccctccaaagctgttttcttaacatttctagattctatataaatcaaagaattatgctttgaagcttgtgggctggtatgtgggtggccttgag

SEQ ID NO: 187 - DNA, 201 bp, Homo sapiens
gaaggaaacttggaaaagtttgacaaatctgctctagagggcctgtgcaatttgaccattgaagaattccgattagcatacttagactactacctcgatggtattattgacttatttaattgtttgacaaatgtttcttcattttccctggtgagtgtgactattgaaagggtaaaagacttttcttataatttcggatgg

SEQ ID NO: 188 - DNA, 201 bp, Homo sapiens
gaaggaaacttggaaaagtttgacaaatctgctctagagggcctgtgcaatttgaccattgaagaattccgattagcatacttagactactacctcgatgatattattgacttatttaattgtttgacaaatgtttcttcattttccctggtgagtgtgactattgaaagggtaaaagacttttcttataatttcggatgg

SEQ ID NO: 189 - DNA, 201 bp, Homo sapiens
gtaagatctccattgaaaattttaaagcagtctttgtttgttaatgagtaatttttctctatttcatatttaaccacagttcttttcccgtaggctgatcgttattcaaaatgggccattcttaatgtaatgggaaaagactttcctgtaaatgaagtatttacacaatttcttgctgacaatcatcaccaagttcgcatg

SEQ ID NO: 190 - DNA, 201 bp, Homo sapiens
gtaagatctccattgaaaattttaaagcagtctttgtttgttaatgagtaatttttctctatttcatatttaaccacagttcttttcccgtaggctgatccttattcaaaatgggccattcttaatgtaatgggaaaagactttcctgtaaatgaagtatttacacaatttcttgctgacaatcatcaccaagttcgcatg

SEQ ID NO: 191 - DNA, 201 bp, Homo sapiens
gcctttcctatgaatttaaatatgcctttaaaataagtcactgttgacagggtcatgagtttccgagtatagttttctttttatcttatttttactcgtctgttgaaaagataatcaaggcctacattttagctgaggataatgaacttttttcctcattcggctgtataatacataaccacagcaagactgacatccact

SEQ ID NO: 192 - DNA, 201 bp, Homo sapiens
gcctttcctatgaatttaaatatgcctttaaaataagtcactgttgacagggtcatgagtttccgagtatagttttctttttatcttatttttactcgtccgttgaaaagataatcaaggcctacattttagctgaggataatgaacttttttcctcattcggctgtataatacataaccacagcaagactgacatccact

SEQ ID NO: 193 - DNA, 201 bp, Homo sapiens
cagcagttttccagcttgcatgtccgagacaccaaaatcgaggtggcccagtttgtaaaggacctgctcttacatttaaagaaactttttcgcgagggacbgttcaactgaaacttcgaaagcatcattatttgcagagacaggacctgactattgaagttgcagattcatttttctttctgatgtcaaaaatgtcttggg

SEQ ID NO: 194 - DNA, 201 bp, Homo sapiens
cagcagttttccagcttgcatgtccgagacaccaaaatcgaggtggcccagtttgtaaaggacctgctcttacatttaaagaaactttttcgcgagggacagttcaactgaaacttcgaaagcatcattatttgcagagacaggacctgactattgaagttgcagattcatttttctttctgatgtcaaaaatgtcttggg

SEQ ID NO: 195 - DNA, 201 bp, Homo sapiens
ggtagagttttaatttggaagacaaaagtgcccataagaaagtcaagaatttcaaatgcaacttttaatactacatatttcaagagctcccatgttcagtgttcctggagaaggaaagctctttttgtttctaagtatgagaaattgttagcgttccttcctttggtcttcccacacccctcaccacaagaggcaaacaaa

SEQ ID NO: 196 - DNA, 201 bp, Homo sapiens
ggtagagttttaatttggaagacaaaagtgcccataagaaagtcaagaatttcaaatgcaacttttaatactacatatttcaagagctcccatgttcagtattcctggagaaggaaagctctttttgtttctaagtatgagaaattgttagcgttccttcctttggtcttcccacacccctcaccacaagaggcaaacaaa

SEQ ID NO: 197 - DNA, 201 bp, Homo sapiens
aagaaaatcatccatgatcttgttctaacacctgccactctagtactatatctgtcacatggtactatgataaagttatctagaaataaaaaagcatacatttgataattcaccaaattgtggagcttcagtattttaaatgtatattaaaattaaattattttaaagatcaaagaaaactttcgtcatactccgtatttg

SEQ ID NO: 198 - DNA, 201 bp, Homo sapiens
aagaaaatcatccatgatcttgttctaacacctgccactctagtactatatctgtcacatggtactatgataaagttatctagaaataaaaaagcatacaattgataattcaccaaattgtggagcttcagtattttaaatgtatattaaaattaaattattttaaagatcaaagaaaactttcgtcatactccgtatttg

(19) United States
(12) Patent Application Publication
(10) Pub. No.: US 2023/0103419 A1
(43) Pub. Date: Apr. 6, 2023

(54) BIOMARKERS FOR PREDICTING TOXICITY OF RADIATION

(71) Applicant: Mira Dx, Inc., Los Angeles, CA (US)

(72) Inventor: Joanne Weidhaas, Los Angeles, CA (US)

(21) Appl. No.: 17/799,507

(22) PCT Filed: Feb. 12, 2021
(86) PCT No.: PCT/US2021/018016

§ 371 Date: Aug. 12, 2022

Related U.S. Application Data
(60) Provisional application No. 62/975,650, filed on Feb. 12, 2020.

(51) Int. Cl. C12Q 1/6886

(52) U.S. Cl. CPC C12Q 1/6886; C12Q 2600/156; A61N 5/10

(57) ABSTRACT

The invention is directed to biomarkers for predicting whether a patient will experience toxicity from radiation treatment or will have an increased risk of mortality, biochemical relapse, or distant failure despite undergoing radiation treatment.

### CROSS REFERENCE TO RELATED APPLICATIONS

This application claims priority to and the benefit of U.S. Provisional Patent Application No. 62/975,650, filed on Feb. 12, 2020, which is incorporated herein by reference in its entirety.

### FIELD OF THE INVENTION

The invention is directed to methods of using biomarkers present in a cancer patient's germline genome to predict the cancer patient's response to radiation.

### BACKGROUND

Many patients experience a toxic response to anti-cancer radiation therapies, resulting in both short and long-term side effects from this therapy; however, it is difficult to predict whether or not a patient will have a toxic response to a therapy before administration.

According to the National Institutes of Health (NIH), the term “biomarker” is defined as “a characteristic that is objectively measured and evaluated as an indicator of normal biologic or pathogenic processes or pharmacological response to a therapeutic intervention” (Biomarkers Definitions Working Group, 2001, Clin. Pharmacol. Ther. 69:89-95)

The development of improved diagnostics based on the discovery of biomarkers has the potential to avoid toxicities resulting from radiation generally or from particular radiation regimens by allowing those likely to develop a toxic response to pursue another therapy or to take steps to prepare for the toxic side effects.

Technologies such as genomics, proteomics, and molecular imaging currently enable rapid, sensitive and reliable detection of specific gene mutations, expression levels of particular genes, and other molecular biomarkers. However, the clinical utilization of cancer biomarkers to predict response or toxicity remains largely unrealized because few cancer biomarkers have been discovered. For example, a recent review article states:
With the emergence of genomic profiling technologies and selective molecular targeted therapies, biomarkers play an increasingly important role in the clinical management of cancer patients. Single gene/protein or multi-gene “signature”-based assays have been introduced to measure specific molecular pathway deregulations that guide therapeutic decision-making as predictive biomarkers. Genome-based prognostic biomarkers are also available for several cancer types for potential incorporation into clinical prognostic staging systems or practice guidelines. However, there is still a large gap between initial biomarker discovery studies and their clinical translation due to the challenges in the process of cancer biomarker development. (Goosens et al., Transl. Cancer Res. 2015 4(3):256-269)
   Comments such as the foregoing illustrate the recognition of a need for the discovery of clinically useful biomarkers that can assist physicians in determining the most optimal course of treatment for cancer patients.

Further, there is a need in the art to identify biomarkers that have the ability to predict whether or not a patient is likely to have a toxic response, and a good or poor systemic response, to a given radiation regimen so that medical professionals can determine the best course of treatment prior to administration and patients can avoid toxic responses considering systemic response to such therapies. For example, there is a need in the art to identify biomarkers that will assist in predicting the toxicity of a given radiation regimen in a patient. Even if a patient would respond to such a therapy, if the therapy would be toxic to that patient, it would be helpful for doctors to know this in advance and take the likely toxicity response into consideration when determining whether a given radiation regimen is appropriate for a patient.

Thus, there is a particular need in the art to identify biomarkers that predict whether a patient is likely to experience toxic side effects, and response, to radiation therapy.

### SUMMARY

The invention is based, in part, on the discovery that cancer patients carrying one or more specified mutations in their genome may have a toxic response, or an altered systemic response (e.g., an increased risk of mortality, distant metastasis or biochemical relapse), to radiation therapy as compared to other cancer patients, for example, patients homozygous for the wild-type allele who do not experience a toxic response or an altered systemic response. The invention is also based, in part, on the discovery that cancer patients carrying one or more specified mutations in their genome may not experience a toxic response or an altered systemic response to radiation therapy as compared to other patients, for example, patients homozygous for the wild-type allele, who do experience a toxic response or an altered systemic response.

In certain aspects, the disclosure relates to a method of treating cancer comprising administering a radiation treatment to a patient identified as carrying or not carrying one or more mutations selected from:
a) a C nucleotide at a position corresponding to position 101 of SEQ ID NO: 1 (miR.99a.promoter);
b) an A nucleotide at a position corresponding to position 101 of SEQ ID NO: 3 (IL1.B_rs4848306);
c) a G nucleotide at a position corresponding to position 101 of SEQ ID NO: 5 (SHC4_rs1062124);
d) a T nucleotide at a position corresponding to position 101 of SEQ ID NO: 7 (rs17599026);
e) an A nucleotide at a position corresponding to position 101 of SEQ ID NO: 9 (RAC1_rs9374);
f) a C nucleotide at a position corresponding to position 101 of SEQ ID NO: 11 (IL24_rs1150258);
g) an A or C nucleotide at a position corresponding to position 101 of SEQ ID NO: 13 (IL1A_rs1800587);
h) a C nucleotide at a position corresponding to position 101 of SEQ ID NO: 15 (ERCC1_rs3212948);
i) a G nucleotide at a position corresponding to position 101 of SEQ ID NO: 17 (IL.6_rs12700386);
j) a G nucleotide at a position corresponding to position 101 of SEQ ID NO: 19 (IL.6_rs2069840);
k) a T nucleotide at a position corresponding to position 101 of SEQ ID NO: 21 (CAMK2G_rs2306327);
l) a T nucleotide at a position corresponding to position 101 of SEQ ID NO: 23 (BMP2_rs1980499);
m) a C nucleotide at a position corresponding to position 101 of SEQ ID NO: 25 (EREG_rs1460008);
n) a C nucleotide at a position corresponding to position 101 of SEQ ID NO: 27 (CD274_rs4143815);
o) an A nucleotide at a position corresponding to position 101 of SEQ ID NO: 29 (IL17D_rs7787);
p) a C nucleotide at a position corresponding to position 101 of SEQ ID NO: 31 (STAT3/rs3744483);
q) a C nucleotide at a position corresponding to position 101 of SEQ ID NO: 33 (BMP4_rs17563)
r) a C nucleotide at a position corresponding to position 101 of SEQ ID NO: 35 (IRF5_rs10488631);
s) a T nucleotide at a position corresponding to position 101 of SEQ ID NO: 37 (UNGC.41.IL1RAP);
t) a G nucleotide at a position corresponding to position 101 of SEQ ID NO: 39 (IL19_rs1798);
u) an A nucleotide at a position corresponding to position 101 of SEQ ID NO: 41 (RAD23A_rs8240);
v) a C nucleotide at a position corresponding to position 101 of SEQ ID NO: 43 (BRCA2_rs15869);
w) a G nucleotide at a position corresponding to position 101 of SEQ ID NO: 45 (CD274_rs2282055);
x) a C nucleotide at a position corresponding to position 101 of SEQ ID NO: 47 (BMP2_rs3178250);
y) a T nucleotide at a position corresponding to position 101 of SEQ ID NO: 49 (CD274_rs1411262);
z) an A nucleotide at a position corresponding to position 101 of SEQ ID NO: 51 (CD274_rs2297136);
aa) a G nucleotide at a position corresponding to position 101 of SEQ ID NO: 53 (BMP2_rs1979855);
bb) a G nucleotide at a position corresponding to position 101 of SEQ ID NO: 55 (PTPN2_rs1893217);
cc) a G nucleotide at a position corresponding to position 101 of SEQ ID NO: 57 (CD274_rs822339);
dd) a G nucleotide at a position corresponding to position 101 of SEQ ID NO: 59 (BATF3_rs6695772);
ee) an A nucleotide at a position corresponding to position 101 of SEQ ID NO: 61 (IL18R1_rs11465660);
ff) a G nucleotide at a position corresponding to position 101 of SEQ ID NO: 63
gg) a C nucleotide at a position corresponding to position 101 of SEQ ID NO: 65 (ABL1_rs11991);
hh) an A nucleotide at a position corresponding to position 101 of SEQ ID NO: 67 (ATM_rs189037);
ii) an A nucleotide at a position corresponding to position 101 of SEQ ID NO: 69 (CD6_rs76677607);
jj) an A nucleotide at a position corresponding to position 101 of SEQ ID NO: 71 (IL1A_rs17561);
kk) a G nucleotide at a position corresponding to position 101 of SEQ ID NO: 73 (BRCA2_rs7334543);
ll) a T nucleotide at a position corresponding to position 101 of SEQ ID NO: 75 (ILF3.58_rs118142475);
mm) a C nucleotide at a position corresponding to position 101 of SEQ ID NO: 77 (BIRC5_rs2239680);
nn) a G nucleotide at a position corresponding to position 101 of SEQ ID NO: 79 (NBN_rs1805794);
oo) a T nucleotide at a position corresponding to position 101 of SEQ ID NO: 81 (TREX_rs11797);
pp) a G nucleotide at a position corresponding to position 101 of SEQ ID NO: 83 (CD274_rs4742098);
qq) a T nucleotide at a position corresponding to position 101 of SEQ ID NO: 85 (ERCC4_rs4781562);
rr) an A, C or G nucleotide at a position corresponding to position 101 of SEQ ID NO: 87 (MSH2_rs2303428);
ss) a C nucleotide at a position corresponding to position 101 of SEQ ID NO: 89 (XRCC4_rs1040363);
tt) a G nucleotide at a position corresponding to position 101 of SEQ ID NO: 91 (IL10RB_rs2834167);
uu) an A nucleotide at a position corresponding to position 101 of SEQ ID NO: 93 (SMAD1_rs11724777);
vv) a G nucleotide at a position corresponding to position 101 of SEQ ID NO: 95 (HAMP_rs10421768);
ww) a C nucleotide at a position corresponding to position 101 of SEQ ID NO: 97 (XRCC1_rs25487);
xx) a G nucleotide at a position corresponding to position 101 of SEQ ID NO: 99 (IL19_rs2243158);
yy) a C nucleotide at a position corresponding to position 101 of SEQ ID NO: 101 (FOXP3_rs2280883);
zz) a G or C nucleotide at a position corresponding to position 101 of SEQ ID NO: 103 (IL10_rs3024496_P1.P2);
aaa) a C nucleotide at a position corresponding to position 101 of SEQ ID NO: 105 (LIG4_rs2232643);
bbb) a T nucleotide at a position corresponding to position 101 of SEQ ID NO: 107 (IL2RA_rs2476491);
ccc) an A nucleotide at a position corresponding to position 101 of SEQ ID NO: 109 (IL12A_rs568408);
ddd) a C nucleotide at a position corresponding to position 101 of SEQ ID NO: 111 (GSK3B_rs2037547);
eee) an A nucleotide at a position corresponding to position 101 of SEQ ID NO: 113 (ATM_rs189037);
fff) a G nucleotide at a position corresponding to position 101 of SEQ ID NO: 115 (IL6_rs1800797);
ggg) an A nucleotide at a position corresponding to position 101 of SEQ ID NO: 117 (RAC1_rs9374);
hhh) a C or G nucleotide at a position corresponding to position 101 of SEQ ID NO: 119 (FCGR2A_rs1801274);
iii) a T nucleotide at a position corresponding to position 101 of SEQ ID NO: 121 (ATM_rs373759);
jjj) an A nucleotide at a position corresponding to position 101 of SEQ ID NO: 123 (CETN2_rs8230);
kkk) an A nucleotide at a position corresponding to position 101 of SEQ ID NO: 125 (XRCC3_rs861539);
lll) a C nucleotide at a position corresponding to position 101 of SEQ ID NO: 127 (miR.34b.c.promoter_rs4938723);
mmm) a G nucleotide at a position corresponding to position 101 of SEQ ID NO: 129 (LIN28A_rs9438623);
nnn) a G or C nucleotide at a position corresponding to position 101 of SEQ ID NO: 131 (FANCC_rs9673);
ooo) an A nucleotide at a position corresponding to position 101 of SEQ ID NO: 133 (REV3L_rs465646);
ppp) a G nucleotide at a position corresponding to position 101 of SEQ ID NO: 135 (IL6_rs1800795);
qqq) a C nucleotide at a position corresponding to position 101 of SEQ ID NO: 137 (SPI1_rs2071304);
rrr) a G or C nucleotide at a position corresponding to position 101 of SEQ ID NO: 139 (IL10_rs3024496);
sss) a deletion of a T nucleotide in the sequence ATTTTG of SEQ ID NO: 142 (wild-type sequence) to give a corresponding sequence of ATTTG as in SEQ ID NO: 141 (mutant) (EXO1_rs4150021);
ttt) an A nucleotide at a position corresponding to position 101 of SEQ ID NO: 143 (FOXP3_rs2232365);
uuu) a C nucleotide at a position corresponding to position 101 of SEQ ID NO: 145 (HAMP_rs1882694);
vvv) a G nucleotide at a position corresponding to position 101 of SEQ ID NO: 147 (IL10_rs1800872);
www) a G nucleotide at a position corresponding to position 101 of SEQ ID NO: 149 (IL16_rs11556218);
xxx) an A nucleotide at a position corresponding to position 101 of SEQ ID NO: 151 (IL2RA_rs11256497);
yyy) a T nucleotide at a position corresponding to position 101 of SEQ ID NO: 153 (IL2RB_rs228942);
zzz) an A nucleotide at a position corresponding to position 101 of SEQ ID NO: 155 (KIT_rs17084733);
aaaa) a C nucleotide at a position corresponding to position 101 of SEQ ID NO: 157 (KRAS_rs61764370);
bbbb) a C nucleotide at a position corresponding to position 101 of SEQ ID NO: 159 (LIG4_rs3093772_P1.P2);
cccc) a G nucleotide at a position corresponding to position 101 of SEQ ID NO: 161 (MDM2_rs769412);
dddd) a C or G nucleotide at a position corresponding to position 101 of SEQ ID NO: 163 (P2RX7_rs3751143);
eeee) a T nucleotide at a position corresponding to position 101 of SEQ ID NO: 165 (rs2187668);
ffff) an A nucleotide at a position corresponding to position 101 of SEQ ID NO: 167 (IL10_rs3024505);
gggg) a G nucleotide at a position corresponding to position 101 of SEQ ID NO: 169 (rs922075);
hhhh) a T nucleotide at a position corresponding to position 101 of SEQ ID NO: 171 (SOS1_rs1059313);
iiii) a C nucleotide at a position corresponding to position 101 of SEQ ID NO: 173 (STAT4_rs7574070);
jjjj) an A nucleotide at a position corresponding to position 101 of SEQ ID NO: 175 (UNGC.96.TGFB2_NA);
kkkk) an A nucleotide at a position corresponding to position 101 of SEQ ID NO: 177 (VEGFA_rs41282644);
llll) an A nucleotide at a position corresponding to position 101 of SEQ ID NO: 179 (IL1A_rs17561);
mmmm) a G nucleotide at a position corresponding to position 101 of SEQ ID NO: 181 (TGFB1_rs1800469);
nnnn) a C nucleotide at a position corresponding to position 101 of SEQ ID NO: 183 (EGFR_rs884225);
oooo) a G nucleotide at a position corresponding to position 101 of SEQ ID NO: 185 (rs17388569);
pppp) a G nucleotide at a position corresponding to position 101 of SEQ ID NO: 187 (TRL4_rs4986790);
qqqq) a G nucleotide at a position corresponding to position 101 of SEQ ID NO: 189 (ATM_rs1800057); and
rrrr) a T nucleotide at a position corresponding to position 101 of SEQ ID NO: 191 (ILRAP_rs79383051).

In certain embodiments, the radiation treatment comprises hypofractionated or traditional radiation, and the patient is identified as carrying or not carrying one or more mutations selected from:
a) a C nucleotide at a position corresponding to position 101 of SEQ ID NO: 1 (miR.99a.promoter);
b) an A nucleotide at a position corresponding to position 101 of SEQ ID NO: 3 (IL1.B_rs4848306);
c) a G nucleotide at a position corresponding to position 101 of SEQ ID NO: 5 (SHC4_rs1062124);
d) a T nucleotide at a position corresponding to position 101 of SEQ ID NO: 7 (rs17599026);
e) an A nucleotide at a position corresponding to position 101 of SEQ ID NO: 9 (RAC1_rs9374);
f) a C nucleotide at a position corresponding to position 101 of SEQ ID NO: 11 (IL24_rs1150258);
g) an A nucleotide at a position corresponding to position 101 of SEQ ID NO: 13 (IL1A_rs1800587);
h) a C nucleotide at a position corresponding to position 101 of SEQ ID NO: 15 (ERCC1_rs3212948);
i) a G nucleotide at a position corresponding to position 101 of SEQ ID NO: 17 (IL.6_rs12700386);
j) a G nucleotide at a position corresponding to position 101 of SEQ ID NO: 19 (IL.6_rs2069840);
k) a T nucleotide at a position corresponding to position 101 of SEQ ID NO: 21 (CAMK2G_rs2306327);
l) a T nucleotide at a position corresponding to position 101 of SEQ ID NO: 23 (BMP2_rs1980499);
m) a C nucleotide at a position corresponding to position 101 of SEQ ID NO: 25 (EREG_rs1460008);
n) a C nucleotide at a position corresponding to position 101 of SEQ ID NO: 27 (CD274_rs4143815);
o) an A nucleotide at a position corresponding to position 101 of SEQ ID NO: 29 (IL17D_rs7787);
p) a C nucleotide at a position corresponding to position 101 of SEQ ID NO: 31 (STAT3/rs3744483);
q) a C nucleotide at a position corresponding to position 101 of SEQ ID NO: 33 (BMP4_rs17563)
r) a C nucleotide at a position corresponding to position 101 of SEQ ID NO: 35 (IRF5_rs10488631); or
s) a T nucleotide at a position corresponding to position 101 of SEQ ID NO: 37 (UNGC.41.IL1RAP).

In certain embodiments, the radiation treatment comprises hypofractionated radiation, and the patient is identified as carrying or not carrying one or more mutations selected from:
a) not carrying or carrying only one copy of a C nucleotide at a position corresponding to position 101 of SEQ ID NO: 1 (miR.99a.promoter);
b) not carrying or carrying only one copy of an A nucleotide at a position corresponding to position 101 of SEQ ID NO: 3 (IL1.B_rs4848306);
c) not carrying or carrying only one copy of a G nucleotide at a position corresponding to position 101 of SEQ ID NO: 5 (SHC4_rs1062124);
d) carrying a T nucleotide at a position corresponding to position 101 of SEQ ID NO: 7 (rs17599026);
e) not carrying or carrying only one copy of an A nucleotide at a position corresponding to position 101 of SEQ ID NO: 9 (RAC1_rs9374);
f) not carrying a C nucleotide at a position corresponding to position 101 of SEQ ID NO: 11 (IL24_rs1150258);
g) not carrying or carrying only one copy of an A nucleotide at a position corresponding to position 101 of SEQ ID NO: 13 (IL1A_rs1800587);
h) not carrying a C nucleotide at a position corresponding to position 101 of SEQ ID NO: 15 (ERCC1_rs3212948);
i) not carrying or carrying only one copy of a G nucleotide at a position corresponding to position 101 of SEQ ID NO: 17 (IL.6_rs12700386);
j) not carrying or carrying only one copy of a G nucleotide at a position corresponding to position 101 of SEQ ID NO: 19 (IL.6_rs2069840);
k) not carrying or carrying only one copy of a T nucleotide at a position corresponding to position 101 of SEQ ID NO: 21 (CAMK2G_rs2306327);
l) not carrying or carrying only one copy of a T nucleotide at a position corresponding to position 101 of SEQ ID NO: 23 (BMP2_rs1980499);
m) not carrying or carrying only one copy of a C nucleotide at a position corresponding to position 101 of SEQ ID NO: 25 (EREG_rs1460008);
n) not carrying or carrying only one copy of a C nucleotide at a position corresponding to position 101 of SEQ ID NO: 27 (CD274_rs4143815);
o) not carrying or carrying only one copy of an A nucleotide at a position corresponding to position 101 of SEQ ID NO: 29 (IL17D_rs7787);
p) not carrying or carrying only one copy of a C nucleotide at a position corresponding to position 101 of SEQ ID NO: 31 (STAT3/rs3744483);
q) not carrying or carrying only one copy of a C nucleotide at a position corresponding to position 101 of SEQ ID NO: 33 (BMP4_rs17563);
r) not carrying a C nucleotide at a position corresponding to position 101 of SEQ ID NO: 35 (IRF5_rs10488631); or
s) not carrying or carrying only one copy of a T nucleotide at a position corresponding to position 101 of SEQ ID NO: 37 (UNGC.41.IL1RAP).

In certain embodiments, the radiation treatment comprises conventionally fractionated (traditional) radiation, and the patient is identified as carrying or not carrying one or more mutations selected from:
a) carrying a C nucleotide at a position corresponding to position 101 of SEQ ID NO: 1 (miR.99a.promoter);
b) carrying an A nucleotide at a position corresponding to position 101 of SEQ ID NO: 3 (IL1.B_rs4848306);
c) carrying a G nucleotide at a position corresponding to position 101 of SEQ ID NO: 5 (SHC4_rs1062124);
d) not carrying a T nucleotide at a position corresponding to position 101 of SEQ ID NO: 7 (rs17599026);
e) carrying an A nucleotide at a position corresponding to position 101 of SEQ ID NO: 9 (RAC1_rs9374);
f) carrying a C nucleotide at a position corresponding to position 101 of SEQ ID NO: 11 (IL24_rs1150258);
g) carrying an A nucleotide at a position corresponding to position 101 of SEQ ID NO: 13 (IL1A_rs1800587);
h) carrying a C nucleotide at a position corresponding to position 101 of SEQ ID NO: 15 (ERCC1_rs3212948);
i) carrying a G nucleotide at a position corresponding to position 101 of SEQ ID NO: 17 (IL.6_rs12700386);
j) carrying a G nucleotide at a position corresponding to position 101 of SEQ ID NO: 19 (IL.6_rs2069840);
k) carrying a T nucleotide at a position corresponding to position 101 of SEQ ID NO: 21 (CAMK2G_rs2306327);
l) carrying a T nucleotide at a position corresponding to position 101 of SEQ ID NO: 23 (BMP2_rs1980499);
m) carrying a C nucleotide at a position corresponding to position 101 of SEQ ID NO: 25 (EREG_rs1460008);
n) carrying a C nucleotide at a position corresponding to position 101 of SEQ ID NO: 27 (CD274_rs4143815);
o) carrying an A nucleotide at a position corresponding to position 101 of SEQ ID NO: 29 (IL17D_rs7787);
p) carrying a C nucleotide at a position corresponding to position 101 of SEQ ID NO: 31 (STAT3/rs3744483);
q) carrying a C nucleotide at a position corresponding to position 101 of SEQ ID NO: 33 (BMP4_rs17563)
r) carrying a C nucleotide at a position corresponding to position 101 of SEQ ID NO: 35 (IRF5_rs10488631); or
s) carrying a T nucleotide at a position corresponding to position 101 of SEQ ID NO: 37 (UNGC.41.IL1RAP).

In certain embodiments, the cancer is selected from adrenal cancer, anal cancer, bile duct cancer, bladder cancer, bone cancer, brain/CNS, basal cell skin cancer, breast cancer, cervical cancer, colorectal cancer, endometrial cancer, esophageal cancer, eye cancer, gallbladder cancer, gastrointestinal carcinoid tumors, gastrointestinal stromal tumor (GIST), gastric cancer, glioma, glioblastoma, head and neck cancer, Hodgkin disease, Kaposi sarcoma, kidney cancer, laryngeal and hypopharyngeal cancer, leukemia, liver cancer, lymphoma, malignant mesothelioma, merkel cell carcinoma, metastatic urothelial carcinoma, multiple myeloma, myeloma, myelodysplastic syndrome, nasal cavity and paranasal sinus cancer, nasopharyngeal cancer, neuroendocrine cancer, neuroblastoma, non-Hodgkin lymphoma, oral cavity and oropharyngeal cancer, osteosarcoma, ovarian cancer, pancreatic cancer, penile cancer, pituitary tumors, prostate cancer, renal cancer, retinoblastoma, rhabdomyosarcoma, salivary gland cancer, sarcoma, squamous cell skin cancer, small intestine cancer, stomach cancer, testicular cancer, thymus cancer, thyroid cancer, uterine cancer, or vaginal cancer.

In certain embodiments, the cancer is sarcoma. In certain embodiments, the toxicity is wound toxicity. In certain embodiments, the patient has a tumor on a lower extremity.

In certain embodiments, radiation treatment comprises hypofractionated radiation or conventionally fractionated (traditional) radiation, and the patient is identified as carrying or not carrying one or more mutations selected from:
a) a G nucleotide at position 101 of SEQ ID NO: 53 (BMP2_rs1979855);
b) a G nucleotide at position 101 of SEQ ID NO: 55 (PTPN2_rs1893217);
c) a C nucleotide at position 101 of SEQ ID NO: 43 (BRCA2_rs15869);
d) a C nucleotide at position 101 of SEQ ID NO: 65 (ABL1_rs11991);
e) a G nucleotide at position 101 of SEQ ID NO: 63 (ERCC1_rs11615);
f) an A nucleotide at position 101 of SEQ ID NO: 41 (RAD23A_rs8240);
g) a G nucleotide at position 101 of SEQ ID NO: 59 (BATF3_rs6695772);
h) a C nucleotide at position 101 of SEQ ID NO: 143 (FOXP3_rs2232365);
i) a G nucleotide at position 101 of SEQ ID NO: 39 (IL19_rs1798);
j) a G or T nucleotide at position 101 of SEQ ID NO: 129 (LIN28A_rs9438623);
k) an A nucleotide at position 101 of SEQ ID NO: 61 (IL18R1_rs11465660);
l) a T nucleotide at position 101 of SEQ ID NO: 153 (IL2RB_rs228942);
m) a T nucleotide at position 101 of SEQ ID NO: 49 (CD274_rs1411262);
n) a C nucleotide at position 101 of SEQ ID NO: 47 (BMP2_rs3178250);
o) a C nucleotide at position 101 of SEQ ID NO: 137 (SPI1_rs2071304);
p) a G nucleotide at position 101 of SEQ ID NO: 99 (IL19_rs2243158);
q) an A nucleotide at position 101 of SEQ ID NO: 167 (IL10_rs3024505);
r) a G nucleotide at position 101 of SEQ ID NO: 187 (TRL4_rs4986790);
s) an A nucleotide at position 101 of SEQ ID NO: 177 (VEGFA_rs41282644);
t) a G or C nucleotide at position 101 of SEQ ID NO: 163 (P2RX7_rs3751143);
u) a C nucleotide at position 101 of SEQ ID NO: 15 (ERCC1_rs3212948);
v) a G or C nucleotide at position 101 of SEQ ID NO: 131 (FANCC_rs9673);
w) a G nucleotide at position 101 of SEQ ID NO: 51 (CD274_rs2297136);
x) a G nucleotide at position 101 of SEQ ID NO: 19 (IL6_rs2069840);
y) a G nucleotide at position 101 of SEQ ID NO: 45 (CD274_rs2282055);
z) a G nucleotide at position 101 of SEQ ID NO: 57 (CD274_rs822339);
aa) a G nucleotide at position 101 of SEQ ID NO: 95 (HAMP_rs10421768);
bb) a C nucleotide at position 101 of SEQ ID NO: 145 (HAMP_rs1882694);
cc) a G nucleotide at position 101 of SEQ ID NO: 189 (ATM_rs1800057);
dd) a C nucleotide at position 101 of SEQ ID NO: 191 (ILRAP_rs79383051);
ee) an A nucleotide at position 101 of SEQ ID NO: 155 (KIT_rs17084733);
ff) a T nucleotide at position 101 of SEQ ID NO: 7 (rs17599026);
gg) an A nucleotide at position 101 of SEQ ID NO: 67 (ATM_rs189037);
hh) a G nucleotide at position 101 of SEQ ID NO: 69 (CD6_rs76677607);
ii) an A nucleotide at position 101 of SEQ ID NO: 13 (IL1A_rs1800587);
jj) an A nucleotide at position 101 of SEQ ID NO: 179 (IL1A_rs17561);
kk) a G nucleotide at position 101 of SEQ ID NO: 73 (BRCA2_rs7334543);
ll) a T nucleotide at position 101 of SEQ ID NO: 75 (ILF3.58_rs118142475);
mm) a C nucleotide at position 101 of SEQ ID NO: 77 (BIRC5_rs2239680);
nn) a T nucleotide at position 101 of SEQ ID NO: 107 (IL2RA_rs2476491);
oo) an A nucleotide at position 101 of SEQ ID NO: 109 (IL12A_rs568408);
pp) a C nucleotide at position 101 of SEQ ID NO: 111 (GSK3B_rs2037547);
qq) a G nucleotide at position 101 of SEQ ID NO: 135 (IL6_rs1800795);
rr) a T nucleotide at position 101 of SEQ ID NO: 105 (LIG4_rs2232643);
ss) a C nucleotide at position 101 of SEQ ID NO: 31 (STAT3_rs3744483);
tt) a G nucleotide at position 101 of SEQ ID NO: 115 (IL6_rs1800797);
uu) a G nucleotide at position 101 of SEQ ID NO: 17 (IL.6_rs12700386);
vv) an A nucleotide at position 101 of SEQ ID NO: 9 (RAC1_rs9374);
ww) a T nucleotide at position 101 of SEQ ID NO: 121 (ATM_rs373759);
xx) an A nucleotide at position 101 of SEQ ID NO: 123 (CETN2_rs8230);
yy) an A nucleotide at position 101 of SEQ ID NO: 126 (XRCC3_rs861539);
zz) a C nucleotide at position 101 of SEQ ID NO: 127 (miR.34b.c.promoter_rs4938723);
aaa) an A nucleotide at position 101 of SEQ ID NO: 3 (IL1.B_rs4848306);
bbb) an A nucleotide at position 101 of SEQ ID NO: 133 (REV3L_rs465646);
ccc) a C nucleotide at position 101 of SEQ ID NO: 183 (EGFR_rs884225);
ddd) a G nucleotide at position 101 of SEQ ID NO: 181 (TGFB1_rs1800469);
eee) a T nucleotide at position 101 of SEQ ID NO: 165 (rs2187668);
fff) a G nucleotide at position 101 of SEQ ID NO: 185 (rs17388569);
ggg) a G nucleotide at position 101 of SEQ ID NO: 79 (NBN_rs1805794);
hhh) a G nucleotide at position 101 of SEQ ID NO: 83 (CD274_rs4742098);
iii) a G nucleotide at position 101 of SEQ ID NO: 85 (ERCC4_rs4781562);
jjj) an A, C, or G nucleotide at position 101 of SEQ ID N: 87 (MSH2_rs2303428);
kkk) an A nucleotide at position 101 of SEQ ID NO: 151 (IL2RA_rs11256497);
lll) a C nucleotide at position 101 of SEQ ID NO: 159 (LIG4_rs3093772_P1.P2);
mmm) an A nucleotide at position 101 of SEQ ID NO: 29 (IL17D_rs7787);
nnn) a C nucleotide at position 101 of SEQ ID NO: (CD274_rs4143815);
ooo) an A nucleotide at position 101 of SEQ ID NO: 175 (UNGC.96.TGFB2_NA);
ppp) a G nucleotide at position 101 of SEQ ID NO: 161 (MDM2_rs769412); and
qqq) a G nucleotide at position 101 of SEQ ID NO: 147 (IL10_rs1800872).

In certain embodiments, the radiation treatment comprises hypofractionated radiation, and the patient is identified as carrying or not carrying one or more mutations selected from:
a) not carrying or carrying only one copy of a G nucleotide at position 101 of SEQ ID NO: 53 (BMP2_rs1979855);
b) not carrying or carrying only one copy of a G nucleotide at position 101 of SEQ ID NO: 55 (PTPN2_rs1893217);
c) not carrying or carrying only one copy of a C nucleotide at position 101 of SEQ ID NO: 43 (BRCA2_rs15869);
d) carrying a C nucleotide at position 101 of SEQ ID NO: 65 (ABL1_rs11991);
e) not carrying or carrying only one copy of a G nucleotide at position 101 of SEQ ID NO: 63 (ERCC1_rs11615);
f) not carrying or carrying only one copy of an A nucleotide at position 101 of SEQ ID NO: 41 (RAD23A_rs8240);
g) carrying a G nucleotide at position 101 of SEQ ID NO: 59 (BATF3_rs6695772);
h) carrying a C nucleotide at position 101 of SEQ ID NO: 143 (FOXP3_rs2232365);
i) not carrying or carrying only one copy of a G nucleotide at position 101 of SEQ ID NO: 39 (IL19_rs1798);
j) not carrying a G or T nucleotide at position 101 of SEQ ID NO: 129 (LIN28A_rs9438623);
k) not carrying or carrying only one copy of an A nucleotide at position 101 of SEQ ID NO: 61 (IL18R1_rs11465660);
l) not carrying a T nucleotide at position 101 of SEQ ID NO: 153 (IL2RB_rs228942);
m) not carrying or carrying only one copy of a T nucleotide at position 101 of SEQ ID NO: 49 (CD274_rs1411262);
n) not carrying or carrying only one copy of a C nucleotide at position 101 of SEQ ID NO: 47 (BMP2_rs3178250);
o) not carrying a C nucleotide at position 101 of SEQ ID NO: 137 (SPIT_rs2071304);
p) not carrying a G nucleotide at position 101 of SEQ ID NO: 99 (IL19_rs2243158);
q) not carrying an A nucleotide at position 101 of SEQ ID NO: 167 (IL10_rs3024505);
r) carrying a G nucleotide at position 101 of SEQ ID NO: 187 (TRL4_rs4986790);
s) carrying an A nucleotide at position 101 of SEQ ID NO: 177 (VEGFA_rs41282644);
t) carrying a G or C nucleotide at position 101 of SEQ ID NO: 163 (P2RX7_rs3751143);
u) not carrying a C nucleotide at position 101 of SEQ ID NO: 15 (ERCC1_rs3212948);
v) not carrying a G or C nucleotide at position 101 of SEQ ID NO: 131 (FANCC_rs9673);
w) not carrying a G nucleotide at position 101 of SEQ ID NO: 51 (CD274_rs2297136);
x) not carrying or carrying only one copy of a G nucleotide at position 101 of SEQ ID NO: 19 (IL6_rs2069840);
y) not carrying or carrying only one copy of a G nucleotide at position 101 of SEQ ID NO: 45 (CD274_rs2282055);
z) carrying a G nucleotide at position 101 of SEQ ID NO: 57 (CD274_rs822339);
aa) carrying a G nucleotide at position 101 of SEQ ID NO: 95 (HAMP_rs10421768);
bb) carrying a C nucleotide at position 101 of SEQ ID NO: 145 (HAMP_rs1882694);
cc) not carrying a G nucleotide at position 101 of SEQ ID NO: 189 (ATM_rs1800057);
dd) not carrying a C nucleotide at position 101 of SEQ ID NO: 191 (ILRAP_rs79383051);
ee) carrying an A nucleotide at position 101 of SEQ ID NO: 155 (KIT_rs17084733); and
ff) carrying a T nucleotide at position 101 of SEQ ID NO: 7 (rs17599026).

In certain embodiments, the cancer is selected from adrenal cancer, anal cancer, bile duct cancer, bladder cancer, bone cancer, brain/CNS, basal cell skin cancer, breast cancer, cervical cancer, colorectal cancer, endometrial cancer, esophageal cancer, eye cancer, gallbladder cancer, gastrointestinal carcinoid tumors, gastrointestinal stromal tumor (GIST), gastric cancer, glioma, glioblastoma, head and neck cancer, Hodgkin disease, Kaposi sarcoma, kidney cancer, laryngeal and hypopharyngeal cancer, leukemia, liver cancer, lymphoma, malignant mesothelioma, merkel cell carcinoma, metastatic urothelial carcinoma, multiple myeloma, myeloma, myelodysplastic syndrome, nasal cavity and paranasal sinus cancer, nasopharyngeal cancer, neuroendocrine cancer, neuroblastoma, non-Hodgkin lymphoma, oral cavity and oropharyngeal cancer, osteosarcoma, ovarian cancer, pancreatic cancer, penile cancer, pituitary tumors, prostate cancer, renal cancer, retinoblastoma, rhabdomyosarcoma, salivary gland cancer, sarcoma, squamous cell skin cancer, small intestine cancer, stomach cancer, testicular cancer, thymus cancer, thyroid cancer, uterine cancer, or vaginal cancer.

In certain embodiments, the cancer is prostate cancer.

In certain embodiments, the hypofractionated radiation regimen comprises administering from about 20 Gy to about 35 Gy radiation over about 5 to about 8 fractions. In certain embodiments, the hypofractionated radiation regimen comprises administering stereotactic body radiation therapy.

In certain embodiments, the radiation treatment comprises conventionally fractionated (traditional) radiation, and the patient is identified as carrying or not carrying one or more mutations selected from:
a) carrying two copies of a G nucleotide at position 101 of SEQ ID NO: 53 (BMP2_rs1979855);
b) carrying two copies of a G nucleotide at position 101 of SEQ ID NO: 55 (PTPN2_rs1893217);
c) carrying two copies of a C nucleotide at position 101 of SEQ ID NO: 43 (BRCA2_rs15869);
d) not carrying a C nucleotide at position 101 of SEQ ID NO: 65 (ABL1_rs11991);
e) carrying two copies of a G nucleotide at position 101 of SEQ ID NO: 63 (ERCC1_rs11615);
f) carrying two copies of an A nucleotide at position 101 of SEQ ID NO: 41 (RAD23A_rs8240);
g) not carrying a G nucleotide at position 101 of SEQ ID NO: 59 (BATF3_rs6695772);
h) not carrying a C nucleotide at position 101 of SEQ ID NO: 143 (FOXP3_rs2232365);
i) carrying two copies of a G nucleotide at position 101 of SEQ ID NO: 39 (IL19_rs1798);
j) carrying a G or T nucleotide at position 101 of SEQ ID NO: 129 (LIN28A_rs9438623);
k) carrying two copies of an A nucleotide at position 101 of SEQ ID NO: 61 (IL18R1_rs11465660);
l) carrying a T nucleotide at position 101 of SEQ ID NO: 153 (IL2RB_rs228942);
m) carrying two copies of a T nucleotide at position 101 of SEQ ID NO: 49 (CD274_rs1411262);
n) carrying two copies of a C nucleotide at position 101 of SEQ ID NO: 47 (BMP2_rs3178250);
o) carrying a C nucleotide at position 101 of SEQ ID NO: 137 (SPI1_rs2071304);
p) carrying a G nucleotide at position 101 of SEQ ID NO: 99 (IL19_rs2243158);
q) carrying an A nucleotide at position 101 of SEQ ID NO: 167 (IL10_rs3024505);
r) not carrying a G nucleotide at position 101 of SEQ ID NO: 187 (TRL4_rs4986790);
s) not carrying an A nucleotide at position 101 of SEQ ID NO: 177 (VEGFA_rs41282644);
t) not carrying a G or C nucleotide at position 101 of SEQ ID NO: 163 (P2RX7_rs3751143);
u) carrying a C nucleotide at position 101 of SEQ ID NO: 15 (ERCC1_rs3212948);
v) carrying a G or C nucleotide at position 101 of SEQ ID NO: 131 (FANCC_rs9673);
w) carrying a G nucleotide at position 101 of SEQ ID NO: 51 (CD274_rs2297136);
x) carrying two copies of a G nucleotide at position 101 of SEQ ID NO: 19 (IL6_rs2069840);
y) carrying two copies of a G nucleotide at position 101 of SEQ ID NO: 45 (CD274_rs2282055);
z) not carrying a G nucleotide at position 101 of SEQ ID NO: 57 (CD274_rs822339);
aa) not carrying a G nucleotide at position 101 of SEQ ID NO: 95 (HAMP_rs10421768);
bb) not carrying a C nucleotide at position 101 of SEQ ID NO: 145 (HAMP_rs1882694);
cc) carrying a G nucleotide at position 101 of SEQ ID NO: 189 (ATM_rs1800057);
dd) carrying a C nucleotide at position 101 of SEQ ID NO: 191 (ILRAP_rs79383051);
ee) not carrying an A nucleotide at position 101 of SEQ ID NO: 155 (KIT_rs17084733); and
ff) not carrying a T nucleotide at position 101 of SEQ ID NO: 7 (rs17599026).

In certain embodiments, the patient is treated with conventionally fractionated (traditional) radiation, and the patient is identified as carrying or not carrying one or more mutations selected from:
a) carrying an A nucleotide at position 101 of SEQ ID NO: 67 (ATM_rs189037);
b) not carrying a G nucleotide at position 101 of SEQ ID NO: 69 (CD6_rs76677607);
c) not carrying or carrying only one copy of an A nucleotide at position 101 of SEQ ID NO: 13 (IL1A_rs1800587);
d) carrying an A nucleotide at position 101 of SEQ ID NO: 179 (IL1A_rs17561);
e) carrying a G nucleotide at position 101 of SEQ ID NO: 73 (BRCA2_rs7334543);
f) not carrying a T nucleotide at position 101 of SEQ ID NO: 75 (ILF3.58_rs118142475);
g) carrying a C nucleotide at position 101 of SEQ ID NO: 77 (BIRC5_rs2239680);
h) carrying a T nucleotide at position 101 of SEQ ID NO: 107 (IL2RA_rs2476491);
i) carrying an A nucleotide at position 101 of SEQ ID NO: 109 (IL12A_rs568408);
j) not carrying a C nucleotide at position 101 of SEQ ID NO: 111 (GSK3B_rs2037547);
k) carrying a G nucleotide at position 101 of SEQ ID NO: 135 (IL6_rs1800795);
l) not carrying a T nucleotide at position 101 of SEQ ID NO: 105 (LIG4_rs2232643);
m) not carrying or carrying only one copy of a C nucleotide at position 101 of SEQ ID NO: 31 (STAT3_rs3744483);
n) carrying a G nucleotide at position 101 of SEQ ID NO: 115 (IL6_rs1800797);
o) not carrying or carrying only one copy of a G nucleotide at position 101 of SEQ ID NO: 17 (IL.6_rs12700386);
p) not carrying or carrying only one copy of an A nucleotide at position 101 of SEQ ID NO: 9 (RAC1_rs9374);
q) not carrying a T nucleotide at position 101 of SEQ ID NO: 121 (ATM_rs373759);
r) not carrying an A nucleotide at position 101 of SEQ ID NO: 123 (CETN2_rs8230);
s) not carrying an A nucleotide at position 101 of SEQ ID NO: 126 (XRCC3_rs861539);
t) carrying a C nucleotide at position 101 of SEQ ID NO: 127 (miR.34b.c.promoter_rs4938723);
u) not carrying or carrying only one copy of an A nucleotide at position 101 of SEQ ID NO: 61 (IL18R1_rs11465660);
v) not carrying or carrying only one copy of an A nucleotide at position 101 of SEQ ID NO: 3 (IL1.B_rs4848306);
w) not carrying a G or C nucleotide at position 101 of SEQ ID NO: 131 (FANCC_rs9673);
x) carrying an A nucleotide at position 101 of SEQ ID NO: 133 (REV3L_rs465646);
y) not carrying a G nucleotide at position 101 of SEQ ID NO: 51 (CD274_rs2297136);
z) carrying a C nucleotide at position 101 of SEQ ID NO: 183 (EGFR_rs884225);
aa) not carrying a G nucleotide at position 101 of SEQ ID NO: 181 (TGFB1_rs1800469);
bb) carrying a T nucleotide at position 101 of SEQ ID NO: 165 (rs2187668);
cc) carrying a G nucleotide at position 101 of SEQ ID NO: 185 (rs17388569);
dd) not carrying a G nucleotide at position 101 of SEQ ID NO: 79 (NBN_rs1805794);
ee) carrying a G nucleotide at position 101 of SEQ ID NO: 83 (CD274_rs4742098);
ff) carrying a G nucleotide at position 101 of SEQ ID NO: 85 (ERCC4_rs4781562);
gg) not carrying an A, C, or G nucleotide at position 101 of SEQ ID N: 87 (MSH2_rs2303428);
hh) carrying a T nucleotide at position 101 of SEQ ID NO: 7 (rs17599026);
ii) not carrying an A nucleotide at position 101 of SEQ ID NO: 151 (IL2RA_rs11256497);
jj) carrying a C nucleotide at position 101 of SEQ ID NO: 159 (LIG4_rs3093772_P1.P2);
kk) not carrying or carrying only one copy of an A nucleotide at position 101 of SEQ ID NO: 29 (IL17D_rs7787);
l) not carrying or carrying only one copy of a C nucleotide at position 101 of SEQ ID NO: (CD274_rs4143815);
mm) not carrying an A nucleotide at position 101 of SEQ ID NO: 175 (UNGC.96.TGFB2_NA);
nn) not carrying a G nucleotide at position 101 of SEQ ID NO: 161 (MDM2_rs769412);
oo) not carrying an A nucleotide at position 101 of SEQ ID NO: 167 (IL10_rs3024505);
pp) not carrying or carrying only one copy of a C nucleotide at position 101 of SEQ ID NO: 43 (BRCA2_rs15869);
qq) not carrying a G nucleotide at position 101 of SEQ ID NO: 147 (IL10_rs1800872);
rr) not carrying or carrying only one copy of a G nucleotide at position 101 of SEQ ID NO: 53 (BMP2_rs1979855);
ss) not carrying or carrying only one copy of a G nucleotide at position 101 of SEQ ID NO: 55 (PTPN2_rs1893217);
tt) carrying a C nucleotide at position 101 of SEQ ID NO: 65 (ABL1_rs11991); and
uu) not carrying or carrying only one copy of a G nucleotide at position 101 of SEQ ID NO: 63 (ERCC1_rs11615).

In certain embodiments, the cancer is selected from adrenal cancer, anal cancer, bile duct cancer, bladder cancer, bone cancer, brain/CNS, basal cell skin cancer, breast cancer, cervical cancer, colorectal cancer, endometrial cancer, esophageal cancer, eye cancer, gallbladder cancer, gastrointestinal carcinoid tumors, gastrointestinal stromal tumor (GIST), gastric cancer, glioma, glioblastoma, head and neck cancer, Hodgkin disease, Kaposi sarcoma, kidney cancer, laryngeal and hypopharyngeal cancer, leukemia, liver cancer, lymphoma, malignant mesothelioma, merkel cell carcinoma, metastatic urothelial carcinoma, multiple myeloma, myeloma, myelodysplastic syndrome, nasal cavity and paranasal sinus cancer, nasopharyngeal cancer, neuroendocrine cancer, neuroblastoma, non-Hodgkin lymphoma, oral cavity and oropharyngeal cancer, osteosarcoma, ovarian cancer, pancreatic cancer, penile cancer, pituitary tumors, prostate cancer, renal cancer, retinoblastoma, rhabdomyosarcoma, salivary gland cancer, sarcoma, squamous cell skin cancer, small intestine cancer, stomach cancer, testicular cancer, thymus cancer, thyroid cancer, uterine cancer, or vaginal cancer.

In certain embodiments, the cancer is prostate cancer.

In certain embodiments, the method is a reduced toxicity method and the toxicity is acute or long-term toxicity. In certain embodiments, the toxicity is GI and/or GU toxicity. In certain embodiments, the method results in less toxicity than a hypofractionated radiation regimen. In certain embodiments, the traditional radiation regimen comprises administering from about 40 Gy to about 60 Gy radiation over a period of about 5 to about 6 weeks. In certain embodiments, the traditional radiation regimen comprises administering from about 40 Gy to about 60 Gy radiation over about 15 to about 30 fractions. In certain embodiments, the radiation therapy is external beam radiation therapy.

In another aspect, the disclosure relates to a method of treating cancer comprising administering a radiation treatment to a cancer patient identified as carrying or not carrying one or more mutations in a SNP selected from: TREX1_rs11797, LIN28A_rs9438623, miR.34b.c.promoter_rs4938723, FCGR2A_rs1801274, CD274_rs4742098, IL.6_rs12700386, SPI1_rs2071304, ERCC4_rs4781562, IL10_rs3024496, IL.6_rs2069840, rs2187668, CD274_rs4143815, KRAS_rs61764370, SHC4_rs1062124, HAMP_rs1882694, rs922075, ERCC1_rs11615, EXO1_rs4150021, STAT4_rs7574070, and SOS1_rs1059313; wherein the presence or the absence of the SNP is associated with a reduced risk of distant failure in response to radiation therapy.

In certain embodiments, the patient is identified as carrying or not carrying one or more mutations in a SNP selected from:
a) carrying a T nucleotide at position 101 of SEQ ID NO: 81 (TREX1_rs11797);
b) carrying a G or T nucleotide at position 101 of SEQ ID NO: 129 (LIN28A_rs9438623);
c) carrying or not carrying a C nucleotide at position 101 of SEQ ID NO: 127 (miR.34b.c.promoter_rs4938723);
d) carrying a C or G nucleotide at position 101 of SEQ ID NO: 119 (FCGR2A_rs1801274);
e) carrying a G nucleotide at position 101 of SEQ ID NO: 83 (CD274_rs4742098);
f) not carrying a G nucleotide at position 101 of SEQ ID NO: 17 (IL.6_rs12700386);
g) not carrying a C nucleotide at position 101 of SEQ ID NO: 137 (SPI1_rs2071304);
h) carrying a G nucleotide at position 101 of SEQ ID NO: 85 (ERCC4_rs4781562);
i) carrying a G nucleotide at position 101 of SEQ ID NO: 103 (IL10_rs3024496_P1.P2);
j) carrying a G nucleotide at position 101 of SEQ ID NO: 139 (IL10_rs3024496);
k) not carrying or carrying only one copy of a G nucleotide at position 101 of SEQ ID NO: 19 (IL.6_rs2069840);
l) not carrying a T nucleotide at position 101 of SEQ ID NO: 165 (rs2187668);
m) not carrying a C nucleotide at position 101 of SEQ ID NO: 27 (CD274_rs4143815);
n) carrying a C nucleotide at position 101 of SEQ ID NO: 157 (KRAS_rs61764370);
o) carrying a G nucleotide at position 101 of SEQ ID NO: 5 (SHC4_rs1062124);
p) carrying a G nucleotide at position 101 of SEQ ID NO: 169 (rs922075);
q) carrying a G nucleotide at position 101 of SEQ ID NO: 63 (ERCC1_rs11615);
r) not carrying the deletion of a T nucleotide at [ATTTTG] of SEQ ID NO: 142 (EXO1_rs4150021);
s) carrying a C nucleotide at position 101 of SEQ ID NO: 173 (STAT4_rs7574070); and
t) carrying a T nucleotide at position 101 of SEQ ID NO: 171 (SOS1_rs1059313).

In certain embodiments, the radiation therapy is hypofractionated radiation. In certain embodiments, the cancer is sarcoma.

In another aspect, the disclosure relates to a method of treating cancer in a cancer patient comprising administering a radiation treatment to a patient identified as carrying or not carrying one or more mutations in a SNP selected from: BIRC5_rs2239680, SMAD1_rs11724777, IL12A_rs568408, and IL13_rs20541, wherein the presence or the absence of the SNP is not associated with an increased risk of mortality.

In certain embodiments, the patient is identified as carrying or not carrying one or more mutations in a SNP selected from:
a) carrying a C nucleotide at position 101 of SEQ ID NO: 77 (BIRC5_rs2239680);
b) not carrying an A nucleotide at position 101 of SEQ ID NO: 93 (SMAD1_rs11724777);
c) not carrying an A nucleotide at position 101 of SEQ ID NO: 109 (IL12A_rs568408); and
d) carrying a T nucleotide at position 101 of SEQ ID NO: 193 (IL13_rs20541).

In certain embodiments, the radiation therapy is conventionally fractionated (traditional) radiation.

In another aspect, the disclosure relates to a method of treating cancer in a cancer patient comprising (i) administering a radiation treatment and a different cancer treatment or (ii) administering a different cancer treatment instead of radiation to a patient identified as carrying or not carrying one or more mutations in a SNP selected from: BIRC5_rs2239680, SMAD1_rs11724777, IL12A_rs568408, and IL13_rs20541, wherein the presence or the absence of the SNP is associated with an increased risk of mortality.

In certain embodiments, the patient is identified as carrying or not carrying one or more mutations in a SNP selected from:
a) not carrying a C nucleotide at position 101 of SEQ ID NO: 77 (BIRC5_rs2239680);
b) carrying an A nucleotide at position 101 of SEQ ID NO: 93 (SMAD1_rs11724777);
c) carrying an A nucleotide at position 101 of SEQ ID NO: 109 (IL12A_rs568408); and
d) not carrying a T nucleotide at position 101 of SEQ ID NO: 193 (IL13_rs20541).

In certain embodiments, the radiation therapy is conventionally fractionated (traditional) radiation. In certain embodiments, the different cancer treatment comprises brachytherapy, chemotherapy, androgen deprivation therapy, immunotherapy, high intensity focused ultrasound, cryotherapy, laser ablation, photodynamic therapy, or surgery.

In certain embodiments, the cancer is prostate cancer.

In another aspect, the disclosure relates to a method of treating cancer in a cancer patient comprising administering a radiation treatment and/or a different cancer treatment to a patient identified as carrying or not carrying one or more mutations in a SNP selected from: BMP2_rs3178250, SMAD1_rs11724777, IL10RB_rs2834167, CD274_rs822339, CD274_rs2282055, IL12A_rs568408, PARP1_rs8679, CD274_rs1411262, BMP2_rs1979855, and P2RX7_rs3751143, wherein the presence or the absence of the SNP is associated with an increased risk of (decreased time to) biochemical relapse or is associated with a reduced risk of biochemical relapse following radiation therapy.

In certain embodiments, if the patient is identified as carrying or not carrying one or more mutations in a SNP associated with increased risk of biochemical relapse and selected from:
a) carrying a C nucleotide at position 101 of SEQ ID NO: 47 (BMP2_rs3178250);
b) not carrying a G nucleotide at position 101 of SEQ ID NO: 91 (IL10RB_rs2834167);
c) not carrying a G nucleotide at position 101 of SEQ ID NO: 57 (CD274_rs822339);
d) carrying a G nucleotide at position 101 of SEQ ID NO: 45 (CD274_rs2282055);
e) carrying an A nucleotide at position 101 of SEQ ID NO: 109 (IL12A_rs568408);
f) carrying a G nucleotide at position 101 of SEQ ID NO: 195 (PARP1_rs8679);
g) carrying a T nucleotide at position 101 of SEQ ID NO: 49 (CD274_rs1411262);
h) carrying a G nucleotide at position 101 of SEQ ID NO: 53 (BMP2_rs1979855); and
i) carrying a C nucleotide at position 101 of SEQ ID NO: 163 (P2RX7_rs3751143);
the patient is treated with a treatment in addition to radiation therapy, or with a different treatment.

In certain embodiments, if the patient is identified as carrying or not carrying one or more mutations in a SNP associated with decreased risk of biochemical relapse and selected from:
a) not carrying a C nucleotide at position 101 of SEQ ID NO: 47 (BMP2_rs3178250);
b) carrying a G nucleotide at position 101 of SEQ ID NO: 91 (IL10RB_rs2834167);
c) carrying a G nucleotide at position 101 of SEQ ID NO: 57 (CD274_rs822339);
d) not carrying a G nucleotide at position 101 of SEQ ID NO: 45 (CD274_rs2282055);
e) not carrying an A nucleotide at position 101 of SEQ ID NO: 109 (IL12A_rs568408);
f) not carrying a G nucleotide at position 101 of SEQ ID NO: 195 (PARP1_rs8679);
g) not carrying a T nucleotide at position 101 of SEQ ID NO: 49 (CD274_rs1411262);
h) not carrying a G nucleotide at position 101 of SEQ ID NO: 53 (BMP2_rs1979855); and
i) carrying a C nucleotide at position 101 of SEQ ID NO: 163 (P2RX7_rs3751143);
the patient is treated with radiation therapy.

In certain embodiments, the radiation therapy is conventionally fractionated (traditional) radiation. In certain embodiments, the additional treatment or the different treatment is hypofractionated radiation, brachytherapy, chemotherapy, androgen deprivation therapy, immunotherapy, high intensity focused ultrasound, cryotherapy, laser ablation, photodynamic therapy, or surgery.

In certain embodiments, the cancer is prostate cancer.

In another aspect, the disclosure relates to a method of treating cancer in a cancer patient comprising administering a radiation treatment and/or a different cancer treatment to a patient identified as carrying or not carrying one or more mutations in a SNP selected from: IL8_rs4073, IL10_rs3024496_P1, BIRC5_rs2239680, RAC1_rs9374, IL10_rs3024496_P1.P2, BMP2_rs3178250, and NBN_rs1805794, wherein the presence or the absence of the SNP is associated with an increased risk of distant metastasis or is associated with a reduced risk of distant metastasis.

In certain embodiments, if the patient is identified as carrying or not carrying one or more of the following mutations in a SNP associated with increased risk of distant metastasis:
a) carrying a T nucleotide at position 101 of SEQ ID NO: 197 (IL8_rs4073);
b) not carrying a G or C nucleotide at position 101 of SEQ ID NO: 103 (IL10_rs3024496_P1);
c) not carrying a C nucleotide occurs at position 101 of SEQ ID NO: 77 (BIRC5_rs2239680);
d) carrying an A nucleotide at position 101 of SEQ ID NO: 9 (RAC1_rs9374);
e) not carrying a G nucleotide occurs at position 101 of SEQ ID NO: 103 (IL10_rs3024496_P1.P2);
f) carrying a G nucleotide at position 101 of SEQ ID NO: 53 (BMP2_rs1979855); and
g) carrying a G nucleotide at position 101 of SEQ ID NO: 79 (NBN_rs1805794);
the patient is treated with a treatment in addition to radiation therapy, or with a different treatment.

In certain embodiments, if the patient is identified as carrying or not carrying one or more of the following mutations in a SNP associated with decreased risk of distant metastasis:
a) not carrying a T nucleotide at position 101 of SEQ ID NO: 197 (IL8_rs4073);
b) carrying a G or C nucleotide at position 101 of SEQ ID NO: 103 (IL10_rs3024496_P1);
c) carrying a C nucleotide occurs at position 101 of SEQ ID NO: 77 (BIRC5_rs2239680);
d) not carrying an A nucleotide at position 101 of SEQ ID NO: 9 (RAC1_rs9374);
e) carrying a G nucleotide occurs at position 101 of SEQ ID NO: 103 (IL10_rs3024496_P1.P2);
f) not carrying a G nucleotide at position 101 of SEQ ID NO: 53 (BMP2_rs1979855); and
g) not carrying a G nucleotide at position 101 of SEQ ID NO: 79 (NBN_rs1805794);
the patient is treated with a radiation treatment.

In certain embodiments, the radiation therapy is conventionally fractionated (traditional) radiation. In certain embodiments, the additional treatment or the different treatment is hypofractionated radiation, brachytherapy, chemotherapy, androgen deprivation therapy, immunotherapy, high intensity focused ultrasound, cryotherapy, laser ablation, photodynamic therapy, or surgery.

In certain embodiments, the cancer is prostate cancer.

In another aspect, the disclosure relates to a method of treating prostate cancer in a patient comprising administering a radiation treatment to a patient identified as carrying or not carrying one or more mutations in a SNP selected from: HAMP_rs10421768, XRCC1_rs25487, IL.6_rs2069840, XRCC4_rs1040363, and IL19_rs2243158, wherein the presence or the absence of the SNP is not associated with a reduced risk of impotency.

In certain embodiments, the patient is identified as carrying or not carrying one or more mutations in a SNP selected from:
a) carrying a G nucleotide at position 101 of SEQ ID NO: 95 (HAMP_rs10421768);
b) not carrying a C nucleotide at position 101 of SEQ ID NO: 97 (XRCC1_rs25487);
c) not carrying a G nucleotide at position 101 of SEQ ID NO: 19 (IL.6_rs2069840);
d) carrying a C nucleotide at position 101 of SEQ ID NO: 89 (XRCC4_rs1040363); and
e) not carrying a G nucleotide at position 101 of SEQ ID NO: 99 (IL19_rs2243158).

In certain embodiments, the radiation therapy is conventionally fractionated (traditional) radiation.

In another aspect, the disclosure relates to a method for determining the toxicity of a radiation treatment in a cancer patient comprising determining whether the patient carries one or more of the following mutations:
a) a C nucleotide at a position corresponding to position 101 of SEQ ID NO: 1 (miR.99a.promoter);
b) an A nucleotide at a position corresponding to position 101 of SEQ ID NO: 3 (IL1.B_rs4848306);
c) a G nucleotide at a position corresponding to position 101 of SEQ ID NO: 5 (SHC4_rs1062124);
d) a T nucleotide at a position corresponding to position 101 of SEQ ID NO: 7 (rs17599026);
e) an A nucleotide at a position corresponding to position 101 of SEQ ID NO: 9 (RAC1_rs9374);
f) an A, C, or G nucleotide at a position corresponding to position 101 of SEQ ID NO: 11 (IL24_rs1150258);
g) an A nucleotide at a position corresponding to position 101 of SEQ ID NO: 13 (IL1A_rs1800587);
h) a C nucleotide at a position corresponding to position 101 of SEQ ID NO: 15 (ERCC1_rs3212948);
i) a G nucleotide at a position corresponding to position 101 of SEQ ID NO: 17 (IL.6_rs12700386);
j) a G nucleotide at a position corresponding to position 101 of SEQ ID NO: 19 (IL.6_rs2069840);
k) a T nucleotide at a position corresponding to position 101 of SEQ ID NO: 21 (CAMK2G_rs2306327);
l) a T nucleotide at a position corresponding to position 101 of SEQ ID NO: 23 (BMP2_rs1980499);
m) a C nucleotide at a position corresponding to position 101 of SEQ ID NO: 25 (EREG_rs1460008);
n) a C nucleotide at a position corresponding to position 101 of SEQ ID NO: 27 (CD274_rs4143815);
o) an A nucleotide at a position corresponding to position 101 of SEQ ID NO: 29 (IL17D_rs7787);
p) a C nucleotide at a position corresponding to position 101 of SEQ ID NO: 31 (STAT3/rs3744483);
q) a C nucleotide at a position corresponding to position 101 of SEQ ID NO: 33 (BMP4_rs17563)
r) a C nucleotide at a position corresponding to position 101 of SEQ ID NO: 35 (IRF5_rs10488631);
s) a T nucleotide at a position corresponding to position 101 of SEQ ID NO: 37 (UNGC.41.IL1RAP);
t) a G nucleotide at a position corresponding to position 101 of SEQ ID NO: 39 (IL19_rs1798);
u) an A nucleotide at a position corresponding to position 101 of SEQ ID NO: 41 (RAD23A_rs8240);
v) a C nucleotide at a position corresponding to position 101 of SEQ ID NO: 43 (BRCA2_rs15869);
w) a G nucleotide at a position corresponding to position 101 of SEQ ID NO: 45 (CD274_rs2282055);
x) a C nucleotide at a position corresponding to position 101 of SEQ ID NO: 47 (BMP2_rs3178250);
y) a T nucleotide at a position corresponding to position 101 of SEQ ID NO: 49 (CD274_rs1411262);
z) an A nucleotide at a position corresponding to position 101 of SEQ ID NO: 51 (CD274_rs2297136);
aa) a G nucleotide at a position corresponding to position 101 of SEQ ID NO: 53 (BMP2_rs1979855);
bb) a G nucleotide at a position corresponding to position 101 of SEQ ID NO: 55 (PTPN2_rs1893217);
cc) a G nucleotide at a position corresponding to position 101 of SEQ ID NO: 57 (CD274_rs822339);
dd) a G nucleotide at a position corresponding to position 101 of SEQ ID NO: 59 (BATF3_rs6695772);
ee) an A nucleotide at a position corresponding to position 101 of SEQ ID NO: 61 (IL18R1_rs11465660);
ff) a G nucleotide at a position corresponding to position 101 of SEQ ID NO: 63 (ERCC1_rs11615);
gg) a C nucleotide at a position corresponding to position 101 of SEQ ID NO: 65 (ABL1_rs11991);
hh) an A nucleotide at a position corresponding to position 101 of SEQ ID NO: 67 (ATM_rs189037);
ii) an A nucleotide at a position corresponding to position 101 of SEQ ID NO: 69 (CD6_rs76677607);
jj) an A nucleotide at a position corresponding to position 101 of SEQ ID NO: 71 (IL1A_rs17561);
kk) a G nucleotide at a position corresponding to position 101 of SEQ ID NO: 73 (BRCA2_rs7334543);
ll) a T nucleotide at a position corresponding to position 101 of SEQ ID NO: 75 (ILF3.58_rs118142475);
mm) a C nucleotide at a position corresponding to position 101 of SEQ ID NO: 77 (BIRC5_rs2239680);
nn) a G nucleotide at a position corresponding to position 101 of SEQ ID NO: 79 (NBN_rs1805794);
oo) a T nucleotide at a position corresponding to position 101 of SEQ ID NO: 81 (TREX_rs11797);
pp) a G nucleotide at a position corresponding to position 101 of SEQ ID NO: 83 (CD274_rs4742098);
qq) a T nucleotide at a position corresponding to position 101 of SEQ ID NO: 85 (ERCC4_rs4781562);
rr) an A, C or G nucleotide at a position corresponding to position 101 of SEQ ID NO: 87 (MSH2_rs2303428);
ss) a C nucleotide at a position corresponding to position 101 of SEQ ID NO: 89 (XRCC4_rs1040363);
tt) a G nucleotide at a position corresponding to position 101 of SEQ ID NO: 91 (IL10RB_rs2834167);
uu) an A nucleotide at a position corresponding to position 101 of SEQ ID NO: 93 (SMAD1_rs11724777);
vv) a G nucleotide at a position corresponding to position 101 of SEQ ID NO: 95 (HAMP_rs10421768);
ww) a C nucleotide at a position corresponding to position 101 of SEQ ID NO: 97 (XRCC1_rs25487);
xx) a G nucleotide at a position corresponding to position 101 of SEQ ID NO: 99 (IL19_rs2243158);
yy) a C nucleotide at a position corresponding to position 101 of SEQ ID NO: 101 (FOXP3_rs2280883);
zz) a G nucleotide at a position corresponding to position 101 of SEQ ID NO: 103 (IL10_rs3024496_P1.P2);
aaa) a C nucleotide at a position corresponding to position 101 of SEQ ID NO: 105 (LIG4_rs2232643);
bbb) a T nucleotide at a position corresponding to position 101 of SEQ ID NO: 107 (IL2RA_rs2476491);
ccc) an A nucleotide at a position corresponding to position 101 of SEQ ID NO: 109 (IL12A_rs568408);
ddd) a C nucleotide at a position corresponding to position 101 of SEQ ID NO: 111 (GSK3B_rs2037547);
eee) an A nucleotide at a position corresponding to position 101 of SEQ ID NO: 113 (ATM_rs189037);
fff) a G nucleotide at a position corresponding to position 101 of SEQ ID NO: 115 (IL6_rs1800797);
ggg) an A nucleotide at a position corresponding to position 101 of SEQ ID NO: 117 (RAC1_rs9374);
hhh) a C or G nucleotide at a position corresponding to position 101 of SEQ ID NO: 119 (FCGR2A_rs1801274);
iii) a T nucleotide at a position corresponding to position 101 of SEQ ID NO: 121 (ATM_rs373759);
jjj) an A nucleotide at a position corresponding to position 101 of SEQ ID NO: 123 (CETN2_rs8230);
kkk) an A nucleotide at a position corresponding to position 101 of SEQ ID NO: 125 (XRCC3_rs861539);
lll) a C nucleotide at a position corresponding to position 101 of SEQ ID NO: 127 (miR.34b.c.promoter_rs4938723);
mmm) a G nucleotide at a position corresponding to position 101 of SEQ ID NO: 129 (LIN28A_rs9438623);
nnn) a G or C nucleotide at a position corresponding to position 101 of SEQ ID NO: 131 (FANCC_rs9673);
ooo) an A nucleotide at a position corresponding to position 101 of SEQ ID NO: 133 (REV3L_rs465646);
ppp) a G nucleotide at a position corresponding to position 101 of SEQ ID NO: 135 (IL6_rs1800795);
qqq) a C nucleotide at a position corresponding to position 101 of SEQ ID NO: 137 (SPI1_rs2071304);
rrr) a G nucleotide at a position corresponding to position 101 of SEQ ID NO: 139 (IL10_rs3024496);
sss) a deletion of a T nucleotide in the sequence ATTTTG of SEQ ID NO: 142 (wild-type sequence) to give a corresponding sequence of ATTTG as in SEQ ID NO: 141 (mutant) (EXO1_rs4150021);
ttt) an A nucleotide at a position corresponding to position 101 of SEQ ID NO: 143 (FOXP3_rs2232365);
uuu) a C nucleotide at a position corresponding to position 101 of SEQ ID NO: 145 (HAMP_rs1882694);
vvv) a G nucleotide at a position corresponding to position 101 of SEQ ID NO: 147 (IL10_rs1800872);
www) a G nucleotide at a position corresponding to position 101 of SEQ ID NO: 149 (IL16_rs11556218);
xxx) an A nucleotide at a position corresponding to position 101 of SEQ ID NO: 151 (IL2RA_rs11256497);
yyy) a T nucleotide at a position corresponding to position 101 of SEQ ID NO: 153 (IL2RB_rs228942);
zzz) an A nucleotide at a position corresponding to position 101 of SEQ ID NO: 155 (KIT_rs17084733);
aaaa) a C nucleotide at a position corresponding to position 101 of SEQ ID NO: 157 (KRAS_rs61764370);
bbbb) a C nucleotide at a position corresponding to position 101 of SEQ ID NO: 159 (LIG4_rs3093772_P1.P2);
cccc) a G nucleotide at a position corresponding to position 101 of SEQ ID NO: 161 (MDM2_rs769412);
dddd) a C or G nucleotide at a position corresponding to position 101 of SEQ ID NO: 163 (P2RX7_rs3751143);
eeee) a T nucleotide at a position corresponding to position 101 of SEQ ID NO: 165 (rs2187668);
ffff) an A nucleotide at a position corresponding to position 101 of SEQ ID NO: 167 (IL10_rs3024505);
gggg) a G nucleotide at a position corresponding to position 101 of SEQ ID NO: 169 (rs922075);
hhhh) a T nucleotide at a position corresponding to position 101 of SEQ ID NO: 171 (SOS1_rs1059313);
iiii) a C nucleotide at a position corresponding to position 101 of SEQ ID NO: 173 (STAT4_rs7574070);
jjjj) an A nucleotide at a position corresponding to position 101 of SEQ ID NO: 175 (UNGC.96.TGFB2_NA);
kkkk) an A nucleotide at a position corresponding to position 101 of SEQ ID NO: 177 (VEGFA_rs41282644);
llll) an A nucleotide at a position corresponding to position 101 of SEQ ID NO: 179 (IL1A_rs17561);
mmmm) a G nucleotide at a position corresponding to position 101 of SEQ ID NO: 181 (TGFB1_rs1800469);
nnnn) a C nucleotide at a position corresponding to position 101 of SEQ ID NO: 183 (EGFR_rs884225); and
oooo) a G nucleotide at a position corresponding to position 101 of SEQ ID NO: 185 (rs17388569).

In certain embodiments, the method comprises determining whether the patient carries one or more of the following mutations:
a) a C nucleotide at a position corresponding to position 101 of SEQ ID NO: 1 (miR.99a.promoter);
b) an A nucleotide at a position corresponding to position 101 of SEQ ID NO: 3 (IL1.B_rs4848306);
c) a G nucleotide at a position corresponding to position 101 of SEQ ID NO: 5 (SHC4_rs1062124);
d) a T nucleotide at a position corresponding to position 101 of SEQ ID NO: 7 (rs17599026);
e) an A nucleotide at a position corresponding to position 101 of SEQ ID NO: 9 (RAC1_rs9374);
f) a C nucleotide at a position corresponding to position 101 of SEQ ID NO: 11 (IL24_rs1150258);
g) an A nucleotide at a position corresponding to position 101 of SEQ ID NO: 13 (IL1A_rs1800587);
h) a C nucleotide at a position corresponding to position 101 of SEQ ID NO: 15 (ERCC1_rs3212948);
i) a G nucleotide at a position corresponding to position 101 of SEQ ID NO: 17 (IL.6_rs12700386);
j) a G nucleotide at a position corresponding to position 101 of SEQ ID NO: 19 (IL.6_rs2069840);
k) a T nucleotide at a position corresponding to position 101 of SEQ ID NO: 21 (CAMK2G_rs2306327);
l) a T nucleotide at a position corresponding to position 101 of SEQ ID NO: 23 (BMP2_rs1980499);
m) a C nucleotide at a position corresponding to position 101 of SEQ ID NO: 25 (EREG_rs1460008);
n) a C nucleotide at a position corresponding to position 101 of SEQ ID NO: 27 (CD274_rs4143815);
o) an A nucleotide at a position corresponding to position 101 of SEQ ID NO: 29 (IL17D_rs7787);
p) a C nucleotide at a position corresponding to position 101 of SEQ ID NO: 31 (STAT3/rs3744483);
q) a C nucleotide at a position corresponding to position 101 of SEQ ID NO: 33 (BMP4_rs17563)
r) a C nucleotide at a position corresponding to position 101 of SEQ ID NO: 35
s) a T nucleotide at a position corresponding to position 101 of SEQ ID NO: 37 (UNGC.41.IL1RAP).

In certain embodiments, the patient has a decreased likelihood of having a toxic response to the radiation treatment if the patient is carrying or not carrying one or more mutations selected from:
a) not carrying or carrying only one copy of a C nucleotide at a position corresponding to position 101 of SEQ ID NO: 1 (miR.99a.promoter);
b) not carrying or carrying only one copy of an A nucleotide at a position corresponding to position 101 of SEQ ID NO: 3 (IL1.B_rs4848306);
c) not carrying or carrying only one copy of a G nucleotide at a position corresponding to position 101 of SEQ ID NO: 5 (SHC4_rs1062124);
d) carrying a T nucleotide at a position corresponding to position 101 of SEQ ID NO: 7 (rs17599026);
e) not carrying or carrying only one copy of an A nucleotide at a position corresponding to position 101 of SEQ ID NO: 9 (RAC1_rs9374);
f) not carrying a C nucleotide at a position corresponding to position 101 of SEQ ID NO: 11 (IL24_rs1150258);
g) not carrying or carrying only one copy of an A nucleotide at a position corresponding to position 101 of SEQ ID NO: 13 (IL1A_rs1800587);
h) not carrying a C nucleotide at a position corresponding to position 101 of SEQ ID NO: 15 (ERCC1_rs3212948);
i) not carrying or carrying only one copy of a G nucleotide at a position corresponding to position 101 of SEQ ID NO: 17 (IL.6_rs12700386);
j) not carrying or carrying only one copy of a G nucleotide at a position corresponding to position 101 of SEQ ID NO: 19 (IL.6_rs2069840);
k) not carrying or carrying only one copy of a T nucleotide at a position corresponding to position 101 of SEQ ID NO: 21 (CAMK2G_rs2306327);
l) not carrying or carrying only one copy of a T nucleotide at a position corresponding to position 101 of SEQ ID NO: 23 (BMP2_rs1980499);
m) not carrying or carrying only one copy of a C nucleotide at a position corresponding to position 101 of SEQ ID NO: 25 (EREG_rs1460008);
n) not carrying or carrying only one copy of a C nucleotide at a position corresponding to position 101 of SEQ ID NO: 27 (CD274_rs4143815);
o) not carrying or carrying only one copy of an A nucleotide at a position corresponding to position 101 of SEQ ID NO: 29 (IL17D_rs7787);
p) not carrying or carrying only one copy of a C nucleotide at a position corresponding to position 101 of SEQ ID NO: 31 (STAT3/rs3744483);
q) not carrying or carrying only one copy of a C nucleotide at a position corresponding to position 101 of SEQ ID NO: 33 (BMP4_rs17563);
r) not carrying a C nucleotide at a position corresponding to position 101 of SEQ ID NO: 35 (IRF5_rs10488631); or
s) not carrying or carrying only one copy of a T nucleotide at a position corresponding to position 101 of SEQ ID NO: 37 (UNGC.41.IL1RAP).

In certain embodiments, the patient has an increased likelihood of having a toxic response to the radiation treatment if the patient carries or does not carry one or more mutations selected from:
a) carrying a C nucleotide at a position corresponding to position 101 of SEQ ID NO: 1 (miR.99a.promoter);
b) carrying an A nucleotide at a position corresponding to position 101 of SEQ ID NO: 3 (IL1.B_rs4848306);
c) carrying a G nucleotide at a position corresponding to position 101 of SEQ ID NO: 5 (SHC4_rs1062124);
d) not carrying a T nucleotide at a position corresponding to position 101 of SEQ ID NO: 7 (rs17599026);
e) carrying an A nucleotide at a position corresponding to position 101 of SEQ ID NO: 9 (RAC1_rs9374);
f) carrying a C nucleotide at a position corresponding to position 101 of SEQ ID NO: 11 (IL24_rs1150258);
g) carrying an A nucleotide at a position corresponding to position 101 of SEQ ID NO: 13 (IL1A_rs1800587);
h) carrying a C nucleotide at a position corresponding to position 101 of SEQ ID NO: 15 (ERCC1_rs3212948);
i) carrying a G nucleotide at a position corresponding to position 101 of SEQ ID NO: 17 (IL.6_rs12700386);
j) carrying a G nucleotide at a position corresponding to position 101 of SEQ ID NO: 19 (IL.6_rs2069840);
k) carrying a T nucleotide at a position corresponding to position 101 of SEQ ID NO: 21 (CAMK2G_rs2306327);
l) carrying a T nucleotide at a position corresponding to position 101 of SEQ ID NO: 23 (BMP2_rs1980499);
m) carrying a C nucleotide at a position corresponding to position 101 of SEQ ID NO: 25 (EREG_rs1460008);
n) carrying a C nucleotide at a position corresponding to position 101 of SEQ ID NO: 27 (CD274_rs4143815);
o) carrying an A nucleotide at a position corresponding to position 101 of SEQ ID NO: 29 (IL17D_rs7787);
p) carrying two copies of a C nucleotide at a position corresponding to position 101 of SEQ ID NO: 31 (STAT3/rs3744483);
q) carrying a C nucleotide at a position corresponding to position 101 of SEQ ID NO: 33 (BMP4_rs17563)
r) carrying a C nucleotide at a position corresponding to position 101 of SEQ ID NO: 35 (IRF5_rs10488631); or
s) carrying a T nucleotide at a position corresponding to position 101 of SEQ ID NO: 37 (UNGC.41.IL1RAP).

In certain embodiments, the cancer is selected from adrenal cancer, anal cancer, bile duct cancer, bladder cancer, bone cancer, brain/CNS, basal cell skin cancer, breast cancer, cervical cancer, colorectal cancer, endometrial cancer, esophageal cancer, eye cancer, gallbladder cancer, gastrointestinal carcinoid tumors, gastrointestinal stromal tumor (GIST), gastric cancer, glioma, glioblastoma, head and neck cancer, Hodgkin disease, Kaposi sarcoma, kidney cancer, laryngeal and hypopharyngeal cancer, leukemia, liver cancer, lymphoma, malignant mesothelioma, merkel cell carcinoma, metastatic urothelial carcinoma, multiple myeloma, myeloma, myelodysplastic syndrome, nasal cavity and paranasal sinus cancer, nasopharyngeal cancer, neuroendocrine cancer, neuroblastoma, non-Hodgkin lymphoma, oral cavity and oropharyngeal cancer, osteosarcoma, ovarian cancer, pancreatic cancer, penile cancer, pituitary tumors, prostate cancer, renal cancer, retinoblastoma, rhabdomyosarcoma, salivary gland cancer, sarcoma, squamous cell skin cancer, small intestine cancer, stomach cancer, testicular cancer, thymus cancer, thyroid cancer, uterine cancer, or vaginal cancer. In certain embodiments, the cancer is sarcoma.

In certain embodiments, the radiation treatment comprises hypofractionated radiation. In certain embodiments, the toxicity comprises wound toxicity. In certain embodiments, the patient has a tumor on a lower extremity.

In certain embodiments, the method comprising determining whether the patient carries one or more mutations selected from:
a) a G nucleotide at position 101 of SEQ ID NO: 53 (BMP2_rs1979855);
b) a G nucleotide at position 101 of SEQ ID NO: 55 (PTPN2_rs1893217);
c) a C nucleotide at position 101 of SEQ ID NO: 43 (BRCA2_rs15869);
d) a C nucleotide at position 101 of SEQ ID NO: 65 (ABL1_rs11991);
e) a G nucleotide at position 101 of SEQ ID NO: 63 (ERCC1_rs11615);
f) an A nucleotide at position 101 of SEQ ID NO: 41 (RAD23A_rs8240);
g) a G nucleotide at position 101 of SEQ ID NO: 59 (BATF3_rs6695772);
h) a C nucleotide at position 101 of SEQ ID NO: 143 (FOXP3_rs2232365);
i) a G nucleotide at position 101 of SEQ ID NO: 39 (IL19_rs1798);
j) a G or T nucleotide at position 101 of SEQ ID NO: 129 (LIN28A_rs9438623);
k) an A nucleotide at position 101 of SEQ ID NO: 61 (IL18R1_rs11465660);
l) a T nucleotide at position 101 of SEQ ID NO: 153 (IL2RB_rs228942);
m) a T nucleotide at position 101 of SEQ ID NO: 49 (CD274_rs1411262);
n) a C nucleotide at position 101 of SEQ ID NO: 47 (BMP2_rs3178250);
o) a C nucleotide at position 101 of SEQ ID NO: 137 (SPI1_rs2071304);
p) a G nucleotide at position 101 of SEQ ID NO: 99 (IL19_rs2243158);
q) an A nucleotide at position 101 of SEQ ID NO: 167 (IL10_rs3024505);
r) a G nucleotide at position 101 of SEQ ID NO: 187 (TRL4_rs4986790);
s) an A nucleotide at position 101 of SEQ ID NO: 177 (VEGFA_rs41282644);
t) a G or C nucleotide at position 101 of SEQ ID NO: 163 (P2RX7_rs3751143);
u) a C nucleotide at position 101 of SEQ ID NO: 15 (ERCC1_rs3212948);
v) a G or C nucleotide at position 101 of SEQ ID NO: 131 (FANCC_rs9673);
w) a G nucleotide at position 101 of SEQ ID NO: 51 (CD274_rs2297136);
x) a G nucleotide at position 101 of SEQ ID NO: 19 (IL6_rs2069840);
y) a G nucleotide at position 101 of SEQ ID NO: 45 (CD274_rs2282055);
z) a G nucleotide at position 101 of SEQ ID NO: 57 (CD274_rs822339);
aa) a G nucleotide at position 101 of SEQ ID NO: 95 (HAMP_rs10421768);
bb) a C nucleotide at position 101 of SEQ ID NO: 145 (HAMP_rs1882694);
cc) a G nucleotide at position 101 of SEQ ID NO: 189 (ATM_rs1800057);
dd) a C nucleotide at position 101 of SEQ ID NO: 191 (ILRAP_rs79383051);
ee) an A nucleotide at position 101 of SEQ ID NO: 155 (KIT_rs17084733);
ff) a T nucleotide at position 101 of SEQ ID NO: 7 (rs17599026);
gg) an A nucleotide at position 101 of SEQ ID NO: 67 (ATM_rs189037);
hh) a G nucleotide at position 101 of SEQ ID NO: 69 (CD6_rs76677607);
ii) an A nucleotide at position 101 of SEQ ID NO: 13 (IL1A_rs1800587);
jj) an A nucleotide at position 101 of SEQ ID NO: 179 (IL1A_rs17561);
kk) a G nucleotide at position 101 of SEQ ID NO: 73 (BRCA2_rs7334543);
ll) a T nucleotide at position 101 of SEQ ID NO: 75 (ILF3.58_rs118142475);
mm) a C nucleotide at position 101 of SEQ ID NO: 77 (BIRC5_rs2239680);
nn) a T nucleotide at position 101 of SEQ ID NO: 107 (IL2RA_rs2476491);
oo) an A nucleotide at position 101 of SEQ ID NO: 109 (IL12A_rs568408);
pp) a C nucleotide at position 101 of SEQ ID NO: 111 (GSK3B_rs2037547);
qq) a G nucleotide at position 101 of SEQ ID NO: 135 (IL6_rs1800795);
rr) a T nucleotide at position 101 of SEQ ID NO: 105 (LIG4_rs2232643);
ss) a C nucleotide at position 101 of SEQ ID NO: 31 (STAT3_rs3744483);
tt) a G nucleotide at position 101 of SEQ ID NO: 115 (IL6_rs1800797);
uu) a G nucleotide at position 101 of SEQ ID NO: 17 (IL.6_rs12700386);
vv) an A nucleotide at position 101 of SEQ ID NO: 9 (RAC1_rs9374);
ww) a T nucleotide at position 101 of SEQ ID NO: 121 (ATM_rs373759);
xx) an A nucleotide at position 101 of SEQ ID NO: 123 (CETN2_rs8230);
yy) an A nucleotide at position 101 of SEQ ID NO: 126 (XRCC3_rs861539);
zz) a C nucleotide at position 101 of SEQ ID NO: 127 (miR.34b.c.promoter_rs4938723);
aaa) an A nucleotide at position 101 of SEQ ID NO: 3 (IL1.B_rs4848306);
bbb) an A nucleotide at position 101 of SEQ ID NO: 133 (REV3L_rs465646);
ccc) a C nucleotide at position 101 of SEQ ID NO: 183 (EGFR_rs884225);
ddd) a G nucleotide at position 101 of SEQ ID NO: 181 (TGFB1_rs1800469);
eee) a T nucleotide at position 101 of SEQ ID NO: 165 (rs2187668);
fff) a G nucleotide at position 101 of SEQ ID NO: 185 (rs17388569);
ggg) a G nucleotide at position 101 of SEQ ID NO: 79 (NBN_rs1805794);
hhh) a G nucleotide at position 101 of SEQ ID NO: 83 (CD274_rs4742098);
iii) a G nucleotide at position 101 of SEQ ID NO: 85 (ERCC4_rs4781562);
jjj) an A, C, or G nucleotide at position 101 of SEQ ID N: 87 (MSH2_rs2303428);
kkk) an A nucleotide at position 101 of SEQ ID NO: 151 (IL2RA_rs11256497);
lll) a C nucleotide at position 101 of SEQ ID NO: 159 (LIG4_rs3093772_P1.P2);
mmm) an A nucleotide at position 101 of SEQ ID NO: 29 (IL17D_rs7787);
nnn) a C nucleotide at position 101 of SEQ ID NO: (CD274_rs4143815);
ooo) an A nucleotide at position 101 of SEQ ID NO: 175 (UNGC.96.TGFB2_NA);
ppp) a G nucleotide at position 101 of SEQ ID NO: 161 (MDM2_rs769412); and
qqq) a G nucleotide at position 101 of SEQ ID NO: 147 (IL10_rs1800872).

In certain embodiments, the patient has a decreased likelihood of having a toxic response if the patient carries or does not carry one or more mutations selected from:
a) not carrying or carrying only one copy of a G nucleotide at position 101 of SEQ ID NO: 53 (BMP2_rs1979855);
b) not carrying or carrying only one copy of a G nucleotide at position 101 of SEQ ID NO: 55 (PTPN2_rs1893217);
c) not carrying or carrying only one copy of a C nucleotide at position 101 of SEQ ID NO: 43 (BRCA2_rs15869);
d) carrying a C nucleotide at position 101 of SEQ ID NO: 65 (ABL1_rs11991);
e) not carrying or carrying only one copy of a G nucleotide at position 101 of SEQ ID NO: 63 (ERCC1_rs11615);
f) not carrying or carrying only one copy of an A nucleotide at position 101 of SEQ ID NO: 41 (RAD23A_rs8240);
g) carrying a G nucleotide at position 101 of SEQ ID NO: 59 (BATF3_rs6695772);
h) carrying a C nucleotide at position 101 of SEQ ID NO: 143 (FOXP3_rs2232365);
i) not carrying or carrying only one copy of a G nucleotide at position 101 of SEQ ID NO: 39 (IL19_rs1798);
j) not carrying a G or T nucleotide at position 101 of SEQ ID NO: 129 (LIN28A_rs9438623);
k) not carrying or carrying only one copy of an A nucleotide at position 101 of SEQ ID NO: 61 (IL18R1_rs11465660);
l) not carrying a T nucleotide at position 101 of SEQ ID NO: 153 (IL2RB_rs228942);
m) not carrying or carrying only one copy of a T nucleotide at position 101 of SEQ ID NO: 49 (CD274_rs1411262);
n) not carrying or carrying only one copy of a C nucleotide at position 101 of SEQ ID NO: 47 (BMP2_rs3178250);
o) not carrying a C nucleotide at position 101 of SEQ ID NO: 137 (SPIT_rs2071304);
p) not carrying a G nucleotide at position 101 of SEQ ID NO: 99 (IL19_rs2243158);
q) not carrying an A nucleotide at position 101 of SEQ ID NO: 167 (IL10_rs3024505);
r) carrying a G nucleotide at position 101 of SEQ ID NO: 187 (TRL4_rs4986790);
s) carrying an A nucleotide at position 101 of SEQ ID NO: 177 (VEGFA_rs41282644);
t) carrying a G or C nucleotide at position 101 of SEQ ID NO: 163 (P2RX7_rs3751143);
u) not carrying a C nucleotide at position 101 of SEQ ID NO: 15 (ERCC1_rs3212948);
v) not carrying a G or C nucleotide at position 101 of SEQ ID NO: 131 (FANCC_rs9673);
w) not carrying a G nucleotide at position 101 of SEQ ID NO: 51 (CD274_rs2297136);
x) not carrying or carrying only one copy of a G nucleotide at position 101 of SEQ ID NO: 19 (IL6_rs2069840);
y) not carrying or carrying only one copy of a G nucleotide at position 101 of SEQ ID NO: 45 (CD274_rs2282055);
z) carrying a G nucleotide at position 101 of SEQ ID NO: 57 (CD274_rs822339);
aa) carrying a G nucleotide at position 101 of SEQ ID NO: 95 (HAMP_rs10421768);
bb) carrying a C nucleotide at position 101 of SEQ ID NO: 145 (HAMP_rs1882694);
cc) not carrying a G nucleotide at position 101 of SEQ ID NO: 189 (ATM_rs1800057);
dd) not carrying a C nucleotide at position 101 of SEQ ID NO: 191 (ILRAP_rs79383051);
ee) carrying an A nucleotide at position 101 of SEQ ID NO: 155 (KIT_rs17084733); and
ff) carrying a T nucleotide at position 101 of SEQ ID NO: 7 (rs17599026).

In certain embodiments, the patient has an increased likelihood of having a toxic response if the patient carries or does not carry one or more mutations selected from:
a) carrying two copies of a G nucleotide at position 101 of SEQ ID NO: 53
b) carrying two copies of a G nucleotide at position 101 of SEQ ID NO: 55 (PTPN2_rs1893217);
c) carrying two copies of a C nucleotide at position 101 of SEQ ID NO: 43 (BRCA2_rs15869);
d) not carrying a C nucleotide at position 101 of SEQ ID NO: 65 (ABL1_rs11991);
e) carrying two copies of a G nucleotide at position 101 of SEQ ID NO: 63 (ERCC1_rs11615);
f) carrying two copies of an A nucleotide at position 101 of SEQ ID NO: 41 (RAD23A_rs8240);
g) not carrying a G nucleotide at position 101 of SEQ ID NO: 59 (BATF3_rs6695772);
h) not carrying a C nucleotide at position 101 of SEQ ID NO: 143 (FOXP3_rs2232365);
i) carrying two copies of a G nucleotide at position 101 of SEQ ID NO: 39 (IL19_rs1798);
j) carrying a G or T nucleotide at position 101 of SEQ ID NO: 129 (LIN28A_rs9438623);
k) carrying two copies of an A nucleotide at position 101 of SEQ ID NO: 61 (IL18R1_rs11465660);
l) carrying a T nucleotide at position 101 of SEQ ID NO: 153 (IL2RB_rs228942);
m) carrying two copies of a T nucleotide at position 101 of SEQ ID NO: 49 (CD274_rs1411262);
n) carrying two copies of a C nucleotide at position 101 of SEQ ID NO: 47 (BMP2_rs3178250);
o) carrying a C nucleotide at position 101 of SEQ ID NO: 137 (SPIT_rs2071304);
p) carrying a G nucleotide at position 101 of SEQ ID NO: 99 (IL19_rs2243158);
q) carrying an A nucleotide at position 101 of SEQ ID NO: 167 (IL10_rs3024505);
r) not carrying a G nucleotide at position 101 of SEQ ID NO: 187 (TRL4_rs4986790);
s) not carrying an A nucleotide at position 101 of SEQ ID NO: 177 (VEGFA_rs41282644);
t) not carrying a G or C nucleotide at position 101 of SEQ ID NO: 163 (P2RX7_rs3751143);
u) carrying a C nucleotide at position 101 of SEQ ID NO: 15 (ERCC1_rs3212948);
v) carrying a G or C nucleotide at position 101 of SEQ ID NO: 131 (FANCC_rs9673);
w) carrying a G nucleotide at position 101 of SEQ ID NO: 51 (CD274_rs2297136);
x) carrying two copies of a G nucleotide at position 101 of SEQ ID NO: 19
y) carrying two copies of a G nucleotide at position 101 of SEQ ID NO: 45 (CD274_rs2282055);
z) not carrying a G nucleotide at position 101 of SEQ ID NO: 57 (CD274_rs822339);
aa) not carrying a G nucleotide at position 101 of SEQ ID NO: 95 (HAMP_rs10421768);
bb) not carrying a C nucleotide at position 101 of SEQ ID NO: 145 (HAMP_rs1882694);
cc) carrying a G nucleotide at position 101 of SEQ ID NO: 189 (ATM_rs1800057);
dd) carrying a C nucleotide at position 101 of SEQ ID NO: 191 (ILRAP_rs79383051);
ee) not carrying an A nucleotide at position 101 of SEQ ID NO: 155 (KIT_rs17084733); and
ff) not carrying a T nucleotide at position 101 of SEQ ID NO: 7 (rs17599026).

In certain embodiments, the radiation treatment is a hypofractionated radiation regimen. In certain embodiments, the hypofractionated radiation regimen comprises administering from about 20 Gy to about 35 Gy radiation over about 5 to about 8 fractions. In certain embodiments, the hypofractionated radiation regimen comprises administering stereotactic body radiation therapy.

In certain embodiments, the cancer is selected from adrenal cancer, anal cancer, bile duct cancer, bladder cancer, bone cancer, brain/CNS, basal cell skin cancer, breast cancer, cervical cancer, colorectal cancer, endometrial cancer, esophageal cancer, eye cancer, gallbladder cancer, gastrointestinal carcinoid tumors, gastrointestinal stromal tumor (GIST), gastric cancer, glioma, glioblastoma, head and neck cancer, Hodgkin disease, Kaposi sarcoma, kidney cancer, laryngeal and hypopharyngeal cancer, leukemia, liver cancer, lymphoma, malignant mesothelioma, merkel cell carcinoma, metastatic urothelial carcinoma, multiple myeloma, myeloma, myelodysplastic syndrome, nasal cavity and paranasal sinus cancer, nasopharyngeal cancer, neuroendocrine cancer, neuroblastoma, non-Hodgkin lymphoma, oral cavity and oropharyngeal cancer, osteosarcoma, ovarian cancer, pancreatic cancer, penile cancer, pituitary tumors, prostate cancer, renal cancer, retinoblastoma, rhabdomyosarcoma, salivary gland cancer, sarcoma, squamous cell skin cancer, small intestine cancer, stomach cancer, testicular cancer, thymus cancer, thyroid cancer, uterine cancer, or vaginal cancer. In certain embodiments, the cancer is prostate cancer.

In certain embodiments, the patient has an decreased likelihood of having a toxic response if the patient carries or does not carry one or more mutations selected from:
a) carrying an A nucleotide at position 101 of SEQ ID NO: 67 (ATM_rs189037);
b) not carrying a G nucleotide at position 101 of SEQ ID NO: 69 (CD6_rs76677607);
c) not carrying or carrying only one copy of an A nucleotide at position 101 of SEQ ID NO: 13 (IL1A_rs1800587);
d) carrying an A nucleotide at position 101 of SEQ ID NO: 179 (IL1A_rs17561);
e) carrying a G nucleotide at position 101 of SEQ ID NO: 73 (BRCA2_rs7334543);
f) not carrying a T nucleotide at position 101 of SEQ ID NO: 75 (ILF3.58_rs118142475);
g) carrying a C nucleotide at position 101 of SEQ ID NO: 77 (BIRC5_rs2239680);
h) carrying a T nucleotide at position 101 of SEQ ID NO: 107 (IL2RA_rs2476491);
i) carrying an A nucleotide at position 101 of SEQ ID NO: 109 (IL12A_rs568408);
j) not carrying a C nucleotide at position 101 of SEQ ID NO: 111 (GSK3B_rs2037547);
k) carrying a G nucleotide at position 101 of SEQ ID NO: 135 (IL6_rs1800795);
l) not carrying a T nucleotide at position 101 of SEQ ID NO: 105 (LIG4_rs2232643);
m) not carrying or carrying only one copy of a C nucleotide at position 101 of SEQ ID NO: 31 (STAT3_rs3744483);
n) carrying a G nucleotide at position 101 of SEQ ID NO: 115 (IL6_rs1800797);
o) not carrying or carrying only one copy of a G nucleotide at position 101 of SEQ ID NO: 17 (IL.6_rs12700386);
p) not carrying or carrying only one copy of an A nucleotide at position 101 of SEQ ID NO: 9 (RAC1_rs9374);
q) not carrying a T nucleotide at position 101 of SEQ ID NO: 121 (ATM_rs373759);
r) not carrying an A nucleotide at position 101 of SEQ ID NO: 123 (CETN2_rs8230);
s) not carrying an A nucleotide at position 101 of SEQ ID NO: 126 (XRCC3_rs861539);
t) carrying a C nucleotide at position 101 of SEQ ID NO: 127 (miR.34b.c.promoter_rs4938723);
u) not carrying or carrying only one copy of an A nucleotide at position 101 of SEQ ID NO: 61 (IL18R1_rs11465660);
v) not carrying or carrying only one copy of an A nucleotide at position 101 of SEQ ID NO: 3 (IL1.B_rs4848306);
w) not carrying a G or C nucleotide at position 101 of SEQ ID NO: 131
x) carrying an A nucleotide at position 101 of SEQ ID NO: 133 (REV3L_rs465646);
y) not carrying a G nucleotide at position 101 of SEQ ID NO: 51 (CD274_rs2297136);
z) carrying a C nucleotide at position 101 of SEQ ID NO: 183 (EGFR_rs884225);
aa) not carrying a G nucleotide at position 101 of SEQ ID NO: 181 (TGFB1_rs1800469);
bb) carrying a T nucleotide at position 101 of SEQ ID NO: 165 (rs2187668);
cc) carrying a G nucleotide at position 101 of SEQ ID NO: 185 (rs17388569);
dd) not carrying a G nucleotide at position 101 of SEQ ID NO: 79 (NBN_rs1805794);
ee) carrying a G nucleotide at position 101 of SEQ ID NO: 83 (CD274_rs4742098);
ff) carrying a G nucleotide at position 101 of SEQ ID NO: 85 (ERCC4_rs4781562);
gg) not carrying an A, C, or G nucleotide at position 101 of SEQ ID N: 87 (MSH2_rs2303428);
hh) carrying a T nucleotide at position 101 of SEQ ID NO: 7 (rs17599026);
ii) not carrying an A nucleotide at position 101 of SEQ ID NO: 151 (IL2RA_rs11256497);
jj) carrying a C nucleotide at position 101 of SEQ ID NO: 159 (LIG4_rs3093772_P1.P2);
kk) not carrying or carrying only one copy of an A nucleotide at position 101 of SEQ ID NO: 29 (IL17D_rs7787);
ll) not carrying or carrying only one copy of a C nucleotide at position 101 of SEQ ID NO: (CD274_rs4143815);
mm) not carrying an A nucleotide at position 101 of SEQ ID NO: 175 (UNGC.96.TGFB2_NA);
nn) not carrying a G nucleotide at position 101 of SEQ ID NO: 161 (MDM2_rs769412);
oo) not carrying an A nucleotide at position 101 of SEQ ID NO: 167 (IL10_rs3024505);
pp) not carrying or carrying only one copy of a C nucleotide at position 101 of SEQ ID NO: 43 (BRCA2_rs15869);
qq) not carrying a G nucleotide at position 101 of SEQ ID NO: 147 (IL10_rs1800872);
rr) not carrying or carrying only one copy of a G nucleotide at position 101 of SEQ ID NO: 53 (BMP2_rs1979855);
ss) not carrying or carrying only one copy of a G nucleotide at position 101 of SEQ ID NO: 55 (PTPN2_rs1893217);
tt) carrying a C nucleotide at position 101 of SEQ ID NO: 65 (ABL1_rs11991); and
uu) not carrying or carrying only one copy of a G nucleotide at position 101 of SEQ ID NO: 63 (ERCC1_rs11615).

In certain embodiments, the patient has an increased likelihood of having a toxic response if the patient carries or does not carry one or more mutations selected from:
a) not carrying an A nucleotide at position 101 of SEQ ID NO: 67 (ATM_rs189037);
b) carrying a G nucleotide at position 101 of SEQ ID NO: 69 (CD6_rs76677607);
c) carrying two copies of an A nucleotide at position 101 of SEQ ID NO: 13 (IL1A_rs1800587);
d) not carrying an A nucleotide at position 101 of SEQ ID NO: 179 (IL1A_rs17561);
e) not carrying a G nucleotide at position 101 of SEQ ID NO: 73 (BRCA2_rs7334543);
f) carrying a T nucleotide at position 101 of SEQ ID NO: 75 (ILF3.58_rs118142475);
g) not carrying a C nucleotide at position 101 of SEQ ID NO: 77 (BIRC5_rs2239680);
h) not carrying a T nucleotide at position 101 of SEQ ID NO: 107 (IL2RA_rs2476491);
i) not carrying an A nucleotide at position 101 of SEQ ID NO: 109 (IL12A_rs568408);
j) carrying a C nucleotide at position 101 of SEQ ID NO: 111 (GSK3B_rs2037547);
k) not carrying a G nucleotide at position 101 of SEQ ID NO: 135 (IL6_rs1800795);
l) carrying a T nucleotide at position 101 of SEQ ID NO: 105 (LIG4_rs2232643);
m) carrying two copies of a C nucleotide at position 101 of SEQ ID NO: 31 (STAT3_rs3744483);
n) not carrying a G nucleotide at position 101 of SEQ ID NO: 115 (IL6_rs1800797);
o) carrying two copies of a G nucleotide at position 101 of SEQ ID NO: 17 (IL.6_rs12700386);
p) carrying two copies of an A nucleotide at position 101 of SEQ ID NO: 9 (RAC1_rs9374);
q) carrying a T nucleotide at position 101 of SEQ ID NO: 121 (ATM_rs373759);
r) carrying an A nucleotide at position 101 of SEQ ID NO: 123 (CETN2_rs8230);
s) carrying an A nucleotide at position 101 of SEQ ID NO: 126 (XRCC3_rs861539);
t) not carrying a C nucleotide at position 101 of SEQ ID NO: 127 (miR.34b.c.promoter_rs4938723);
u) carrying two copies of an A nucleotide at position 101 of SEQ ID NO: 61 (IL18R1_rs11465660);
v) carrying two copies of an A nucleotide at position 101 of SEQ ID NO: 3 rs4848306);
w) carrying a G or C nucleotide at position 101 of SEQ ID NO: 131 (FANCC_rs9673);
x) not carrying an A nucleotide at position 101 of SEQ ID NO: 133 (REV3L_rs465646);
y) carrying a G nucleotide at position 101 of SEQ ID NO: 51 (CD274_rs2297136);
z) not carrying a C nucleotide at position 101 of SEQ ID NO: 183 (EGFR_rs884225);
aa) carrying a G nucleotide at position 101 of SEQ ID NO: 181 (TGFB1_rs1800469);
bb) not carrying a T nucleotide at position 101 of SEQ ID NO: 165 (rs2187668);
cc) not carrying a G nucleotide at position 101 of SEQ ID NO: 185 (rs17388569);
dd) carrying a G nucleotide at position 101 of SEQ ID NO: 79 (NBN_rs1805794);
ee) not carrying a G nucleotide at position 101 of SEQ ID NO: 83 (CD274_rs4742098);
ff) not carrying a G nucleotide at position 101 of SEQ ID NO: 85 (ERCC4_rs4781562);
gg) carrying an A, C, or G nucleotide at position 101 of SEQ ID N: 87 (MSH2_rs2303428);
hh) not carrying a T nucleotide at position 101 of SEQ ID NO: 7 (rs17599026);
ii) carrying an A nucleotide at position 101 of SEQ ID NO: 151 (IL2RA_rs11256497);
jj) not carrying a C nucleotide at position 101 of SEQ ID NO: 159 (LIG4_rs3093772_P1.P2);
kk) carrying two copies of an A nucleotide at position 101 of SEQ ID NO: 29 (IL17D_rs7787);
ll) carrying two copies of a C nucleotide at position 101 of SEQ ID NO: (CD274_rs4143815);
mm) carrying an A nucleotide at position 101 of SEQ ID NO: 175 (UNGC.96.TGFB2_NA);
nn) carrying a G nucleotide at position 101 of SEQ ID NO: 161 (MDM2_rs769412);
oo) carrying an A nucleotide at position 101 of SEQ ID NO: 167 (IL10_rs3024505);
pp) carrying two copies of a C nucleotide at position 101 of SEQ ID NO: 43 (BRCA2_rs15869);
qq) carrying a G nucleotide at position 101 of SEQ ID NO: 147 (IL10_rs1800872);
rr) carrying two copies of a G nucleotide at position 101 of SEQ ID NO: 53 (BMP2_rs1979855);
ss) carrying two copies of a G nucleotide at position 101 of SEQ ID NO: 55 (PTPN2_rs1893217);
tt) not carrying a C nucleotide at position 101 of SEQ ID NO: 65 (ABL1_rs11991); and
uu) carrying two copies of a G nucleotide at position 101 of SEQ ID NO: 63 (ERCC1_rs11615).

In certain embodiments, the cancer is selected from adrenal cancer, anal cancer, bile duct cancer, bladder cancer, bone cancer, brain/CNS, basal cell skin cancer, breast cancer, cervical cancer, colorectal cancer, endometrial cancer, esophageal cancer, eye cancer, gallbladder cancer, gastrointestinal carcinoid tumors, gastrointestinal stromal tumor (GIST), gastric cancer, glioma, glioblastoma, head and neck cancer, Hodgkin disease, Kaposi sarcoma, kidney cancer, laryngeal and hypopharyngeal cancer, leukemia, liver cancer, lymphoma, malignant mesothelioma, merkel cell carcinoma, metastatic urothelial carcinoma, multiple myeloma, myeloma, myelodysplastic syndrome, nasal cavity and paranasal sinus cancer, nasopharyngeal cancer, neuroendocrine cancer, neuroblastoma, non-Hodgkin lymphoma, oral cavity and oropharyngeal cancer, osteosarcoma, ovarian cancer, pancreatic cancer, penile cancer, pituitary tumors, prostate cancer, renal cancer, retinoblastoma, rhabdomyosarcoma, salivary gland cancer, sarcoma, squamous cell skin cancer, small intestine cancer, stomach cancer, testicular cancer, thymus cancer, thyroid cancer, uterine cancer, or vaginal cancer. In certain embodiments, the cancer is prostate cancer.

In certain embodiments, the toxicity is acute or long-term toxicity. In certain embodiments, the toxicity is GI and/or GU toxicity.

In certain embodiments, the radiation treatment is a conventionally fractionated (traditional) radiation regimen. In certain embodiments, the traditional radiation regimen comprises administering from about 40 Gy to about 60 Gy radiation over a period of about 5 to about 6 weeks. In certain embodiments, the traditional radiation regimen comprises administering from about 40 Gy to about 60 Gy radiation over about 15 to about 30 fractions. In certain embodiments, the radiation treatment is external beam radiation therapy.

In another aspect, the disclosure relates to a method for determining whether a cancer patient is likely to experience distant failure in response to radiation, the method comprising determining whether the patient carries one or more mutations selected from: TREX1_rs11797, LIN28A_rs9438623, miR.34b.c.promoter_rs4938723, FCGR2A_rs1801274, CD274_rs4742098, IL.6_rs12700386, SPI1_rs2071304, ERCC4_rs4781562, IL10_rs3024496, IL.6_rs2069840, rs2187668, CD274_rs4143815, KRAS_rs61764370, SHC4_rs1062124, HAMP_rs1882694, rs922075, ERCC1_rs11615, EXO1_rs4150021, STAT4_rs7574070, and SOS1_rs1059313.

In certain embodiments, the method comprises determining whether a patient is carrying or not carrying one or more mutations in a SNP selected from:
a) carrying a T nucleotide at position 101 of SEQ ID NO: 81 (TREX1_rs11797);
b) carrying a G or T nucleotide at position 101 of SEQ ID NO: 129 (LIN28A_rs9438623);
c) carrying a C or G nucleotide at position 101 of SEQ ID NO: 119 (FCGR2A_rs1801274);
d) carrying a G nucleotide at position 101 of SEQ ID NO: 83 (CD274_rs4742098);
e) not carrying a G nucleotide at position 101 of SEQ ID NO: 17 (IL.6_rs12700386);
f) not carrying a C nucleotide at position 101 of SEQ ID NO: 137 (SPI1_rs2071304);
g) carrying a G nucleotide at position 101 of SEQ ID NO: 85 (ERCC4_rs4781562);
h) carrying a G nucleotide at position 101 of SEQ ID NO: 103 (IL10_rs3024496_P1.P2);
i) carrying a G nucleotide at position 101 of SEQ ID NO: 139 (IL10_rs3024496);
j) not carrying or carrying only one copy of a G nucleotide at position 101 of SEQ ID NO: 19 (IL.6_rs2069840);
k) not carrying a T nucleotide at position 101 of SEQ ID NO: 165 (rs2187668);
l) not carrying a C nucleotide at position 101 of SEQ ID NO: 27 (CD274_rs4143815);
m) carrying a C nucleotide at position 101 of SEQ ID NO: 157 (KRAS_rs61764370);
n) carrying a G nucleotide at position 101 of SEQ ID NO: 5 (SHC4_rs1062124);
o) carrying a G nucleotide at position 101 of SEQ ID NO: 169 (rs922075);
p) carrying a G nucleotide at position 101 of SEQ ID NO: 63 (ERCC1_rs11615);
q) not carrying the deletion of a T nucleotide at [ATTTTG] of SEQ ID NO: 142 (EXO1_rs4150021);
r) carrying a C nucleotide at position 101 of SEQ ID NO: 173 (STAT4_rs7574070); and
s) carrying a T nucleotide at position 101 of SEQ ID NO: 171 (SOS1_rs1059313).

In certain embodiments, the radiation therapy is conventionally fractionated (traditional) radiation. In certain embodiments, the cancer is prostate cancer.

In another aspect, the disclosure relates to a method for determining whether a cancer patient has an increased or decreased risk of mortality (e.g., prostate cancer specific mortality) following a radiation treatment, the method comprising determining whether the patient carries one or more mutations selected from: BIRC5_rs2239680, SMAD1_rs11724777, IL12A_rs568408, and IL13_rs20541, and, wherein the presence or the absence of the SNP is associated with an increased or decreased risk of mortality.

In certain embodiments, the patient is identified as carrying or not carrying one or more mutations in a SNP selected from:
a) carrying a C nucleotide at position 101 of SEQ ID NO: 77 (BIRC5_rs2239680);
b) not carrying an A nucleotide at position 101 of SEQ ID NO: 93 (SMAD1_rs11724777);
c) not carrying an A nucleotide at position 101 of SEQ ID NO: 109 (IL12A_rs568408); and
d) not carrying a T nucleotide at position 101 of SEQ ID NO: 193 (IL13_rs20541);

wherein the presence or the absence of the SNP is associated with a decreased risk of mortality.

In certain embodiments, the patient is identified as carrying or not carrying one or more mutations in a SNP selected from:
a) not carrying a C nucleotide at position 101 of SEQ ID NO: 77 (BIRC5_rs2239680);
b) carrying an A nucleotide at position 101 of SEQ ID NO: 93 (SMAD1_rs11724777);
c) carrying an A nucleotide at position 101 of SEQ ID NO: 109 (IL12A_rs568408); and
d) carrying a T nucleotide at position 101 of SEQ ID NO: 193 (IL13_rs20541);

wherein the presence or the absence of the SNP is associated with an increased risk of mortality.

In certain embodiments, the radiation treatment is conventionally fractionated (traditional) radiation. In certain embodiments, the cancer is prostate cancer.

In another aspect, the disclosure relates to a method for determining whether a cancer patient has an increased or decreased risk of biochemical relapse following a radiation treatment, the method comprising determining whether the patient carries one or more mutations selected from: BMP2_rs3178250, SMAD1_rs11724777, IL10RB_rs2834167, CD274_rs822339, CD274_rs2282055, IL12A_rs568408, PARP1_rs8679, CD274_rs1411262, BMP2_rs1979855, and P2RX7_rs3751143.

In certain embodiments, the patient has an increased risk of biochemical relapse following a radiation treatment if the patient is identified as carrying or not carrying one or more mutations in a SNP selected from:
a) carrying a C nucleotide at position 101 of SEQ ID NO: 47 (BMP2_rs3178250);
b) not carrying a G nucleotide at position 101 of SEQ ID NO: 91 (IL10RB_rs2834167);
c) not carrying a G nucleotide at position 101 of SEQ ID NO: 57 (CD274_rs822339);
d) carrying a G nucleotide at position 101 of SEQ ID NO: 45 (CD274_rs2282055);
e) carrying an A nucleotide at position 101 of SEQ ID NO: 109 (IL12A_rs568408);
f) carrying a G nucleotide at position 101 of SEQ ID NO: 195 (PARP1_rs8679);
g) carrying a T nucleotide at position 101 of SEQ ID NO: 49 (CD274_rs1411262);
h) carrying a G nucleotide at position 101 of SEQ ID NO: 53 (BMP2_rs1979855); and
i) carrying a C nucleotide at position 101 of SEQ ID NO: 163 (P2RX7_rs3751143);

In certain embodiments, the patient has a decreased risk of biochemical relapse following a radiation treatment if the patient is identified as carrying or not carrying one or more of the following mutations in a SNP:
a) not carrying a C nucleotide at position 101 of SEQ ID NO: 47 (BMP2_rs3178250);
b) carrying a G nucleotide at position 101 of SEQ ID NO: 91 (IL10RB_rs2834167);
c) carrying a G nucleotide at position 101 of SEQ ID NO: 57 (CD274_rs822339);
d) not carrying a G nucleotide at position 101 of SEQ ID NO: 45 (CD274_rs2282055);
e) not carrying an A nucleotide at position 101 of SEQ ID NO: 109 (IL12A_rs568408);
f) not carrying a G nucleotide at position 101 of SEQ ID NO: 195 (PARP1_rs8679);
g) not carrying a T nucleotide at position 101 of SEQ ID NO: 49 (CD274_rs1411262);
h) not carrying a G nucleotide at position 101 of SEQ ID NO: 53 (BMP2_rs1979855); and
i) carrying a C nucleotide at position 101 of SEQ ID NO: 163 (P2RX7_rs3751143);

In certain embodiments, the radiation therapy is conventionally fractionated (traditional) radiation. In certain embodiments, the cancer is prostate cancer.

In another aspect, the disclosure relates to a method for determining whether a cancer patient has an increased or decreased risk of distant metastasis following a radiation treatment, the method comprising determining whether the patient is carrying or not carrying one or more mutations in a SNP selected from: IL8_rs4073, IL10_rs3024496_P1, BIRC5_rs2239680, RAC1_rs9374, IL10_rs3024496_P1.P2, BMP2_rs3178250, and NBN_rs1805794, wherein the presence or the absence of the SNP is associated with an increased or decreased risk of distant metastasis.

In certain embodiments, the patient has an increased risk of distant metastasis following a radiation treatment if the patient is identified as carrying or not carrying one or more mutations in a SNP selected from:
a) carrying a T nucleotide at position 101 of SEQ ID NO: 197 (IL8_rs4073);
b) not carrying a G or C nucleotide at position 101 of SEQ ID NO: 103 (IL10_rs3024496_P1);
c) not carrying a C nucleotide occurs at position 101 of SEQ ID NO: 77 (BIRC5_rs2239680);
d) carrying an A nucleotide at position 101 of SEQ ID NO: 9 (RAC1_rs9374);
e) not carrying a G nucleotide occurs at position 101 of SEQ ID NO: 103 (IL10_rs3024496_P1.P2);
f) carrying a G nucleotide at position 101 of SEQ ID NO: 53 (BMP2_rs1979855); and
g) carrying a G nucleotide at position 101 of SEQ ID NO: 79 (NBN_rs1805794).

In certain embodiments, the patient has a decreased risk of distant metastasis following a radiation treatment if the patient is identified as carrying or not carrying one or more of the following mutations in a SNP:
a) not carrying a T nucleotide at position 101 of SEQ ID NO: 197 (IL8_rs4073);
b) carrying a G or C nucleotide at position 101 of SEQ ID NO: 103 (IL10_rs3024496_P1);
c) carrying a C nucleotide occurs at position 101 of SEQ ID NO: 77 (BIRC5_rs2239680);
d) not carrying an A nucleotide at position 101 of SEQ ID NO: 9 (RAC1_rs9374);
e) carrying a G nucleotide occurs at position 101 of SEQ ID NO: 103 (IL10_rs3024496_P1.P2);
f) not carrying a G nucleotide at position 101 of SEQ ID NO: 53 (BMP2_rs1979855); and
g) not carrying a G nucleotide at position 101 of SEQ ID NO: 79 (NBN_rs1805794).

In certain embodiments, the radiation therapy is conventionally fractionated (traditional) radiation. In certain embodiments, the cancer is prostate cancer.

In another aspect, the disclosure relates to a method for determining whether a prostate cancer patient has an increased or decreased risk of impotency following treatment with radiation, the method comprising determining whether the patient is carrying or not carrying one or more mutations in a SNP selected from: HAMP_rs10421768, XRCC1_rs25487, IL.6_rs2069840, XRCC4_rs1040363, and IL19_rs2243158.

In certain embodiments, the patient is identified as carrying or not carrying one or more mutations in a SNP associated with a increased risk of impotency following treatment with radiation and selected from:
a) not carrying a G nucleotide at position 101 of SEQ ID NO: 95 (HAMP_rs10421768);
b) carrying a C nucleotide at position 101 of SEQ ID NO: 97 (XRCC1_rs25487);
c) carrying a G nucleotide at position 101 of SEQ ID NO: 19 (IL.6_rs2069840);
d) not carrying a C nucleotide at position 101 of SEQ ID NO: 89 (XRCC4_rs1040363); and
e) carrying a G nucleotide at position 101 of SEQ ID NO: 99 (IL19_rs2243158).

In certain embodiments, the patient is identified as carrying or not carrying one or more mutations in a SNP associated with a decreased risk of impotency following treatment with radiation and selected from:
a) carrying a G nucleotide at position 101 of SEQ ID NO: 95 (HAMP_rs10421768);
b) not carrying a C nucleotide at position 101 of SEQ ID NO: 97 (XRCC1_rs25487);
c) not carrying a G nucleotide at position 101 of SEQ ID NO: 19 (IL.6_rs2069840);
d) carrying a C nucleotide at position 101 of SEQ ID NO: 89 (XRCC4_rs1040363); and
e) not carrying a G nucleotide at position 101 of SEQ ID NO: 99 (IL19_rs2243158).

In certain embodiments, the radiation treatment is conventionally fractionated (traditional) radiation.

In another aspect, the disclosure relates to a method for determining the toxicity of a radiation treatment in a cancer patient, wherein the is patient identified as carrying or not carrying a G nucleotide at a position corresponding to position 101 of SEQ ID NO: 19 (IL.6_rs2069840).

In certain embodiments, the patient is further identified as carrying or not carrying an A nucleotide at a position corresponding to position 101 of SEQ ID NO: 69 (CD6_rs76677607). In certain embodiments, the patient is further identified as carrying or not carrying a C nucleotide at a position corresponding to position 101 of SEQ ID NO: 27 (CD274_rs4143815).

In certain embodiments, the patient is further identified as carrying or not carrying a G nucleotide at a position corresponding to position 101 of SEQ ID NO: 5 (SHC4_rs1062124). In certain embodiments, the patient is further identified as carrying or not carrying a C nucleotide at a position corresponding to position 101 of SEQ ID NO: 15 (ERCC1_rs3212948). In certain embodiments, the patient is further identified as carrying or not carrying an A nucleotide at a position corresponding to position 101 of SEQ ID NO: 117 (RAC1_rs9374). In certain embodiments, the method further comprises determining whether the patient has a lower extremity tumor. In certain embodiments, the patient is further identified as carrying or not carrying a T nucleotide at a position corresponding to position 101 of SEQ ID NO: 37 (UNGC.41.IL1RAP). In certain embodiments, the patient is further identified as carrying or not carrying a C nucleotide at a position corresponding to position 101 of SEQ ID NO: 27 (CD274_rs4143815). In certain embodiments, the patient is further identified as carrying or not carrying a C nucleotide at a position corresponding to position 101 of SEQ ID NO: 1 (miR.99a.promoter). In certain embodiments, the patient is further identified as carrying or not carrying an A nucleotide at a position corresponding to position 101 of SEQ ID NO: 13 (IL1A_rs1800587).

In certain embodiments, the method further comprises administering a reduced toxicity radiation treatment to the patient.

In certain embodiments, the cancer is selected from adrenal cancer, anal cancer, bile duct cancer, bladder cancer, bone cancer, brain/CNS, basal cell skin cancer, breast cancer, cervical cancer, colorectal cancer, endometrial cancer, esophageal cancer, eye cancer, gallbladder cancer, gastrointestinal carcinoid tumors, gastrointestinal stromal tumor (GIST), gastric cancer, glioma, glioblastoma, head and neck cancer, Hodgkin disease, Kaposi sarcoma, kidney cancer, laryngeal and hypopharyngeal cancer, leukemia, liver cancer, lymphoma, malignant mesothelioma, merkel cell carcinoma, metastatic urothelial carcinoma, multiple myeloma, myeloma, myelodysplastic syndrome, nasal cavity and paranasal sinus cancer, nasopharyngeal cancer, neuroendocrine cancer, neuroblastoma, non-Hodgkin lymphoma, oral cavity and oropharyngeal cancer, osteosarcoma, ovarian cancer, pancreatic cancer, penile cancer, pituitary tumors, prostate cancer, renal cancer, retinoblastoma, rhabdomyosarcoma, salivary gland cancer, sarcoma, squamous cell skin cancer, small intestine cancer, stomach cancer, testicular cancer, thymus cancer, thyroid cancer, uterine cancer, or vaginal cancer. In certain embodiments, the cancer is sarcoma.

In certain embodiments, the toxicity is wound toxicity.

In certain embodiments, the radiation treatment comprises a hypofractionated radiation regimen. In certain embodiments, the hypofractionated radiation regimen comprises administering from about 20 Gy to about 35 Gy radiation over about 5 to about 8 fractions. In certain embodiments, the hypofractionated radiation regimen comprises administering stereotactic body radiation therapy.

In certain embodiments, the radiation treatment comprises a conventionally fractionated (traditional) radiation regimen. In certain embodiments, the traditional radiation regimen comprises administering from about 40 Gy to about 60 Gy radiation over a period of about 5 to about 6 weeks. In certain embodiments, the traditional radiation regimen comprises administering from about 40 Gy to about 60 Gy radiation over about 15 to about 30 fractions.

### BRIEF DESCRIPTION OF THE DRAWINGS

FIG. 1 is a table showing single nucleotide mutations found in various human genes and the corresponding wild-type sequences. As described herein, these mutations are biomarkers that are relevant to predicting a patient's toxic response to radiation treatment. The sequences shown are segments of the human gene's nucleotide sequence; 100 nucleotides upstream (5′) and 100 nucleotides downstream (3′) of the mutation are shown. The mutation is shown in square brackets “[ ]” in the variant sequence and is at position 101; the corresponding wild-type nucleotide is shown in square brackets “[ ]” at position 101 in the wild-type sequence. SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 135, 137, 139, 141, 143, 145, 147, 149, 151, 153, 155, 157, 159, 161, 163, 165, 167, 169, 171, 173, 175, and 177 are the variant sequences while SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178 are the wild-type version of the sequence (i.e., without the mutation).

FIG. 2 is a CONSORT (Consolidated Standards of Reporting Trials) diagram showing the progress through phases of a clinical trial for hypofractionated radiation treatment for sarcoma.

FIG. 3 is a tree-based classification rule providing a prognostic indication of a patient's response to radiation treatment. Each leaf of the tree provides a probability of response to treatment based on the presence or absence of the particular biomarker as indicated therein, i.e., IL6_rs2069840, SHC4_rs1062124, ERCC1_rs3212948, RAC1_rs9374, UNGC.41.IL1RAP, CD274_rs4143815, miR99a promoter, IL1A_rs1800587, and CD6_rs76677607. “0” means the patient is homozygous wild-type. “1” means the patient carries one copy of the mutation, i.e., the patient is heterozygous for the mutation.

FIG. 4 is a Variable Importance diagram showing the top predictors of late toxicity for stereotactic body radiation treatment (SBRT) for prostate cancer based on Random Forest classification.

FIG. 5 is a Variable Importance diagram showing the top predictors of late toxicity for stereotactic body radiation treatment (SBRT) for prostate cancer based on Elastic Net classification.

FIG. 6 is Variable Importance diagram showing the top predictors of late toxicity for stereotactic body radiation treatment (SBRT) for prostate cancer based on Boosted Trees classification.

FIG. 7 is Variable Importance diagram showing the top predictors of acute toxicity for conventional radiation treatment, using data from the OSLO cohort, for prostate cancer based on Random Forest classification.

FIG. 8 is Variable Importance diagram showing the top predictors of acute toxicity for conventional radiation treatment, using data from the OSLO cohort, for prostate cancer based on Elastic Net classification.

FIG. 9 is Variable Importance diagram showing the top predictors of acute toxicity for conventional radiation treatment, using data from the OSLO cohort, for prostate cancer based on Boosted Trees classification.

FIG. 10 is Variable Importance diagram showing the top predictors of late toxicity for conventional radiation treatment, using data from the OSLO cohort, for prostate cancer based on Random Forest classification.

FIG. 11 is Variable Importance diagram showing the top predictors of late toxicity for conventional radiation treatment, using data from the OSLO cohort, for prostate cancer based on Elastic Net classification.

FIG. 12 is Variable Importance diagram showing the top predictors of late toxicity for conventional radiation treatment, using data from the OSLO cohort, for prostate cancer based on Boosted Trees classification.

FIG. 13 shows a Variable Importance diagram using a Random Forest classification strategy for acute toxicity in the OSLO cohort.

FIG. 14 shows a Variable Importance diagram using an Elastic Net classification strategy for acute toxicity in the OSLO cohort.

FIG. 15 shows a Variable Importance diagram using a Boosted Trees classification strategy for acute toxicity in the OSLO cohort.

FIG. 16 shows a Variable Importance diagram using a Random Forest classification strategy for late-term toxicity in the OSLO cohort.

FIG. 17 shows a Variable Importance diagram using an Elastic Net classification strategy for late-term toxicity in the OSLO cohort.

FIG. 18 shows a Variable Importance diagram using a Boosted Trees classification strategy for late-term toxicity in the OSLO cohort.

FIG. 19 shows a Variable Importance diagram using a Random Forest classification strategy for late-term toxicity in the SBRT cohort.

FIG. 20 shows a Variable Importance diagram using an Elastic Net classification strategy for late-term toxicity in the SBRT cohort.

FIG. 21 shows a Variable Importance diagram using a Boosted Trees classification strategy for late-term toxicity in the SBRT cohort.

### DETAILED DESCRIPTION OF THE INVENTION

### Single Nucleotide Germline Mutations as Biomarkers

The invention is based, in part, on the discovery that a cancer patient carrying one or more specified mutations in their genome may exhibit increased or decreased toxicity to radiation therapy (e.g., hypofractionated radiation) as compared to other patients, e.g., wild-type patients. The mutations may commonly be referred to as single nucleotide polymorphisms or “SNPs,” and certain mutations disclosed herein are functional mutations that are present in the germline. The mutations are generally to a single nucleotide, for example, substitution of a nucleotide or deletion of a nucleotide, although in specific instances the mutations described herein relate to the deletion of more than one nucleotide.

In certain embodiments, the increased or decreased toxicity may comprise short-term (acute) toxicity. In certain embodiments, the increased or decreased toxicity may comprise long-term (late) toxicity. The terms short-term toxicity and acute toxicity are used interchangeably herein. The terms long-term and late or late-term toxicity are used interchangeably herein.

In certain embodiments, for example, when the cancer is sarcoma, the toxicity comprises wound toxicity. In certain embodiments, for example, when the cancer is prostate cancer, the toxicity comprises gastrointestinal (GI) and/or genitourinary (GU) toxicity.

The invention is based, in part, on the discovery that a cancer patient carrying one or more specified mutations in their genome may exhibit increased likelihood of experiencing mortality, biochemical relapse, or distant failure (i.e., distant metastasis) despite undergoing treatment with radiation therapy.

The mutations referred to herein include functional mutations that disrupt microRNA pathways, and include microRNA binding site mutations. A microRNA (miRNA) is a small non-coding RNA molecule containing about 22 nucleotides found in plants, animals, and some viruses that functions in RNA silencing and post-transcription regulation of gene expression. These functions are integral to miRNAs' role as critical stress response mediators, including mediating the immune and inflammatory response. DNA damage is also known to cause changes in the global profile of miRNA expression (Weidhaas et al. (2007) CANCER RES. 67:11111) and stress-induced miRNA deregulation has been observed at the level of transcription, processing, subcellular localization and functioning. Accordingly, biomarkers predictive of disruption in microRNA pathways may be useful for predicting toxicity of radiation therapies, particularly because of how such pathways influence immune and inflammatory response.

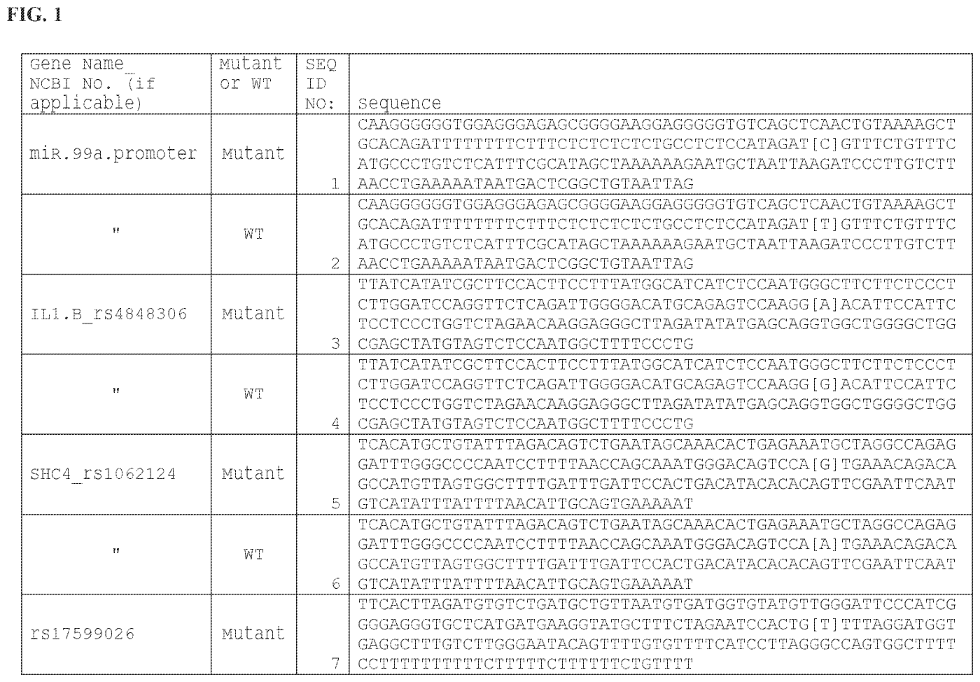

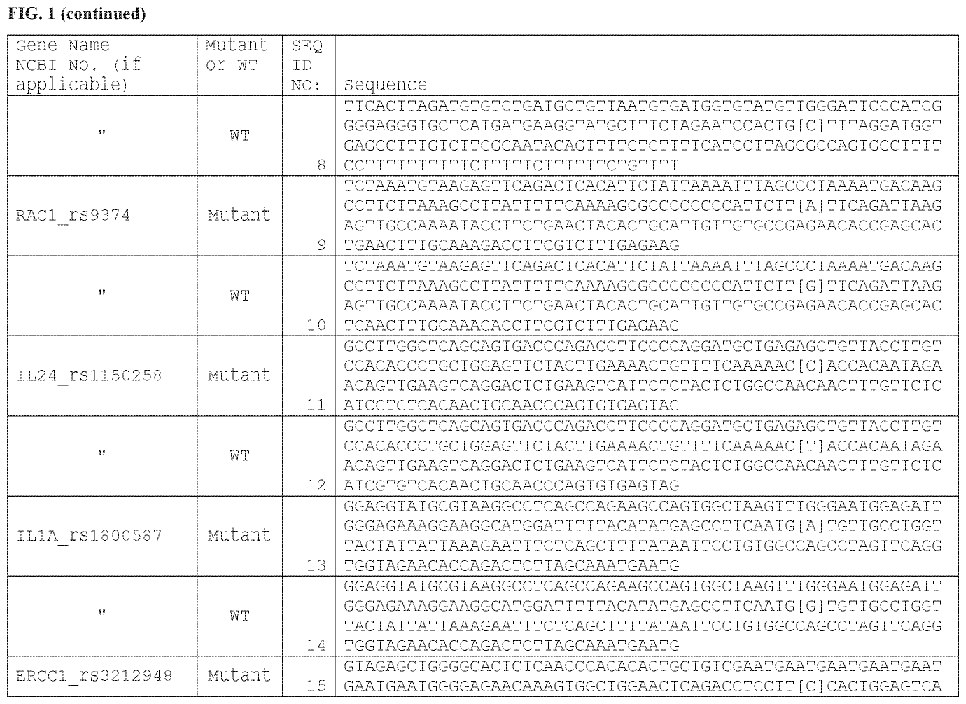

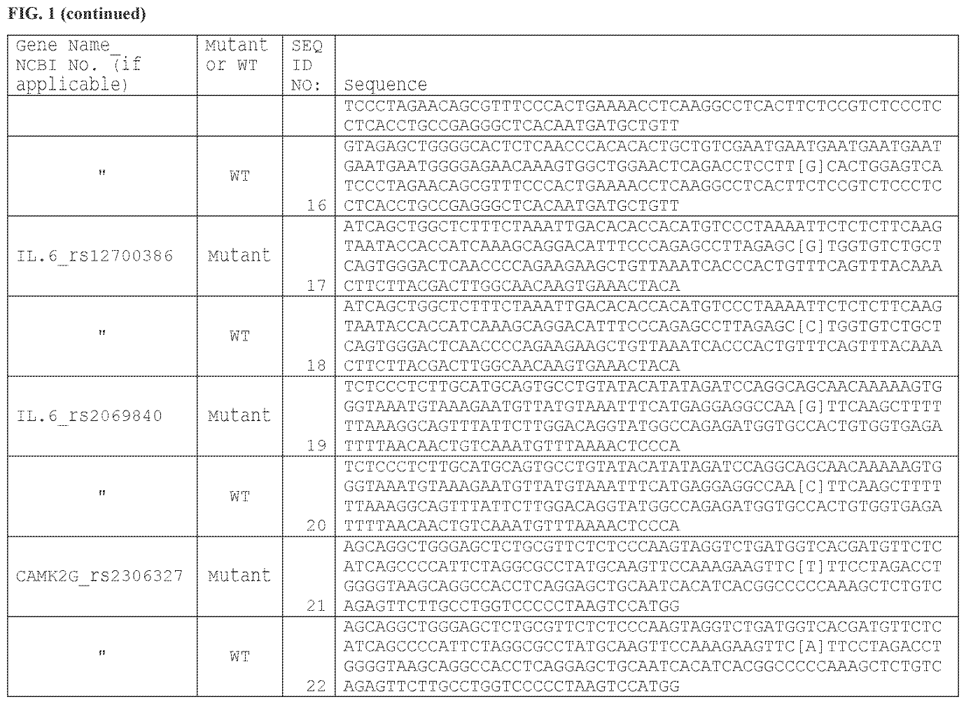

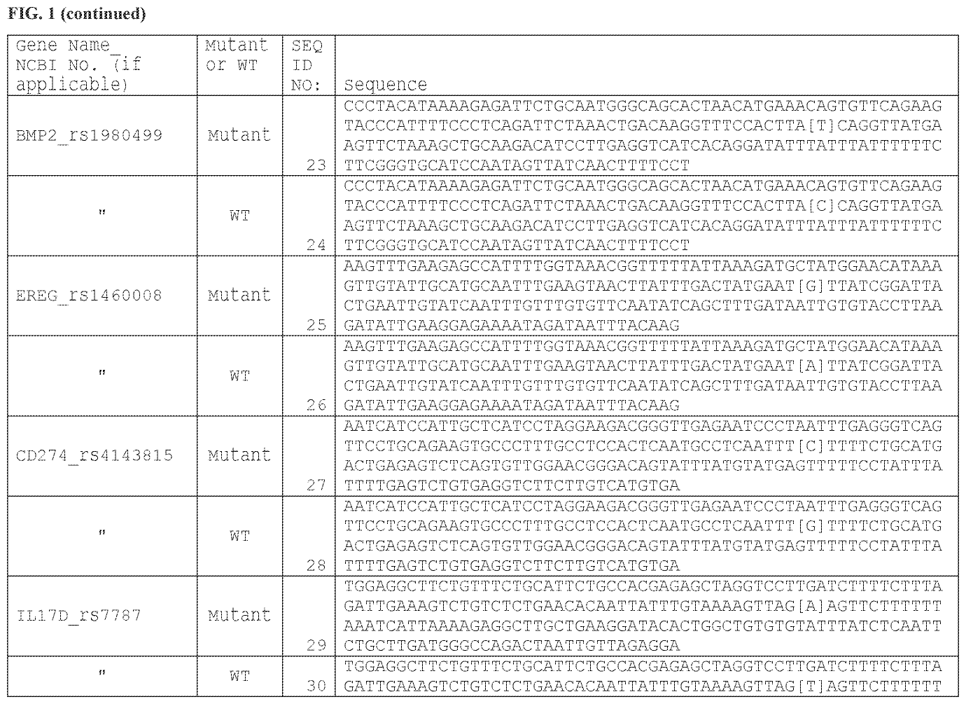

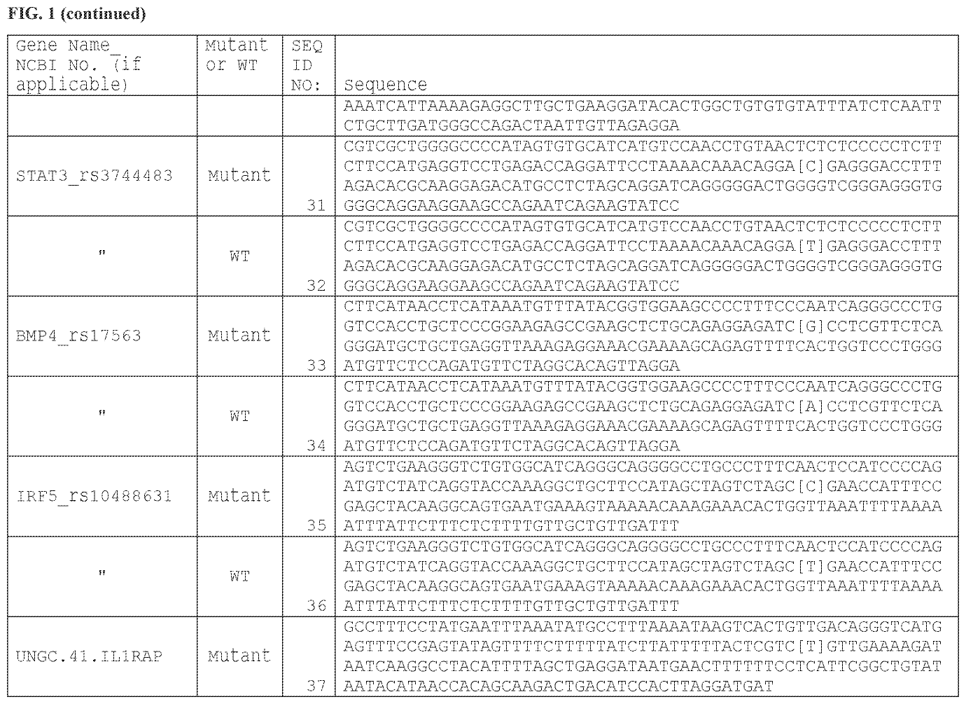

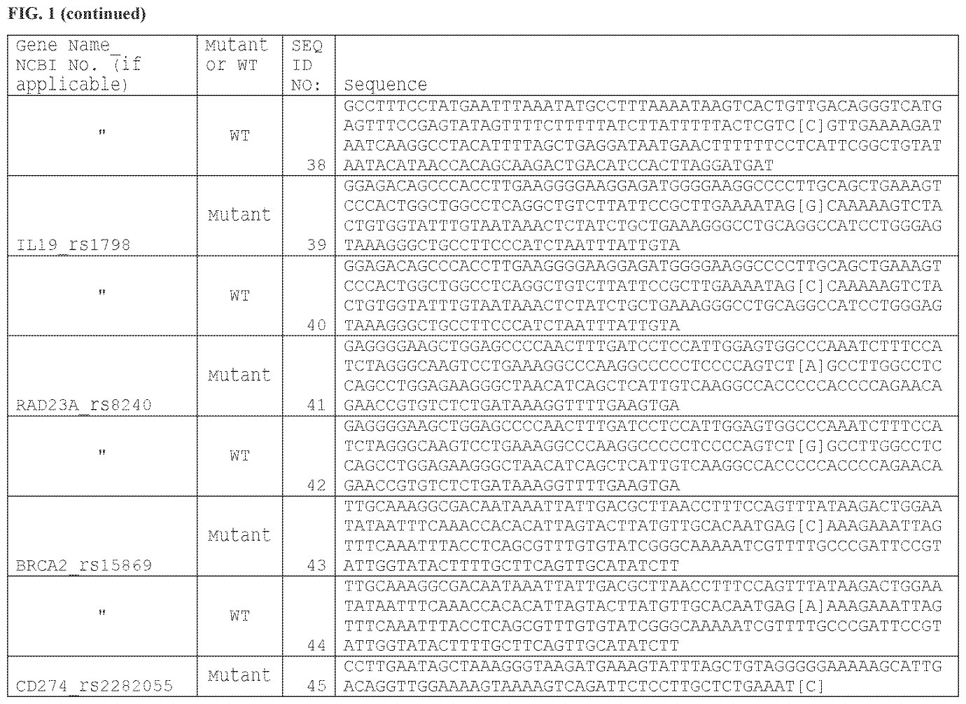

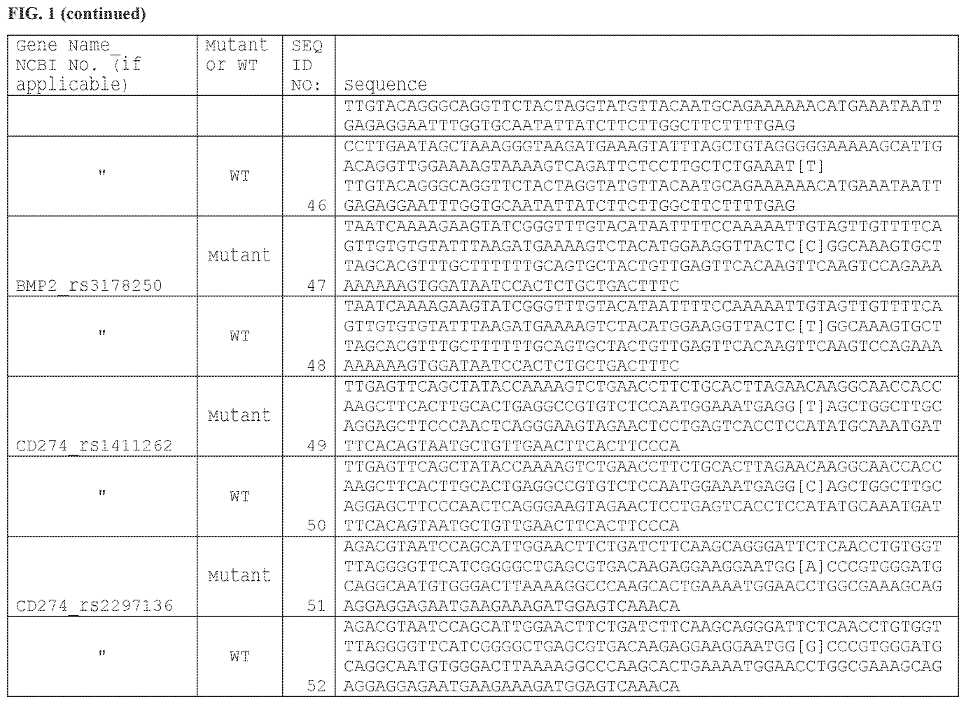

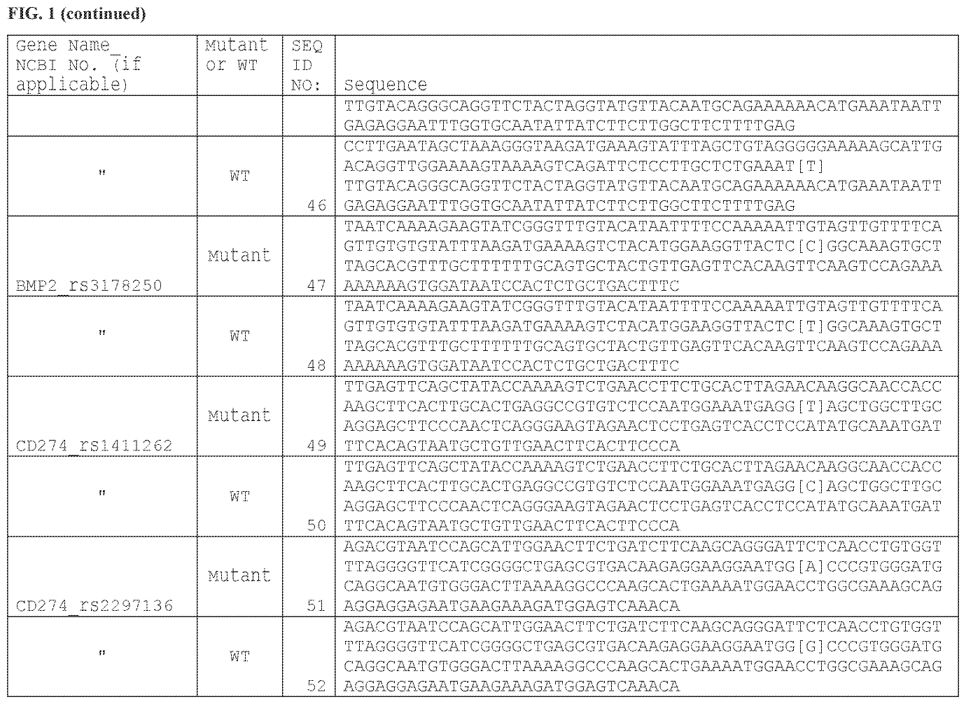

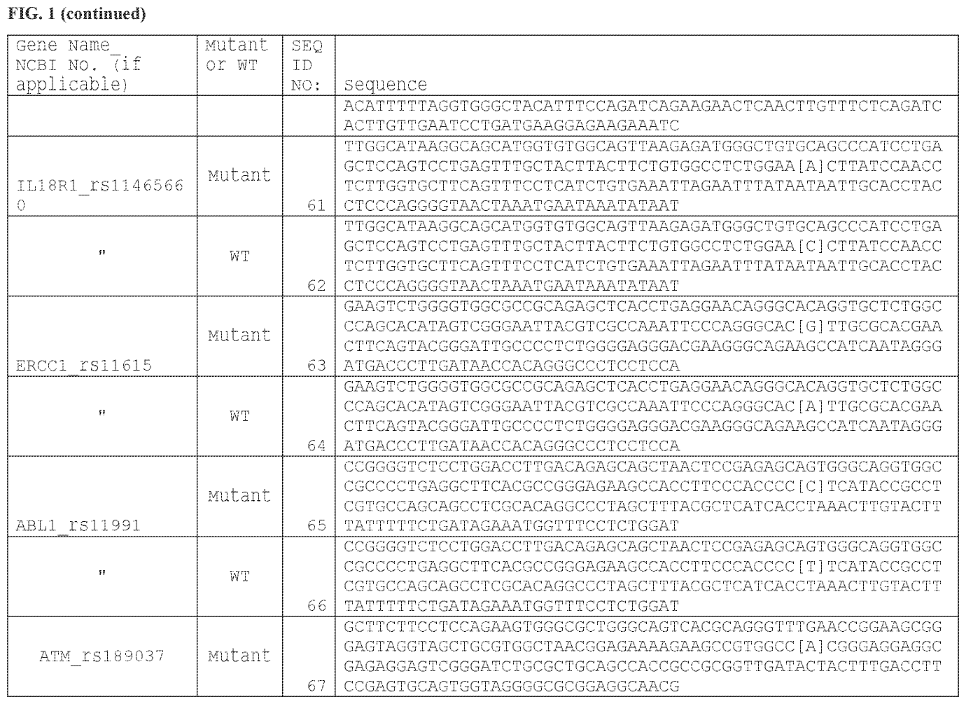

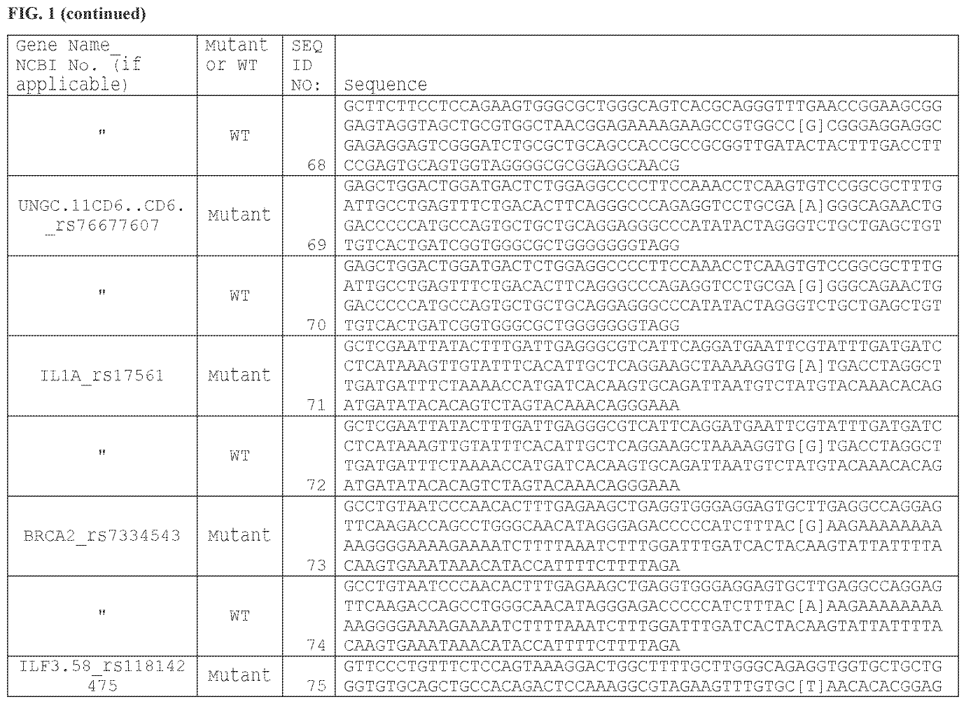

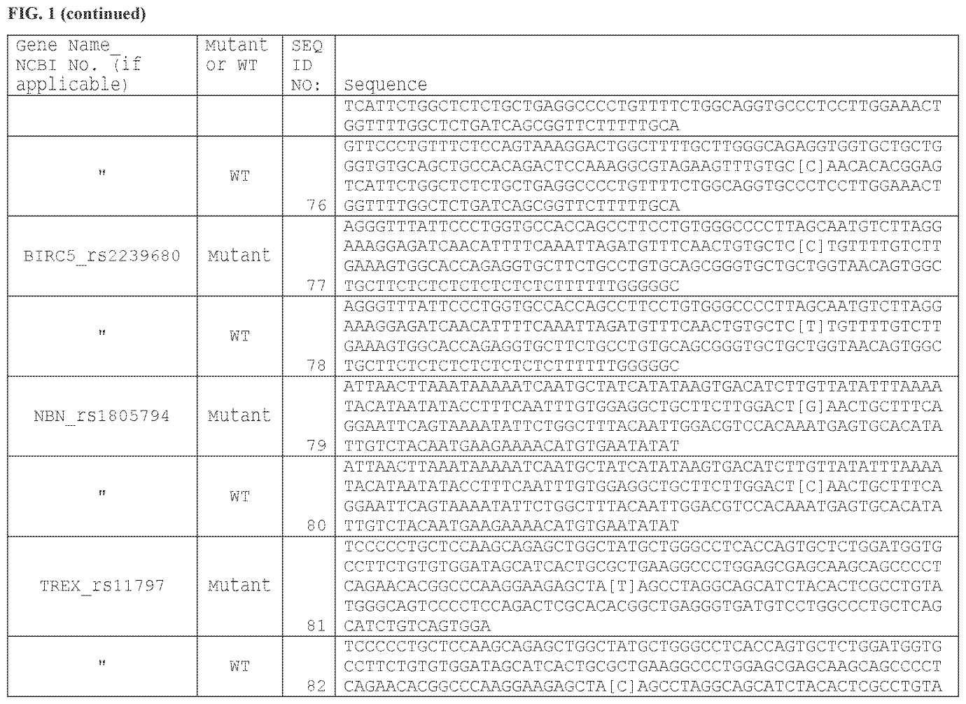

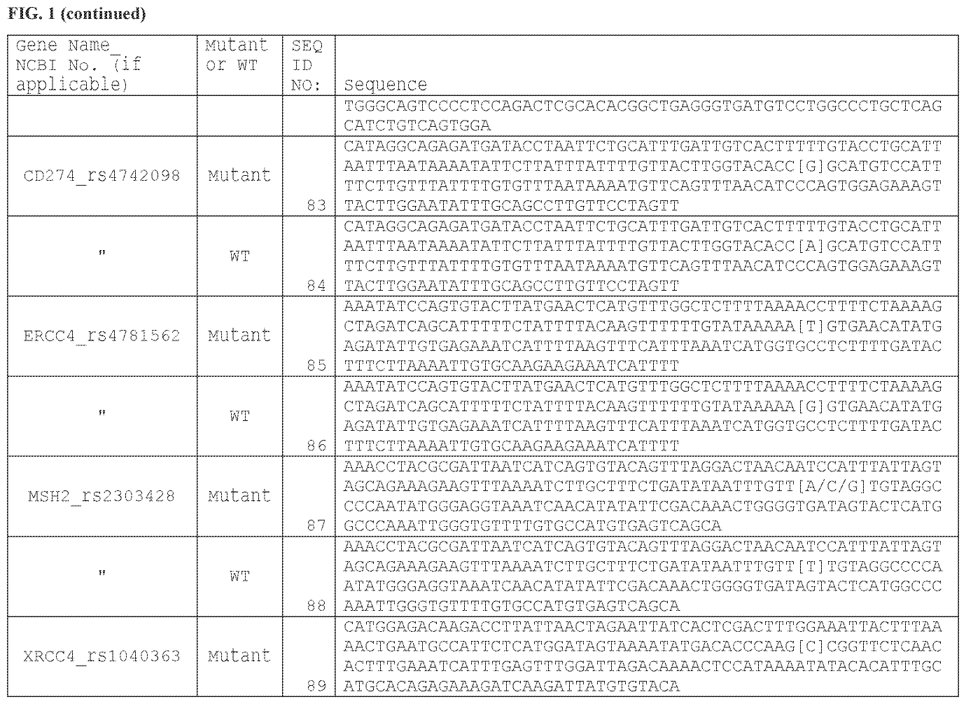

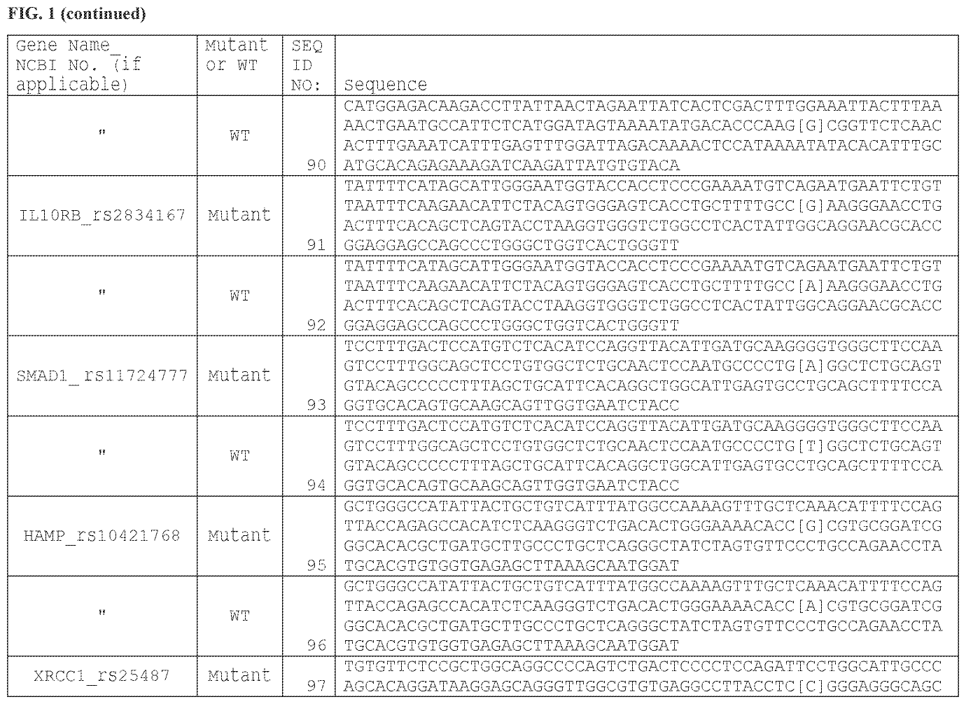

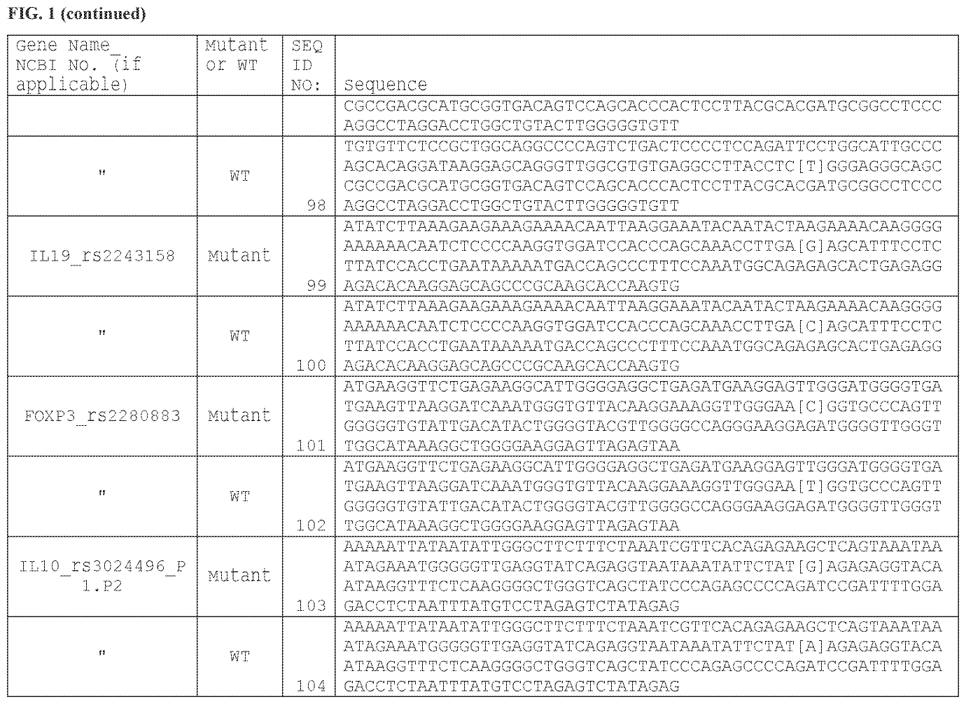

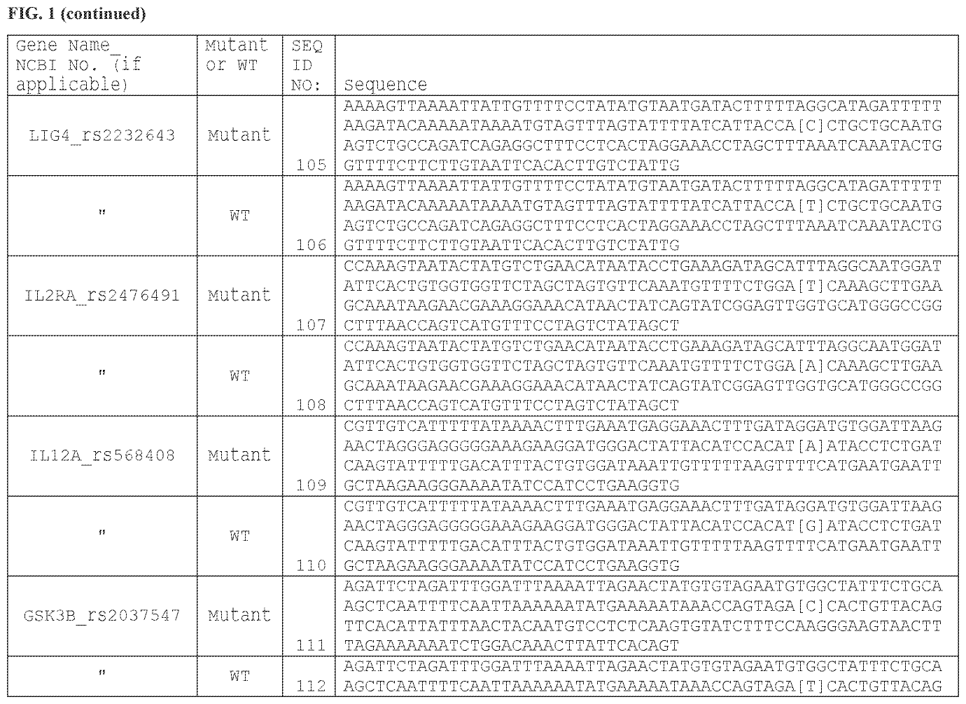

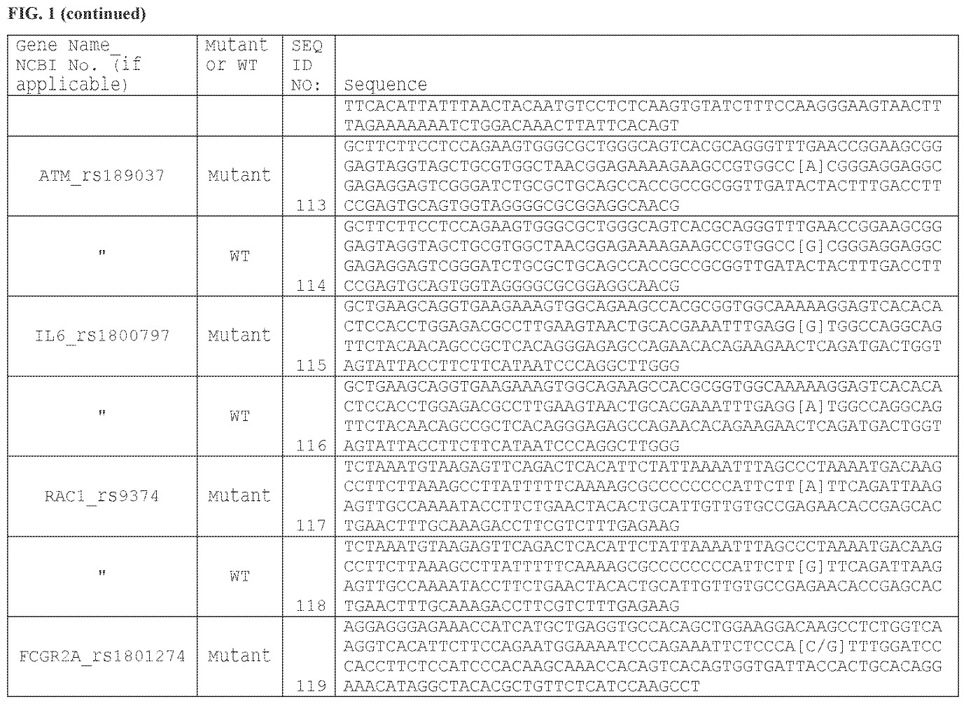

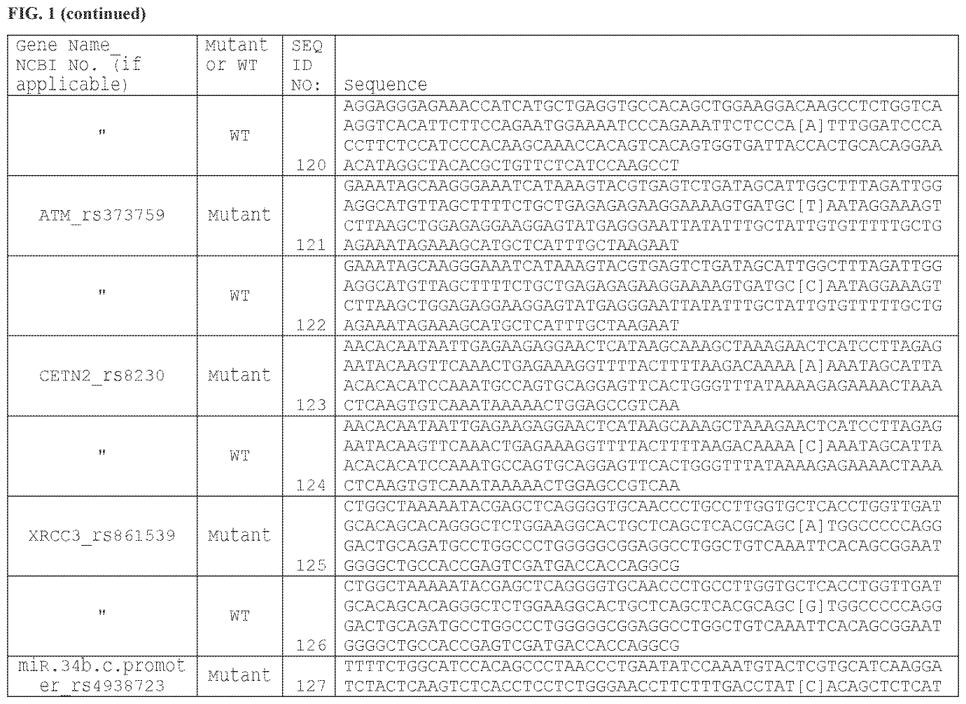

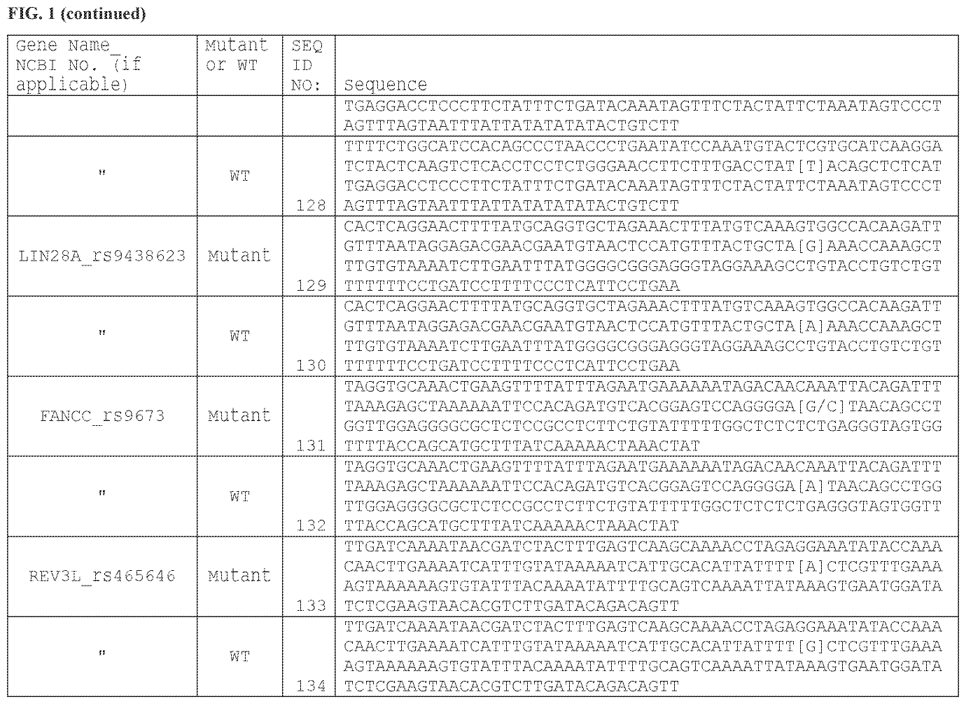

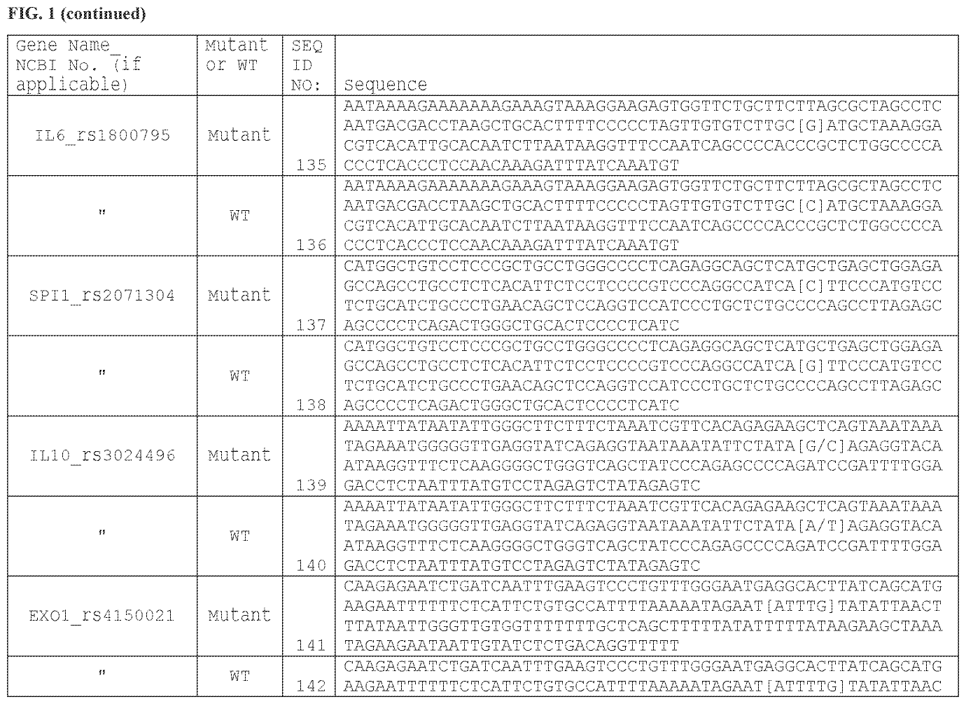

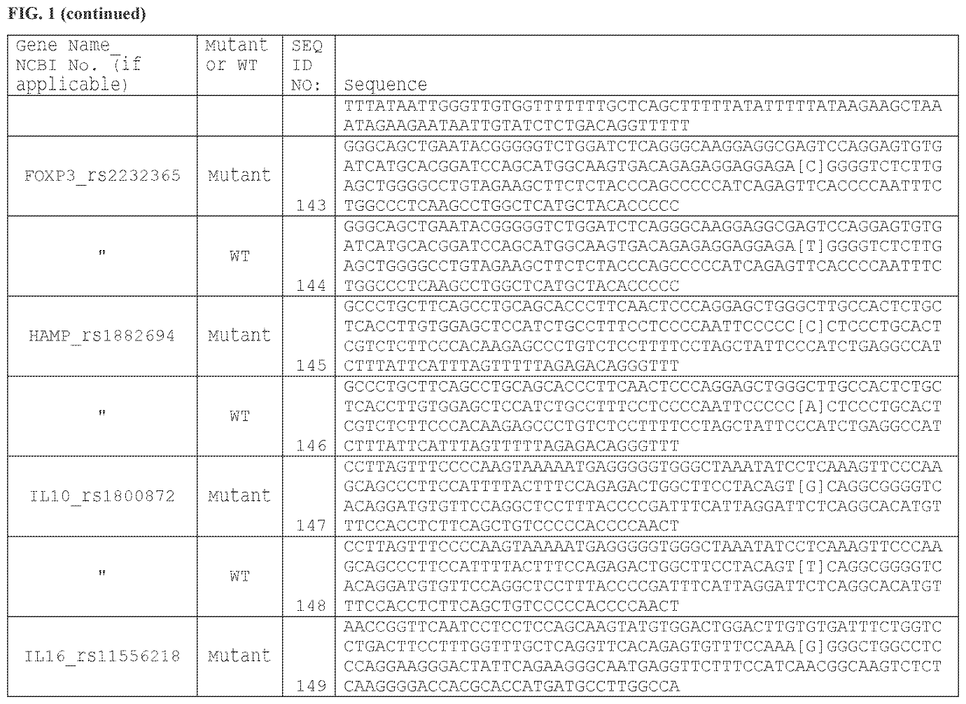

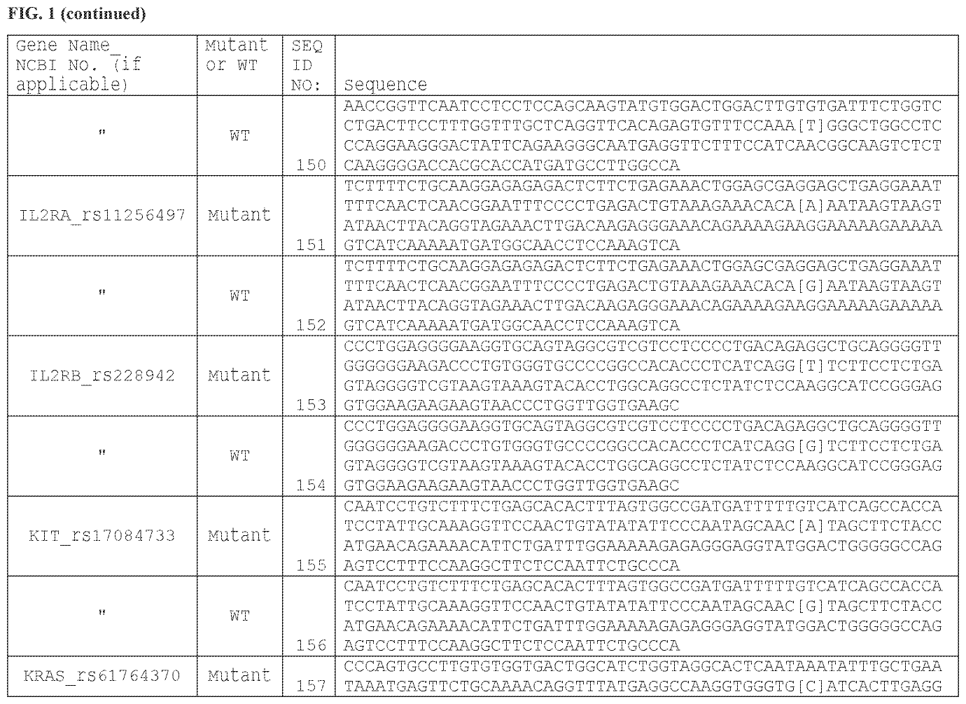

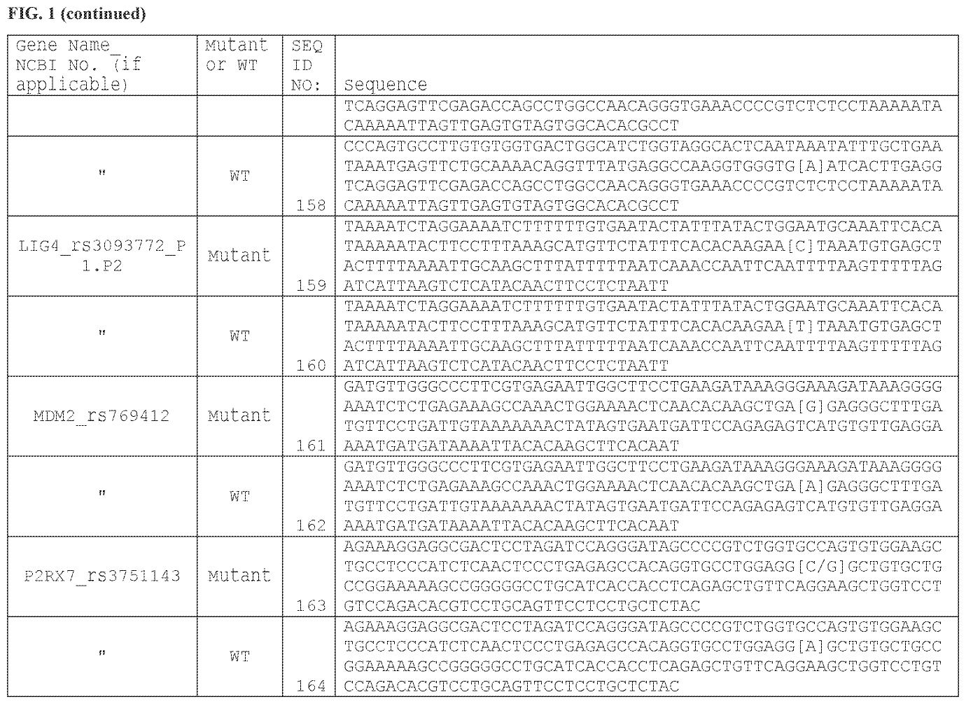

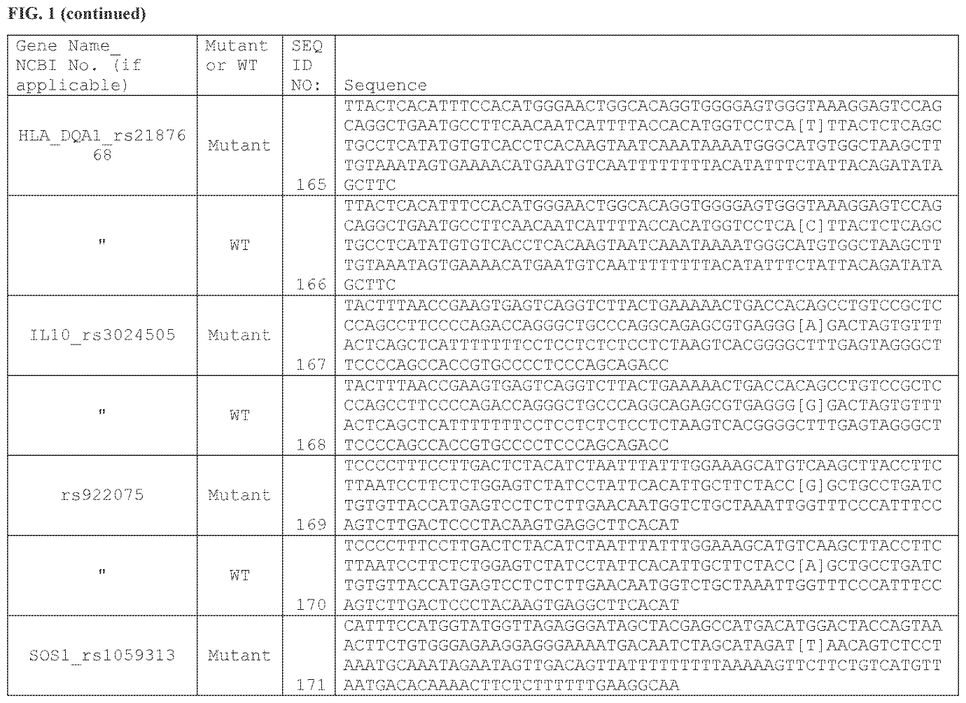

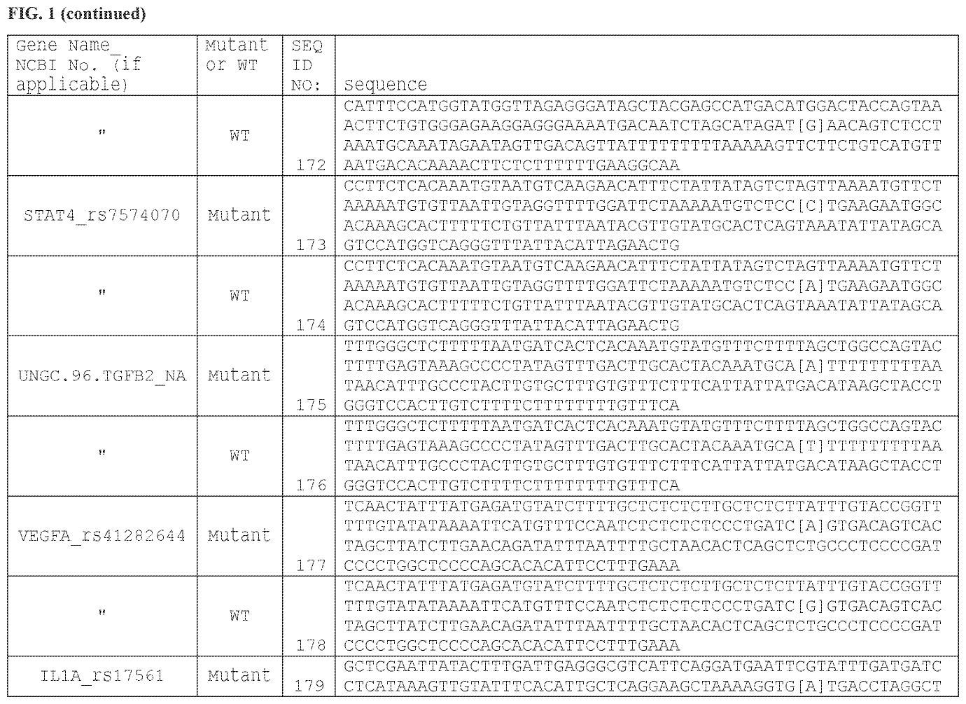

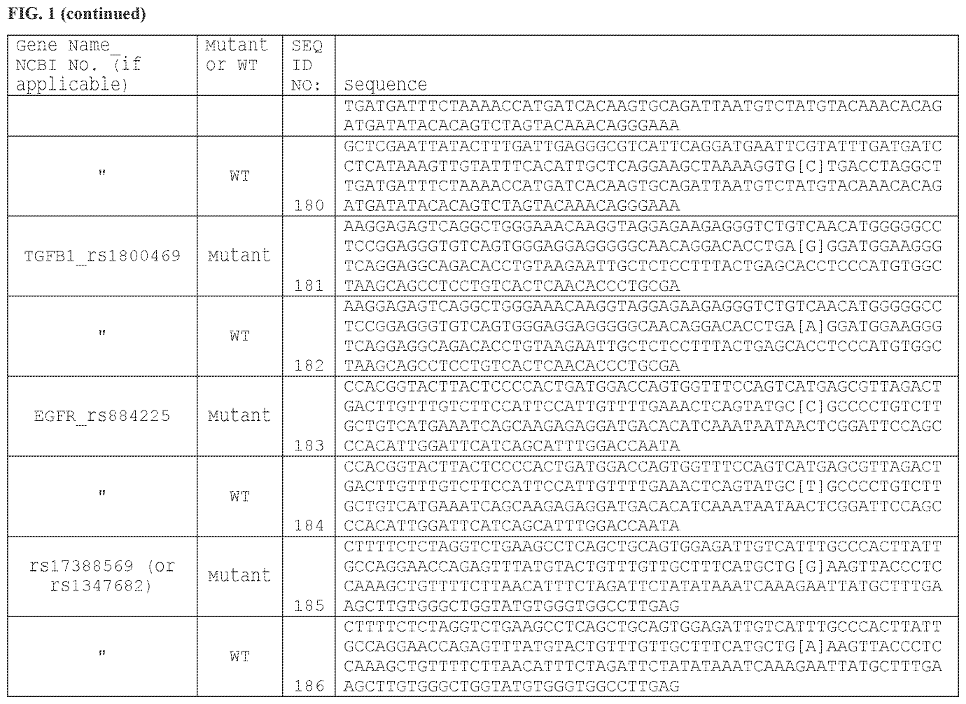

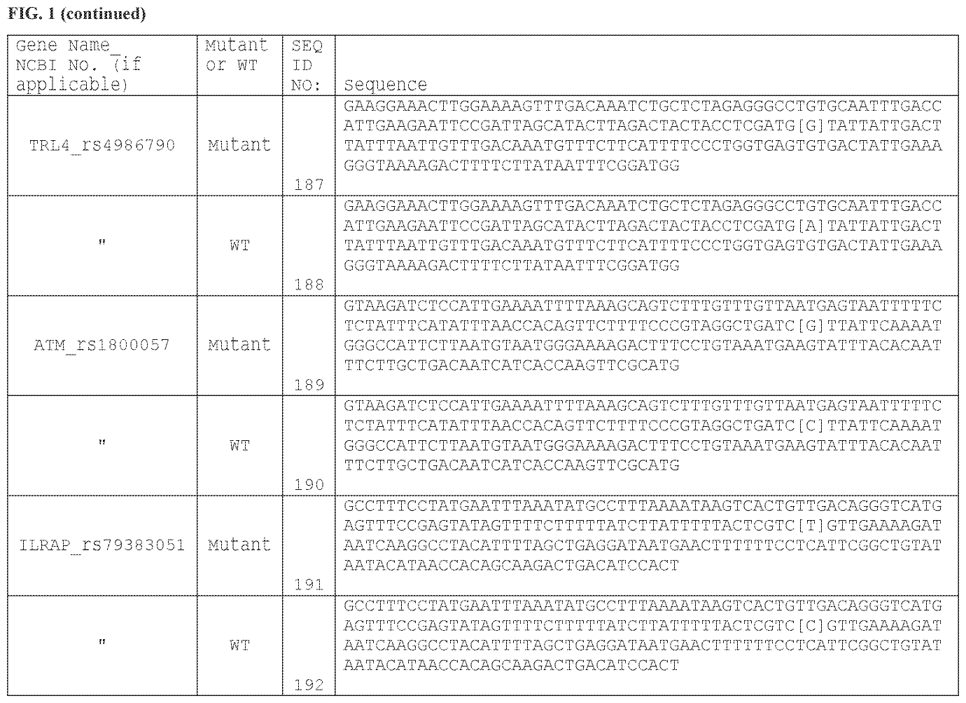

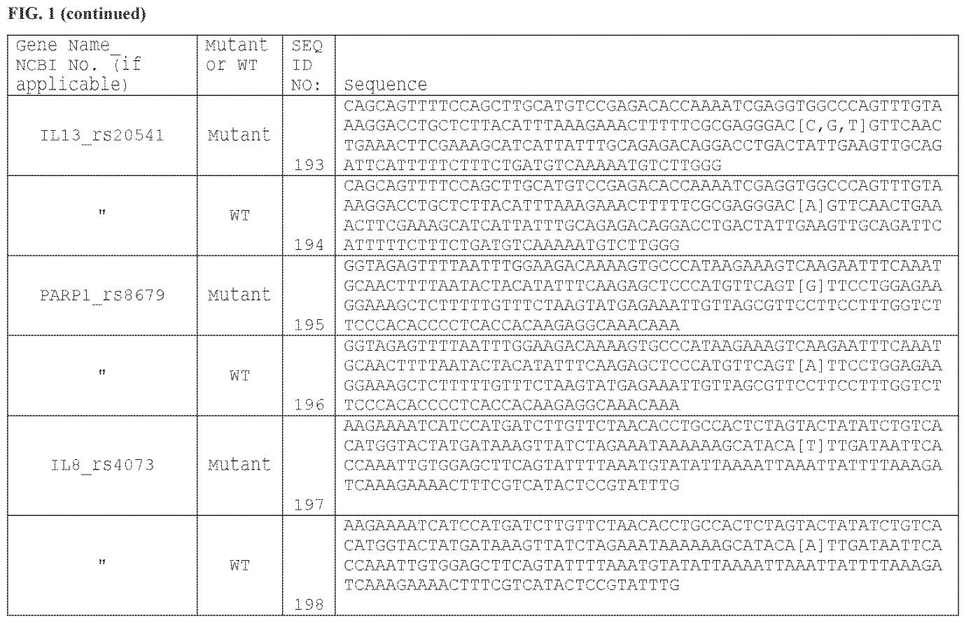

As described in the examples herein, several mutations have been identified as being pertinent to determining a cancer patient's likelihood of exhibiting a toxic response to a radiation treatment or to having an increased risk for mortality, distant failure or metastasis, or impotency. These mutations (also referred to herein as “markers,” “biomarkers,” or “variants”) are shown in SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 135, 137, 139, 141, 143, 145, 147, 149, 151, 153, 155, 157, 159, 161, 163, 165, 167, 169, 171, 173, 175, and 177 of FIG. 1 as the nucleotides in square brackets.

As the skilled artisan recognizes, double-stranded DNA comprises two strands of DNA, wherein each nucleotide of one strand is paired with its complementary nucleotide on the opposite strand. According to Watson-Crick base pairing for DNA, Adenine (A) pairs with Thymine (T), Thymine (T) pairs with Adenine (A), Guanine (G) pairs with Cytosine (C), and Cytosine (C) pairs with Guanine (G). DNA sequences are typically represented with an upper strand sequence oriented in a 5′ to 3′ direction, and a complementary lower strand oriented in a 3′ to 5′ direction. The 5′ to 3′ sequence of a complementary strand of DNA (e.g., a lower strand sequence) can be determined by reversing the direction of the sequence (e.g., an upper strand sequence) and interchanging each nucleotide with its complementary nucleotide, i.e., generating the reverse complement. Accordingly, in the following embodiments, it is understood that where a patient is identified as carrying or not carrying a mutation by detecting a variant allele occurring at position 101 of a given SEQ ID NO., that variant allele can equally be detected by detecting the complementary nucleotide of the variant allele on the opposite strand, i.e., the complementary nucleotide in the reverse complement sequence of that SEQ ID NO.

For example, where a C nucleotide at a position corresponding to position 101 of SEQ ID NO: 1 (miR.99a.promoter) can be detected, a G nucleotide at a corresponding position of the reverse complement sequence of SEQ ID NO: 1 can alternatively be detected. Stated another way, a C nucleotide at a position corresponding to position 101 of SEQ ID NO: 1 can be detected by detecting a G nucleotide at a corresponding position of the reverse complement sequence of SEQ ID NO: 1. As is understood by the skilled person, for each variant provided herein, the variant can alternatively be detected by detecting the complementary nucleotide at a corresponding position of the reverse complement sequence in which the variant is present.

The SNPs described herein can be detected to determine whether a cancer patient is likely to have a toxic response to radiation, or to having an increased risk for mortality, distant failure or metastasis, or impotency. Accordingly, the disclosure relates in part to a method for determining whether a cancer patient is likely to have a toxic response to radiation, or to having an increased risk for mortality, distant failure or metastasis, or impotency, the method comprising detecting a SNPs described herein, wherein the detection of a mutant (variant) or wild-type allele of a given SNP associated with a given outcome (e.g., a toxic response to radiation, or to having an increased risk for mortality, distant failure or metastasis, or impotency) is indicative of the patient's likelihood of having a toxic response to radiation, increased risk of mortality, increased risk of distant failure or metastasis, or increased risk of impotency.

The disclosure further relates to a method (e.g., a reduced toxicity method) of treating a cancer patient with radiation, the method comprising administering radiation to a patient, wherein the patient has been determined to carry one or more copies of a mutant (variant) or wild-type allele of a SNP described herein, wherein mutant (variant) or wild-type allele is associated with a lower risk of a toxic response, mortality, distant failure or metastasis, or impotency. In certain embodiments, the radiation is hypofractionated radiation, stereotactic body radiation treatment (SBRT), or traditional radiation. In certain embodiments, the cancer is sarcoma. In certain embodiments, the toxic response is wound toxicity. In certain embodiments, the cancer is prostate cancer. In certain embodiments, the toxic response is acute toxicity, late toxicity, gastrointestinal (GI) toxicity, or genitourinary toxicity (GU).

A biomarker relevant to determining a cancer patient's likelihood of having a toxic response to radiation is found in the promoter region of the human miR99a gene (“miR.99a.promoter”). In FIG. 1, 100 nucleotides upstream (5′) of the mutation and 100 nucleotides downstream (3′) of the mutation are shown. The mutation is a C nucleotide (variant) substituted in place of a T nucleotide (wild-type). The mutation occurs at position 101 of SEQ ID NO: 1 (variant sequence) or SEQ ID NO: 2 (wild-type sequence). In one embodiment, a patient identified as carrying this mutation has an increased likelihood of having a toxic response (e.g., wound toxicity) to radiation (e.g., hypofractionated radiation), whereas a patient identified as not carrying the mutation has a decreased likelihood of having a toxic response to radiation. In another embodiment, a patient who does not carry the mir99a mutation or who is heterozygous for the mir99a mutation has a decreased likelihood of having a toxic response to radiation as compared to a patient who is homozygous for the mir99a mutation, whereas a patient who is homozygous for the mir99a mutation has an increased likelihood of having a toxic response to radiation as compared to a patient who does not carry the mir99a mutation or who is heterozygous for the mir99a mutation. In certain embodiments, the cancer is sarcoma.

Another biomarker relevant to determining a cancer patient's likelihood of having a toxic response to radiation is the rs4848306 SNP found in the promoter region of the human interleukin 1beta (IL1B) gene (“IL1.B_rs4848306”). In FIG. 1, 100 nucleotides upstream (5′) of the mutation and 100 nucleotides downstream (3′) of the mutation are shown. The mutation is an A nucleotide (variant) substituted in place of a G nucleotide (wild-type). The mutation occurs at position 101 of SEQ ID NO: 3 (variant sequence) or SEQ ID NO: 4 (wild-type sequence). In one embodiment, a patient identified as carrying this mutation has an increased likelihood of having a toxic response (e.g., wound toxicity, short-term (acute) and/or long-term (late)) to radiation, whereas a patient identified as not carrying the mutation has a decreased likelihood of having a toxic response to radiation. In another embodiment, a patient who does not carry the IL1.B_rs4848306 variant or who is heterozygous for the IL1.B_rs4848306 variant has a decreased likelihood of having a toxic response to radiation as compared to a patient who is homozygous for the IL1.B_rs4848306 variant, whereas a patient who is homozygous for the IL1.B_rs4848306 mutation has an increased likelihood of having a toxic response to radiation as compared to a patient who does not carry the IL1.B_rs4848306 mutation or who is heterozygous for the IL1.B_rs4848306 mutation. In certain embodiments, the cancer is sarcoma. In certain embodiments, the cancer is prostate cancer.

Another biomarker relevant to determining a cancer patient's likelihood of having a toxic response to radiation is the rs1062124 SNP found in the 3′ UTR region of the human SHC adaptor protein 4 gene (“SHC4_rs1062124”). In FIG. 1, 100 nucleotides upstream (5′) of the mutation and 100 nucleotides downstream (3′) of the mutation are shown. The mutation is a G nucleotide (variant) substituted in place of an A nucleotide (wild-type). The mutation occurs at position 101 of SEQ ID NO: 5 (variant sequence) or SEQ ID NO: 6 (wild-type sequence). In one embodiment, a patient identified as carrying this mutation has an increased likelihood of having a toxic response (e.g., wound toxicity) to radiation, whereas a patient identified as not carrying the mutation has a decreased likelihood of having a toxic response to radiation. In another embodiment, a patient who does not carry the SHC4_rs1062124 mutation or who is heterozygous for the SHC4_rs1062124 mutation has a decreased likelihood of having a toxic response to radiation as compared to a patient who is homozygous for the SHC4_rs1062124 mutation, whereas a patient who is homozygous for the SHC4_rs1062124 mutation has an increased likelihood of having a toxic response to radiation as compared to a patient who does not carry the SHC4_rs1062124 mutation or who is heterozygous for the SHC4_rs1062124 mutation. In some embodiments, the SHC4_rs1062124 SNP is associated with distant failure. In certain embodiments, the cancer is sarcoma.

Another biomarker relevant to determining a cancer patient's likelihood of having a toxic response to radiation is the rs17599026 SNP found in an intronic region of the human lysine demethylase 3B (KDM3B) gene (“rs17599026”). In FIG. 1, 100 nucleotides upstream (5′) of the mutation and 100 nucleotides downstream (3′) of the mutation are shown. The mutation is a T nucleotide (variant) substituted in place of a C nucleotide (wild-type). The mutation occurs at position 101 of SEQ ID NO: 7 (variant sequence) or SEQ ID NO: 8 (wild-type sequence). In one embodiment, a patient identified as carrying this mutation (either as a heterozygote or homozygote) has a decreased likelihood of having a toxic response (e.g., wound toxicity and/or long-term (late)) to radiation, whereas a patient identified as not carrying the mutation has an increased likelihood of having a toxic response to radiation. In certain embodiments, the cancer is sarcoma. In certain embodiments, the cancer is prostate cancer.

Another biomarker relevant to determining a cancer patient's likelihood of having a toxic response to radiation, biochemical relapse and/or distant metastasis is the rs9374 SNP found in the 3′ UTR of the human Rac family small GTPase 1 gene (“RAC1_rs9374”). In FIG. 1, 100 nucleotides upstream (5′) of the mutation and 100 nucleotides downstream (3′) of the mutation are shown. The mutation is an A nucleotide (variant) substituted in place of a G nucleotide (wild-type). The mutation occurs at position 101 of SEQ ID NO: 9 (or SEQ ID NO: 117) (variant sequence) or SEQ ID NO: 10 (or SEQ ID NO: 118) (wild-type sequence). In one embodiment, a patient identified as carrying this mutation has an increased likelihood of having a toxic response (e.g., wound toxicity and/or short-term (acute)) to radiation, whereas a patient identified as not carrying the mutation has a decreased likelihood of having a toxic response to radiation. In another embodiment, a patient who does not carry the RAC1_rs9374 mutation or who is heterozygous for the RAC1_rs9374 mutation has a decreased likelihood of having a toxic response to radiation as compared to a patient who is homozygous for the RAC1_rs9374 mutation, whereas a patient who is homozygous for the RAC1_rs9374 mutation has an increased likelihood of having a toxic response to radiation as compared to a patient who does not carry the RAC1_rs9374 mutation or who is heterozygous for the RAC1_rs9374 mutation. In one embodiment, a patient identified as carrying the RAC1_rs9374 mutation has an increased likelihood of biochemical relapse, whereas a patient identified as not carrying the mutation has a decreased likelihood of biochemical relapse. In one embodiment, a patient identified as carrying the RAC1_rs9374 mutation has an increased likelihood of distant metastasis, whereas a patient identified as not carrying the mutation has a decreased likelihood of distant metastasis. SNP In certain embodiments, the cancer is sarcoma. In certain embodiments, the cancer is prostate cancer.

Another biomarker relevant to determining a cancer patient's likelihood of having a toxic response to radiation is the rs1150258 SNP found in the human interleukin 24 (IL24) gene (“IL24_rs1150258”). In FIG. 1, 100 nucleotides upstream (5′) of the mutation and 100 nucleotides downstream (3′) of the mutation are shown. The mutation is a C nucleotide (variant) substituted in place of a T nucleotide (wild-type). The mutation occurs at position 101 of SEQ ID NO: 11 (variant sequence) or SEQ ID NO: 12 (wild-type sequence). In one embodiment, a patient identified as carrying the mutation has an increased likelihood of having a toxic response (e.g., wound toxicity) to radiation, whereas a patient identified as not carrying the mutation has a decreased likelihood of having a toxic response to radiation. In another embodiment, a patient who does not carry the IL24_rs1150258 mutation or who is heterozygous for the IL24_rs1150258 mutation has a decreased likelihood of having a toxic response to radiation as compared to a patient who is homozygous for the IL24_rs1150258 mutation, whereas a patient who is homozygous for the IL24_rs1150258 mutation has an increased likelihood of having a toxic response to radiation as compared to a patient who does not carry the IL24_rs1150258 mutation or who is heterozygous for the IL24_rs1150258 mutation. In certain embodiments, the cancer is sarcoma.

Another biomarker relevant to determining a cancer patient's likelihood of having a toxic response to radiation is the rs1800587 SNP found in the promoter region of the human interleukin 1 alpha (IL1A) gene (“IL1A_rs1800587”). In FIG. 1, 100 nucleotides upstream (5′) of the mutation and 100 nucleotides downstream (3′) of the mutation are shown. The mutation is an A nucleotide (variant) substituted in place of a G nucleotide (wild-type). The mutation occurs at position 101 of SEQ ID NO: 13 (variant sequence) or SEQ ID NO: 14 (wild-type sequence). In one embodiment, a patient identified as carrying the IL1A_rs1800587 mutation (A nucleotide) has an increased likelihood of having a toxic response (e.g., wound toxicity) to radiation, whereas a patient identified as not carrying the mutation has a decreased likelihood of having a toxic response to radiation. In another embodiment, a patient who does not carry the IL1A_rs1800587 mutation or who is heterozygous for the IL1A_rs1800587 mutation has a decreased likelihood of having a toxic response to radiation as compared to a patient who is homozygous for the IL1A_rs1800587 mutation, whereas a patient who is homozygous for the IL1A_rs1800587 mutation has an increased likelihood of having a toxic response to radiation as compared to a patient who does not carry the IL1A_rs1800587 mutation or who is heterozygous for the IL1A_rs1800587 mutation. In some embodiments, the cancer patient has sarcoma. In one embodiment, a patient identified as not carrying the IL1A_rs1800587 mutation has an increased likelihood of having a toxic response (e.g., short-term (acute)) to radiation, whereas a patient identified as carrying the mutation has a decreased likelihood of having a toxic response to radiation. In some embodiments, the cancer patient has prostate cancer. In one embodiment, a prostate cancer patient identified as not carrying the IL1A_rs1800587 mutation has an increased likelihood of having a toxic response to radiation, whereas a prostate cancer patient identified as carrying the mutation has a decreased likelihood of having a toxic response to radiation.

Another biomarker relevant to determining a cancer patient's likelihood of having a toxic response to radiation is the rs3212948 SNP found in an intronic region of the human ERCC excision repair 1, endonuclease non-catalytic subunit gene (“ERCC1_rs3212948”). In FIG. 1, 100 nucleotides upstream (5′) of the mutation and 100 nucleotides downstream (3′) of the mutation are shown. The mutation is a C nucleotide (variant) substituted in place of a G nucleotide (wild-type). The mutation occurs at position 101 of SEQ ID NO: 15 (variant sequence) or SEQ ID NO: 16 (wild-type sequence). In one embodiment, a patient identified as not carrying this mutation (i.e., homozygous wild type) has a decreased likelihood of having a toxic response (e.g., wound toxicity) to radiation. In certain embodiments, the cancer is sarcoma.

Another biomarker relevant to determining a cancer patient's likelihood of having a toxic response to radiation or exhibiting distant failure is the rs12700386 SNP found near the human interleukin-6 (IL-6) gene (“IL.6_rs12700386”). In FIG. 1, 100 nucleotides upstream (5′) of the mutation and 100 nucleotides downstream (3′) of the mutation are shown. The mutation is a G nucleotide (variant) substituted in place of a C nucleotide (wild-type). The mutation occurs at position 101 of SEQ ID NO: 17 (variant sequence) or SEQ ID NO: 18 (wild-type sequence). In one embodiment, a patient identified as carrying the mutation has an increased likelihood of having a toxic response to radiation (e.g., wound toxicity and/or short-term (acute toxicity), whereas a patient identified as not carrying the mutation has a decreased likelihood of having a toxic response to radiation. In another embodiment, a patient who does not carry the IL.6_rs12700386 mutation or who is heterozygous for the IL.6_rs12700386 mutation has a decreased likelihood of having a toxic response to radiation as compared to a patient who is homozygous for the IL.6_rs12700386 mutation, whereas a patient who is homozygous for the IL.6_rs12700386 mutation has an increased likelihood of having a toxic response to radiation as compared to a patient who does not carry the IL.6_rs12700386 mutation or who is heterozygous for the IL.6_rs12700386 mutation. In certain embodiments, a patient identified as carrying the mutation (G) has an increased likelihood of exhibiting distant failure, whereas a patient identified as not carrying the mutation has a decreased likelihood of exhibiting distant failure. SNP In certain embodiments, the cancer is sarcoma. In certain embodiments, the cancer is prostate cancer.

Another biomarker relevant to determining a cancer patient's likelihood of having a toxic response to radiation, exhibiting distant failure, or is the rs2069840 SNP found near the human interleukin-6 (IL-6) gene (“IL.6_rs2069840”). In FIG. 1, 100 nucleotides upstream (5′) of the mutation and 100 nucleotides downstream (3′) of the mutation are shown. The mutation is a G nucleotide (variant) substituted in place of a C nucleotide (wild-type). The mutation occurs at position 101 of SEQ ID NO: 19 (variant sequence) or SEQ ID NO: 20 (wild-type sequence). In one embodiment, a patient identified as carrying this mutation has an increased likelihood of having a toxic response to radiation, whereas a patient identified as not carrying the mutation has a decreased likelihood of having a toxic response (e.g., wound toxicity) to radiation. In another embodiment, a patient who does not carry the IL.6_rs2069840 mutation or who is heterozygous for the IL.6_rs2069840 mutation has a decreased likelihood of having a toxic response to radiation as compared to a patient who is homozygous for the IL.6_rs2069840 mutation, whereas a patient who is homozygous for the IL.6_rs2069840 mutation has an increased likelihood of having a toxic response to radiation as compared to a patient who does not carry the IL.6_rs2069840 mutation or who is heterozygous for the IL.6_rs2069840 mutation. In certain embodiments, a patient identified as being heterozygous for the mutation has a decreased likelihood of exhibiting distant failure, whereas a patient identified as being homozygous for the mutation has an increased likelihood of exhibiting distant failure. In certain embodiments, a patient identified as carrying this mutation has an increased likelihood of impotency following radiation treatment as compared to a patient identified as not carrying the mutation.

Another biomarker relevant to determining a cancer patient's likelihood of having a toxic response to radiation is the rs2306327 SNP found in the human calcium/calmodulin dependent protein kinase II gamma (CAMK2G) gene (“CAMK2G_rs2306327”). In FIG. 1, 100 nucleotides upstream (5′) of the mutation and 100 nucleotides downstream (3′) of the mutation are shown. The mutation is a T nucleotide (variant) substituted in place of an A nucleotide (wild-type). The mutation occurs at position 101 of SEQ ID NO: 21 (variant sequence) or SEQ ID NO: 22 (wild-type sequence). In one embodiment, a patient identified as carrying this mutation has an increased likelihood of having a toxic response (e.g., wound toxicity) to radiation, whereas a patient identified as not carrying the mutation has a decreased likelihood of having a toxic response to radiation. In another embodiment, a patient who does not carry the CAMK2G_rs2306327 variant or who is heterozygous for the CAMK2G_rs2306327 mutation has a decreased likelihood of having a toxic response to radiation as compared to a patient who is homozygous for the CAMK2G_rs2306327 mutation, whereas a patient who is homozygous for the CAMK2G_rs2306327 mutation has an increased likelihood of having a toxic response to radiation as compared to a patient who does not carry the CAMK2G_rs2306327 mutation or who is heterozygous for the CAMK2G_rs2306327 mutation. In certain embodiments, the cancer is sarcoma.

Another biomarker relevant to determining a cancer patient's likelihood of having a toxic response to radiation is the rs1980499 SNP found upstream of the human bone morphogenetic protein 2 (BMP2) gene (“BMP2_rs1980499”). In FIG. 1, 100 nucleotides upstream (5′) of the mutation and 100 nucleotides downstream (3′) of the mutation are shown. The mutation is a T nucleotide (variant) substituted in place of a C nucleotide (wild-type). The mutation occurs at position 101 of SEQ ID NO: 23 (variant sequence) or SEQ ID NO: 24 (wild-type sequence). In one embodiment, a patient identified as carrying the mutation (T) has an increased likelihood of having a toxic response (e.g., wound toxicity) to radiation, whereas a patient identified as not carrying the mutation has a decreased likelihood of having a toxic response to radiation. In another embodiment, a patient who does not carry the BMP2_rs1980499 mutation or who is heterozygous for the BMP2_rs1980499 mutation has a decreased likelihood of having a toxic response to radiation as compared to a patient who is homozygous for the BMP2_rs1980499 mutation, whereas a patient who is homozygous for the BMP2_rs1980499 mutation has an increased likelihood of having a toxic response to radiation as compared to a patient who does not carry the BMP2_rs1980499 mutation or who is heterozygous for the BMP2_rs1980499 mutation. In certain embodiments, the cancer is sarcoma.

Another biomarker relevant to determining a cancer patient's likelihood of having a toxic response to radiation is the rs1460008 SNP found in the 3′ UTR region of the human Epiregulin (EREG) gene (“EREG_rs1460008”). In FIG. 1, 100 nucleotides upstream (5′) of the mutation and 100 nucleotides downstream (3′) of the mutation are shown. The mutation is a G nucleotide (variant) substituted in place of an A nucleotide (wild-type). The mutation occurs at position 101 of SEQ ID NO: 25 (variant sequence) or SEQ ID NO: 26 (wild-type sequence). In one embodiment, a patient identified as carrying this mutation has an increased likelihood of having a toxic response (e.g., wound toxicity) to radiation, whereas a patient identified as not carrying the mutation has a decreased likelihood of having a toxic response to radiation. In another embodiment, a patient who does not carry the EREG_rs1460008 mutation or who is heterozygous for the EREG_rs1460008 mutation has a decreased likelihood of having a toxic response to radiation as compared to a patient who is homozygous for the EREG_rs1460008 mutation, whereas a patient who is homozygous for the EREG_rs1460008 mutation has an increased likelihood of having a toxic response to radiation as compared to a patient who does not carry the EREG_rs1460008 mutation or who is heterozygous for the EREG_rs1460008 mutation. In certain embodiments, the cancer is sarcoma.

Another biomarker relevant to determining a cancer patient's likelihood of having a toxic response to radiation or exhibiting distant failure is the rs4143815 SNP found in the human CD274 gene (“CD274_rs4143815”). In FIG. 1, 100 nucleotides upstream (5′) of the mutation and 100 nucleotides downstream (3′) of the mutation are shown. The mutation is a C nucleotide (variant) substituted in place of a G nucleotide (wild-type). The mutation occurs at position 101 of SEQ ID NO: 27 (variant sequence) or SEQ ID NO: 28 (wild-type sequence). In one embodiment, a patient identified as carrying this mutation has an increased likelihood of having a toxic response to radiation (e.g., wound toxicity or long-term (late)), whereas a patient identified as not carrying the mutation has a decreased likelihood of having a toxic response to radiation. In another embodiment, a patient who does not carry the CD274_rs4143815 mutation or who is heterozygous for the CD274_rs4143815 mutation has a decreased likelihood of having a toxic response to radiation as compared to a patient who is homozygous for the CD274_rs4143815 mutation, whereas a patient who is homozygous for the CD274_rs4143815 mutation has an increased likelihood of having a toxic response to radiation as compared to a patient who does not carry the CD274_rs4143815 mutation or who is heterozygous for the CD274_rs4143815 mutation. In some embodiments, the rs4143815 SNP is associated with distant failure. In certain embodiments, a patient identified as carrying the mutation (C) has an increased likelihood of exhibiting distant failure, whereas a patient identified as not carrying the mutation has a decreased likelihood of exhibiting distant failure. In certain embodiments, the cancer is sarcoma. In certain embodiments, the cancer is prostate cancer.

Another biomarker relevant to determining a cancer patient's likelihood of having a toxic response to radiation is the rs7787 SNP found in the 3′ UTR of the human interleukin 17D (IL17D) gene (“IL17D_rs7787”). In FIG. 1, 100 nucleotides upstream (5′) of the mutation and 100 nucleotides downstream (3′) of the mutation are shown. The mutation is an A nucleotide (variant) substituted in place of a T nucleotide (wild-type). The mutation occurs at position 101 of SEQ ID NO: 29 (variant sequence) or SEQ ID NO: 30 (wild-type sequence). In one embodiment, a patient identified as carrying this mutation has an increased likelihood of having a toxic response (e.g., wound toxicity and/or long-term (late)) to radiation, whereas a patient identified as not carrying the mutation has a decreased likelihood of having a toxic response to radiation. In another embodiment, a patient who does not carry the IL17D_rs7787 mutation or who is heterozygous for the IL17D_rs7787 mutation has a decreased likelihood of having a toxic response (e.g., long-term (late)) to radiation as compared to a patient who is homozygous for the IL17D_rs7787 mutation, whereas a patient who is homozygous for the IL17D_rs7787 mutation has an increased likelihood of having a toxic response to radiation as compared to a patient who does not carry the IL17D_rs7787 mutation or who is heterozygous for the IL17D_rs7787 mutation. In one embodiment, a patient identified as not carrying the IL17D_rs7787 mutation (T nucleotide) has an increased likelihood of biochemical relapse, whereas a patient identified as carrying the mutation (A nucleotide) has a decreased likelihood of biochemical relapse. In certain embodiments, the cancer is sarcoma. In certain embodiments, the cancer is prostate cancer.

Another biomarker relevant to determining a cancer patient's likelihood of having a toxic response to radiation or biochemical relapse following a radiation treatment is the rs3744483SNP found in the 3′ UTR of the human signal transducer and activator of transcription 3 (STAT3) gene (“STAT3_rs3744483”). In FIG. 1, 100 nucleotides upstream (5′) of the mutation and 100 nucleotides downstream (3′) of the mutation are shown. The mutation is a C nucleotide (variant) substituted in place of a T nucleotide (wild-type). The mutation occurs at position 101 of SEQ ID NO: 31 (variant sequence) or SEQ ID NO: 32 (wild-type sequence). In one embodiment, a patient identified as carrying this mutation has an increased likelihood of having a toxic response (e.g., wound toxicity and/or short-term (acute)) to radiation, whereas a patient identified as not carrying the mutation has a decreased likelihood of having a toxic response to radiation. In another embodiment, a patient who does not carry the STAT3_rs3744483 mutation or who is heterozygous for the STAT3_rs3744483 mutation has a decreased likelihood of having a toxic response to radiation as compared to a patient who is homozygous for the STAT3_rs3744483 mutation, whereas a patient who is homozygous for the STAT3_rs3744483 mutation has an increased likelihood of having a toxic response to radiation as compared to a patient who does not carry the STAT3_rs3744483 mutation or who is heterozygous for the STAT3_rs3744483 mutation. In one embodiment, a patient identified as not carrying the STAT3_rs3744483 mutation has an increased likelihood of biochemical relapse, whereas a patient identified as carrying the mutation has a decreased likelihood of biochemical relapse. In certain embodiments, the cancer is sarcoma. In certain embodiments, the cancer is prostate cancer.

Another biomarker relevant to determining a cancer patient's likelihood of having a toxic response to radiation is the rs17563 SNP found in the human bone morphogenetic protein 4 (BMP4) gene (“BMP4_rs17563”). In FIG. 1, 100 nucleotides upstream (5′) of the mutation and 100 nucleotides downstream (3′) of the mutation are shown. The mutation is a G nucleotide (variant) substituted in place of an A nucleotide (wild-type). The mutation occurs at position 101 of SEQ ID NO: 33 (variant sequence) or SEQ ID NO: 34 (wild-type sequence). In one embodiment, a patient identified as carrying this mutation has an increased likelihood of having a toxic response (e.g., wound toxicity) to radiation, whereas a patient identified as not carrying the mutation has a decreased likelihood of having a toxic response to radiation. In another embodiment, a patient who does not carry the BMP4_rs17563 mutation or who is heterozygous for the BMP4_rs17563 mutation has a decreased likelihood of having a toxic response to radiation as compared to a patient who is homozygous for the BMP4_rs17563 mutation, whereas a patient who is homozygous for the BMP4_rs17563 mutation has an increased likelihood of having a toxic response to radiation as compared to a patient who does not carry the BMP4_rs17563 mutation or who is heterozygous for the BMP4_rs17563 mutation. In certain embodiments, the cancer is sarcoma.

Another biomarker relevant to determining a cancer patient's likelihood of having a toxic response to radiation is the rs10488631 SNP found in the interferon regulatory factor 5 (IRF5) gene (“IRF5_rs10488631”). In FIG. 1, 100 nucleotides upstream (5′) of the mutation and 100 nucleotides downstream (3′) of the mutation are shown. The mutation is a C nucleotide (variant) substituted in place of a T nucleotide (wild-type). The mutation occurs at position 101 of SEQ ID NO: 35 (variant sequence) or SEQ ID NO: 36 (wild-type sequence). In one embodiment, a patient identified as carrying this mutation has an increased likelihood of having a toxic response (e.g., wound toxicity) to radiation, whereas a patient identified as not carrying the mutation has a decreased likelihood of having a toxic response to radiation. In certain embodiments, the cancer is sarcoma.

Another biomarker relevant to determining a cancer patient's likelihood of having a toxic response to radiation is the UNGC.41.IL1RAP SNP found in the non-coding region of the human ILRAP gene (“UNGC.41.IL1RAP”). In FIG. 1, 100 nucleotides upstream (5′) of the mutation and 100 nucleotides downstream (3′) of the mutation are shown. The mutation is a T nucleotide (variant) substituted in place of a C nucleotide (wild-type). The mutation occurs at position 101 of SEQ ID NO: 37 (variant sequence) or SEQ ID NO: 38 (wild-type sequence). In one embodiment, a patient identified as carrying this mutation has an increased likelihood of having a toxic response (e.g., wound toxicity) to radiation, whereas a patient identified as not carrying the mutation has a decreased likelihood of having a toxic response to radiation. In another embodiment, a patient who does not carry the UNGC.41.IL1RAP mutation or who is heterozygous for the UNGC.41.IL1RAP mutation has a decreased likelihood of having a toxic response to radiation as compared to a patient who is homozygous for the UNGC.41.IL1RAP mutation, whereas a patient who is homozygous for the UNGC.41.IL1RAP mutation has an increased likelihood of having a toxic response to radiation as compared to a patient who does not carry the UNGC.41.IL1RAP mutation or who is heterozygous for the UNGC.41.IL1RAP mutation. In certain embodiments, the cancer is sarcoma.

Another biomarker relevant to determining a cancer patient's likelihood of having a toxic response to radiation is the rs1798 SNP found in the 3′ UTR of the human interleukin-19 (IL19) gene (“IL19_rs1798”). In FIG. 1, 100 nucleotides upstream (5′) of the mutation and 100 nucleotides downstream (3′) of the mutation are shown. The mutation is a G nucleotide (variant) substituted in place of a C nucleotide (wild-type). The mutation occurs at position 101 of SEQ ID NO: 39 (variant sequence) or SEQ ID NO: 40 (wild-type sequence). In one embodiment, a patient identified as carrying this mutation has an increased likelihood of having a toxic response (e.g., long-term (late)) to radiation, whereas a patient identified as not carrying the mutation has a decreased likelihood of having a toxic response to radiation. In another embodiment, a patient who does not carry the IL19_rs1798 mutation or who is heterozygous for the IL19_rs1798 mutation has a decreased likelihood of having a toxic response to radiation as compared to a patient who is homozygous for the IL19_rs1798 mutation, whereas a patient who is homozygous for the IL19_rs1798 mutation has an increased likelihood of having a toxic response to radiation as compared to a patient who does not carry the IL19_rs1798 mutation or who is heterozygous for the IL19_rs1798 mutation. In certain embodiments, the cancer is prostate cancer.

Another biomarker relevant to determining a cancer patient's likelihood of having a toxic response to radiation is the rs8240 SNP found in the human RAD23 homolog A, nucleotide excision repair protein gene (“RAD23A_rs8240”). In FIG. 1, 100 nucleotides upstream (5′) of the mutation and 100 nucleotides downstream (3′) of the mutation are shown. The mutation is an A nucleotide (variant) substituted in place of a G nucleotide (wild-type). The mutation occurs at position 101 of SEQ ID NO: 41 (variant sequence) or SEQ ID NO: 42 (wild-type sequence). In one embodiment, a patient identified as carrying this mutation has an increased likelihood of having a toxic response (e.g., long-term (late)) to radiation, whereas a patient identified as not carrying the mutation has a decreased likelihood of having a toxic response to radiation. In another embodiment, a patient who does not carry the RAD23A_rs8240 mutation or who is heterozygous for the RAD23A_rs8240 mutation has a decreased likelihood of having a toxic response to radiation as compared to a patient who is homozygous for the RAD23A_rs8240 mutation, whereas a patient who is homozygous for the RAD23A_rs8240 mutation has an increased likelihood of having a toxic response to radiation as compared to a patient who does not carry the RAD23A_rs8240 mutation or who is heterozygous for the RAD23A_rs8240 mutation. In certain embodiments, the cancer is prostate cancer.

Another biomarker relevant to determining a cancer patient's likelihood of having a toxic response to radiation is the rs15869 SNP found in the 3′ UTR of the human BRCA2 DNA repair associated gene (“BRCA2_rs15869”). In FIG. 1, 100 nucleotides upstream (5′) of the mutation and 100 nucleotides downstream (3′) of the mutation are shown. The mutation is a C nucleotide (variant) substituted in place of an A nucleotide (wild-type). The mutation occurs at position 101 of SEQ ID NO: 43 (variant sequence) or SEQ ID NO: 44 (wild-type sequence). In one embodiment, a patient identified as carrying this mutation has an increased likelihood of having a toxic response (e.g., long-term (late)) to radiation, whereas a patient identified as not carrying the mutation has a decreased likelihood of having a toxic response to radiation. In another embodiment, a patient who does not carry the BRCA2_rs15869 mutation or who is heterozygous for the BRCA2_rs15869 mutation has a decreased likelihood of having a toxic response to radiation as compared to a patient who is homozygous for the BRCA2_rs15869 mutation, whereas a patient who is homozygous for the BRCA2_rs15869 mutation has an increased likelihood of having a toxic response to radiation as compared to a patient who does not carry the BRCA2_rs15869 mutation or who is heterozygous for the BRCA2_rs15869 mutation. In certain embodiments, the cancer is prostate cancer. In certain embodiments, the radiation is stereotactic or traditional radiation.

Another biomarker relevant to determining a cancer patient's likelihood of having a toxic response to radiation or to experiencing a shorter time to biochemical relapse is the rs2282055 SNP found in an intronic region of the human CD274 gene (“CD274_rs2282055”). In FIG. 1, 100 nucleotides upstream (5′) of the mutation and 100 nucleotides downstream (3′) of the mutation are shown. The mutation is a G nucleotide (variant) substituted in place of a T nucleotide (wild-type). The mutation occurs at position 101 of SEQ ID NO: 45 (variant sequence) or SEQ ID NO: 46 (wild-type sequence). In one embodiment, a patient identified as carrying this mutation has an increased likelihood of having a toxic response (e.g., long-term (late)) to radiation, whereas a patient identified as not carrying the mutation has a decreased likelihood of having a toxic response to radiation. In another embodiment, a patient who does not carry the CD274_rs2282055 mutation or who is heterozygous for the CD274_rs2282055 mutation has a decreased likelihood of having a toxic response to radiation as compared to a patient who is homozygous for the CD274_rs2282055 mutation, whereas a patient who is homozygous for the CD274_rs2282055 mutation has an increased likelihood of having a toxic response to radiation as compared to a patient who does not carry the CD274_rs2282055 mutation or who is heterozygous for the CD274_rs2282055 mutation. In some embodiments, the CD274 rs2282055 SNP is associated with biochemical relapse. In one embodiment, a patient identified as carrying the CD274_rs2282055 mutation has an increased likelihood of biochemical relapse, whereas a patient identified as not carrying the mutation has a decreased likelihood biochemical relapse. In certain embodiments, the cancer is prostate cancer.

Another biomarker relevant to determining a cancer patient's likelihood of having a toxic response to radiation or likelihood of biochemical relapse following a radiation treatment is the rs3178250 SNP found in the 3′ UTR of the human bone morphogenetic protein (BMP2) gene (“BMP2_rs3178250”). In FIG. 1, 100 nucleotides upstream (5′) of the mutation and 100 nucleotides downstream (3′) of the mutation are shown. The mutation is a C nucleotide (variant) substituted in place of a T nucleotide (wild-type). The mutation occurs at position 101 of SEQ ID NO: 47 (variant sequence) or SEQ ID NO: 48 (wild-type sequence). In one embodiment, a patient identified as carrying this mutation has an increased likelihood of having a toxic response (e.g., long-term (late)) to radiation, whereas a patient identified as not carrying the mutation has a decreased likelihood of having a toxic response to radiation. In another embodiment, a patient who does not carry the BMP2_rs3178250 mutation or who is heterozygous for the BMP2_rs3178250 mutation has a decreased likelihood of having a toxic response to radiation as compared to a patient who is homozygous for the BMP2_rs3178250 mutation, whereas a patient who is homozygous for the BMP2_rs3178250 mutation has an increased likelihood of having a toxic response to radiation as compared to a patient who does not carry the BMP2_rs3178250 mutation or who is heterozygous for the BMP2_rs3178250 mutation. In certain embodiments, a patient who does not carry the BMP2_rs3178250 mutation has an decreased likelihood of biochemical relapse than does a patient who carries the BMP2_rs3178250 mutation. In certain embodiments, a patient who does carry the BMP2_rs3178250 mutation has an increased likelihood of biochemical relapse than does a patient who does not carry the BMP2_rs3178250 mutation. In certain embodiments, the cancer is prostate cancer.

Another biomarker relevant to determining a cancer patient's likelihood of having a toxic response to radiation and likelihood of biochemical relapse following radiation treatment is the rs1411262 SNP found in an intronic region of the human CD274 gene (“CD274_rs1411262”). In FIG. 1, 100 nucleotides upstream (5′) of the mutation and 100 nucleotides downstream (3′) of the mutation are shown. The mutation is a T nucleotide (variant) substituted in place of an C nucleotide (wild-type). The mutation occurs at position 101 of SEQ ID NO: 49 (variant sequence) or SEQ ID NO: 50 (wild-type sequence). In one embodiment, a patient identified as carrying this mutation has an increased likelihood of having a toxic response (e.g., long-term (late)) to radiation, whereas a patient identified as not carrying the mutation has a decreased likelihood of having a toxic response to radiation. In another embodiment, a patient who does not carry the CD274_rs1411262 mutation or who is heterozygous for the CD274_rs1411262 mutation has a decreased likelihood of having a toxic response to radiation as compared to a patient who is homozygous for the CD274_rs1411262 mutation, whereas a patient who is homozygous for the CD274_rs1411262 mutation has an increased likelihood of having a toxic response to radiation as compared to a patient who does not carry the CD274_rs1411262 mutation or who is heterozygous for the CD274_rs1411262 mutation. In some embodiments, the SNP is associated with biochemical relapse. In one embodiment, a patient identified as not carrying the mutation has an increased likelihood of biochemical relapse (time to biochemical relapse), whereas a patient identified as carrying the mutation has a decreased likelihood of biochemical relapse. In certain embodiments, the cancer is prostate cancer.

Another biomarker relevant to determining a cancer patient's likelihood of having a toxic response to radiation and likelihood of biochemical relapse following radiation treatment is the rs2297136 SNP found in an intronic region of the human CD274 gene (“CD274_rs2297136”). In FIG. 1, 100 nucleotides upstream (5′) of the mutation and 100 nucleotides downstream (3′) of the mutation are shown. The mutation is a G nucleotide (variant) substituted in place of an A nucleotide (wild-type). The mutation occurs at position 101 of SEQ ID NO: 51 (variant sequence) or SEQ ID NO: 52 (wild-type sequence). In one embodiment, a patient identified as carrying this mutation has an increased likelihood of having a toxic response (e.g., long-term (late)) to radiation, whereas a patient identified as not carrying the mutation has a decreased likelihood of having a toxic response to radiation. In one embodiment, a patient identified as not carrying the CD274_rs2297136 mutation (A nucleotide) has an increased likelihood of biochemical relapse, whereas a patient identified as carrying the mutation (G nucleotide) has a decreased likelihood of biochemical relapse. In certain embodiments, the cancer is prostate cancer.

Another biomarker relevant to determining a cancer patient's likelihood of having a toxic response to radiation or likelihood of biochemical relapse is the rs1979855 SNP found upstream of the human bone morphogenetic protein (BMP2) gene (“BMP2_rs1979855”). In FIG. 1, 100 nucleotides upstream (5′) of the mutation and 100 nucleotides downstream (3′) of the mutation are shown. The mutation is a G nucleotide (variant) substituted in place of an A nucleotide (wild-type). The mutation occurs at position 101 of SEQ ID NO: 53 (variant sequence) or SEQ ID NO: 54 (wild-type sequence). In one embodiment, a patient identified as carrying this mutation has an increased likelihood of having a toxic response (e.g., long-term (late)) to radiation, whereas a patient identified as not carrying the mutation has a decreased likelihood of having a toxic response to radiation. In another embodiment, a patient who does not carry the BMP2_rs1979855 mutation or who is heterozygous for the BMP2_rs1979855 mutation has a decreased likelihood of having a toxic response to radiation as compared to a patient who is homozygous for the BMP2_rs1979855 mutation, whereas a patient who is homozygous for the BMP2_rs1979855 mutation has an increased likelihood of having a toxic response to radiation as compared to a patient who does not carry the BMP2_rs1979855 mutation or who is heterozygous for the BMP2_rs1979855 mutation. In one embodiment, a patient identified as carrying the BMP2_rs1979855 mutation has an increased likelihood of biochemical relapse (shorter time until biochemical relapse), whereas a patient identified as not carrying the mutation has a decreased likelihood of biochemical relapse. In certain embodiments, the radiation is SBRT. In certain embodiments, the radiation is traditional radiation. In certain embodiments, the cancer is prostate cancer.

Another biomarker relevant to determining a cancer patient's likelihood of having a toxic response to radiation is the rs1893217 SNP found upstream of the human protein tyrosine phosphatase non-receptor type 2 (PTPN2) gene (“PTPN2_rs1893217”). In FIG. 1, 100 nucleotides upstream (5′) of the mutation and 100 nucleotides downstream (3′) of the mutation are shown. The mutation is a G nucleotide (variant) substituted in place of an A nucleotide (wild-type). The mutation occurs at position 101 of SEQ ID NO: 55 (variant sequence) or SEQ ID NO: 56 (wild-type sequence). In one embodiment, a patient identified as carrying this mutation has an increased likelihood of having a toxic response (e.g., long-term (late)) to radiation, whereas a patient identified as not carrying the mutation has a decreased likelihood of having a toxic response to radiation. In another embodiment, a patient who does not carry the PTPN2_rs1893217 mutation or who is heterozygous for the PTPN2_rs1893217 mutation has a decreased likelihood of having a toxic response to radiation as compared to a patient who is homozygous for the PTPN2_rs1893217 mutation, whereas a patient who is homozygous for the PTPN2_rs1893217 mutation has an increased likelihood of having a toxic response to radiation as compared to a patient who does not carry the PTPN2_rs1893217 mutation or who is heterozygous for the PTPN2_rs1893217 mutation. In certain embodiments, the cancer is prostate cancer.

Another biomarker relevant to determining a cancer patient's likelihood of having a toxic response to radiation and likelihood of biochemical relapse following a radiation treatment is the rs822339 SNP found in an intronic region of the human CD274 gene (“CD274_rs822339”). In FIG. 1, 100 nucleotides upstream (5′) of the mutation and 100 nucleotides downstream (3′) of the mutation are shown. The mutation is a G nucleotide (variant) substituted in place of an A nucleotide (wild-type). The mutation occurs at position 101 of SEQ ID NO: 57 (variant sequence) or SEQ ID NO: 58 (wild-type sequence). In one embodiment, a patient identified as not carrying this mutation has an increased likelihood of having a toxic response (e.g., long-term (late)) to radiation, whereas a patient identified as carrying the mutation has a decreased likelihood of having a toxic response to radiation. In another embodiment, a patient who carries the CD274_rs822339 mutation or who is heterozygous for the CD274_rs822339 mutation has a decreased likelihood of having a toxic response to radiation as compared to a patient who is homozygous wild-type for the CD274_rs822339 mutation, whereas a patient who is homozygous wild-type for the CD274_rs822339 mutation has an increased likelihood of having a toxic response to radiation as compared to a patient who carries the CD274_rs822339 mutation or who is heterozygous for the CD274_rs822339 mutation. In some embodiments, the CD274_rs822339 SNP is associated with biochemical relapse. In one embodiment, a patient identified as carrying the CD274_rs822339 mutation has a decreased likelihood of biochemical relapse, whereas a patient identified as not carrying the mutation has an increased likelihood of biochemical relapse. In certain embodiments, the cancer is prostate cancer.

Another biomarker relevant to determining a cancer patient's likelihood of having a toxic response to radiation is the rs6695772 SNP found upstream of the human basic leucine zipper ATF-like transcription factor 3 (BATF3) gene (“BATF3_rs6695772”). In FIG. 1, 100 nucleotides upstream (5′) of the mutation and 100 nucleotides downstream (3′) of the mutation are shown. The mutation is a G nucleotide (variant) substituted in place of a C nucleotide (wild-type). The mutation occurs at position 101 of SEQ ID NO: 59 (variant sequence) or SEQ ID NO: 60 (wild-type sequence). In one embodiment, a patient identified as not carrying this mutation has an increased likelihood of having a toxic response (e.g., long-term (late)) to radiation, whereas a patient identified as carrying the mutation has a decreased likelihood of having a toxic response to radiation. In another embodiment, a patient who carries the BATF3_rs6695772 mutation or who is heterozygous for the BATF3_rs6695772 mutation has an decreased likelihood of having a toxic response to radiation as compared to a patient who is homozygous wild-type for the BATF3_rs6695772 mutation, whereas a patient who is homozygous wild-type for the BATF3_rs6695772 mutation has an increased likelihood of having a toxic response to radiation as compared to a patient who carries the BATF3_rs6695772 mutation or who is heterozygous for the BATF3_rs6695772 mutation. In certain embodiments, the cancer is prostate cancer.

Another biomarker relevant to determining a cancer patient's likelihood of having a toxic response to radiation is the rs11465660 SNP found in the 3′ UTR of the interleukin-18 receptor 1 (IL18R1) gene (“IL18R1_rs11465660”). In FIG. 1, 100 nucleotides upstream (5′) of the mutation and 100 nucleotides downstream (3′) of the mutation are shown. The mutation is an A nucleotide (variant) substituted in place of a C nucleotide (wild-type). The mutation occurs at position 101 of SEQ ID NO: 61 (variant sequence) or SEQ ID NO: 62 (wild-type sequence). In one embodiment, a patient identified as carrying this mutation has an increased likelihood of having a toxic response (e.g., short-term (acute) and/or long-term (late)) to radiation, whereas a patient identified as not carrying the mutation has a decreased likelihood of having a toxic response to radiation. In another embodiment, a patient who does not carry the IL18R1_rs11465660 mutation or who is heterozygous for the IL18R1_rs11465660 mutation has a decreased likelihood of having a toxic response to radiation as compared to a patient who is homozygous for the IL18R1_rs11465660 mutation, whereas a patient who is homozygous for the IL18R1_rs11465660 mutation has an increased likelihood of having a toxic response to radiation as compared to a patient who does not carry the IL18R1_rs11465660 mutation or who is heterozygous for the IL18R1_rs11465660 mutation. In some embodiments, the rs11465660 SNP is associated with biochemical relapse. In certain embodiments, the cancer is prostate cancer.

Another biomarker relevant to determining a cancer patient's likelihood of having a toxic response to radiation is the rs11615 SNP found in the 3′ UTR of the ERCC excision repair 1, endonuclease non-catalytic subunit (ERCC1) gene (“ERCC1_rs11615”). In FIG. 1, 100 nucleotides upstream (5′) of the mutation and 100 nucleotides downstream (3′) of the mutation are shown. The mutation is a G nucleotide (variant) substituted in place of an A nucleotide (wild-type). The mutation occurs at position 101 of SEQ ID NO: 63 (variant sequence) or SEQ ID NO: 64 (wild-type sequence). In one embodiment, a patient identified as not carrying this mutation has an decreased likelihood of having a toxic response (e.g., long-term (late)) to radiation, whereas a patient identified as carrying the mutation has an increased likelihood of having a toxic response to radiation. In another embodiment, a patient who carries the ERCC1_rs11615 mutation has an increased likelihood of having a toxic response to radiation as compared to a patient who is heterozygous or homozygous wild-type for the ERCC1_rs11615 mutation, whereas a patient who is heterozygous or homozygous wild-type for the ERCC1_rs11615 mutation has an decreased likelihood of having a toxic response to radiation as compared to a patient who is homozygous mutant for ERCC1_rs11615. In some embodiments, the ERCC1_rs11615 SNP is associated with distant failure. In certain embodiments, the cancer is sarcoma. In certain embodiments, the cancer is prostate cancer.

Another biomarker relevant to determining a cancer patient's likelihood of having a toxic response to radiation is the rs11991 SNP found in the 3′ UTR of the ABL proto-oncogene 1, non-receptor tyrosine kinase (ABL1) gene (“ABL1_rs11991”). In FIG. 1, 100 nucleotides upstream (5′) of the mutation and 100 nucleotides downstream (3′) of the mutation are shown. The mutation is a C nucleotide (variant) substituted in place of a T nucleotide (wild-type). The mutation occurs at position 101 of SEQ ID NO: 65 (variant sequence) or SEQ ID NO: 66 (wild-type sequence). In one embodiment, a patient identified as carrying this mutation has a decreased likelihood of having a toxic response (e.g., long-term (late)) to radiation, whereas a patient identified as not carrying the mutation has an increased likelihood of having a toxic response to radiation. In another embodiment, a patient who does not carry the ABL1_rs11991 mutation has an increased likelihood of having a toxic response to radiation as compared to a patient who is heterozygous or homozygous for the ABL1_rs11991 mutation, whereas a patient who is heterozygous or homozygous for the ABL1_rs11991 mutation has a decreased likelihood of having a toxic response to radiation as compared to a patient who does not carry the ABL1_rs11991 mutation. In certain embodiments, the cancer is prostate cancer.

Another biomarker relevant to determining a cancer patient's likelihood of having a toxic response to radiation is the rs189037 SNP found in the noncoding transcript of the human ATM gene (“ATM_rs189037”). In FIG. 1, 100 nucleotides upstream (5′) of the mutation and 100 nucleotides downstream (3′) of the mutation are shown. The mutation is an A nucleotide (variant) substituted in place of a G nucleotide (wild-type). The mutation occurs at position 101 of SEQ ID NO: 67 (or SEQ ID NO: 113) (variant sequence) or SEQ ID NO: 68 (or SEQ ID NO: 114) (wild-type sequence). In one embodiment, a patient identified as carrying this mutation has a decreased likelihood of having a toxic response (e.g., short-term (acute)) to radiation, whereas a patient identified as not carrying the mutation has an increased likelihood of having a toxic response to radiation. In certain embodiments, the cancer is prostate cancer.

Another biomarker relevant to determining a cancer patient's likelihood of having a toxic response to radiation is the rs76677607 SNP found in the 3′ UTR of the human CD6 gene (“CD6_rs76677607” or “CD6_rs76677607”). In FIG. 1, 100 nucleotides upstream (5′) of the mutation and 100 nucleotides downstream (3′) of the mutation are shown. The mutation is an A nucleotide (variant) substituted in place of a G nucleotide (wild-type). The mutation occurs at position 101 of SEQ ID NO: 69 (variant sequence) or SEQ ID NO: 70 (wild-type sequence). In one embodiment, a patient identified as not carrying this mutation has a decreased likelihood of having a toxic response (e.g., wound toxicity, short-term (acute) and/or long-term (late)) to radiation, whereas a patient identified as carrying the mutation has an increased likelihood of having a toxic response to radiation. In one embodiment, a patient identified as carrying the CD6_rs76677607 mutation (A nucleotide) has an increased likelihood of having a toxic response to radiation, whereas a patient identified as not carrying the mutation (G nucleotide) has a decreased likelihood of having a toxic response to radiation. In one embodiment, a patient identified as carrying the CD6_rs76677607 mutation (A nucleotide) has an increased likelihood of mortality from cancer, whereas a patient identified as not carrying the mutation (G nucleotide) has a decreased likelihood of mortality from cancer. In one embodiment, a patient identified as carrying the CD6_rs76677607 mutation (A nucleotide) has an increased likelihood of biochemical relapse, whereas a patient identified as not carrying the mutation (G nucleotide) has a decreased likelihood biochemical relapse. In some embodiments, CD6_rs76677607 is associated with an increased risk of mortality and/or biochemical relapse. In some embodiments, the cancer is sarcoma. In some embodiments, the cancer is prostate cancer.

Another biomarker relevant to determining a cancer patient's likelihood of having a toxic response to radiation is the rs17561 SNP, which causes a missense mutation in the coding sequence of the human interleukin 1 alpha (IL1A) gene (“IL1A_rs17561”). In FIG. 1, 100 nucleotides upstream (5′) of the mutation and 100 nucleotides downstream (3′) of the mutation are shown. The mutation is an A nucleotide (variant) substituted in place of a C nucleotide (wild-type). The mutation occurs at position 101 of SEQ ID NO: 71 (and SEQ ID NO: 179) (variant sequence) or SEQ ID NO: 72 (and SEQ ID NO: 180) (wild-type sequence). In one embodiment, a patient identified as not carrying the IL1A_rs17561 mutation has an increased likelihood of having a toxic response (e.g., short-term (acute)) to radiation, whereas a patient identified as carrying the mutation has a decreased likelihood of having a toxic response to radiation. In certain embodiments, the cancer is prostate cancer.

Another biomarker relevant to determining a cancer patient's likelihood of having a toxic response to radiation is the rs7334543 SNP found in the 3′ UTR of the human BRCA2 gene (“BRCA2_rs7334543”). In FIG. 1, 100 nucleotides upstream (5′) of the mutation and 100 nucleotides downstream (3′) of the mutation are shown. The mutation is a G nucleotide (variant) substituted in place of an A nucleotide (wild-type). The mutation occurs at position 101 of SEQ ID NO: 73 (variant sequence) or SEQ ID NO: 74 (wild-type sequence). In one embodiment, a patient identified as not carrying the BRCA2_rs7334543 mutation has an increased likelihood of having a toxic response (e.g., short-term (acute) and/or long-term (late)) to radiation, whereas a patient identified as carrying the mutation has a decreased likelihood of having a toxic response to radiation. In certain embodiments, the cancer is prostate cancer.

Another biomarker relevant to determining a cancer patient's likelihood of having a toxic response to radiation is the rs118142475 SNP found in the intron region of the human ILF3 gene (“ILF3_rs118142475”). In FIG. 1, 100 nucleotides upstream (5′) of the mutation and 100 nucleotides downstream (3′) of the mutation are shown. The mutation is a T nucleotide (variant) substituted in place of a C nucleotide (wild-type). The mutation occurs at position 101 of SEQ ID NO: 75 (variant sequence) or SEQ ID NO: 76 (wild-type sequence). In one embodiment, a patient identified as carrying the ILF3_rs118142475 mutation has an increased likelihood of having a toxic response (e.g., short-term (acute)) to radiation, whereas a patient identified as not carrying the mutation has a decreased likelihood of having a toxic response to radiation. In certain embodiments, the cancer is prostate cancer.

Another biomarker relevant to determining a cancer patient's likelihood of having a toxic response to radiation is the rs2239680 SNP found in the non-coding region of the human BIRC5 gene (“BIRC5_rs2239680”). In FIG. 1, 100 nucleotides upstream (5′) of the mutation and 100 nucleotides downstream (3′) of the mutation are shown. The mutation is a C nucleotide (variant) substituted in place of a T nucleotide (wild-type). The mutation occurs at position 101 of SEQ ID NO: 77 (variant sequence) or SEQ ID NO: 78 (wild-type sequence). In some embodiments, the SNP is associated with mortality. In some embodiments, the SNP is associated with distant metastasis. In one embodiment, a patient identified as carrying the BIRC5_rs2239680 mutation has an increased likelihood of having a toxic response (e.g., short-term (acute)) to radiation, whereas a patient identified as not carrying the mutation has a decreased likelihood of having a toxic response to radiation. In one embodiment, a patient identified as carrying the BIRC5_rs2239680 mutation has a decreased likelihood of mortality from cancer, whereas a patient identified as not carrying the mutation has an increased likelihood of mortality from cancer. In one embodiment, a patient identified as carrying the BIRC5_rs2239680 mutation has a decreased likelihood of mortality from cancer, whereas a patient identified as not carrying the mutation has an increased likelihood of mortality from cancer. In one embodiment, a patient identified as carrying the BIRC5_rs2239680 mutation has a decreased likelihood of distant metastasis, whereas a patient identified as not carrying the mutation has an increased likelihood of distant metastasis. In some embodiments, the cancer is prostate cancer.

Another biomarker relevant to determining a cancer patient's likelihood of having a toxic response to radiation is the rs1805794 SNP found in the coding region of the human NBN gene (“NBN_rs1805794”). In FIG. 1, 100 nucleotides upstream (5′) of the mutation and 100 nucleotides downstream (3′) of the mutation are shown. The mutation is a G nucleotide (variant) substituted in place of a C nucleotide (wild-type). The mutation occurs at position 101 of SEQ ID NO: 79 (variant sequence) or SEQ ID NO: 80 (wild-type sequence). In one embodiment, a patient identified as carrying the NBN_rs1805794 mutation has an increased likelihood of having a toxic response (e.g., long-term (late)) to radiation, whereas a patient identified as not carrying the mutation has a decreased likelihood of having a toxic response to radiation. In one embodiment, a patient identified as carrying the NBN_rs1805794 mutation has an increased likelihood of distant metastasis following radiation treatment, whereas a patient identified as not carrying the mutation has a decreased likelihood of distant metastasis following radiation treatment. In certain embodiments, the radiation is traditional radiation. In certain embodiments, the cancer is prostate cancer.

A biomarker relevant to determining a cancer patient's likelihood of experiencing distant failure despite undergoing radiation therapy (e.g., hypofractionated radiation) is the rs11797 SNP found in the coding region of the human TREX gene (“TREX_rs11797”). In FIG. 1, 100 nucleotides upstream (5′) of the mutation and 100 nucleotides downstream (3′) of the mutation are shown. The mutation is a T nucleotide (variant) substituted in place of a C nucleotide (wild-type). The mutation occurs at position 101 of SEQ ID NO: 81 (variant sequence) or SEQ ID NO: 82 (wild-type sequence). In some embodiments, the SNP is associated with distant failure. In one embodiment, a patient identified as not carrying the TREX_rs11797 mutation has an increased likelihood of distant failure, whereas a patient identified as carrying the mutation has a decreased likelihood of distant failure. In certain embodiments, the cancer patient has sarcoma.

Another biomarker relevant to determining a cancer patient's likelihood of having a toxic response to radiation is the rs4742098 SNP found in the intron region of the human CD274 gene (“CD274_rs4742098”). In FIG. 1, 100 nucleotides upstream (5′) of the mutation and 100 nucleotides downstream (3′) of the mutation are shown. The mutation is a G nucleotide (variant) substituted in place of an A nucleotide (wild-type). The mutation occurs at position 101 of SEQ ID NO: 83 (variant sequence) or SEQ ID NO: 84 (wild-type sequence). In one embodiment, a patient identified as not carrying the CD274_rs4742098 mutation has an increased likelihood of having a toxic response (e.g., long-term (late)) to radiation, whereas a patient identified as carrying the mutation has a decreased likelihood of having a toxic response to radiation. In some embodiments, the SNP is associated with distant failure. In one embodiment, a patient identified as not carrying the CD274_rs4742098 mutation has an increased likelihood of distant failure, whereas a patient identified as carrying the mutation has a decreased likelihood of distant failure. In certain embodiments, the cancer patient has sarcoma. In certain embodiments, the cancer patient has prostate cancer.

Another biomarker relevant to determining a cancer patient's likelihood of having a toxic response to radiation is the rs4781562 SNP found in the 3′ UTR region of the human ERCC4 gene (“ERCC4_rs4781562”). In FIG. 1, 100 nucleotides upstream (5′) of the mutation and 100 nucleotides downstream (3′) of the mutation are shown. The mutation is a G nucleotide (variant) substituted in place of a T nucleotide (wild-type). The mutation occurs at position 101 of SEQ ID NO: 85 (variant sequence) or SEQ ID NO: 86 (wild-type sequence). In one embodiment, a patient identified as not carrying the ERCC4_rs4781562 mutation (T nucleotide) has an increased likelihood of having a toxic response (e.g., long-term (late)) to radiation, whereas a patient identified as carrying the mutation has a decreased likelihood of having a toxic response to radiation. In some embodiments, the SNP is associated with distant failure. In one embodiment, a patient identified as not carrying the ERCC4_rs4781562 mutation has an increased likelihood of distant failure, whereas a patient identified as carrying the mutation has a decreased likelihood of distant failure. In certain embodiments, the cancer patient has sarcoma.

Another biomarker relevant to determining a cancer patient's likelihood of having a toxic response to radiation is the rs2303428 SNP found in the intron region of the human MSH2 gene (“MSH2_rs2303428”). In FIG. 1, 100 nucleotides upstream (5′) of the mutation and 100 nucleotides downstream (3′) of the mutation are shown. The mutation is an A, C, or G nucleotide (variant) substituted in place of a T nucleotide (wild-type). The mutation occurs at position 101 of SEQ ID NO: 87 (variant sequence) or SEQ ID NO: 88 (wild-type sequence). In one embodiment, a patient identified as carrying the MSH2_rs2303428 mutation has an increased likelihood of having a toxic response (e.g., long-term (late)) to radiation, whereas a patient identified as not carrying the mutation has a decreased likelihood of having a toxic response to radiation. In certain embodiments, the cancer is prostate cancer.

Another biomarker relevant to determining a cancer patient's likelihood of having a toxic response to radiation is the rs1040363 SNP found in the intron region of the human XRCC4 gene (“XRCC4_rs1040363”). In FIG. 1, 100 nucleotides upstream (5′) of the mutation and 100 nucleotides downstream (3′) of the mutation are shown. The mutation is a C nucleotide (variant) substituted in place of a G nucleotide (wild-type). The mutation occurs at position 101 of SEQ ID NO: 89 (variant sequence) or SEQ ID NO: 90 (wild-type sequence). In some embodiments, the SNP is associated with mortality. In one embodiment, a patient identified as not carrying the XRCC4_rs1040363 mutation has an increased likelihood of having a toxic response to radiation, whereas a patient identified as carrying the mutation (C nucleotide) has a decreased likelihood of having a toxic response to radiation. In one embodiment, a patient identified as carrying the XRCC4_rs1040363 mutation (G nucleotide) has an increased likelihood of mortality from cancer, whereas a patient identified as not carrying the mutation has a decreased likelihood of mortality from cancer. In one embodiment, a patient identified as not carrying this mutation has an increased likelihood of impotency following radiation treatment, whereas a patient identified as carrying the mutation has a decreased likelihood of impotency following radiation treatment. In certain embodiments, the cancer is prostate cancer.

A biomarker relevant to determining a cancer patient's likelihood of having biochemical relapse despite undergoing radiation therapy (e.g., conventionally-fractionated radiation) is the rs2834167 SNP found in the coding region of the human IL10RB gene (“IL10RB_rs2834167”). In FIG. 1, 100 nucleotides upstream (5′) of the mutation and 100 nucleotides downstream (3′) of the mutation are shown. The mutation is a G nucleotide (variant) substituted in place of an A nucleotide (wild-type). The mutation occurs at position 101 of SEQ ID NO: 91 (variant sequence) or SEQ ID NO: 92 (wild-type sequence). In one embodiment, a patient identified as carrying the IL10RB_rs2834167 mutation has an decreased likelihood of biochemical relapse from cancer, whereas a patient identified as not carrying the mutation has an increased likelihood of biochemical relapse from cancer. In some embodiments, the cancer is prostate cancer.

A biomarker relevant to determining a cancer patient's likelihood of mortality or having a biochemical relapse despite undergoing radiation therapy (e.g., hypofractionated radiation) is the rs11724777 SNP found upstream of the human SMAD1 gene (“SMAD1_rs11724777”). In FIG. 1, 100 nucleotides upstream (5′) of the mutation and 100 nucleotides downstream (3′) of the mutation are shown. The mutation is an A nucleotide (variant) substituted in place of a T nucleotide (wild-type). The mutation occurs at position 101 of SEQ ID NO: 93 (variant sequence) or SEQ ID NO: 94 (wild-type sequence). In certain embodiments, the cancer is prostate cancer. In one embodiment, a patient identified as carrying the SMAD1_rs11724777 mutation has an increased likelihood of biochemical relapse and/or mortality from cancer, whereas a patient identified as not carrying the mutation has a decreased likelihood of biochemical relapse and/or mortality from cancer. In some embodiments, the cancer is prostate cancer.

Another biomarker relevant to determining a cancer patient's likelihood of having a toxic response to radiation is the rs10421768 SNP found upstream of the human HAMP gene (“HAMP_rs10421768”). In FIG. 1, 100 nucleotides upstream (5′) of the mutation and 100 nucleotides downstream (3′) of the mutation are shown. The mutation is a G nucleotide (variant) substituted in place of an A nucleotide (wild-type). The mutation occurs at position 101 of SEQ ID NO: 95 (variant sequence) or SEQ ID NO: 96 (wild-type sequence). In certain embodiments, a patient identified as not carrying this mutation has an increased likelihood of having a toxic response (e.g., long-term (late)) to radiation, whereas a patient identified as carrying this mutation has a decreased likelihood of having a toxic response to radiation. In some embodiments, the SNP is associated with potency. In one embodiment, a patient identified as not carrying this mutation has an increased likelihood of impotency following radiation treatment, whereas a patient identified as carrying the mutation has a decreased likelihood of impotency following radiation treatment. In certain embodiments, the cancer is prostate cancer.

A biomarker relevant to determining a cancer patient's likelihood of experiencing impotency after undergoing radiation therapy (e.g., hypofractionated radiation) is the rs25487 SNP found in the coding region of the human XRCC1 gene (“XRCC1_rs25487”). In FIG. 1, 100 nucleotides upstream (5′) of the mutation and 100 nucleotides downstream (3′) of the mutation are shown. The mutation is a C nucleotide (variant) substituted in place of a T nucleotide (wild-type). The mutation occurs at position 101 of SEQ ID NO: 97 (variant sequence) or SEQ ID NO: 98 (wild-type sequence). In some embodiments, the SNP is associated with potency. In one embodiment, a patient identified as carrying this mutation has an increased likelihood of impotency following radiation treatment, whereas a patient identified as not carrying the mutation has a decreased likelihood of impotency following radiation treatment. In certain embodiments, the cancer is prostate cancer.

Another biomarker relevant to determining a cancer patient's likelihood of having a toxic response to radiation is the rs2243158 SNP found in the intron region of the human IL19 gene (“IL19_rs2243158”). In FIG. 1, 100 nucleotides upstream (5′) of the mutation and 100 nucleotides downstream (3′) of the mutation are shown. The mutation is a G nucleotide (variant) substituted in place of a C nucleotide (wild-type). The mutation occurs at position 101 of SEQ ID NO: 99 (variant sequence) or SEQ ID NO: 100 (wild-type sequence). In some embodiments, the SNP is associated with potency. In one embodiment, a patient identified as carrying the IL19_rs2243158 mutation has an increased likelihood of having a toxic response (e.g., long-term (late)) to radiation, whereas a patient identified as not carrying the mutation has a decreased likelihood of having a toxic response to radiation. In one embodiment, a patient identified as carrying this mutation has an increased likelihood of impotency following radiation treatment as compared to a patient identified as not carrying the mutation, whereas a patient identified as not carrying the mutation has a decreased likelihood of impotency following radiation treatment. In certain embodiments, the cancer is prostate cancer.

Another biomarker relevant to determining a cancer patient's likelihood of having a toxic response to radiation is the rs2280883 SNP found in the intron region of the human FOXP3 gene (“FOXP3_rs2280883”). In FIG. 1, 100 nucleotides upstream (5′) of the mutation and 100 nucleotides downstream (3′) of the mutation are shown. The mutation is a C nucleotide (variant) substituted in place of a T nucleotide (wild-type). The mutation occurs at position 101 of SEQ ID NO: 101 (variant sequence) or SEQ ID NO: 102 (wild-type sequence). In some embodiments, the SNP is associated with distant metastasis. In one embodiment, a patient identified as carrying the FOXP3_rs2280883 mutation has a decreased likelihood of distant metastasis, whereas a patient identified as not carrying the mutation has an increased likelihood of distant metastasis.

Another biomarker relevant to determining a cancer patient's likelihood of having a toxic response to radiation is the rs3024496 SNP found in the 3′UTR region of the human IL10 gene (“IL10_rs3024496_P1.P2”). In FIG. 1, 100 nucleotides upstream (5′) of the mutation and 100 nucleotides downstream (3′) of the mutation are shown. The mutation is a G nucleotide (variant) substituted in place of an A nucleotide (wild-type). The mutation occurs at position 101 of SEQ ID NO: 103 (variant sequence) or SEQ ID NO: 104 (wild-type sequence). In some embodiments, the SNP is associated with distant metastasis. In some embodiments, the SNP is associated with distant failure. In one embodiment, a patient identified as carrying this mutation has a decreased likelihood of having a toxic response to radiation, whereas a patient identified as not carrying the mutation has an increased likelihood of having a toxic response to radiation. In another embodiment, a patient who does not carry the IL10_rs3024496_P1.P2 mutation has an increased likelihood of having a toxic response to radiation as compared to a patient who is heterozygous or homozygous for the IL10_rs3024496_P1.P2 mutation, whereas a patient who is heterozygous or homozygous for the IL10_rs3024496_P1.P2 mutation has a decreased likelihood of having a toxic response to radiation as compared to a patient who does not carry the IL10_rs3024496_P1.P2 mutation. In one embodiment, a patient identified as carrying the IL10_rs3024496_P1.P2 mutation has a decreased likelihood of distant metastasis, whereas a patient identified as not carrying the mutation has an increased likelihood of distant metastasis. In some embodiments, the cancer is prostate cancer.

Another biomarker relevant to determining a cancer patient's likelihood of having a toxic response to radiation is the rs2232643 SNP found in the 3′ UTR region of the human LIG4 gene (“LIG4_rs2232643”). In FIG. 1, 100 nucleotides upstream (5′) of the mutation and 100 nucleotides downstream (3′) of the mutation are shown. The mutation is a T nucleotide (variant) substituted in place of a C nucleotide (wild-type). The mutation occurs at position 101 of SEQ ID NO: 105 (variant sequence) or SEQ ID NO: 106 (wild-type sequence). In one embodiment, a patient identified as carrying this mutation has an increased likelihood of having a toxic response (e.g., short-term (acute)) to radiation, whereas a patient identified as not carrying the mutation has a decreased likelihood of having a toxic response to radiation. In certain embodiments, the toxicity is acute GU toxicity. In one embodiment, a patient identified as carrying the LIG4_rs2232643 mutation has an increased likelihood of distant metastasis, whereas a patient identified as not carrying the mutation has a decreased likelihood of distant metastasis.

Another biomarker relevant to determining a cancer patient's likelihood of having a toxic response to radiation is the rs2476491 SNP found in the intron region of the human IL2RA gene (“IL2RA_rs2476491”). In FIG. 1, 100 nucleotides upstream (5′) of the mutation and 100 nucleotides downstream (3′) of the mutation are shown. The mutation is a T nucleotide (variant) substituted in place of an A nucleotide (wild-type). The mutation occurs at position 101 of SEQ ID NO: 107 (variant sequence) or SEQ ID NO: 108 (wild-type sequence). In one embodiment, a patient identified as not carrying this mutation has an increased likelihood of having a toxic response (e.g., short-term (acute)) to radiation, whereas a patient identified as carrying the mutation has a decreased likelihood of having a toxic response to radiation. In certain embodiments, the cancer is prostate cancer.

Another biomarker relevant to determining a cancer patient's likelihood of having a toxic response to radiation, likelihood of mortality, or likelihood of developing impotency following radiation treatment is the rs568408 SNP found in the 3′ UTR region of the human IL12A gene (“IL12A_rs568408”). In FIG. 1, 100 nucleotides upstream (5′) of the mutation and 100 nucleotides downstream (3′) of the mutation are shown. The mutation is an A nucleotide (variant) substituted in place of a G nucleotide (wild-type). The mutation occurs at position 101 of SEQ ID NO: 109 (variant sequence) or SEQ ID NO: 110 (wild-type sequence). In one embodiment, a patient identified as not carrying this mutation has an increased likelihood of having a toxic response (e.g., short-term (acute)) to radiation, whereas a patient identified as carrying the mutation has a decreased likelihood of having a toxic response to radiation. In one embodiment, a patient identified as carrying this mutation has an increased likelihood of mortality (time until death is shorter) when compared to wild-type. In one embodiment, a patient identified as not carrying this mutation has a decreased likelihood of mortality when compared to a patient identified as carrying the mutation. In some embodiments, the IL12A_rs568408 SNP is associated with biochemical relapse. In one embodiment, a patient identified as carrying the IL12A_rs568408 mutation has an increased likelihood of biochemical relapse, whereas a patient identified as not carrying the mutation has a decreased likelihood biochemical relapse. In some embodiments, the SNP is associated with potency. In one embodiment, a patient identified as not carrying this mutation has an increased likelihood of impotency following radiation treatment, whereas a patient identified as carrying the mutation has a decreased likelihood of impotency following radiation treatment. In certain embodiments, the cancer is prostate cancer.

Another biomarker relevant to determining a cancer patient's likelihood of having a toxic response to radiation is the rs2037547 SNP found in the 3′ UTR region of the human GSK3B gene (“GSK3B_rs2037547”). In FIG. 1, 100 nucleotides upstream (5′) of the mutation and 100 nucleotides downstream (3′) of the mutation are shown. The mutation is a C nucleotide (variant) substituted in place of a T nucleotide (wild-type). The mutation occurs at position 101 of SEQ ID NO: 111 (variant sequence) or SEQ ID NO: 112 (wild-type sequence). In one embodiment, a patient identified as carrying this mutation has an increased likelihood of having a toxic response (e.g., short-term (acute)) to radiation, whereas a patient identified as not carrying the mutation has a decreased likelihood of having a toxic response to radiation. In certain embodiments, the cancer is prostate cancer.

Another biomarker relevant to determining a cancer patient's likelihood of having a toxic response to radiation is the rs1800797 SNP found in the intron region of the human IL6 gene (“IL6_rs1800797”). In FIG. 1, 100 nucleotides upstream (5′) of the mutation and 100 nucleotides downstream (3′) of the mutation are shown. The mutation is a G nucleotide (variant) substituted in place of an A nucleotide (wild-type). The mutation occurs at position 101 of SEQ ID NO: 115 (variant sequence) or SEQ ID NO: 116 (wild-type sequence). In one embodiment, a patient identified as not carrying this mutation has an increased likelihood of having a toxic response (e.g., short-term (acute)) to radiation, whereas a patient identified as carrying the mutation has a decreased likelihood of having a toxic response to radiation. In certain embodiments, the cancer is prostate cancer.

A biomarker relevant to determining a cancer patient's likelihood of experiencing distant failure despite undergoing radiation therapy (e.g., hypofractionated radiation) is the rs1801274 SNP found in the coding region of the human FCGR2A gene (“FCGR2A_rs1801274”). In FIG. 1, 100 nucleotides upstream (5′) of the mutation and 100 nucleotides downstream (3′) of the mutation are shown. The mutation is a C or G nucleotide (variant) substituted in place of an A nucleotide (wild-type). The mutation occurs at position 101 of SEQ ID NO: 119 (variant sequence) or SEQ ID NO: 120 (wild-type sequence). In one embodiment, a patient identified as not carrying the mutation has an increased likelihood of distant failure, whereas a patient identified as carrying the mutation has a decreased likelihood of distant failure. In some embodiments, the cancer is sarcoma.

Another biomarker relevant to determining a cancer patient's likelihood of having a toxic response to radiation is the rs373759 SNP found in the intron region of the human ATM gene (“ATM_rs373759”). In FIG. 1, 100 nucleotides upstream (5′) of the mutation and 100 nucleotides downstream (3′) of the mutation are shown. The mutation is a T nucleotide (variant) substituted in place of a C nucleotide (wild-type). The mutation occurs at position 101 of SEQ ID NO: 121 (variant sequence) or SEQ ID NO: 122 (wild-type sequence). In one embodiment, a patient identified as not carrying this mutation has a decreased likelihood of having a toxic response (e.g., short-term (acute)) to radiation, whereas a patient identified as carrying the mutation has an increased likelihood of having a toxic response to radiation. In certain embodiments, the cancer is prostate cancer.

Another biomarker relevant to determining a cancer patient's likelihood of having a toxic response to radiation is the rs8230 SNP found in the 3′ UTR region of the human CETN2 gene (“CETN2_rs8230”). In FIG. 1, 100 nucleotides upstream (5′) of the mutation and 100 nucleotides downstream (3′) of the mutation are shown. The mutation is an A nucleotide (variant) substituted in place of a C nucleotide (wild-type). The mutation occurs at position 101 of SEQ ID NO: 123 (variant sequence) or SEQ ID NO: 124 (wild-type sequence). In one embodiment, a patient identified as carrying this mutation has an increased likelihood of having a toxic response (e.g., a long-term toxic response) to radiation, whereas a patient identified as not carrying the mutation has a decreased likelihood of having a toxic response to radiation. In certain embodiments, the cancer is prostate cancer.

Another biomarker relevant to determining a cancer patient's likelihood of having a toxic response to radiation is the rs861539 SNP found in the coding region of the human XRCC3 gene (“XRCC3_rs861539”). In FIG. 1, 100 nucleotides upstream (5′) of the mutation and 100 nucleotides downstream (3′) of the mutation are shown. The mutation is an A nucleotide (variant) substituted in place of a G nucleotide (wild-type). The mutation occurs at position 101 of SEQ ID NO: 125 (variant sequence) or SEQ ID NO: 126 (wild-type sequence). In one embodiment, a patient identified as carrying this mutation has an increased likelihood of having a toxic response (e.g., short-term (acute)) to radiation, whereas a patient identified as not carrying the mutation has a decreased likelihood of having a toxic response to radiation. In certain embodiments, the cancer is prostate cancer.

Another biomarker relevant to determining a cancer patient's likelihood of having a toxic response to radiation is the rs4938723 SNP found within the human mir34.b.c. promoter (“miR.34b.c.promoter_rs4938723”). In FIG. 1, 100 nucleotides upstream (5′) of the mutation and 100 nucleotides downstream (3′) of the mutation are shown. The mutation is a C nucleotide (variant) substituted in place of a T nucleotide (wild-type). The mutation occurs at position 101 of SEQ ID NO: 127 (variant sequence) or SEQ ID NO: 128 (wild-type sequence). In one embodiment, a patient identified as not carrying this mutation has an increased likelihood of having a toxic response to radiation, whereas a patient identified as carrying the mutation has a decreased likelihood of having a toxic response to radiation. In another embodiment, a patient who does not carry the miR.34b.c.promoter_rs4938723 mutation has an increased likelihood of having a toxic response (e.g., a long-term (late)) to radiation as compared to a patient who is heterozygous or homozygous for the miR.34b.c.promoter_rs4938723 mutation, whereas a patient who is heterozygous or homozygous for the miR.34b.c.promoter_rs4938723 mutation has a decreased likelihood of having a toxic response to radiation as compared to a patient who does not carry the miR.34b.c.promoter_rs4938723 mutation. In some embodiments, the SNP is associated with distant failure.

Another biomarker relevant to determining a cancer patient's likelihood of having a toxic response to radiation is the rs9438623 SNP found within the 3′ UTR region of the human LIN28A gene (“LIN28A_rs9438623”). In FIG. 1, 100 nucleotides upstream (5′) of the mutation and 100 nucleotides downstream (3′) of the mutation are shown. The mutation is a G or T nucleotide (variant) substituted in place of an A nucleotide (wild-type). The mutation occurs at position 101 of SEQ ID NO: 129 (variant sequence) or SEQ ID NO: 130 (wild-type sequence). In one embodiment, a patient identified as carrying this mutation has an increased likelihood of having a toxic response (e.g., long-term (late)) to radiation, whereas a patient identified as not carrying the mutation has a decreased likelihood of having a toxic response to radiation. In one embodiment, a patient identified as not carrying this mutation has an increased likelihood of distant failure, whereas a patient identified as carrying the mutation has a decreased likelihood of distant failure. In certain embodiments, the cancer is prostate cancer.

Another biomarker relevant to determining a cancer patient's likelihood of having a toxic response to radiation is the rs9673 SNP found within the 3′ UTR region of the human FANCC gene (“FANCC_rs9673”). In FIG. 1, 100 nucleotides upstream (5′) of the mutation and 100 nucleotides downstream (3′) of the mutation are shown. The mutation is a G or C nucleotide (variant) substituted in place of an A nucleotide (wild-type). The mutation occurs at position 101 of SEQ ID NO: 131 (variant sequence) or SEQ ID NO: 132 (wild-type sequence). In one embodiment, a patient identified as carrying this mutation has an increased likelihood of having a toxic response (e.g., short-term (acute) or long-term (late)) to radiation, whereas a patient identified as not carrying the mutation has a decreased likelihood of having a toxic response to radiation. In certain embodiments, the cancer is prostate cancer.

Another biomarker relevant to determining a cancer patient's likelihood of having a toxic response to radiation is the rs465646 SNP found within the promoter region of the human REV3L gene (“REV3L_rs465646”). In FIG. 1, 100 nucleotides upstream (5′) of the mutation and 100 nucleotides downstream (3′) of the mutation are shown. The mutation is an A nucleotide (variant) substituted in place of a G nucleotide (wild-type). The mutation occurs at position 101 of SEQ ID NO: 133 (variant sequence) or SEQ ID NO: 134 (wild-type sequence). In one embodiment, a patient identified as not carrying this mutation has an increased likelihood of having a toxic response (e.g., short-term (acute)) to radiation, whereas a patient identified as carrying the mutation has a decreased likelihood of having a toxic response to radiation. In certain embodiments, the cancer is prostate cancer.

Another biomarker relevant to determining a cancer patient's likelihood of having a toxic response to radiation is the rs1800795 SNP found within the intron region of the human IL6 gene (“IL6_rs1800795”). In FIG. 1, 100 nucleotides upstream (5′) of the mutation and 100 nucleotides downstream (3′) of the mutation are shown. The mutation is a G nucleotide (variant) substituted in place of a C nucleotide (wild-type). The mutation occurs at position 101 of SEQ ID NO: 135 (variant sequence) or SEQ ID NO: 136 (wild-type sequence). In one embodiment, a patient identified as not carrying this mutation has an increased likelihood of having a toxic response (e.g., short-term (acute) or long-term (late)) to radiation, whereas a patient identified as carrying the mutation has a decreased likelihood of having a toxic response to radiation. In certain embodiments, the cancer is prostate cancer.

A biomarker relevant to determining a cancer patient's likelihood of having a toxic response to radiation or experiencing distant failure despite undergoing radiation therapy (e.g., hypofractionated radiation) is the rs2071304 SNP found within the intron region of the human SPI1 gene (“SPI1_rs2071304”). In FIG. 1, 100 nucleotides upstream (5′) of the mutation and 100 nucleotides downstream (3′) of the mutation are shown. The mutation is a C nucleotide (variant) substituted in place of a G nucleotide (wild-type). The mutation occurs at position 101 of SEQ ID NO: 137 (variant sequence) or SEQ ID NO: 138 (wild-type sequence). In one embodiment, a patient identified as carrying this mutation has an increased likelihood of having a toxic response (e.g., long-term (late)) to radiation (e.g., SBRT radiation), whereas a patient identified as not carrying the mutation has a decreased likelihood of having a toxic response to radiation. In one embodiment, a patient identified as carrying this mutation has an increased likelihood of distant failure, whereas a patient identified as not carrying the mutation has a decreased likelihood of distant failure. In certain embodiments, the cancer is prostate cancer. In certain embodiments, the cancer is sarcoma.

Another biomarker relevant to determining a cancer patient's likelihood of having a toxic response to radiation or experiencing distant failure or metastasis is the rs3024496 SNP found within the 3′ UTR region of the human IL10 gene (“IL10_rs3024496”). In FIG. 1, 100 nucleotides upstream (5′) of the mutation and 100 nucleotides downstream (3′) of the mutation are shown. The mutation is a G or C nucleotide (variant) substituted in place of an A or T nucleotide (wild-type). The mutation occurs at position 101 of SEQ ID NO: 139 (variant sequence) or SEQ ID NO: 140 (wild-type sequence). In some embodiments, the SNP is associated with distant failure. In certain embodiments the radiation is hypofractionated radiation. In some embodiments, the cancer is sarcoma. In one embodiment, a patient identified as not carrying this mutation has an increased likelihood of having a toxic response to radiation, whereas a patient identified as carrying the mutation has a decreased likelihood of having a toxic response to radiation. In some embodiments, the SNP is associated with distant metastasis. In certain embodiments, a patient identified as carrying this mutation has a decreased likelihood of distant metastasis following radiation relative to a patient not carrying this mutation, whereas a patient not carrying this mutation has an increased likelihood of distant metastasis following radiation relative to a patient carrying this mutation. In certain embodiments the radiation is traditional radiation. In certain embodiments, the cancer is prostate cancer.

Another biomarker relevant to determining a cancer patient's likelihood of experiencing distant failure despite undergoing radiation therapy (e.g., hypofractionated radiation) is the rs4150021 SNP found within the 3′ UTR region of the human EXO1 gene (“EXO1_rs4150021”). In FIG. 1, 100 nucleotides upstream (5′) of the mutation and 100 nucleotides downstream (3′) of the mutation are shown. The mutation is a deletion (variant) of a T nucleotide (wild-type). The deletion occurs at the T of [ATTTTG] of SEQ ID NO: 142 (wild-type sequence), resulting in SEQ ID NO: 141 (variant sequence). In one embodiment, a patient identified as carrying the mutation has an increased likelihood of distant failure, whereas a patient identified as not carrying the mutation has a decreased likelihood of distant failure. In some embodiments, the cancer is sarcoma.

Another biomarker relevant to determining a cancer patient's likelihood of having a toxic response to radiation is the rs2232365 SNP found within the intron region of the human FOXP3 gene (“FOXP3_rs2232365”). In FIG. 1, 100 nucleotides upstream (5′) of the mutation and 100 nucleotides downstream (3′) of the mutation are shown. The mutation is a C nucleotide (variant) substituted in place of a T nucleotide (wild-type). The mutation occurs at position 101 of SEQ ID NO: 143 (variant sequence) or SEQ ID NO: 144 (wild-type sequence). In one embodiment, a patient identified as not carrying this mutation has an increased likelihood of having a toxic response (e.g., long-term (late)) to radiation, whereas a patient identified as carrying the mutation has a decreased likelihood of having a toxic response to radiation. In certain embodiments, the cancer is prostate cancer.

Another biomarker relevant to determining a cancer patient's likelihood of experiencing distant failure despite undergoing radiation therapy (e.g., hypofractionated radiation) is the rs1882694 SNP found within the 5′ upstream region of the human HAMP gene (“HAMP_rs1882694”). In FIG. 1, 100 nucleotides upstream (5′) of the mutation and 100 nucleotides downstream (3′) of the mutation are shown. The mutation is a C nucleotide (variant) substituted in place of an A nucleotide (wild-type). The mutation occurs at position 101 of SEQ ID NO: 145 (variant sequence) or SEQ ID NO: 146 (wild-type sequence). In one embodiment, a patient identified as not carrying this mutation has an increased likelihood of having a toxic response (e.g., long-term (late)) to radiation, whereas a patient identified as carrying the mutation has a decreased likelihood of having a toxic response to radiation. In one embodiment, a patient identified as not carrying this mutation has an increased likelihood of distant failure, whereas a patient identified as carrying the mutation has a decreased likelihood of distant failure. In certain embodiments, the cancer is prostate cancer. In some embodiments, the cancer is sarcoma.

Another biomarker relevant to determining a cancer patient's likelihood of having a toxic response to radiation is the rs1800872 SNP found within the 5′ upstream region of the human IL10 gene (“IL10_rs1800872”). In FIG. 1, 100 nucleotides upstream (5′) of the mutation and 100 nucleotides downstream (3′) of the mutation are shown. The mutation is a G nucleotide (variant) substituted in place of an A nucleotide (wild-type). The mutation occurs at position 101 of SEQ ID NO: 147 (variant sequence) or SEQ ID NO: 148 (wild-type sequence). In one embodiment, a patient identified as not carrying this mutation has an decreased likelihood of having a toxic response (e.g., long-term (late)) to radiation, whereas a patient identified as carrying this mutation has an increased likelihood of having a toxic response to radiation. In certain embodiments, the cancer is prostate cancer.

Another biomarker relevant to determining a cancer patient's risk of biochemical relapse despite undergoing radiation therapy (e.g., hypofractionated radiation) is the rs11556218 SNP found within the coding region of the human IL16 gene (“IL16_rs11556218”). In FIG. 1, 100 nucleotides upstream (5′) of the mutation and 100 nucleotides downstream (3′) of the mutation are shown. The mutation is a G nucleotide (variant) substituted in place of a T nucleotide (wild-type). The mutation occurs at position 101 of SEQ ID NO: 149 (variant sequence) or SEQ ID NO: 150 (wild-type sequence). In some embodiments, the cancer is prostate cancer.

Another biomarker relevant to determining a cancer patient's likelihood of having a toxic response to radiation or biochemical relapse following radiation treatment is the rs11256497 SNP found within the intron region of the human IL2RA gene (“IL2RA_rs11256497”). In FIG. 1, 100 nucleotides upstream (5′) of the mutation and 100 nucleotides downstream (3′) of the mutation are shown. The mutation is an A nucleotide (variant) substituted in place of a G nucleotide (wild-type). The mutation occurs at position 101 of SEQ ID NO: 151 (variant sequence) or SEQ ID NO: 152 (wild-type sequence). In one embodiment, a patient identified as carrying this mutation has an increased likelihood of having a toxic response (e.g., long-term (late)) to radiation, whereas a patient identified as not carrying the mutation has a decreased likelihood of having a toxic response to radiation. In one embodiment, a patient identified as not carrying the IL2RA_rs11256497 mutation has an increased likelihood of biochemical relapse following radiation treatment, whereas a patient identified as carrying the mutation has a decreased likelihood of biochemical relapse. In certain embodiments the radiation is traditional radiation. In certain embodiments, the cancer is prostate cancer.

Another biomarker relevant to determining a cancer patient's likelihood of having a toxic response to radiation is the rs228942 SNP found within the coding region of the human IL2RB gene (“IL2RB_rs228942”). In FIG. 1, 100 nucleotides upstream (5′) of the mutation and 100 nucleotides downstream (3′) of the mutation are shown. The mutation is a T nucleotide (variant) substituted in place of a G nucleotide (wild-type). The mutation occurs at position 101 of SEQ ID NO: 153 (variant sequence) or SEQ ID NO: 154 (wild-type sequence). In one embodiment, a patient identified as carrying this mutation has an increased likelihood of having a toxic response (e.g., long-term (late)) to radiation, whereas a patient identified as not carrying the mutation has a decreased likelihood of having a toxic response to radiation. In certain embodiments, the cancer is prostate cancer.

Another biomarker relevant to determining a cancer patient's likelihood of having a toxic response to radiation is the rs17084733 SNP found within the 3′ UTR region of the human KIT gene (“KIT_rs17084733”). In FIG. 1, 100 nucleotides upstream (5′) of the mutation and 100 nucleotides downstream (3′) of the mutation are shown. The mutation is an A nucleotide (variant) substituted in place of a G nucleotide (wild-type). The mutation occurs at position 101 of SEQ ID NO: 155 (variant sequence) or SEQ ID NO: 156 (wild-type sequence). In one embodiment, a patient identified as not carrying this mutation has an increased likelihood of having a toxic response (e.g., long-term (late)) to radiation, whereas a patient identified as carrying the mutation has a decreased likelihood of having a toxic response to radiation. In certain embodiments, the cancer is prostate cancer.

Another biomarker relevant to determining a cancer patient's likelihood of experiencing distant failure despite undergoing radiation therapy (e.g., hypofractionated radiation) is the rs61764370 SNP found within the 3′ UTR region of the human KRAS gene (“KRAS_rs61764370”). In FIG. 1, 100 nucleotides upstream (5′) of the mutation and 100 nucleotides downstream (3′) of the mutation are shown. The mutation is a C nucleotide (variant) substituted in place of an A nucleotide (wild-type). The mutation occurs at position 101 of SEQ ID NO: 157 (variant sequence) or SEQ ID NO: 158 (wild-type sequence). In one embodiment, a patient identified as not carrying the mutation has an increased likelihood of distant failure, whereas a patient identified as carrying the mutation has a decreased likelihood of distant failure. In certain embodiments, the cancer is sarcoma.

Another biomarker relevant to determining a cancer patient's likelihood of having a toxic response to radiation is the rs3093772 SNP found within the 3′ UTR region of the human LIG4 gene (“LIG4_rs3093772_P1.P2”). In FIG. 1, 100 nucleotides upstream (5′) of the mutation and 100 nucleotides downstream (3′) of the mutation are shown. The mutation is a C nucleotide (variant) substituted in place of a T nucleotide (wild-type). The mutation occurs at position 101 of SEQ ID NO: 159 (variant sequence) or SEQ ID NO: 160 (wild-type sequence). In one embodiment, a patient identified as not carrying this mutation has an increased likelihood of having a toxic response (e.g., long-term (late)) to radiation, whereas a patient identified as carrying the mutation has a decreased likelihood of having a toxic response to radiation. In some embodiments, the SNP is associated with potency. In one embodiment, a patient identified as carrying this mutation has an increased likelihood of impotency following radiation treatment as compared to a patient identified as carrying the mutation, whereas a patient identified as not carrying the mutation has a decreased likelihood of impotency following radiation treatment. In certain embodiments, the cancer is prostate cancer.

Another biomarker relevant to determining a cancer patient's likelihood of having a toxic response to radiation is the rs769412 SNP found within the coding region of the human MDM2 gene (“MDM2_rs769412”). In FIG. 1, 100 nucleotides upstream (5′) of the mutation and 100 nucleotides downstream (3′) of the mutation are shown. The mutation is a G nucleotide (variant) substituted in place of an A nucleotide (wild-type). The mutation occurs at position 101 of SEQ ID NO: 161 (variant sequence) or SEQ ID NO: 162 (wild-type sequence). In one embodiment, a patient identified as carrying this mutation has an increased likelihood of having a toxic response (e.g., long-term (late)) to radiation, whereas a patient identified as not carrying this mutation has a decreased likelihood of having a toxic response to radiation. In certain embodiments, the cancer is prostate cancer.

Another biomarker relevant to determining a cancer patient's likelihood of having a toxic response to radiation is the rs3751143 SNP found within the coding region of the human P2RX7 gene (“P2RX7_rs3751143”). In FIG. 1, 100 nucleotides upstream (5′) of the mutation and 100 nucleotides downstream (3′) of the mutation are shown. The mutation is a G or C nucleotide (variant) substituted in place of an A nucleotide (wild-type). The mutation occurs at position 101 of SEQ ID NO: 163 (variant sequence) or SEQ ID NO: 164 (wild-type sequence). In one embodiment, a patient identified as not carrying this mutation has an increased likelihood of having a toxic response (e.g., long-term (late)) to radiation, whereas a patient identified as carrying the mutation has a decreased likelihood of having a toxic response to radiation. In one embodiment, a patient identified as carrying the P2RX7_rs3751143 mutation has an increased likelihood of biochemical relapse (shorter time until biochemical relapse) as compared to a patient identified as not carrying the mutation. In certain embodiments, the cancer is prostate cancer.

Another biomarker relevant to determining a cancer patient's likelihood of having a toxic response to radiation or experiencing distant failure is the rs2187668 SNP found within the intron region of the human HLA-DQA1 gene (“rs2187668”). In FIG. 1, 100 nucleotides upstream (5′) of the mutation and 100 nucleotides downstream (3′) of the mutation are shown. The mutation is a T nucleotide (variant) substituted in place of a C nucleotide (wild-type). The mutation occurs at position 101 of SEQ ID NO: 165 (variant sequence) or SEQ ID NO: 166 (wild-type sequence). In one embodiment, a patient identified as not carrying this mutation has an increased likelihood of having a toxic response (e.g., short-term (acute)) to radiation, whereas a patient identified as carrying the mutation has a decreased likelihood of having a toxic response to radiation. In one embodiment, a patient identified as carrying the mutation has an increased likelihood of distant failure, whereas a patient identified as not carrying the mutation has a decreased likelihood of distant failure. In certain embodiments, the cancer is prostate cancer.

Another biomarker relevant to determining a cancer patient's likelihood of having a toxic response to radiation is the rs3024505 SNP found within the IL10 gene (“IL10_rs3024505”). In FIG. 1, 100 nucleotides upstream (5′) of the mutation and 100 nucleotides downstream (3′) of the mutation are shown. The mutation is an A nucleotide (variant) substituted in place of a G nucleotide (wild-type). The mutation occurs at position 101 of SEQ ID NO: 167 (variant sequence) or SEQ ID NO: 168 (wild-type sequence). In one embodiment, a patient identified as carrying this mutation has an increased likelihood of having a toxic response (e.g., long-term (late)) to radiation (e.g., SBRT), whereas a patient identified as not carrying the mutation has a decreased likelihood of having a toxic response to radiation. In certain embodiments, the cancer is prostate cancer.

Another biomarker relevant to determining a cancer patient's likelihood of experiencing distant failure despite undergoing radiation therapy (e.g., hypofractionated radiation) is the rs922075 SNP found within the intron region of the human ATRIP gene and upstream of the CCDC51 gene (“rs922075”). In FIG. 1, 100 nucleotides upstream (5′) of the mutation and 100 nucleotides downstream (3′) of the mutation are shown. The mutation is a G nucleotide (variant) substituted in place of an A nucleotide (wild-type). The mutation occurs at position 101 of SEQ ID NO: 169 (variant sequence) or SEQ ID NO: 170 (wild-type sequence). In one embodiment, a patient identified as not carrying the mutation has an increased likelihood of distant failure, whereas a patient identified as carrying the mutation has a decreased likelihood of distant failure. In some embodiments, the cancer is sarcoma.

Another biomarker relevant to determining a cancer patient's likelihood of experiencing distant failure despite undergoing radiation therapy (e.g., hypofractionated radiation) is the rs1059313 SNP found within the 3′ UTR region of the human SOS1 gene (“SOS1_rs1059313”). In FIG. 1, 100 nucleotides upstream (5′) of the mutation and 100 nucleotides downstream (3′) of the mutation are shown. The mutation is a T nucleotide (variant) substituted in place of a G nucleotide (wild-type). The mutation occurs at position 101 of SEQ ID NO: 171 (variant sequence) or SEQ ID NO: 172 (wild-type sequence). In one embodiment, a patient identified as not carrying the mutation has an increased likelihood of distant failure, whereas a patient identified as carrying the mutation has a decreased likelihood of distant failure. In some embodiments, the cancer is sarcoma.

Another biomarker relevant to determining a cancer patient's likelihood of experiencing distant failure despite undergoing radiation therapy (e.g., hypofractionated radiation) is the rs7574070 SNP found within the intron region of the human STAT4 gene (“STAT4_rs7574070”). In FIG. 1, 100 nucleotides upstream (5′) of the mutation and 100 nucleotides downstream (3′) of the mutation are shown. The mutation is a C nucleotide (variant) substituted in place of an A nucleotide (wild-type). The mutation occurs at position 101 of SEQ ID NO: 173 (variant sequence) or SEQ ID NO: 174 (wild-type sequence). In one embodiment, a patient identified as not carrying the mutation has an increased likelihood of distant failure, whereas a patient identified as carrying the mutation has a decreased likelihood of distant failure. In some embodiments, the cancer is sarcoma.

Another biomarker relevant to determining a cancer patient's likelihood of having a toxic response to radiation is the UNGC.96 SNP found within the human TGFB2 gene (“UNGC.96.TGFB2_NA”). In FIG. 1, 100 nucleotides upstream (5′) of the mutation and 100 nucleotides downstream (3′) of the mutation are shown. The mutation is an A nucleotide (variant) substituted in place of a T nucleotide (wild-type). The mutation occurs at position 101 of SEQ ID NO: 175 (variant sequence) or SEQ ID NO: 176 (wild-type sequence). In one embodiment, a patient identified as carrying this mutation has an increased likelihood of having a toxic response (e.g., long-term (late)) to radiation, whereas a patient identified as not carrying the mutation has a decreased likelihood of having a toxic response to radiation. In certain embodiments, the cancer is prostate cancer.

Another biomarker relevant to determining a cancer patient's likelihood of having a toxic response to radiation is the rs41282644 SNP found within the 3′ UTR region of the human VEGFA gene (“VEGFA_rs41282644”). In FIG. 1, 100 nucleotides upstream (5′) of the mutation and 100 nucleotides downstream (3′) of the mutation are shown. The mutation is an A nucleotide (variant) substituted in place of a G nucleotide (wild-type). The mutation occurs at position 101 of SEQ ID NO: 177 (variant sequence) or SEQ ID NO: 178 (wild-type sequence). In one embodiment, a patient identified as not carrying this mutation has an increased likelihood of having a toxic response (e.g., long-term (late)) to radiation, whereas a patient identified as carrying the mutation has a decreased likelihood of having a toxic response to radiation. In certain embodiments, the cancer is prostate cancer.

Another biomarker relevant to determining a cancer patient's likelihood of having a toxic response to radiation is the rs1800469 SNP found within the upstream region of the human transforming growth factor beta 1 TGFB1 gene (“TGFB1_rs1800469”). In FIG. 1, 100 nucleotides upstream (5′) of the mutation and 100 nucleotides downstream (3′) of the mutation are shown. The mutation is a G nucleotide (variant) substituted in place of an A nucleotide (wild-type). The mutation occurs at position 101 of SEQ ID NO: 181 (variant sequence) or SEQ ID NO: 182 (wild-type sequence). In one embodiment, a patient identified as being heterozygous for this mutation has an increased likelihood of having a toxic response (e.g., short-term (acute)) to radiation, whereas a patient identified as being homozygous for the wild-type allele has a decreased likelihood of having a toxic response to radiation. In certain embodiments, the cancer is prostate cancer.

Another biomarker relevant to determining a cancer patient's likelihood of having a toxic response to radiation is the rs884225 SNP found within the 3′ UTR region of the human epidermal growth factor (EGFR) gene (“EGFR_rs884225”). In FIG. 1, 100 nucleotides upstream (5′) of the mutation and 100 nucleotides downstream (3′) of the mutation are shown. The mutation is a C nucleotide (variant) substituted in place of a T nucleotide (wild-type). The mutation occurs at position 101 of SEQ ID NO: 183 (variant sequence) or SEQ ID NO: 184 (wild-type sequence). In one embodiment, a patient identified as not carrying this mutation has an increased likelihood of having a toxic response (e.g., short-term (acute)) to radiation, whereas a patient identified as carrying the mutation has a decreased likelihood of having a toxic response to radiation. In certain embodiments, the cancer is prostate cancer.

Another biomarker relevant to determining a cancer patient's likelihood of having a toxic response to radiation is the rs17388569 SNP found in sequence GRCh37.p13 on human chromosome 2 (“rs17388569” also referred to as “rs1347682”). In FIG. 1, 100 nucleotides upstream (5′) of the mutation and 100 nucleotides downstream (3′) of the mutation are shown. The mutation is a G nucleotide (variant) substituted in place of an A nucleotide (wild-type). The mutation occurs at position 101 of SEQ ID NO: 185 (variant sequence) or SEQ ID NO: 186 (wild-type sequence). In one embodiment, a patient identified as not carrying this mutation has an increased likelihood of having a toxic response (e.g., short-term (acute)) to radiation, whereas a patient identified as carrying the mutation has a decreased likelihood of having a toxic response to radiation. In certain embodiments, the cancer is prostate cancer.

Another biomarker relevant to determining a cancer patient's likelihood of having a toxic response to radiation is the rs4986790 SNP, which is a missense variant of the toll like receptor 4 (TLR4) (“TRL4_rs4986790”). In FIG. 1, 100 nucleotides upstream (5′) of the mutation and 100 nucleotides downstream (3′) of the mutation are shown. The mutation is a G nucleotide (variant) substituted in place of an A nucleotide (wild-type). The mutation occurs at position 101 of SEQ ID NO: 187 (variant sequence) or SEQ ID NO: 188 (wild-type sequence). In one embodiment, a patient identified as not carrying this mutation has an increased likelihood of having a toxic response (e.g., long-term (late)) to radiation, whereas a patient identified as carrying the mutation has a decreased likelihood of having a toxic response to radiation. In certain embodiments, the cancer is prostate cancer.

Another biomarker relevant to determining a cancer patient's likelihood of having a toxic response to radiation is the rs1800057 SNP, which is a missense variant of the ATM serine/threonine kinase (“ATM_rs1800057”). In FIG. 1, 100 nucleotides upstream (5′) of the mutation and 100 nucleotides downstream (3′) of the mutation are shown. The mutation is a G nucleotide (variant) substituted in place of a C nucleotide (wild-type). The mutation occurs at position 101 of SEQ ID NO: 189 (variant sequence) or SEQ ID NO: 190 (wild-type sequence). In one embodiment, a patient identified as carrying this mutation has an increased likelihood of having a toxic response (e.g., long-term (late)) to radiation, whereas a patient identified as not carrying the mutation has a decreased likelihood of having a toxic response to radiation. In certain embodiments, the cancer is prostate cancer.

Another biomarker relevant to determining a cancer patient's likelihood of having a toxic response to radiation is the rs79383051 SNP, which is a noncoding transcript variant of the interleukin 1 receptor accessory protein (plus strand) (“ILRAP_rs79383051”). In FIG. 1, 100 nucleotides upstream (5′) of the mutation and 100 nucleotides downstream (3′) of the mutation are shown. The mutation is a T nucleotide (variant) substituted in place of a C nucleotide (wild-type). The mutation occurs at position 101 of SEQ ID NO: 191 (variant sequence) or SEQ ID NO: 192 (wild-type sequence). In one embodiment, a patient identified as carrying this mutation has an increased likelihood of having a toxic response (e.g., long-term (late)) to radiation, whereas a patient identified as not carrying the mutation has a decreased likelihood of having a toxic response to radiation. In certain embodiments, the cancer is prostate cancer.

Another biomarker relevant to determining a cancer patient's time to mortality in response to radiation is the rs20541 SNP, which is a missense variant of the interleukin 13 protein (plus strand) (“IL3_rs20541”). In FIG. 1, 100 nucleotides upstream (5′) of the mutation and 100 nucleotides downstream (3′) of the mutation are shown. The mutation is a G nucleotide (variant) substituted in place of an A nucleotide (wild-type). The mutation occurs at position 101 of SEQ ID NO: 193 (variant sequence) or SEQ ID NO: 194 (wild-type sequence). In one embodiment, a patient identified as carrying this mutation has an decreased likelihood of mortality whereas a patient identified as not carrying the mutation has an increased likelihood of morality (time to death is shorter). In certain embodiments, the cancer is prostate cancer and the mortality is prostate cancer specific mortality.

Another biomarker relevant to determining a cancer patient's time to biochemical relapse in response to radiation is the rs8679 SNP, which is a 3′ UTR variant of the poly(ADP-ribose) polymerase 1 (minus strand) (“PARP1_rs8679”). In FIG. 1, 100 nucleotides upstream (5′) of the mutation and 100 nucleotides downstream (3′) of the mutation are shown. The mutation is a G nucleotide (variant) substituted in place of an A nucleotide (wild-type). The mutation occurs at position 101 of SEQ ID NO: 195 (variant sequence) or SEQ ID NO: 196 (wild-type sequence). In one embodiment, a patient identified as carrying this mutation has an increased likelihood of biochemical relapse (time to biochemical relapse is shorter) relative to a patient not carrying the mutation, whereas a patent identified as not carrying the mutation has a decreased likelihood of biochemical relapse. In certain embodiments, the cancer is prostate cancer.

Another biomarker relevant to determining a cancer patient's time to distant metastasis in response to radiation is the rs4073 SNP, which is an upstream variant of the interleukin-8 (IL-8) gene (also called C-X-C motif chemokine ligand 8 (CXCL8)) (plus strand) (“IL8_rs4073”). In FIG. 1, 100 nucleotides upstream (5′) of the mutation and 100 nucleotides downstream (3′) of the mutation are shown. The mutation is a T nucleotide (variant) substituted in place of an A nucleotide (wild-type). The mutation occurs at position 101 of SEQ ID NO: 197 (variant sequence) or SEQ ID NO: 198 (wild-type sequence). In one embodiment, a patient identified as carrying this mutation has an increased likelihood of distant metastasis (time to distant metastasis is shorter) relative to a patient not carrying the mutation, whereas a patent identified as not carrying the mutation has a decreased likelihood of distant metastasis. In certain embodiments, the radiation treatment is traditional radiation. In certain embodiments, the cancer is prostate cancer.

Each of the biomarkers disclosed herein as being predictive of toxicity may be used either alone or in combination with one or more of the other markers disclosed herein as predictive of toxicity in order to predict whether or not a patient will have a toxic response to radiation therapy. For example, one may use one, two, three, four, five, six, seven, eight, nine, or ten of the biomarkers in determining a patient's predicted toxicity to a radiation therapy. In particular, determining whether a patient is homozygous or heterozygous for a particular marker associated with toxicity to radiation (or does not carry the marker, i.e., the patient is wild-type) can be useful in determining the patient's predicted toxicity to a radiation therapy. For example, one may determine the zygosity of a patient with respect to one, two, three, four, five, six, seven, eight, nine or ten of the biomarkers disclosed herein as predictive of toxicity in order to determine the patient's predicted toxicity to a radiation therapy. For example, one may determine the zygosity of at least one, at least two, at least three, at least four, at least five, at least six, at least seven, at least eight, or at least nine of the aforementioned markers associated with toxicity.

In one embodiment of the invention, determining whether a patient is likely to have a toxic or non-toxic response to a radiation treatment, includes identifying whether the patient carries or does not carry one or more of the following mutations:
a) a C nucleotide at a position corresponding to position 101 of SEQ ID NO: 1 (miR.99a.promoter);
b) an A nucleotide at a position corresponding to position 101 of SEQ ID NO: 3 (IL1.B_rs4848306);
c) a G nucleotide at a position corresponding to position 101 of SEQ ID NO: 5 (SHC4_rs1062124);
d) a T nucleotide at a position corresponding to position 101 of SEQ ID NO: 7 (rs17599026);
e) an A nucleotide at a position corresponding to position 101 of SEQ ID NO: 9 (RAC1_rs9374);
f) a C nucleotide at a position corresponding to position 101 of SEQ ID NO: 11 (IL24_rs1150258);
g) an A or C nucleotide at a position corresponding to position 101 of SEQ ID NO: 13 (IL1A_rs1800587);
h) a C nucleotide at a position corresponding to position 101 of SEQ ID NO: 15 (ERCC1_rs3212948);
i) a G nucleotide at a position corresponding to position 101 of SEQ ID NO: 17 (IL.6_rs12700386);
j) a G nucleotide at a position corresponding to position 101 of SEQ ID NO: 19 (IL.6_rs2069840);
k) a T nucleotide at a position corresponding to position 101 of SEQ ID NO: 21 (CAMK2G_rs2306327);
l) a T nucleotide at a position corresponding to position 101 of SEQ ID NO: 23 (BMP2_rs1980499);
m) a C nucleotide at a position corresponding to position 101 of SEQ ID NO: 25 (EREG_rs1460008);
n) a C nucleotide at a position corresponding to position 101 of SEQ ID NO: 27 (CD274_rs4143815);
o) an A nucleotide at a position corresponding to position 101 of SEQ ID NO: 29 (IL17D_rs7787);
p) a C nucleotide at a position corresponding to position 101 of SEQ ID NO: 31 (STAT3/rs3744483);
q) a C nucleotide at a position corresponding to position 101 of SEQ ID NO: 33 (BMP4_rs17563)
r) a C nucleotide at a position corresponding to position 101 of SEQ ID NO: 35 (IRF5_rs10488631);
s) a T nucleotide at a position corresponding to position 101 of SEQ ID NO: 37 (UNGC.41.IL1RAP);
t) a G nucleotide at a position corresponding to position 101 of SEQ ID NO: 39 (IL19_rs1798);
u) an A nucleotide at a position corresponding to position 101 of SEQ ID NO: 41 (RAD23A_rs8240);
v) a C nucleotide at a position corresponding to position 101 of SEQ ID NO: 43 (BRCA2_rs15869);
w) a G nucleotide at a position corresponding to position 101 of SEQ ID NO: 45
x) a C nucleotide at a position corresponding to position 101 of SEQ ID NO: 47 (BMP2_rs3178250);
y) a T nucleotide at a position corresponding to position 101 of SEQ ID NO: 49 (CD274_rs1411262);
z) an A nucleotide at a position corresponding to position 101 of SEQ ID NO: 51 (CD274_rs2297136);
aa) a G nucleotide at a position corresponding to position 101 of SEQ ID NO: 53 (BMP2_rs1979855);
bb) a G nucleotide at a position corresponding to position 101 of SEQ ID NO: 55 (PTPN2_rs1893217);
cc) a G nucleotide at a position corresponding to position 101 of SEQ ID NO: 57 (CD274_rs822339);
dd) a G nucleotide at a position corresponding to position 101 of SEQ ID NO: 59 (BATF3_rs6695772);
ee) an A nucleotide at a position corresponding to position 101 of SEQ ID NO: 61 (IL18R1_rs11465660);
ff) a G nucleotide at a position corresponding to position 101 of SEQ ID NO: 63 (ERCC1_rs11615);
gg) a C nucleotide at a position corresponding to position 101 of SEQ ID NO: 65 (ABL1_rs11991);
hh) an A nucleotide at a position corresponding to position 101 of SEQ ID NO: 67 (ATM_rs189037);
ii) an A nucleotide at a position corresponding to position 101 of SEQ ID NO: 69 (CD6_rs76677607);
jj) an A nucleotide at a position corresponding to position 101 of SEQ ID NO: 71 (IL1A_rs17561);
kk) a G nucleotide at a position corresponding to position 101 of SEQ ID NO: 73 (BRCA2_rs7334543);
ll) a T nucleotide at a position corresponding to position 101 of SEQ ID NO: 75 (ILF3.58_rs118142475);
mm) a C nucleotide at a position corresponding to position 101 of SEQ ID NO: 77 (BIRC5_rs2239680);
nn) a G nucleotide at a position corresponding to position 101 of SEQ ID NO: 79 (NBN_rs1805794);
oo) a T nucleotide at a position corresponding to position 101 of SEQ ID NO: 81 (TREX_rs11797);
pp) a G nucleotide at a position corresponding to position 101 of SEQ ID NO: 83 (CD274_rs4742098);
qq) a T nucleotide at a position corresponding to position 101 of SEQ ID NO: 85 (ERCC4_rs4781562);
rr) an A, C or G nucleotide at a position corresponding to position 101 of SEQ ID NO: 87 (MSH2_rs2303428);
ss) a C nucleotide at a position corresponding to position 101 of SEQ ID NO: 89 (XRCC4_rs1040363);
tt) a G nucleotide at a position corresponding to position 101 of SEQ ID NO: 91 (IL10RB_rs2834167);
uu) an A nucleotide at a position corresponding to position 101 of SEQ ID NO: 93 (SMAD1_rs11724777);
vv) a G nucleotide at a position corresponding to position 101 of SEQ ID NO: 95 (HAMP_rs10421768);
ww) a C nucleotide at a position corresponding to position 101 of SEQ ID NO: 97 (XRCC1_rs25487);
xx) a G nucleotide at a position corresponding to position 101 of SEQ ID NO: 99 (IL19_rs2243158);
yy) a C nucleotide at a position corresponding to position 101 of SEQ ID NO: 101 (FOXP3_rs2280883);
zz) a G nucleotide at a position corresponding to position 101 of SEQ ID NO: 103 (IL10_rs3024496_P1.P2);
aaa) a C nucleotide at a position corresponding to position 101 of SEQ ID NO: 105 (LIG4_rs2232643);
bbb) a T nucleotide at a position corresponding to position 101 of SEQ ID NO: 107 (IL2RA_rs2476491);
ccc) an A nucleotide at a position corresponding to position 101 of SEQ ID NO: 109 (IL12A_rs568408);
ddd) a C nucleotide at a position corresponding to position 101 of SEQ ID NO: 111 (GSK3B_rs2037547);
eee) an A nucleotide at a position corresponding to position 101 of SEQ ID NO: 113 (ATM_rs189037);
fff) a G nucleotide at a position corresponding to position 101 of SEQ ID NO: 115 (IL6_rs1800797);
ggg) an A nucleotide at a position corresponding to position 101 of SEQ ID NO: 117 (RAC1_rs9374);
hhh) a C or G nucleotide at a position corresponding to position 101 of SEQ ID NO: 119 (FCGR2A_rs1801274);
iii) a T nucleotide at a position corresponding to position 101 of SEQ ID NO: 121 (ATM_rs373759);
jjj) an A nucleotide at a position corresponding to position 101 of SEQ ID NO: 123 (CETN2_rs8230);
kkk) an A nucleotide at a position corresponding to position 101 of SEQ ID NO: 125 (XRCC3_rs861539);
lll) a C nucleotide at a position corresponding to position 101 of SEQ ID NO: 127 (miR.34b.c.promoter_rs4938723);
mmm) a G nucleotide at a position corresponding to position 101 of SEQ ID NO: 129 (LIN28A_rs9438623);
nnn) a G or C nucleotide at a position corresponding to position 101 of SEQ ID NO: 131 (FANCC_rs9673);
ooo) an A nucleotide at a position corresponding to position 101 of SEQ ID NO: 133 (REV3L_rs465646);
ppp) a G nucleotide at a position corresponding to position 101 of SEQ ID NO: 135 (IL6_rs1800795);
qqq) a C nucleotide at a position corresponding to position 101 of SEQ ID NO: 137 (SPI1_rs2071304);
rrr) a G nucleotide at a position corresponding to position 101 of SEQ ID NO: 139 (IL10_rs3024496);
sss) a deletion of a T nucleotide in the sequence ATTTTG of SEQ ID NO: 142 (wild-type sequence) to give a corresponding sequence of ATTTG as in SEQ ID NO: 141 (mutant) (EXO1_rs4150021);
ttt) an A nucleotide at a position corresponding to position 101 of SEQ ID NO: 143 (FOXP3_rs2232365);
uuu) a C nucleotide at a position corresponding to position 101 of SEQ ID NO: 145 (HAMP_rs1882694);
vvv) a G nucleotide at a position corresponding to position 101 of SEQ ID NO: 147 (IL10_rs1800872);
www) a G nucleotide at a position corresponding to position 101 of SEQ ID NO: 149 (IL16_rs11556218);
xxx) an A nucleotide at a position corresponding to position 101 of SEQ ID NO: 151 (IL2RA_rs11256497);
yyy) a T nucleotide at a position corresponding to position 101 of SEQ ID NO: 153 (IL2RB_rs228942);
zzz) an A nucleotide at a position corresponding to position 101 of SEQ ID NO: 155 (KIT_rs17084733);
aaaa) a C nucleotide at a position corresponding to position 101 of SEQ ID NO: 157 (KRAS_rs61764370);
bbbb) a C nucleotide at a position corresponding to position 101 of SEQ ID NO: 159 (LIG4_rs3093772_P1.P2);
cccc) a G nucleotide at a position corresponding to position 101 of SEQ ID NO: 161 (MDM2_rs769412);
dddd) a C or G nucleotide at a position corresponding to position 101 of SEQ ID NO: 163 (P2RX7_rs3751143);
eeee) a T nucleotide at a position corresponding to position 101 of SEQ ID NO: 165 (rs2187668);
ffff) an A nucleotide at a position corresponding to position 101 of SEQ ID NO: 167 (IL10_rs3024505);
gggg) a G nucleotide at a position corresponding to position 101 of SEQ ID NO: 169 (rs922075);
hhhh) a T nucleotide at a position corresponding to position 101 of SEQ ID NO: 171 (SOS1_rs1059313);
iiii) a C nucleotide at a position corresponding to position 101 of SEQ ID NO: 173 (STAT4_rs7574070);
jjjj) an A nucleotide at a position corresponding to position 101 of SEQ ID NO: 175 (UNGC.96.TGFB2_NA);
kkkk) an A nucleotide at a position corresponding to position 101 of SEQ ID NO: 177 (VEGFA_rs41282644);
llll) an A nucleotide at a position corresponding to position 101 of SEQ ID NO: 179 (IL1A_rs17561);
mmmm) a G nucleotide at a position corresponding to position 101 of SEQ ID NO: 181 (TGFB1_rs1800469);
nnnn) a C nucleotide at a position corresponding to position 101 of SEQ ID NO: 183 (EGFR_rs884225);
oooo) a G nucleotide at a position corresponding to position 101 of SEQ ID NO: 185 (rs17388569);
pppp) a G nucleotide at a position corresponding to position 101 of SEQ ID NO: 187 (TRL4_rs4986790);
qqqq) a G nucleotide at a position corresponding to position 101 of SEQ ID NO: 189 (ATM_rs1800057); and rrrr) a T nucleotide at a position corresponding to position 101 of SEQ ID NO: 191 (ILRAP_rs79383051).

In certain aspects, the disclosure relates to a method of treating a patient with a sarcoma, wherein said sarcoma is preferably treated by hypofractionated or conventionally fractionated radiation treatment (CF-RT), wherein if said patient is carrying or not carrying one or more mutations from group (i), said patient is treated with hypofractionated radiation, and if said patient is carrying or not carrying one or more mutations from group (ii), said patient is treated with a conventionally fractionated radiation treatment (CF-RT), wherein group (i) is:
a) not carrying or carrying only one copy of a C nucleotide at a position corresponding to position 101 of SEQ ID NO: 1 (miR.99a.promoter);
b) not carrying or carrying only one copy of an A nucleotide at a position corresponding to position 101 of SEQ ID NO: 3 (IL1.B_rs4848306);
c) not carrying or carrying only one copy of a G nucleotide at a position corresponding to position 101 of SEQ ID NO: 5 (SHC4_rs1062124);
d) carrying a T nucleotide at a position corresponding to position 101 of SEQ ID NO: 7 (rs17599026);
e) not carrying or carrying only one copy of an A nucleotide at a position corresponding to position 101 of SEQ ID NO: 9 (RAC1_rs9374);
f) not carrying a C nucleotide at a position corresponding to position 101 of SEQ ID NO: 11 (IL24_rs1150258);
g) not carrying or carrying only one copy of an A nucleotide at a position corresponding to position 101 of SEQ ID NO: 13 (IL1A_rs1800587);
h) not carrying a C nucleotide at a position corresponding to position 101 of SEQ ID NO: 15 (ERCC1_rs3212948);
i) not carrying or carrying only one copy of a G nucleotide at a position corresponding to position 101 of SEQ ID NO: 17 (IL.6_rs12700386);
j) not carrying or carrying only one copy of a G nucleotide at a position corresponding to position 101 of SEQ ID NO: 19 (IL.6_rs2069840);
k) not carrying or carrying only one copy of a T nucleotide at a position corresponding to position 101 of SEQ ID NO: 21 (CAMK2G_rs2306327);
l) not carrying or carrying only one copy of a T nucleotide at a position corresponding to position 101 of SEQ ID NO: 23 (BMP2_rs1980499);
m) not carrying or carrying only one copy of a C nucleotide at a position corresponding to position 101 of SEQ ID NO: 25 (EREG_rs1460008);
n) not carrying or carrying only one copy of a C nucleotide at a position corresponding to position 101 of SEQ ID NO: 27 (CD274_rs4143815);
o) not carrying or carrying only one copy of an A nucleotide at a position corresponding to position 101 of SEQ ID NO: 29 (IL17D_rs7787);
p) not carrying or carrying only one copy a C nucleotide at a position corresponding to position 101 of SEQ ID NO: 31 (STAT3/rs3744483);
q) not carrying or carrying only one copy of a C nucleotide at a position corresponding to position 101 of SEQ ID NO: 33 (BMP4_rs17563)
r) not carrying a C nucleotide at a position corresponding to position 101 of SEQ ID NO: 35 (IRF5_rs10488631); and
s) not carrying or carrying only one copy of a T nucleotide at a position corresponding to position 101 of SEQ ID NO: 37 (UNGC.41.IL1RAP),

and group (ii) is:
a) carrying a C nucleotide at a position corresponding to position 101 of SEQ ID NO: 1 (miR.99a.promoter);
b) carrying an A nucleotide at a position corresponding to position 101 of SEQ ID NO: 3 (IL1.B_rs4848306);
c) carrying a G nucleotide at a position corresponding to position 101 of SEQ ID NO: 5 (SHC4_rs1062124);
d) not carrying a T nucleotide at a position corresponding to position 101 of SEQ ID NO: 7 (rs17599026);
e) carrying an A nucleotide at a position corresponding to position 101 of SEQ ID NO: 9 (RAC1_rs9374);
f) carrying a C nucleotide at a position corresponding to position 101 of SEQ ID NO: 11 (IL24_rs1150258);
g) carrying an A nucleotide at a position corresponding to position 101 of SEQ ID NO: 13 (IL1A_rs1800587);
h) carrying a C nucleotide at a position corresponding to position 101 of SEQ ID NO: 15 (ERCC1_rs3212948);
i) carrying a G nucleotide at a position corresponding to position 101 of SEQ ID NO: 17 (IL.6_rs12700386);
j) carrying a G nucleotide at a position corresponding to position 101 of SEQ ID NO: 19 (IL.6_rs2069840);
k) carrying a T nucleotide at a position corresponding to position 101 of SEQ ID NO: 21 (CAMK2G_rs2306327);
l) carrying a T nucleotide at a position corresponding to position 101 of SEQ ID NO: 23 (BMP2_rs1980499);
m) carrying a C nucleotide at a position corresponding to position 101 of SEQ ID NO: 25 (EREG_rs1460008);
n) carrying a C nucleotide at a position corresponding to position 101 of SEQ ID NO: 27 (CD274_rs4143815);
o) carrying an A nucleotide at a position corresponding to position 101 of SEQ ID NO: 29 (IL17D_rs7787);
p) carrying two copies of a C nucleotide at a position corresponding to position 101 of SEQ ID NO: 31 (STAT3/rs3744483);
q) carrying a C nucleotide at a position corresponding to position 101 of SEQ ID NO: 33 (BMP4_rs17563)
r) carrying a C nucleotide at a position corresponding to position 101 of SEQ ID NO: 35 (IRF5_rs10488631); and
s) carrying a T nucleotide at a position corresponding to position 101 of SEQ ID NO: 37 (UNGC.41.IL1RAP);
wherein if said patient is a patient in group (ii), treatment with hypofractionated radiation is more likely to result in toxicity.

In certain embodiments, the toxicity is wound toxicity. In certain embodiments, the sarcoma is present on a lower extremity.

In certain embodiments, the hypofractionated radiation is administration of radiation from about 20 Gy to about 35 Gy radiation over about 5 to about 8 fractions.

In certain embodiments, the CF-RT is administration of radiation from about 40 Gy to about 60 Gy radiation over a period of about 5 to about 6 weeks. In certain embodiments, the CF-RT regimen comprises administering from about 40 Gy to about 60 Gy radiation over about 15 to about 30 fractions.

The disclosure further relates to a method of treating a patient with a prostate cancer, wherein said prostate cancer is preferably treated by hypofractionated radiation, CF-RT, or a different treatment, wherein if said patient is identified as carrying or not carrying one or more mutations from group (i), said patient is treated with hypofractionated radiation, and if said patient is identified as carrying or not carrying one or more mutations from group (ii), said patient is treated with a conventionally fractionated radiation treatment (CF-RT) radiation, and wherein if said patient is identified as carrying or not carrying one or more mutations from group (iii), said patient is treated with a different treatment, wherein group (i) is:
a) not carrying or carrying only one copy of a G nucleotide at position 101 of SEQ ID NO: 53 (BMP2_rs1979855);
b) not carrying or carrying only one copy of a G nucleotide at position 101 of SEQ ID NO: 55 (PTPN2_rs1893217);
c) not carrying or carrying only one copy of a C nucleotide at position 101 of SEQ ID NO: 43 (BRCA2_rs15869);
d) carrying a C nucleotide at position 101 of SEQ ID NO: 65 (ABL1_rs11991);
e) not carrying or carrying only one copy of a G nucleotide at position 101 of SEQ ID NO: 63 (ERCC1_rs11615);
f) not carrying or carrying only one copy of an A nucleotide at position 101 of SEQ ID NO: 41 (RAD23A_rs8240);
g) carrying a G nucleotide at position 101 of SEQ ID NO: 59 (BATF3_rs6695772);
h) carrying a C nucleotide at position 101 of SEQ ID NO: 143 (FOXP3_rs2232365);
i) not carrying or carrying only one copy of a G nucleotide at position 101 of SEQ ID NO: 39 (IL19_rs1798);
j) not carrying a G or T nucleotide at position 101 of SEQ ID NO: 129 (LIN28A_rs9438623);
k) not carrying or carrying only one copy of an A nucleotide at position 101 of SEQ ID NO: 61 (IL18R1_rs11465660);
l) not carrying a T nucleotide at position 101 of SEQ ID NO: 153 (IL2RB_rs228942);
m) not carrying or carrying only one copy of a T nucleotide at position 101 of SEQ ID NO: 49 (CD274_rs1411262);
n) not carrying or carrying only one copy of a C nucleotide at position 101 of SEQ ID NO: 47 (BMP2_rs3178250);
o) not carrying a C nucleotide at position 101 of SEQ ID NO: 137 (SPI1_rs2071304);
p) not carrying a G nucleotide at position 101 of SEQ ID NO: 99 (IL19_rs2243158);
q) not carrying an A nucleotide at position 101 of SEQ ID NO: 167 (IL10_rs3024505);
r) carrying a G nucleotide at position 101 of SEQ ID NO: 187 (TRL4_rs4986790);
s) carrying an A nucleotide at position 101 of SEQ ID NO: 177 (VEGFA_rs41282644);
t) carrying a G or C nucleotide at position 101 of SEQ ID NO: 163 (P2RX7_rs3751143);
u) not carrying a C nucleotide at position 101 of SEQ ID NO: 15 (ERCC1_rs3212948);
v) not carrying a G or C nucleotide at position 101 of SEQ ID NO: 131 (FANCC_rs9673);
w) not carrying a G nucleotide at position 101 of SEQ ID NO: 51 (CD274_rs2297136);
x) not carrying or carrying only one copy of a G nucleotide at position 101 of SEQ ID NO: 19 (IL6_rs2069840);
y) not carrying or carrying only one copy of a G nucleotide at position 101 of SEQ ID NO: 45 (CD274_rs2282055);
z) carrying a G nucleotide at position 101 of SEQ ID NO: 57 (CD274_rs822339);
aa) carrying a G nucleotide at position 101 of SEQ ID NO: 95 (HAMP_rs10421768);
bb) carrying a C nucleotide at position 101 of SEQ ID NO: 145 (HAMP_rs1882694);
cc) not carrying a G nucleotide at position 101 of SEQ ID NO: 189 (ATM_rs1800057);
dd) not carrying a C nucleotide at position 101 of SEQ ID NO: 191 (ILRAP_rs79383051);
ee) carrying an A nucleotide at position 101 of SEQ ID NO: 155 (KIT_rs17084733); and
ff) carrying a T nucleotide at position 101 of SEQ ID NO: 7 (rs17599026).

group (ii) is:
a) carrying two copies of a G nucleotide at position 101 of SEQ ID NO: 53 (BMP2_rs1979855);
b) carrying two copies of a G nucleotide at position 101 of SEQ ID NO: 55 (PTPN2_rs1893217);
c) carrying two copies of a C nucleotide at position 101 of SEQ ID NO: 43 (BRCA2_rs15869);
d) not carrying a C nucleotide at position 101 of SEQ ID NO: 65 (ABL1_rs11991);
e) carrying two copies of a G nucleotide at position 101 of SEQ ID NO: 63 (ERCC1_rs11615);
f) carrying two copies of an A nucleotide at position 101 of SEQ ID NO: 41 (RAD23A_rs8240);
g) not carrying a G nucleotide at position 101 of SEQ ID NO: 59 (BATF3_rs6695772);
h) not carrying a C nucleotide at position 101 of SEQ ID NO: 143 (FOXP3_rs2232365);
i) carrying two copies of a G nucleotide at position 101 of SEQ ID NO: 39 (IL19_rs1798);
j) carrying a G or T nucleotide at position 101 of SEQ ID NO: 129 (LIN28A_rs9438623);
k) carrying two copies of an A nucleotide at position 101 of SEQ ID NO: 61 (IL18R1_rs11465660);
l) carrying a T nucleotide at position 101 of SEQ ID NO: 153 (IL2RB_rs228942);
m) carrying two copies of a T nucleotide at position 101 of SEQ ID NO: 49 (CD274_rs1411262);
n) carrying two copies of a C nucleotide at position 101 of SEQ ID NO: 47 (BMP2_rs3178250);
o) carrying a C nucleotide at position 101 of SEQ ID NO: 137 (SPI1_rs2071304);
p) carrying a G nucleotide at position 101 of SEQ ID NO: 99 (IL19_rs2243158);
q) carrying an A nucleotide at position 101 of SEQ ID NO: 167 (IL10_rs3024505);
r) not carrying a G nucleotide at position 101 of SEQ ID NO: 187 (TRL4_rs4986790);
s) not carrying an A nucleotide at position 101 of SEQ ID NO: 177 (VEGFA_rs41282644);
t) not carrying a G or C nucleotide at position 101 of SEQ ID NO: 163 (P2RX7_rs3751143);
u) carrying a C nucleotide at position 101 of SEQ ID NO: 15 (ERCC1_rs3212948);
v) carrying a G or C nucleotide at position 101 of SEQ ID NO: 131 (FANCC_rs9673);
w) carrying a G nucleotide at position 101 of SEQ ID NO: 51 (CD274_rs2297136);
x) carrying two copies of a G nucleotide at position 101 of SEQ ID NO: 19 (IL6_rs2069840);
y) carrying two copies of a G nucleotide at position 101 of SEQ ID NO: 45 (CD274_rs2282055);
z) not carrying a G nucleotide at position 101 of SEQ ID NO: 57 (CD274_rs822339);
aa) not carrying a G nucleotide at position 101 of SEQ ID NO: 95 (HAMP_rs10421768);
bb) not carrying a C nucleotide at position 101 of SEQ ID NO: 145 (HAMP_rs1882694);
cc) carrying a G nucleotide at position 101 of SEQ ID NO: 189 (ATM_rs1800057);
dd) carrying a C nucleotide at position 101 of SEQ ID NO: 191 (ILRAP_rs79383051);
ee) not carrying an A nucleotide at position 101 of SEQ ID NO: 155 (KIT_rs17084733); and
ff) not carrying a T nucleotide at position 101 of SEQ ID NO: 7 (rs17599026);

and group (iii) is:
a) carrying two copies of a G nucleotide at position 101 of SEQ ID NO: 53 (BMP2_rs1979855);
b) carrying two copies of a G nucleotide at position 101 of SEQ ID NO: 55
c) carrying two copies of a C nucleotide at position 101 of SEQ ID NO: 43 (BRCA2_rs15869);
d) not carrying a C nucleotide at position 101 of SEQ ID NO: 65 (ABL1_rs11991);
e) carrying two copies of a G nucleotide at position 101 of SEQ ID NO: 63 (ERCC1_rs11615);
f) carrying two copies of an A nucleotide at position 101 of SEQ ID NO: 41 (RAD23A_rs8240);
g) not carrying a G nucleotide at position 101 of SEQ ID NO: 59 (BATF3_rs6695772);
h) not carrying a C nucleotide at position 101 of SEQ ID NO: 143 (FOXP3_rs2232365);
i) carrying two copies of a G nucleotide at position 101 of SEQ ID NO: 39 (IL19_rs1798);
j) carrying a G or T nucleotide at position 101 of SEQ ID NO: 129 (LIN28A_rs9438623);
k) carrying two copies of an A nucleotide at position 101 of SEQ ID NO: 61 (IL18R1_rs11465660);
l) carrying a T nucleotide at position 101 of SEQ ID NO: 153 (IL2RB_rs228942);
m) carrying two copies of a T nucleotide at position 101 of SEQ ID NO: 49 (CD274_rs1411262);
n) carrying two copies of a C nucleotide at position 101 of SEQ ID NO: 47 (BMP2_rs3178250);
o) carrying a C nucleotide at position 101 of SEQ ID NO: 137 (SPIT_rs2071304);
p) carrying a G nucleotide at position 101 of SEQ ID NO: 99 (IL19_rs2243158);
q) carrying an A nucleotide at position 101 of SEQ ID NO: 167 (IL10_rs3024505);
r) not carrying a G nucleotide at position 101 of SEQ ID NO: 187 (TRL4_rs4986790);
s) not carrying an A nucleotide at position 101 of SEQ ID NO: 177 (VEGFA_rs41282644);
t) not carrying a G or C nucleotide at position 101 of SEQ ID NO: 163 (P2RX7_rs3751143);
u) carrying a C nucleotide at position 101 of SEQ ID NO: 15 (ERCC1_rs3212948);
v) carrying a G or C nucleotide at position 101 of SEQ ID NO: 131 (FANCC_rs9673);
w) carrying a G nucleotide at position 101 of SEQ ID NO: 51 (CD274_rs2297136);
x) carrying two copies of a G nucleotide at position 101 of SEQ ID NO: 19 (IL6_rs2069840);
y) carrying two copies of a G nucleotide at position 101 of SEQ ID NO: 45 (CD274_rs2282055);
z) not carrying a G nucleotide at position 101 of SEQ ID NO: 57 (CD274_rs822339);
aa) not carrying a G nucleotide at position 101 of SEQ ID NO: 95 (HAMP_rs10421768);
bb) not carrying a C nucleotide at position 101 of SEQ ID NO: 145 (HAMP_rs1882694);
cc) carrying a G nucleotide at position 101 of SEQ ID NO: 189 (ATM_rs1800057);
dd) carrying a C nucleotide at position 101 of SEQ ID NO: 191 (ILRAP_rs79383051);
ee) not carrying an A nucleotide at position 101 of SEQ ID NO: 155 (KIT_rs17084733); and
ff) not carrying a T nucleotide at position 101 of SEQ ID NO: 7 (rs17599026).

In certain embodiments, the hypofractionated radiation regimen comprises administering from about 20 Gy to about 35 Gy radiation over about 5 to about 8 fractions. In certain embodiments, the hypofractionated radiation regimen comprises administering stereotactic body radiation therapy. In certain embodiments, the method is a reduced toxicity method and the toxicity comprises acute or long-term toxicity. In certain embodiments, the toxicity comprises GI and/or GU toxicity.

In certain embodiments, the CF-RT regimen comprises administering from about 40 Gy to about 60 Gy radiation over a period of about 5 to about 6 weeks. In certain embodiments, the CF-RT regimen comprises administering from about 40 Gy to about 60 Gy radiation over about 15 to about 30 fractions.

In certain embodiments, the radiation therapy comprises external beam radiation therapy.

In certain embodiments, the different treatment comprises brachytherapy, chemotherapy, androgen deprivation therapy, immunotherapy, high intensity focused ultrasound, cryotherapy, laser ablation, photodynamic therapy, or surgery.

In another aspect, the disclosure relates to a method of treating a patient with a sarcoma, wherein said sarcoma is preferably treated by hypofractionated radiation, CF-RT, or a different treatment, wherein if said patient is identified as carrying or not carrying one or more mutations from group (i), said patient is treated with hypofractionated radiation, and if said patient is identified as carrying or not carrying one or more mutations from group (ii), said patient is treated with a conventionally fractionated radiation treatment (CF-RT) radiation and/or a different treatment, thereby to avoid distant failure, wherein group (i) is:
a) carrying a T nucleotide at position 101 of SEQ ID NO: 81 (TREX1_rs11797);
b) carrying a G or T nucleotide at position 101 of SEQ ID NO: 129 (LIN28A_rs9438623);
c) carrying or not carrying a C nucleotide at position 101 of SEQ ID NO: 127 (miR.34b.c.promoter_rs4938723);
d) carrying a C or G nucleotide at position 101 of SEQ ID NO: 119 (FCGR2A_rs1801274);
e) carrying a G nucleotide at position 101 of SEQ ID NO: 83 (CD274_rs4742098);
f) not carrying a G nucleotide at position 101 of SEQ ID NO: 17 (IL.6_rs12700386);
g) not carrying a C nucleotide at position 101 of SEQ ID NO: 137 (SPI1_rs2071304);
h) carrying a G nucleotide at position 101 of SEQ ID NO: 85 (ERCC4_rs4781562);
i) carrying a G nucleotide at position 101 of SEQ ID NO: 103 (IL10_rs3024496_P1.P2);
j) carrying a G nucleotide at position 101 of SEQ ID NO: 139 (IL10_rs3024496);
k) not carrying or carrying only one copy of a G nucleotide at position 101 of SEQ ID NO: 19 (IL.6_rs2069840);
l) carrying a T nucleotide at position 101 of SEQ ID NO: 165 (rs2187668);
m) not carrying a C nucleotide at position 101 of SEQ ID NO: 27 (CD274_rs4143815);
n) carrying a C nucleotide at position 101 of SEQ ID NO: 157 (KRAS_rs61764370);
o) carrying a G nucleotide at position 101 of SEQ ID NO: 5 (SHC4_rs1062124);
p) carrying a G nucleotide at position 101 of SEQ ID NO: 169 (rs922075);
q) carrying a G nucleotide at position 101 of SEQ ID NO: 63 (ERCC1_rs11615);
r) not carrying the deletion of a T nucleotide at [ATTTTG] of SEQ ID NO: 142 (EXO1_rs4150021);
s) carrying a C nucleotide at position 101 of SEQ ID NO: 173 (STAT4_rs7574070); and
t) carrying a T nucleotide at position 101 of SEQ ID NO: 171 (SOS1_rs1059313).

and group (ii) is:
t) not carrying a T nucleotide at position 101 of SEQ ID NO: 81 (TREX1_rs11797);
u) not carrying a G or T nucleotide at position 101 of SEQ ID NO: 129 (LIN28A_rs9438623);
v) carrying or not carrying a C nucleotide at position 101 of SEQ ID NO: 127 (miR.34b.c.promoter_rs4938723);
w) not carrying a C or G nucleotide at position 101 of SEQ ID NO: 119 (FCGR2A_rs1801274);
x) not carrying a G nucleotide at position 101 of SEQ ID NO: 83 (CD274_rs4742098);
y) carrying a G nucleotide at position 101 of SEQ ID NO: 17 (IL.6_rs12700386);
z) carrying a C nucleotide at position 101 of SEQ ID NO: 137 (SPI1_rs2071304);
aa) not carrying a G nucleotide at position 101 of SEQ ID NO: 85 (ERCC4_rs4781562);
bb) not carrying a G nucleotide at position 101 of SEQ ID NO: 103 (IL10_rs3024496_P1.P2);
cc) not carrying a G nucleotide at position 101 of SEQ ID NO: 139 (IL10_rs3024496),
dd) carrying two copies of a G nucleotide at position 101 of SEQ ID NO: 19 (IL.6_rs2069840);
ee) not carrying a T nucleotide at position 101 of SEQ ID NO: 165 (rs2187668);
ff) carrying a C nucleotide at position 101 of SEQ ID NO: 27 (CD274_rs4143815);
gg) not carrying a C nucleotide at position 101 of SEQ ID NO: 157 (KRAS_rs61764370);
hh) not carrying a G nucleotide at position 101 of SEQ ID NO: 5 (SHC4_rs1062124);
ii) not carrying a G nucleotide at position 101 of SEQ ID NO: 169 (rs922075);
jj) not carrying a G nucleotide at position 101 of SEQ ID NO: 63 (ERCC1_rs11615);
kk) carrying the deletion of a T nucleotide at [ATTTTG] of SEQ ID NO: 142 (EXO1_rs4150021);
ll) not carrying a C nucleotide at position 101 of SEQ ID NO: 173 (STAT4_rs7574070); and
mm) not carrying a T nucleotide at position 101 of SEQ ID NO: 171 (SOS1_rs1059313).

In another aspect, the disclosure relates to a method of treating a patient with a prostate cancer, wherein said prostate cancer is preferably treated by conventionally fractionated radiation treatment (CF-RT) or a different treatment, wherein if said patient is identified as carrying or not carrying one or more mutations from group (i), said patient is treated with a conventionally fractionated radiation treatment (CF-RT) radiation, whereas if said patient is identified as carrying or not carrying one or more mutations from group (ii), said patient is treated with a different treatment, thereby to avoid an increased risk of mortality, wherein group (i) is:
a) not carrying a C nucleotide at position 101 of SEQ ID NO: 77 (BIRC5_rs2239680);
b) carrying an A nucleotide at position 101 of SEQ ID NO: 93 (SMAD1_rs11724777);
c) carrying an A nucleotide at position 101 of SEQ ID NO: 109 (IL12A_rs568408); and
d) carrying a T nucleotide at position 101 of SEQ ID NO: 193 (IL13_rs20541);

and group (ii) is:
a) carrying a C nucleotide at position 101 of SEQ ID NO: 77 (BIRC5_rs2239680);
b) not carrying an A nucleotide at position 101 of SEQ ID NO: 93 (SMAD1_rs11724777);
c) not carrying an A nucleotide at position 101 of SEQ ID NO: 109 (IL12A_rs568408); and
d) not carrying a T nucleotide at position 101 of SEQ ID NO: 193 (IL13_rs20541).

In another aspect, the disclosure relates to a method of treating a patient with a prostate cancer, wherein said prostate cancer is preferably treated by conventionally fractionated radiation treatment (CF-RT) or a different treatment, wherein if said patient is identified as carrying or not carrying one or more mutations from group (i), said patient is treated with a conventionally fractionated radiation treatment (CF-RT) radiation, whereas if said patient is identified as carrying or not carrying one or more mutations from group (ii), said patient is treated with a different treatment, thereby to avoid an increased risk of biochemical relapse, wherein group (i) is:
a) not carrying a C nucleotide at position 101 of SEQ ID NO: 47 (BMP2_rs3178250);
b) carrying a G nucleotide at position 101 of SEQ ID NO: 91 (IL10RB_rs2834167);
c) carrying a G nucleotide at position 101 of SEQ ID NO: 57 (CD274_rs822339);
d) not carrying a G nucleotide at position 101 of SEQ ID NO: 45 (CD274_rs2282055);
e) not carrying an A nucleotide at position 101 of SEQ ID NO: 109 (IL12A_rs568408);
f) not carrying a G nucleotide at position 101 of SEQ ID NO: 195 (PARP1_rs8679);
g) not carrying a T nucleotide at position 101 of SEQ ID NO: 49 (CD274_rs1411262);
h) not carrying a G nucleotide at position 101 of SEQ ID NO: 53 (BMP2_rs1979855); and
i) carrying a C nucleotide at position 101 of SEQ ID NO: 163 (P2RX7_rs3751143);

and group (ii) is:
a) carrying a C nucleotide at position 101 of SEQ ID NO: 47 (BMP2_rs3178250);
b) not carrying a G nucleotide at position 101 of SEQ ID NO: 91 (IL10RB_rs2834167);
c) not carrying a G nucleotide at position 101 of SEQ ID NO: 57 (CD274_rs822339);
d) carrying a G nucleotide at position 101 of SEQ ID NO: 45 (CD274_rs2282055);
e) carrying an A nucleotide at position 101 of SEQ ID NO: 109 (IL12A_rs568408);
f) carrying a G nucleotide at position 101 of SEQ ID NO: 195 (PARP1_rs8679);
g) carrying a T nucleotide at position 101 of SEQ ID NO: 49 (CD274_rs1411262);
h) carrying a G nucleotide at position 101 of SEQ ID NO: 53 (BMP2_rs1979855); and
i) carrying a C nucleotide at position 101 of SEQ ID NO: 163 (P2RX7_rs3751143).

In another aspect, the disclosure relates to a method of treating a patient with a prostate cancer, wherein said prostate cancer is preferably treated by conventionally fractionated radiation treatment (CF-RT) or a different treatment, wherein if said patient is identified as carrying or not carrying one or more mutations from group (i), said patient is treated with a conventionally fractionated radiation treatment (CF-RT) radiation, whereas if said patient is identified as carrying or not carrying one or more mutations from group (ii), said patient is treated with a different treatment and optionally CF-RT, thereby to avoid an increased risk of distant metastasis, wherein group (i) is:
a) not carrying a T nucleotide at position 101 of SEQ ID NO: 197 (IL8_rs4073);
b) carrying a G or C nucleotide at position 101 of SEQ ID NO: 103 (IL10_rs3024496_P1);
c) carrying a C nucleotide occurs at position 101 of SEQ ID NO: 77 (BIRC5_rs2239680);
d) not carrying an A nucleotide at position 101 of SEQ ID NO: 9 (RAC1_rs9374);
e) carrying a G nucleotide occurs at position 101 of SEQ ID NO: 103 (IL10_rs3024496_P1.P2);
f) not carrying a G nucleotide at position 101 of SEQ ID NO: 53 (BMP2_rs1979855); and
g) not carrying a G nucleotide at position 101 of SEQ ID NO: 79 (NBN_rs1805794);

and group (ii) is:
a) carrying a T nucleotide at position 101 of SEQ ID NO: 197 (IL8_rs4073);
b) not carrying a G or C nucleotide at position 101 of SEQ ID NO: 103 (IL10_rs3024496_P1);
c) not carrying a C nucleotide occurs at position 101 of SEQ ID NO: 77 (BIRC5_rs2239680);
d) carrying an A nucleotide at position 101 of SEQ ID NO: 9 (RAC1_rs9374);
e) not carrying a G nucleotide occurs at position 101 of SEQ ID NO: 103 (IL10_rs3024496_P1.P2);
f) carrying a G nucleotide at position 101 of SEQ ID NO: 53 (BMP2_rs1979855); and
g) carrying a G nucleotide at position 101 of SEQ ID NO: 79 (NBN_rs1805794).

In another aspect, the disclosure relates to a method of treating a patient with a prostate cancer, wherein said prostate cancer is preferably treated by conventionally fractionated radiation treatment (CF-RT) or a different treatment, wherein if said patient is identified as carrying or not carrying one or more mutations from group (i), said patient is treated with a conventionally fractionated radiation treatment (CF-RT) radiation, whereas if said patient is identified as carrying or not carrying one or more mutations from group (ii), said patient is treated with a different treatment, thereby to avoid an increased risk of impotency, wherein group (i) is:
a) carrying a G nucleotide at position 101 of SEQ ID NO: 95 (HAMP_rs10421768);
b) not carrying a C nucleotide at position 101 of SEQ ID NO: 97 (XRCC1_rs25487);
c) not carrying a G nucleotide at position 101 of SEQ ID NO: 19 (IL.6_rs2069840);
d) carrying a C nucleotide at position 101 of SEQ ID NO: 89 (XRCC4_rs1040363); and
e) not carrying a G nucleotide at position 101 of SEQ ID NO: 99 (IL19_rs2243158);

and group (ii) is:
a) not carrying a G nucleotide at position 101 of SEQ ID NO: 95 (HAMP_rs10421768);
b) carrying a C nucleotide at position 101 of SEQ ID NO: 97 (XRCC1_rs25487);
c) carrying a G nucleotide at position 101 of SEQ ID NO: 19 (IL.6_rs2069840);
d) not carrying a C nucleotide at position 101 of SEQ ID NO: 89 (XRCC4_rs1040363); and
e) carrying a G nucleotide at position 101 of SEQ ID NO: 99 (IL19_rs2243158).

In certain embodiments, the CF-RT regimen comprises administering from about 40 Gy to about 60 Gy radiation over a period of about 5 to about 6 weeks. In certain embodiments, the CF-RT regimen comprises administering from about 40 Gy to about 60 Gy radiation over about 15 to about 30 fractions.

In certain embodiments, the radiation therapy comprises external beam radiation therapy.

In certain embodiments, the different treatment comprises hypofractionated radiation, brachytherapy, chemotherapy, androgen deprivation therapy, immunotherapy, high intensity focused ultrasound, cryotherapy, laser ablation, photodynamic therapy, or surgery.

In certain embodiments, the hypofractionated radiation regimen comprises administering from about 20 Gy to about 35 Gy radiation over about 5 to about 8 fractions.

In certain embodiments, the hypofractionated radiation regimen comprises administering stereotactic body radiation therapy.

In another aspect, the disclosure relates to a method for determining whether a cancer patient with a sarcoma has a decreased likelihood of having a toxic response to a hypofractionated radiation treatment, the method comprising determining if the patient is carrying or not carrying one or more of the following mutations:
a) not carrying or carrying only one copy of a C nucleotide at a position corresponding to position 101 of SEQ ID NO: 1 (miR.99a.promoter);
b) not carrying or carrying only one copy of an A nucleotide at a position corresponding to position 101 of SEQ ID NO: 3 (IL1.B_rs4848306);
c) not carrying or carrying only one copy of a G nucleotide at a position corresponding to position 101 of SEQ ID NO: 5 (SHC4_rs1062124);
d) carrying a T nucleotide at a position corresponding to position 101 of SEQ ID NO: 7 (rs17599026);
e) not carrying or carrying only one copy of an A nucleotide at a position corresponding to position 101 of SEQ ID NO: 9 (RAC1_rs9374);
f) not carrying a C nucleotide at a position corresponding to position 101 of SEQ ID NO: 11 (IL24_rs1150258);
g) not carrying or carrying only one copy of an A nucleotide at a position corresponding to position 101 of SEQ ID NO: 13 (IL1A_rs1800587);
h) not carrying a C nucleotide at a position corresponding to position 101 of SEQ ID NO: 15 (ERCC1_rs3212948);
i) not carrying or carrying only one copy of a G nucleotide at a position corresponding to position 101 of SEQ ID NO: 17 (IL.6_rs12700386);
j) not carrying or carrying only one copy of a G nucleotide at a position corresponding to position 101 of SEQ ID NO: 19 (IL.6_rs2069840);
k) not carrying or carrying only one copy of a T nucleotide at a position corresponding to position 101 of SEQ ID NO: 21 (CAMK2G_rs2306327);
l) not carrying or carrying only one copy of a T nucleotide at a position corresponding to position 101 of SEQ ID NO: 23 (BMP2_rs1980499);
m) not carrying or carrying only one copy of a C nucleotide at a position corresponding to position 101 of SEQ ID NO: 25 (EREG_rs1460008);
n) not carrying or carrying only one copy of a C nucleotide at a position corresponding to position 101 of SEQ ID NO: 27 (CD274_rs4143815);
o) not carrying or carrying only one copy of an A nucleotide at a position corresponding to position 101 of SEQ ID NO: 29 (IL17D_rs7787);
p) not carrying or carrying only one copy of a C nucleotide at a position corresponding to position 101 of SEQ ID NO: 31 (STAT3/rs3744483);
q) not carrying or carrying only one copy of a C nucleotide at a position corresponding to position 101 of SEQ ID NO: 33 (BMP4_rs17563)
r) not carrying a C nucleotide at a position corresponding to position 101 of SEQ ID NO: 35 (IRF5_rs10488631); and
s) not carrying or carrying only one copy of a T nucleotide at a position corresponding to position 101 of SEQ ID NO: 37 (UNGC.41.IL1RAP).

In another aspect, the disclosure relates to a method for determining whether a cancer patient with a sarcoma has an increased likelihood of having a toxic response to a hypofractionated radiation treatment, the method comprising determining if the patient is carrying or not carrying one or more of the following mutations:
t) carrying a C nucleotide at a position corresponding to position 101 of SEQ ID NO: 1 (miR.99a.promoter);
u) carrying an A nucleotide at a position corresponding to position 101 of SEQ ID NO: 3 (IL1.B_rs4848306);
v) carrying a G nucleotide at a position corresponding to position 101 of SEQ ID NO: 5 (SHC4_rs1062124);
w) not carrying a T nucleotide at a position corresponding to position 101 of SEQ ID NO: 7 (rs17599026);
x) carrying an A nucleotide at a position corresponding to position 101 of SEQ ID NO: 9 (RAC1_rs9374);
y) carrying a C nucleotide at a position corresponding to position 101 of SEQ ID NO: 11 (IL24_rs1150258);
z) carrying an A nucleotide at a position corresponding to position 101 of SEQ ID NO: 13 (IL1A_rs1800587);
aa) carrying a C nucleotide at a position corresponding to position 101 of SEQ ID NO: 15 (ERCC1_rs3212948);
bb) carrying a G nucleotide at a position corresponding to position 101 of SEQ ID NO: 17 (IL.6_rs12700386);
cc) carrying a G nucleotide at a position corresponding to position 101 of SEQ ID NO: 19 (IL.6_rs2069840);
dd) carrying a T nucleotide at a position corresponding to position 101 of SEQ ID NO: 21 (CAMK2G_rs2306327);
ee) carrying a T nucleotide at a position corresponding to position 101 of SEQ ID NO: 23 (BMP2_rs1980499);
ff) carrying a C nucleotide at a position corresponding to position 101 of SEQ ID NO: 25 (EREG_rs1460008);
gg) carrying a C nucleotide at a position corresponding to position 101 of SEQ ID NO: 27 (CD274_rs4143815);
hh) carrying an A nucleotide at a position corresponding to position 101 of SEQ ID NO: 29 (IL17D_rs7787);
ii) carrying two copies of a C nucleotide at a position corresponding to position 101 of SEQ ID NO: 31 (STAT3/rs3744483);
jj) carrying a C nucleotide at a position corresponding to position 101 of SEQ ID NO: 33 (BMP4_rs17563)
kk) carrying a C nucleotide at a position corresponding to position 101 of SEQ ID NO: 35 (IRF5_rs10488631); and
ll) carrying a T nucleotide at a position corresponding to position 101 of SEQ ID NO: 37 (UNGC.41.IL1RAP).

In certain embodiments, the toxic response comprises wound toxicity. In certain embodiments, the patient has a tumor on a lower extremity.

In another aspect, the disclosure relates to a method for determining whether a prostate cancer patient has a decreased likelihood of having a toxic response to a hypofractionated radiation treatment, the method comprising determining if the patient is carrying or not carrying one or more of the following mutations:
a) not carrying or carrying only one copy of a G nucleotide at position 101 of SEQ ID NO: 53 (BMP2_rs1979855);
b) not carrying or carrying only one copy of a G nucleotide at position 101 of SEQ ID NO: 55 (PTPN2_rs1893217);
c) not carrying or carrying only one copy of a C nucleotide at position 101 of SEQ ID NO: 43 (BRCA2_rs15869);
d) carrying a C nucleotide at position 101 of SEQ ID NO: 65 (ABL1_rs11991);
e) not carrying or carrying only one copy of a G nucleotide at position 101 of SEQ ID NO: 63 (ERCC1_rs11615);
f) not carrying or carrying only one copy of an A nucleotide at position 101 of SEQ ID NO: 41 (RAD23A_rs8240);
g) carrying a G nucleotide at position 101 of SEQ ID NO: 59 (BATF3_rs6695772);
h) carrying a C nucleotide at position 101 of SEQ ID NO: 143 (FOXP3_rs2232365);
i) not carrying or carrying only one copy of a G nucleotide at position 101 of SEQ ID NO: 39 (IL19_rs1798);
j) not carrying a G or T nucleotide at position 101 of SEQ ID NO: 129 (LIN28A_rs9438623);
k) not carrying or carrying only one copy of an A nucleotide at position 101 of SEQ ID NO: 61 (IL18R1_rs11465660);
l) not carrying a T nucleotide at position 101 of SEQ ID NO: 153 (IL2RB_rs228942);
m) not carrying or carrying only one copy of a T nucleotide at position 101 of SEQ ID NO: 49 (CD274_rs1411262);
n) not carrying or carrying only one copy of a C nucleotide at position 101 of SEQ ID NO: 47 (BMP2_rs3178250);
o) not carrying a C nucleotide at position 101 of SEQ ID NO: 137 (SPIT_rs2071304);
p) not carrying a G nucleotide at position 101 of SEQ ID NO: 99 (IL19_rs2243158);
q) not carrying an A nucleotide at position 101 of SEQ ID NO: 167 (IL10_rs3024505);
r) carrying a G nucleotide at position 101 of SEQ ID NO: 187 (TRL4_rs4986790);
s) carrying an A nucleotide at position 101 of SEQ ID NO: 177 (VEGFA_rs41282644);
t) carrying a G or C nucleotide at position 101 of SEQ ID NO: 163 (P2RX7_rs3751143);
u) not carrying a C nucleotide at position 101 of SEQ ID NO: 15 (ERCC1_rs3212948);
v) not carrying a G or C nucleotide at position 101 of SEQ ID NO: 131
w) not carrying a G nucleotide at position 101 of SEQ ID NO: 51 (CD274_rs2297136);
x) not carrying or carrying only one copy of a G nucleotide at position 101 of SEQ ID NO: 19 (IL6_rs2069840);
y) not carrying or carrying only one copy of a G nucleotide at position 101 of SEQ ID NO: 45 (CD274_rs2282055);
z) carrying a G nucleotide at position 101 of SEQ ID NO: 57 (CD274_rs822339);
aa) carrying a G nucleotide at position 101 of SEQ ID NO: 95 (HAMP_rs10421768);
bb) carrying a C nucleotide at position 101 of SEQ ID NO: 145 (HAMP_rs1882694);
cc) not carrying a G nucleotide at position 101 of SEQ ID NO: 189 (ATM_rs1800057);
dd) not carrying a C nucleotide at position 101 of SEQ ID NO: 191 (ILRAP_rs79383051);
ee) carrying an A nucleotide at position 101 of SEQ ID NO: 155 (KIT_rs17084733); and
ff) carrying a T nucleotide at position 101 of SEQ ID NO: 7 (rs17599026).

In another aspect, the disclosure relates to a method for determining whether a prostate cancer patient has an increased likelihood of having a toxic response to a hypofractionated radiation treatment, the method comprising determining if the patient is carrying or not carrying one or more mutations selected from:
a) carrying two copies of a G nucleotide at position 101 of SEQ ID NO: 53 (BMP2_rs1979855);
b) carrying two copies of a G nucleotide at position 101 of SEQ ID NO: 55 (PTPN2_rs1893217);
c) carrying two copies of a C nucleotide at position 101 of SEQ ID NO: 43 (BRCA2_rs15869);
d) not carrying a C nucleotide at position 101 of SEQ ID NO: 65 (ABL1_rs11991);
e) carrying two copies of a G nucleotide at position 101 of SEQ ID NO: 63 (ERCC1_rs11615);
f) carrying two copies of an A nucleotide at position 101 of SEQ ID NO: 41 (RAD23A_rs8240);
g) not carrying a G nucleotide at position 101 of SEQ ID NO: 59 (BATF3_rs6695772);
h) not carrying a C nucleotide at position 101 of SEQ ID NO: 143 (FOXP3_rs2232365);
i) carrying two copies of a G nucleotide at position 101 of SEQ ID NO: 39 (IL19_rs1798);
j) carrying a G or T nucleotide at position 101 of SEQ ID NO: 129 (LIN28A_rs9438623);
k) carrying two copies of an A nucleotide at position 101 of SEQ ID NO: 61 (IL18R1_rs11465660);
l) carrying a T nucleotide at position 101 of SEQ ID NO: 153 (IL2RB_rs228942);
m) carrying two copies of a T nucleotide at position 101 of SEQ ID NO: 49 (CD274_rs1411262);
n) carrying two copies of a C nucleotide at position 101 of SEQ ID NO: 47 (BMP2_rs3178250);
o) carrying a C nucleotide at position 101 of SEQ ID NO: 137 (SPIT_rs2071304);
p) carrying a G nucleotide at position 101 of SEQ ID NO: 99 (IL19_rs2243158);
q) carrying an A nucleotide at position 101 of SEQ ID NO: 167 (IL10_rs3024505);
r) not carrying a G nucleotide at position 101 of SEQ ID NO: 187 (TRL4_rs4986790);
s) not carrying an A nucleotide at position 101 of SEQ ID NO: 177 (VEGFA_rs41282644);
t) not carrying a G or C nucleotide at position 101 of SEQ ID NO: 163 (P2RX7_rs3751143);
u) carrying a C nucleotide at position 101 of SEQ ID NO: 15 (ERCC1_rs3212948);
v) carrying a G or C nucleotide at position 101 of SEQ ID NO: 131 (FANCC_rs9673);
w) carrying a G nucleotide at position 101 of SEQ ID NO: 51 (CD274_rs2297136);
x) carrying two copies of a G nucleotide at position 101 of SEQ ID NO: 19 (IL6_rs2069840);
y) carrying two copies of a G nucleotide at position 101 of SEQ ID NO: 45 (CD274_rs2282055);
z) not carrying a G nucleotide at position 101 of SEQ ID NO: 57 (CD274_rs822339);
aa) not carrying a G nucleotide at position 101 of SEQ ID NO: 95
bb) not carrying a C nucleotide at position 101 of SEQ ID NO: 145 (HAMP_rs1882694);
cc) carrying a G nucleotide at position 101 of SEQ ID NO: 189 (ATM_rs1800057);
dd) carrying a C nucleotide at position 101 of SEQ ID NO: 191 (ILRAP_rs79383051);
ee) not carrying an A nucleotide at position 101 of SEQ ID NO: 155 (KIT_rs17084733); and
ff) not carrying a T nucleotide at position 101 of SEQ ID NO: 7 (rs17599026).

In certain embodiments, the hypofractionated radiation regimen comprises administering from about 20 Gy to about 35 Gy radiation over about 5 to about 8 fractions. In certain embodiments, the hypofractionated radiation regimen comprises administering stereotactic body radiation therapy.

In another aspect, the disclosure relates to a method for determining whether a prostate cancer patient has an increased likelihood of having a toxic response to CF-RT, the method comprising determining if the patient is carrying or not carrying one or more mutations selected from:
a) not carrying an A nucleotide at position 101 of SEQ ID NO: 67 (ATM_rs189037);
b) carrying a G nucleotide at position 101 of SEQ ID NO: 69 (CD6_rs76677607);
c) carrying two copies of an A nucleotide at position 101 of SEQ ID NO: 13 (IL1A_rs1800587);
d) not carrying an A nucleotide at position 101 of SEQ ID NO: 179 (IL1A_rs17561);
e) not carrying a G nucleotide at position 101 of SEQ ID NO: 73 (BRCA2_rs7334543);
f) carrying a T nucleotide at position 101 of SEQ ID NO: 75 (ILF3.58_rs118142475);
g) not carrying a C nucleotide at position 101 of SEQ ID NO: 77 (BIRC5_rs2239680);
h) not carrying a T nucleotide at position 101 of SEQ ID NO: 107 (IL2RA_rs2476491);
i) not carrying an A nucleotide at position 101 of SEQ ID NO: 109 (IL12A_rs568408);
j) carrying a C nucleotide at position 101 of SEQ ID NO: 111 (GSK3B_rs2037547);
k) not carrying a G nucleotide at position 101 of SEQ ID NO: 135 (IL6_rs1800795);
l) carrying a T nucleotide at position 101 of SEQ ID NO: 105 (LIG4_rs2232643);
m) carrying two copies of a C nucleotide at position 101 of SEQ ID NO: 31 (STAT3_rs3744483);
n) not carrying a G nucleotide at position 101 of SEQ ID NO: 115 (IL6_rs1800797);
o) carrying two copies of a G nucleotide at position 101 of SEQ ID NO: 17 (IL.6_rs12700386);
p) carrying two copies of an A nucleotide at position 101 of SEQ ID NO: 9 (RAC1_rs9374);
q) carrying a T nucleotide at position 101 of SEQ ID NO: 121 (ATM_rs373759);
r) carrying an A nucleotide at position 101 of SEQ ID NO: 123 (CETN2_rs8230);
s) carrying an A nucleotide at position 101 of SEQ ID NO: 126 (XRCC3_rs861539);
t) not carrying a C nucleotide at position 101 of SEQ ID NO: 127 (miR.34b.c.promoter_rs4938723);
u) carrying two copies of an A nucleotide at position 101 of SEQ ID NO: 61 (IL18R1_rs11465660);
v) carrying two copies of an A nucleotide at position 101 of SEQ ID NO: 3 rs4848306);
w) carrying a G or C nucleotide at position 101 of SEQ ID NO: 131 (FANCC_rs9673);
x) not carrying an A nucleotide at position 101 of SEQ ID NO: 133 (REV3L_rs465646);
y) carrying a G nucleotide at position 101 of SEQ ID NO: 51 (CD274_rs2297136);
z) not carrying a C nucleotide at position 101 of SEQ ID NO: 183 (EGFR_rs884225);
aa) carrying a G nucleotide at position 101 of SEQ ID NO: 181 (TGFB1_rs1800469);
bb) not carrying a T nucleotide at position 101 of SEQ ID NO: 165 (rs2187668);
cc) not carrying a G nucleotide at position 101 of SEQ ID NO: 185 (rs17388569);
dd) carrying a G nucleotide at position 101 of SEQ ID NO: 79 (NBN_rs1805794);
ee) not carrying a G nucleotide at position 101 of SEQ ID NO: 83 (CD274_rs4742098);
ff) not carrying a G nucleotide at position 101 of SEQ ID NO: 85 (ERCC4_rs4781562);
gg) carrying an A, C, or G nucleotide at position 101 of SEQ ID N: 87 (MSH2_rs2303428);
hh) not carrying a T nucleotide at position 101 of SEQ ID NO: 7 (rs17599026);
ii) carrying an A nucleotide at position 101 of SEQ ID NO: 151 (IL2RA_rs11256497);
jj) not carrying a C nucleotide at position 101 of SEQ ID NO: 159 (LIG4_rs3093772_P1.P2);
kk) carrying two copies of an A nucleotide at position 101 of SEQ ID NO: 29 (IL17D_rs7787);
ll) carrying two copies of a C nucleotide at position 101 of SEQ ID NO: (CD274_rs4143815);
mm) carrying an A nucleotide at position 101 of SEQ ID NO: 175 (UNGC.96.TGFB2_NA);
nn) carrying a G nucleotide at position 101 of SEQ ID NO: 161 (MDM2_rs769412);
oo) carrying an A nucleotide at position 101 of SEQ ID NO: 167 (IL10_rs3024505);
pp) carrying two copies of a C nucleotide at position 101 of SEQ ID NO: 43 (BRCA2_rs15869);
qq) carrying a G nucleotide at position 101 of SEQ ID NO: 147 (IL10_rs1800872);
rr) carrying two copies of a G nucleotide at position 101 of SEQ ID NO: 53 (BMP2_rs1979855);
ss) carrying two copies of a G nucleotide at position 101 of SEQ ID NO: 55 (PTPN2_rs1893217);
tt) not carrying a C nucleotide at position 101 of SEQ ID NO: 65 (ABL1_rs11991); and
uu) carrying two copies of a G nucleotide at position 101 of SEQ ID NO: 63 (ERCC1_rs11615).

In certain embodiments, the toxicity is acute or long-term toxicity. In certain embodiments, the toxicity is GI and/or GU toxicity.

In certain embodiments, the CF-RT regimen comprises administering from about 40 Gy to about 60 Gy radiation over a period of about 5 to about 6 weeks. In certain embodiments, the CF-RT regimen comprises administering from about 40 Gy to about 60 Gy radiation over about 15 to about 30 fractions.

In certain embodiments, the radiation treatment is external beam radiation therapy.

In another aspect, the disclosure relates to a method for determining whether a cancer patient having a sarcoma has a decreased likelihood of distant failure in response to hypofractionated radiation, the method comprising determining whether the patient is carrying or not carrying one or more mutations in the following SNPs:
a) not carrying a T nucleotide at position 101 of SEQ ID NO: 81 (TREX1_rs11797);
b) not carrying a G or T nucleotide at position 101 of SEQ ID NO: 129 (LIN28A_rs9438623);
c) carrying or not carrying a C nucleotide at position 101 of SEQ ID NO: 127 (miR.34b.c.promoter_rs4938723);
d) not carrying a C or G nucleotide at position 101 of SEQ ID NO: 119 (FCGR2A_rs1801274);
e) not carrying a G nucleotide at position 101 of SEQ ID NO: 83 (CD274_rs4742098);
f) carrying a G nucleotide at position 101 of SEQ ID NO: 17 (IL.6_rs12700386);
g) carrying a C nucleotide at position 101 of SEQ ID NO: 137 (SPI1_rs2071304);
h) not carrying a G nucleotide at position 101 of SEQ ID NO: 85 (ERCC4_rs4781562);
i) not carrying a G nucleotide at position 101 of SEQ ID NO: 103 (IL10_rs3024496_P1.P2);
j) not carrying a G nucleotide at position 101 of SEQ ID NO: 139 (IL10_rs3024496);
k) carrying two copies of a G nucleotide at position 101 of SEQ ID NO: 19 (IL.6_rs2069840);
l) carrying a T nucleotide at position 101 of SEQ ID NO: 165 (rs2187668);
m) carrying a C nucleotide at position 101 of SEQ ID NO: 27 (CD274_rs4143815);
n) not carrying a C nucleotide at position 101 of SEQ ID NO: 157 (KRAS_rs61764370);
o) not carrying a G nucleotide at position 101 of SEQ ID NO: 5 (SHC4_rs1062124);
p) not carrying a G nucleotide at position 101 of SEQ ID NO: 169 (rs922075);
q) not carrying a G nucleotide at position 101 of SEQ ID NO: 63 (ERCC1_rs11615);
r) carrying the deletion of a T nucleotide at [ATTTTG] of SEQ ID NO: 142 (EXO1_rs4150021);
s) not carrying a C nucleotide at position 101 of SEQ ID NO: 173 (STAT4_rs7574070); and
t) not carrying a T nucleotide at position 101 of SEQ ID NO: 171 (SOS1_rs1059313).

In certain embodiments, the hypofractionated radiation regimen comprises administering from about 20 Gy to about 35 Gy radiation over about 5 to about 8 fractions.

In another aspect, the disclosure relates to a method for determining whether a prostate cancer patient has an increased risk of mortality following CF-RT, the method comprising identifying the patient as carrying or not carrying one or more mutations in a SNP selected from:
a) not carrying a C nucleotide at position 101 of SEQ ID NO: 77 (BIRC5_rs2239680);
b) carrying an A nucleotide at position 101 of SEQ ID NO: 93 (SMAD1_rs11724777);
c) carrying an A nucleotide at position 101 of SEQ ID NO: 109 (IL12A_rs568408); and
d) carrying a T nucleotide at position 101 of SEQ ID NO: 193 (IL13_rs20541).

In another aspect, the disclosure relates to a method for determining whether a prostate cancer patient has an increased risk of biochemical relapse following CF-RT, the method comprising identifying the patient as carrying or not carrying one or more mutations in a SNP selected from:
a) not carrying a C nucleotide at position 101 of SEQ ID NO: 47 (BMP2_rs3178250);
b) carrying a G nucleotide at position 101 of SEQ ID NO: 91 (IL10RB_rs2834167);
c) carrying a G nucleotide at position 101 of SEQ ID NO: 57 (CD274_rs822339);
d) not carrying a G nucleotide at position 101 of SEQ ID NO: 45 (CD274_rs2282055);
e) not carrying an A nucleotide at position 101 of SEQ ID NO: 109 (IL12A_rs568408);
f) not carrying a G nucleotide at position 101 of SEQ ID NO: 195 (PARP1_rs8679);
g) not carrying a T nucleotide at position 101 of SEQ ID NO: 49 (CD274_rs1411262);
h) not carrying a G nucleotide at position 101 of SEQ ID NO: 53 (BMP2_rs1979855); and
i) not carrying a C nucleotide at position 101 of SEQ ID NO: 163 (P2RX7_rs3751143).

In another aspect, the disclosure relates to a method for determining whether a prostate cancer patient has an increased risk of distant metastasis following CF-RT, the method comprising identifying the patient as carrying or not carrying one or more of the following mutations in a SNP:
a) not carrying a G nucleotide at position 101 of SEQ ID NO: 103 (IL10_rs3024496);
b) not carrying a C nucleotide occurs at position 101 of SEQ ID NO: 101 (FOXP3_rs2280883);
c) not carrying a C nucleotide occurs at position 101 of SEQ ID NO: 77 (BIRC5_rs2239680);
d) not carrying a G nucleotide occurs at position 101 of SEQ ID NO: 103 (IL10_rs3024496_P1.P2);
e) carrying a T nucleotide at position 101 of SEQ ID NO: 105 (LIG4_rs2232643); and
f) carrying an A nucleotide at position 101 of SEQ ID NO: 9 (RAC1_rs9374).

In another aspect, the disclosure relates to a method for determining whether a prostate cancer patient has a decreased risk of impotency following treatment with radiation, the method comprising identifying the patient as carrying or not carrying one or more mutations in a SNP selected from:
a) carrying a G nucleotide at position 101 of SEQ ID NO: 95 (HAMP_rs10421768);
b) not carrying a C nucleotide at position 101 of SEQ ID NO: 97 (XRCC1_rs25487);
c) not carrying a G nucleotide at position 101 of SEQ ID NO: 19 (IL.6_rs2069840);
d) carrying a C nucleotide at position 101 of SEQ ID NO: 89 (XRCC4_rs1040363); and
e) not carrying a G nucleotide at position 101 of SEQ ID NO: 99 (IL19_rs2243158).

In certain embodiments, a determining step includes reviewing a medical record of the patient containing the results of a SNP genotyping test. In certain embodiments, the determining step includes performing a SNP genotyping method as described herein. The assessment of the patient's likelihood of a toxic response to a radiation therapy may be based on determining the presence or absence of only one, only two, only three, only four, only five, only six, only seven, only eight, only nine or all ten of these markers, but does not necessarily require assessment of all ten markers. For example, the assessment may be based on determining the presence or absence of at least one, at least two, at least three, at least four, at least five, at least six, at least seven, at least eight, or at least nine of the aforementioned markers associated with toxicity.

If the patient carries or does not carry one or more of these mutations, the patient is predicted as having a decreased probability of having a toxic response to the agent. In one embodiment, it is determined whether the patient carries one, two, three, four or all five of the aforementioned biomarkers. For example, the determination of whether a patient has a decreased probability of a toxic response to the agent may be based on whether the patient carries or does not carry only one, only two, only three, only four, or all five biomarkers, but may not require assessment of all five markers. For example, the assessment may be based on determining the presence or absence of at least one, at least two, at least three, or at least four of the aforementioned markers associated with toxicity.

In certain embodiments, the disclosure provides a method for determining a patient's likelihood of developing toxicity based on a clinical variable (presence of lower extremity tumor) and 9 SNPs—IL6_rs2069840, SHC4_rs1062124, ERCC1_rs3212948, RAC1_rs9374, UNGC.41.IL1RAP, CD274_rs4143815, miR99a promoter, IL1A_rs1800587, and CD6_rs76677607. Accordingly, in certain embodiments, the disclosure relates to a method of administering radiation to a cancer patient, wherein the cancer patient is identified as having a lower extremity tumor and/or is carrying or not carrying one or more mutations selected from a G nucleotide at a position corresponding to position 101 of SEQ ID NO: 19 (IL.6_rs2069840), a G nucleotide at a position corresponding to position 101 of SEQ ID NO: 5 (SHC4_rs1062124), a C nucleotide at a position corresponding to position 101 of SEQ ID NO: 15 (ERCC1_rs3212948), an A nucleotide at a position corresponding to position 101 of SEQ ID NO: 117 (RAC1_rs9374), a T nucleotide at a position corresponding to position 101 of SEQ ID NO: 37 (UNGC.41.IL1RAP), a C nucleotide at a position corresponding to position 101 of SEQ ID NO: 27 (CD274_rs4143815), a C nucleotide at a position corresponding to position 101 of SEQ ID NO: 1 (miR.99a.promoter), an A nucleotide at a position corresponding to position 101 of SEQ ID NO: 13 (IL1A_rs1800587), and an A nucleotide at a position corresponding to position 101 of SEQ ID NO: 69 (CD6_rs76677607).

In certain embodiments, if a patient is identified as not homozygous wild-type for IL6_rs2069840, the marker CD6_rs76677607 is considered. In certain embodiments, a patient who is identified as homozygous wild-type (0) for CD6_rs76677607 is not predicted to have a toxic response to radiation. If the patient is identified as not homozygous wild-type (0) for CD6_rs76677607, the marker CD274_rs4143815 is considered. If the patient is identified as homozygous wild-type (0) for CD274_rs4143815, the patient is not predicted to have a toxic response. If the patient is identified as not homozygous wild-type (0) for CD274_rs4143815, the patient is predicted to have a toxic response.

If the patient is identified as homozygous wild-type (0) for the first variable of predictive value, IL6_rs2069840, the marker SHC4_rs1062124 is considered. If the patient is identified as homozygous wild-type (0) for SHC4_rs1062124, the marker ERCC1_rs3212948 is considered. If the patient is identified as homozygous wild-type (0) for ERCC1_rs3212948, the patient is not predicted to have a toxic response. If the patient is identified as not homozygous wild-type (0) for ERCC1_rs3212948, the marker RAC1_rs9374 should be considered. If the patient is identified as homozygous wild-type (0) for RAC1_rs9374, the patient is not predicted to have a toxic response. If the patient is identified as not homozygous wild-type (0) for RAC1_rs9374, the patient is predicted to have a toxic response.

If the patient is identified as not homozygous wild-type (0) for SHC4_rs1062124, the presence of a lower extremity tumor should be considered. If the patient does not have a tumor on a lower extremity, the patient is not predicted to have a toxic response. If the patient does have a tumor on a lower extremity, the marker UNGC.41.IL1RAP is considered. If the patient is identified as not homozygous wild-type (0) for UNGC.41.IL1RAP, the patient is not predicted to have a toxic response. If the patient is identified as homozygous wild-type (0) for UNGC.41.IL1RAP, the marker CD274_rs4143815 should be considered. If the patient is identified as not homozygous wild-type (0) or heterozygous (1) for CD274_rs4143815, the patient is not predicted to have a toxic response. If the patient is identified as homozygous wild-type (0) or heterozygous (1) for CD274_rs4143815, the marker miR.99a.promoter is considered. If the patient is identified as homozygous wild-type (0) for miR.99a.promoter, the patient is predicted to have a toxic response. If the patient is identified as not homozygous wild-type (0) for miR.99a.promoter, the marker IL1A_rs1800587 is considered. If the patient is heterozygous (1) for IL1A_rs1800587, the patient is not predicted to have a toxic response. If the patient is identified as not heterozygous (1) for IL1A_rs1800587, the patient is predicted to have a toxic response.

In certain embodiments, if the patient is predicted not to have a toxic response, the method further comprises administering radiation to the patient. In certain embodiments, if the patient is predicted to have a toxic response, the method further comprises administering a different treatment to the patient, for example, a different form of radiation or brachytherapy, chemotherapy, androgen deprivation therapy, immunotherapy, high intensity focused ultrasound, cryotherapy, laser ablation, photodynamic therapy, or surgery. In certain embodiments, the method further comprises determining whether the patient is homozygous wild type or heterozygous for a recited SNP.

Each of the biomarkers disclosed herein as being predictive of increased risk for mortality, distant failure or metastasis, or impotency may be used either alone or in combination with one or more of the other markers disclosed herein as predictive of increased risk for mortality, distant failure or metastasis, or impotency in order to predict whether or not a patient has an increased risk for mortality, distant failure or metastasis, or impotency regardless of whether radiation therapy is administered. For example, one may use one, two, three, four, five, six, seven, eight, nine, or ten of the biomarkers in determining a patient's predicted risk for mortality, distant failure or metastasis, or impotency. In particular, determining whether a patient is homozygous or heterozygous for a particular marker associated with increased risk for mortality, distant failure or metastasis, or impotency (or does not carry the marker, i.e., the patient is wild-type) can be useful in determining the patient's predicted risk for mortality, distant failure or metastasis, or impotency following radiation treatment. For example, one may determine the zygosity of a patient with respect to one, two, three, four, five, six, seven, eight, nine or ten of the biomarkers disclosed herein as predictive of increased risk for mortality, distant failure or metastasis, or impotency in order to determine the patient's predicted increased risk for mortality, distant failure or metastasis, or impotency following radiation therapy. For example, one may determine the zygosity of at least one, at least two, at least three, at least four, at least five, at least six, at least seven, at least eight, or at least nine of the aforementioned markers associated with increased risk for mortality, distant failure or metastasis, or impotency.

In one embodiment of the invention, determining whether a patient has an increased risk of distant failure following a radiation treatment includes determining whether the patient carries or does not carry one or more of the following mutations:
a) a T nucleotide at position 101 of SEQ ID NO: 81 (TREX1_rs11797);
b) a G or T nucleotide at position 101 of SEQ ID NO: 129 (LIN28A_rs9438623);
c) a C nucleotide at position 101 of SEQ ID NO: 127 (miR.34b.c.promoter_rs4938723);
d) a C or G nucleotide at position 101 of SEQ ID NO: 119 (FCGR2A_rs1801274);
e) a G nucleotide at position 101 of SEQ ID NO: 83 (CD274_rs4742098);
f) a G nucleotide at position 101 of SEQ ID NO: 17 (IL.6_rs12700386);
g) a C nucleotide at position 101 of SEQ ID NO: 137 (SPI1_rs2071304);
h) a G nucleotide at position 101 of SEQ ID NO: 85 (ERCC4_rs4781562);
i) a G nucleotide at position 101 of SEQ ID NO: 103 (IL10_rs3024496_P1.P2);
j) a G nucleotide at position 101 of SEQ ID NO: 139 (IL10_rs3024496);
k) a G nucleotide at position 101 of SEQ ID NO: 19 (IL.6_rs2069840);
l) a T nucleotide at position 101 of SEQ ID NO: 165 (rs2187668);
m) a C nucleotide at position 101 of SEQ ID NO: 27 (CD274_rs4143815);
n) a C nucleotide at position 101 of SEQ ID NO: 157 (KRAS_rs61764370);
o) a G nucleotide at position 101 of SEQ ID NO: 5 (SHC4_rs1062124);
p) a G nucleotide at position 101 of SEQ ID NO: 169 (rs922075);
q) a G nucleotide at position 101 of SEQ ID NO: 63 (ERCC1_rs11615);
r) deletion of a T nucleotide at [ATTTTG] of SEQ ID NO: 142 (EXO1_rs4150021);
s) a C nucleotide at position 101 of SEQ ID NO: 173 (STAT4_rs7574070); and
t) carrying a T nucleotide at position 101 of SEQ ID NO: 171 (SOS1_rs1059313).

In one embodiment of the invention, determining whether a patient has an increased risk of mortality following a radiation treatment includes determining whether the patient carries or does not carry one or more of the following mutations:
a) carrying a C nucleotide at position 101 of SEQ ID NO: 77 (BIRC5_rs2239680);
b) carrying an A nucleotide at position 101 of SEQ ID NO: 93 (SMAD1_rs11724777);
c) carrying an A nucleotide at position 101 of SEQ ID NO: 109 (IL12A_rs568408); and
d) carrying a T nucleotide at position 101 of SEQ ID NO: 193 (IL13_rs20541).

In one embodiment of the invention, determining whether a patient has an increased risk of distant metastasis following a radiation treatment includes determining whether the patient carries or does not carry one or more of the following mutations:
a) a G nucleotide at position 101 of SEQ ID NO: 103 (IL10_rs3024496);
b) a C nucleotide occurs at position 101 of SEQ ID NO: 101 (FOXP3_rs2280883);
c) a C nucleotide occurs at position 101 of SEQ ID NO: 77 (BIRC5_rs2239680);
d) a G nucleotide occurs at position 101 of SEQ ID NO: 103 (IL10_rs3024496_P1.P2);
e) a T nucleotide at position 101 of SEQ ID NO: 105 (LIG4_rs2232643); and
f) an A nucleotide at position 101 of SEQ ID NO: 9 (RAC1_rs9374).

In one embodiment of the invention, determining whether a patient has an increased risk of impotency following a radiation treatment includes determining whether the patient carries or does not carry one or more of the following mutations:
a) a G nucleotide at position 101 of SEQ ID NO: 95 (HAMP_rs10421768);
b) a C nucleotide at position 101 of SEQ ID NO: 97 (XRCC1_rs25487);
c) a G nucleotide at position 101 of SEQ ID NO: 19 (IL.6_rs2069840);
d) a C nucleotide at position 101 of SEQ ID NO: 89 (XRCC4_rs1040363); and
e) a G nucleotide at position 101 of SEQ ID NO: 99 (IL19_rs2243158).

In certain embodiments, a determining step includes reviewing a medical record of the patient containing the results of a SNP genotyping test. In certain embodiments, the determining step includes performing a SNP genotyping method as described herein. The assessment of the patient's likelihood of experiencing morality, distant failure or metastasis, or impotency following a radiation therapy may be based on determining the presence or absence of only one, only two, only three, only four, only five, only six, only seven, only eight, only nine or all ten of these markers, but does not necessarily require assessment of all ten markers. For example, the assessment may be based on determining the presence or absence of at least one, at least two, at least three, at least four, at least five, at least six, at least seven, at least eight, or at least nine of the aforementioned markers associated with morality, distant failure or metastasis, or impotency.

If the patient carries or does not carry one or more of these mutations, the patient is predicted as having a decreased probability of experiencing morality, distant failure or metastasis, or impotency following a radiation therapy. In one embodiment, it is determined whether the patient carries one, two, three, four or all five of the aforementioned biomarkers. For example, the determination of whether a patient has a decreased probability of experiencing morality, distant failure or metastasis, or impotency following a radiation therapy may be based on whether the patient does not carry only one, only two, only three, only four, or all five biomarkers, but may not require assessment of all five markers. For example, the assessment may be based on determining the presence or absence of at least one, at least two, at least three, or at least four of the aforementioned markers associated with morality, distant failure or metastasis, or impotency.

### Definitions

As used herein, the terms “treat,” “treating,” or “treatment” in the context of cancer refer to (a) slowing of growth of a tumor, (b) cessation of growth of a tumor, (c) regression, or (d) improvement in one or more the patient's symptoms. According to one embodiment of the invention, “treating” or “treat” may refer to patient outcomes where a patient receiving radiation therapy exhibits a response to that therapy. The terms “radiation treatment” and “radiation therapy” are used interchangeably herein.

As used herein, the term “toxicity” or “toxic response” refers to the occurrence of one or more adverse reactions a patient may experience in response to a cancer therapy, for example, radiation. In certain embodiments, the Radiation Therapy Oncology Group (RTOG) morbidity grading system is used to assess toxicity. (See, (Cox et al. (1995) Int J Radiat Oncol Biol Phys 31(5):1341-6.) In certain embodiments, the toxicity is short term toxicity. In certain embodiments, the toxicity is long term toxicity (i.e., toxicities occurring at least three months after radiation treatment). In certain embodiments, the toxicity is wound toxicity. In certain embodiments, the toxicity is gastrointestinal (GI) toxicity (e.g., nausea, diarrhea, vomiting, bleeding). In certain embodiments, the toxicity is genitourinary (GU) toxicity (e.g., macrohematuria, frequent urination, and urinary retention). In certain embodiments, the toxicity is skin toxicity (e.g., contact dermatitis, photosensitive dermatitis, contact urticaria, chemical-induced acne, pigmentary disturbance, drug rash (cutaneous reaction), hair disturbance, and nail disturbance). In certain embodiments, the toxicity is fibrosis. In certain embodiments, the toxicity is dysphagia. In certain embodiments, the toxicity is impotency. In certain embodiments, long term (late) toxicity is determined using the RTOG/EORTC Late Radiation Morbidity Scoring Schema as shown in TABLE 1. In certain embodiments, short term (acute) toxicity is determined using the RTOG Scoring Schema for Cooperative Group Common Toxicity Criteria as shown in TABLE 2.

TABLE 1
RTOG/EORTC Late Radiation Morbidity Scoring Schema

| Organ Tissue | 0 | Grade 1 | Grade 2 | Grade 3 | Grade 4 | 5 |
|---|---|---|---|---|---|---|
| Skin | None | Slight atrophy | Patch atrophy; | Marked | Ulceration | DEATH |
| | | Pigmentation | Moderate | atrophy; Gross | | DIRECTLY |
| | | change | telangiectasia; | telangiectasia | | RELATED |
| | | Some hair loss | Total hair loss | | | TO |
| Subcutaneous | None | Slight | Moderate | Severe | Necrosis | RADIATION |
| tissue | | induration | fibrosis but | induration and | | LATE |
| | | (fibrosia) and | asymptomatic | loss of | | EFFECTS |
| | | loss of | Slight field | subcutaneous | | |
| | | subcutaneous | contracture | tissue | | |
| | | fat | <10% linear | Field | | |
| | | | reduction | contracture | | |
| | | | | >10% linear | | |
| | | | | measurement | | |
| Mucous | None | Slight atrophy | Moderate | Marked | Ulceration | |
| membrane | | and dryness | atrophy and | atrophy with | | |
| | | | telangiectasia | complete | | |
| | | | Little mucous | dryness | | |
| | | | | Severe | | |
| | | | | telangiectasia | | |
| Salivary | None | Slight dryness | Moderate | Complete | Fibrosis | |
| glands | | of mouth | dryness of | dryness of | | |
| | | Good response | mouth | mouth | | |
| | | on stimulation | Poor response | No response on | | |
| | | | on stimulation | stimulation | | |
| Spinal cord | None | Mild | Severe | Objective | Monoplegia, | |
| | | L’Hermitte’s | L’Hermitte’s | neurological | paraplegia, | |
| | | syndrome | syndrome | findings at or | quadraplegia | |
| | | | | below cord | | |
| | | | | level treated | | |
| Brain | None | Mild headache | Moderate | Severe | Seizures or | |
| | | Slight lethargy | headache | headaches | paralysis | |
| | | | Great lethargy | Severe CNS | Coma | |
| | | | | dysfunction | | |
| | | | | (partial loss of | | |
| | | | | power or | | |
| | | | | dyskinesia) | | |
| Eye | None | Asymptomatic | Symptomatic | Severe keratitis | Panopthalmitis/ | |
| | | cataract | cataract | Severe | Blindness | |
| | | Minor comeal | Moderate | retinopathy or | | |
| | | ulceration or | comeal | detachment | | |
| | | keratitis | ulceration | Severe | | |
| | | | Minor | glaucoma | | |
| | | | retinopathy or | | | |
| | | | glaucoma | | | |
| Larynx | None | Hoarseness | Moderate | Severe edema | Necrosis | |
| | | Slight | arytenoid | Severe | | |
| | | arytenoid | edema | chondritis | | |
| | | edema | Chondritis | | | |
| Lung | None | Asymptomatic | Moderate | Severe | Severe | |
| | | or mild | symptomatic | symptomatic | respiratory | |
| | | symptoms | fibrosis or | fibrosis or | insufficiency/ | |
| | | (dry cough) | pneumonitis | pneumonitis | Continuous O₂/ | |
| | | Slight | (severe | Dense | Assisted | |
| | | radiographic | cough) | radiographic | ventilation | |
| | | appearances | Low grade | changes | | |
| | | | fever | | | |
| | | | Patchy | | | |
| | | | radiographic | | | |
| | | | appearances | | | |
| Heart | None | Asymptomatic | Moderate | Severe angina | Tamponade/ | |
| | | or mild | angina on | Pericardial | Severe heart | |
| | | symptoms | effort | effusion | failure/ Severe | |
| | | Transient T | Mild | Constrictive | constrictive | |
| | | wave inversion | pericarditis | pericarditis | pericarditis | |
| | | & ST changes | Normal heart | Moderate heart | | |
| | | Sinus | size | failure | | |
| | | tachycardia | Persistent | Cardiac | | |
| | | >110 (at rest) | abnormal T | enlargement | | |
| | | | wave and ST | EKG | | |
| | | | changes | abnormalities | | |
| | | | Low ORS | | | |
| Esophagus | None | Mild fibrosis | Unable to take | Severe fibrosis | Necrosis/ | |
| | | Slight | solid food | Able to | Perforation | |
| | | difficulty in | normally | swallow only | Fistula | |
| | | swallowing | Swallowing | liquids | | |
| | | solids | semi-solid | May have pain | | |
| | | | food | on swallowing | | |
| | | No pain on | Dilatation | Dilation | | |
| | | swallowing | may be | required | | |
| | | | indicated | | | |
| Small/Large | None | Mild diarrhea | Moderate | Obstruction or | Necrosis/ | |
| Intestine | | Mild cramping | diarrhea and | bleeding | Perforation | |
| | | Bowel | colic | requiring | Fistula | |
| | | movement 5 | Bowel | surgery | | |
| | | times daily | movement >5 | | | |
| | | Slight rectal | times daily | | | |
| | | discharge or | Excessive | | | |
| | | bleeding | rectal mucus | | | |
| | | | or intermittent | | | |
| | | | bleeding | | | |
| Liver | None | Mild lassitude | Moderate | Disabling | Necrosis/ | |
| | | Nausea, | symptoms | hepatitic | Hepatic coma | |
| | | dyspepsia | Some | insufficiency | or encephala- | |
| | | Slightly | abnormal | Liver function | opathy | |
| | | abnormal liver | liver function | tests grossly | | |
| | | function | tests | abnormal | | |
| | | | Serum | Low albumin | | |
| | | | albumin | Edema or | | |
| | | | normal | ascites | | |
| Kidney | None | Transient | Persistent | Severe | Malignant | |
| | | albuminuria | moderate | albuminuria | hypertension | |
| | | No | albuminuria | Severe | Uremic | |
| | | hypertension | (2+) | hypertension | coma/Urea | |
| | | Mild | Mild | Persistent | >100% | |
| | | impairment of | hypertension | anemia | | |
| | | renal function | No related | (<10 g %) | | |
| | | Urea 25-35 | anemia | Severe renal | | |
| | | mg % | Moderate | failure | | |
| | | Creatinine | impairment of | Urea >60 mg % | | |
| | | 1.5-2.0 mg % | renal function | Creatinine | | |
| | | Creatinine | Urea >36-60 | >4.0 mg % | | |
| | | clearance | mg % | Creatinine | | |
| | | >75% | Creatinine | clearance | | |
| | | | clearance | <50% | | |
| | | | (50-74%) | | | |
| Bladder | None | Slight | Moderate | Severe | Necrosis/ | |
| | | epithelial | frequency | frequency and | Contracted | |
| | | atrophy | Generalized | dysuria | bladder | |
| | | Minor | telangiectasia | Severe | (capacity | |
| | | telangiectasia | Intermittent | generalized | <100 cc) | |
| | | (microscopic | macroscopic | telangiectasia | Severe | |
| | | hematuria) | hematuria | (often with | hemorrhagic | |
| | | | | petechiae) | cystitis | |
| | | | | Frequent | | |
| | | | | hematuria | | |
| | | | | Reduction in | | |
| | | | | bladder | | |
| | | | | capacity | | |
| | | | | (<150 cc) | | |
| Bone | None | Asymptomatic | Moderate pain | Severe pain or | Necrosis/ | |
| | | No growth | or tenderness | tenderness | Spontaneous | |
| | | retardation | Growth | Complete | fracture | |
| | | Reduced bone | retardation | arrest of bone | | |
| | | density | Irregular bone | growth | | |
| | | | sclerosis | Dense bone | | |
| | | | | sclerosis | | |
| Joint | None | Mild joint | Moderate | Severe joint | Necrosis/ | |
| | | stiffness | stiffness | stiffness | Complete | |
| | | Slight | Intermittent or | Pain with | fixation | |
| | | limitation of | moderate joint | severe | | |
| | | movement | pain | limitation of | | |
| | | | Moderate | movement | | |
| | | | limitation of | | | |
| | | | movement | | | |

TABLE 2
RTOG Cooperative Group Common Toxicity Criteria (Acute Toxicity)

| | Toxicity | -0- | -1- | -2- | -3- | -4- |
|---|---|---|---|---|---|---|
| Blood/ | WBC | >=4.0 | 3.0-3.9 | 2.0-2.9 | 1.0-1.9 | <1.0 |
| Bone | Platelets | WNL | 75.0-normal | 50.0-74.9 | 25.0-49.9 | <25.0 |
| Marrow | Hemoglobin | WNL | 10.0-normal | 8.0-10.0 | 6.5-7.9 | <6.5 |
| | Granulocytes/ | >=2.0 | 1.5-1.9 | 1.0-1.2 | 0.5-0.9 | <0.5 |
| | Bands | | | | | |
| | Lymphocytes | >=2.0 | 1.5-1.9 | 1.0-1.2 | 0.5-0.9 | <0.5 |
| | Hemorrhage | None | Mild, no | Gross, | Gross, | Massive, |
| | (Clinical) | | transfusion | 1-2 units | 3-4 units | 3-4 units |
| | | | | transfusion | transfusion | transfusion |
| | | | | per episode | per episode | per episode |
| | Infection | None | Mild | Moderate | Severe | Life- |
| | | | | | | threatening |
| Gastro- | Nausea | None | Able to eat/ | Intake | No | — |
| intestinal | | | reasonable | significantly | significant | |
| | | | intake | decreased but | intake | |
| | | | | can eat | | |
| | Vomiting | None | 1 episode | 2-5 episodes | 6-10 episodes | >10 |
| | | | in 24 hours | in 24 hours | in 24 hours | episodes in |
| | | | | | | 24 hours or |
| | | | | | | requiring |
| | | | | | | parenteral |
| | | | | | | support |
| | Diarrhea | None | Increase of | Increase of 4- | Increase of 7- | Increase of |
| | | | 2-3 stools | 6 stools/day, | 9 stools/day | >=10 |
| | | | per day | or nocturnal | or | stools/day |
| | | | over pre- | stools, or | incontinence | or grossly |
| | | | Rx | moderate | or severe | bloody |
| | | | | cramping | cramping | diarrhea, or |
| | | | | | | need for |
| | | | | | | parenteral |
| | | | | | | support |
| | Stomatitis | None | Painless | Painful | Painful | Requires |
| | | | ulcers, | erythema, | erythema, | parenteral or |
| | | | erythema | edema or | edema or | enteral |
| | | | or mild | ulcers but can | ulcers and | support |
| | | | soreness | eat | cannot eat | |
| Liver | Bilirubin | WNL | — | <1.5 × N | 1.5-3.0 × N | >3.0 × N |
| | Trans- | WNL | <=2.5 × N | 2.6-5.0 × N | 5.1-20.0 × N | >20.0 × N |
| | aminase | | | | | |
| | (SGOT, | | | | | |
| | SGPT) | | | | | |
| | Alkaline | WNL | <=2.5 × N | 2.6-5.0 × N | 5.1-20.0 × N | >20.0 × N |
| | Phosphatase | | | | | |
| | or | | | | | |
| | S'nucleotidase | | | | | |
| | Liver/clinical | No | — | — | Precoma | Hepatic |
| | | change | | | | coma |
| | | from | | | | |
| | | baseline | | | | |
| Kidney/ | Creatinine | WNL | <1.5 × N | 1.5-3.0 × N | 3.1-6.0 × N | >6.0 × N |
| bladder | Proteinuria | No | 1+ or <0.3 | 2-3+ or 0.3- | 4+ or >1.0 | Nephrotic |
| | | change | g % or | 1.0 g % or | g % or >10 | syndrome |
| | | | <3 g/l | 3-10 g/l | g/l | |
| | Hematuria | Negative | Micro only | Gross/no | Gross + clots | Requires |
| | | | | clots | | transfusion |
| | Alopecia | No | Mild hair | Pronounced | — | — |
| | | loss | loss | or total hair | | |
| | | | | loss | | |
| | Pulmonary | None | Asymptomatic | Dyspnea on | Dyspnea at | Dyspnea at |
| | | or no | with | significant | normal level | rest |
| | | change | abnormality | exertion | of activity | |
| | | | in PFTUs | | | |
| Heart | Cardiac | None | Asymptomatic/ | Recurrent or | Requires | Requires |
| | dysrhythmias | | transient/ | persistent/no | treatment | monitoring or |
| | | | requiring | therapy | | hypotension or |
| | | | no therapy | required | | ventricular |
| | | | | | | tachycardia or |
| | | | | | | fibrillation |
| | Cardiac | None | Asymptomatic/ | Asymptomatic/ | Mild CHF, | Severe or |
| | function | | decline of | decline | responsive to | refractory |
| | | | resting | of resting | therapy | CHF |
| | | | ejection | ejection | | |
| | | | fraction by | friction by | | |
| | | | <20% of | >20% of | | |
| | | | baseline value | baseline value | | |
| | Cardiac/ | None | Non- | Asymptomatic/ | Angina | Acute |
| | ischemia | | specific T- | ST and T | without | myocardial |
| | | | wave | wave | evidence for | infarction |
| | | | flattening | changes | infarction | |
| | | | | suggesting | | |
| | | | | ischemia | | |
| | Cardiac/ | None | Asymptomatic | Pericarditis | Symptomatic | Tamponade/ |
| | pericardial | | effusion/no | (rub, chest | effusion: | drainage |
| | | | intervention | pain, ECG | drainage | urgently |
| | | | required | changes | required | required |
| Blood | Hypertension | None | Asymptomatic/ | Recurrent or | Requires | Hypertensive |
| | | or no | transient | persistent | therapy | crisis |
| | | change | increase by | increase by | | |
| | | | >20 mm | >20 mm Hg | | |
| | | | Hg (d) or to | (D) or to | | |
| | | | >150/100 if | >150/100 if | | |
| | | | previously | previously | | |
| | | | WNL/No | WNL/No | | |
| | | | treatment | treatment | | |
| | | | required | required | | |
| Pressure | Hypotension | None | Changes | Requires | Requires | Requires |
| | | or no | requiring | fluid | therapy and | therapy and |
| | | change | no therapy/ | replacement | hospitalization/ | hospitali- |
| | | | including | or other | resolves | zation for >48 |
| | | | transient | therapy but | within 48 | hrs after |
| | | | orthostatic | not | hours of | stopping the |
| | | | hypotension | hospitalization | stopping the | agent |
| | | | | | agent | |
| Neuro- | Neurological/ | None | Mild | Mild or | Severe | — |
| logic | sensory | or no | paresthesias/ | moderate | objective | |
| | | change | loss of | objective | sensory loss | |
| | | | deep tendon | sensory loss/ | or | |
| | | | reflexes | moderate | paresthesias | |
| | | | | paresthesias | that interfere | |
| | | | | | with function | |
| | Neurological/ | None | Subjective | Mild | Objective | Paralysis |
| | motor | or no | weakness/ | objective | weakness | |
| | | change | no objective | weakness | with | |
| | | | findings | without | impairment | |
| | | | | significant | of function | |
| | | | | impairment | | |
| | | | | of function | | |
| | Neurological/ | None | Mild | Moderate | Severe | Coma, |
| | cortical | | somnolence | somnolence | somnolence, | seizures, |
| | | | or agitation | or agitation | agitation, | toxic |
| | | | | | confusion, | paralysis |
| | | | | | disorientation or | |
| | | | | | hallucinations | |
| | Neurological/ | None | Slight | Intention | Locomotor | Cerebellar |
| | cerebellar | | incoordination/ | tremor, | ataxia | necrosis |
| | | | dysdiado- | dysmetria, | | |
| | | | kinesis | slurred | | |
| | | | | speech, | | |
| | | | | nystagmus | | |
| | Neurological/ | No | Mild | Moderate | Severe | Suicidal |
| | mood | change | anxiety or | anxiety or | anxiety or | ideation |
| | | | depression | depression | depression | |
| | Neurological/ | None | Mild | Moderate or | Unrelenting | — |
| | headache | | | severe but | and severe | |
| | | | | transient | | |
| | Neurological/ | None | Mild | Moderate | Severe | Ileus >96 |
| | constipation | or no | | | | hours |
| | | change | | | | |
| | Neurological/ | None | Asymptomatic/ | Tinnitus | Hearing loss | Deafness |
| | hearing | or no | hearing | | interfering | not |
| | | change | loss on | | with function | correctable |
| | | | audiometry | | but correctable | |
| | | | only | | with hearing | |
| | | | | | aid | |
| | Neurological/ | None | — | — | Symptomatic | Blindness |
| | vision | or no | | | subtotal loss | |
| | | change | | | of vision | |
| | Skin | None | Scattered | Scattered | Generalized | Exfoliative |
| | | or no | macular or | macular or | symptomatic | dermatitis or |
| | | change | papular | papular | macular, | ulcerating |
| | | | eruption or | eruption or | papular, or | dermatitis |
| | | | erythema | erythema | vesicular | |
| | | | that is | with pruritis | eruption | |
| | | | asymptomatic | or other | | |
| | | | | associated | | |
| | | | | symptoms | | |
| | Allergy | None | Transient | Urticaria, | Serum | Anaphylaxis |
| | | | rash/drug | drug fever = | sickness, | |
| | | | fever | 38° C., | broncho- | |
| | | | <38° C., | 100.4° F./ mild | spasm, | |
| | | | 100.4° F. | broncho- | requiring | |
| | | | | spasm | parenteral | |
| | | | | | medication | |
| | Fever in | None | 37.1-38.0° C., | 38.1-40.0° C., | >40.0° C./ | >40.0° C./ |
| | absence of | | 98.7-100.4° F. | 100.5-104.0° F. | >104.0° F. | 104.0° F. |
| | infection | | | | for less | for more |
| | | | | | than 24 hours | than 24 hrs |
| | | | | | | or fever |
| | | | | | | accompanied |
| | | | | | | by |
| | | | | | | hypertension |
| | Local | None | Pain | Pain and | Ulceration | Plastic |
| | | | | swelling with | | surgery |
| | | | | inflammation | | indicated |
| | | | | or phlebitis | | |
| | Weight | <5.0% | 5.0-9.9% | 10.0-19.9% | >=20.0% | — |
| | gain/loss | | | | | |
| Metabolic | Hyper- | <116 | 116-160 | 161-250 | 251-500 | >500 or |
| | glycemia | | | | | ketoacidosis |
| | Hypo- | >64 | 55-64 | 40-54 | 30-39 | <30 |
| | glycemia | | | | | |
| | Amylase | WNL | <1.5 × N | 1.5-2.0 × N | 2.1-5.0 × N | >5.1 × N |
| | Hyper- | <10.6 | 10.6-11.5 | 11.6-12.5 | 12.6-13.5 | >=13.5 |
| | calcemia | | | | | |
| | Hypo- | >8.4 | 8.4-7.8 | 7.7-7.0 | 6.9-6.1 | <=6.0 |
| | calcemia | | | | | |
| | Hypo- | >1.4 | 1.4-1.2 | 1.1-0.9 | 0.8-0.6 | <=0.5 |
| | magnesemia | | | | | |
| Coagulation | Fibrinogen | WNL | 0.99-0.75 × N | 0.74-0.50 × N | 0.49-0.25 × N | <=0.24 × N |
| | Prothrombin | WNL | 1.01-1.25 × N | 1.26-1.50 × N | 1.51-2.00 × N | >=2.00 × N |
| | time | | | | | |
| | Partial | WNL | 1.01-1.66 × N | 1.67-2.33 × N | 2.34-3.00 × N | >3.00 × N |
| | thromboplast | | | | | |
| | in time | | | | | |

The term “increased probability/likelihood” or “reduced probability/likelihood” in the context of the present invention, relates to the increased or reduced probability that an event will occur over a specific time period, and can mean a subject's “absolute” probability or “relative” probability. Absolute probability can be measured with reference to either actual observation post-measurement for the relevant time cohort, or with reference to index values developed from statistically valid historical cohorts that have been followed for the relevant time period. Relative probability refers to the ratio of absolute probabilities of a subject compared either to the absolute probabilities of low probability cohorts or an average population probability, which can vary by how clinical probabilities are assessed. Odds ratios, the proportion of positive events to negative events for a given test result, are also commonly used (odds are according to the formula p/(1−p) where p is the probability of event and (1−p) is the probability of no event) to no-conversion.

A patient's “increased likelihood, risk, or probability” of having a toxic response to a therapy or may be based on a comparison to the rate of toxic response (or predicted rate of toxic response) of a cohort of patients having a different genotype than the patient's genotype with respect to a particular biomarker or biomarkers. An “increased likelihood, risk, or probability” of having a toxic response to a therapy may also be based on a comparison to the rate of toxic response (or predicted rate of response) for a cohort of patients without taking the marker (or markers) into consideration.

A patient's “decreased likelihood, risk, or probability” of having a toxic response to a therapy may be based on a comparison to the rate of toxic response (or predicted rate of toxic response) of a cohort of patients having a different genotype than the patient's genotype with respect to a particular biomarker or biomarkers. A “decreased likelihood, risk, or probability” of having a toxic response to a therapy may also be based on a comparison to the rate of toxic response (or predicted rate of response) for a cohort of patients without taking the marker (or markers) into consideration.

A patient's “increased likelihood, risk, or probability” of experiencing mortality, distant failure or metastasis, or impotency following a therapy or may be based on a comparison to the rate or timing of (e.g. a shorter time to) mortality, distant failure or metastasis, or impotency experienced by a cohort of patients having a different genotype than the patient's genotype with respect to a particular biomarker or biomarkers. An “increased likelihood, risk, or probability” of experiencing mortality, distant failure or metastasis, or impotency following a therapy may also be based on a comparison to the mortality, distant failure or metastasis, or impotency experienced by a cohort of patients without taking the marker (or markers) into consideration.

A patient's “decreased likelihood, risk, or probability” of experiencing mortality, distant failure or metastasis, or impotency following a therapy or may be based on a comparison to the rate or timing of (e.g. a longer time to) mortality, distant failure or metastasis, or impotency experienced by a cohort of patients having a different genotype than the patient's genotype with respect to a particular biomarker or biomarkers. An “decreased likelihood, risk, or probability” of experiencing mortality, distant failure or metastasis, or impotency following a therapy may also be based on a comparison to the mortality, distant failure or metastasis, or impotency experienced by a cohort of patients without taking the marker (or markers) into consideration.

As used herein, the terms “mutant” or “wildtype” refer to the allele status of the SNP, as determined by The Single Nucleotide Polymorphism Database (National Center for Biotechnology Information (NCBI)).

As used herein, the term “mortality” refers to death. Accordingly, a patient carrying a mutant or wild type SNP associated with a shortened time to mortality is more likely to experience death sooner than a patient who does not carry that mutant or wild type SNP, following a radiation treatment (e.g., a traditional radiation treatment).

As used herein, the term “biochemical relapse” refers to elevated blood levels of Prostate Specific Antigen (PSA). Accordingly, a patient carrying a mutant or wild type SNP associated with a shortened time to biochemical relapse is more likely to experience elevated blood levels sooner than a patient who does not carry that mutant or wild type SNP, following a radiation treatment (e.g., a traditional radiation treatment).

As used herein, the terms “distant failure” or “distant metastasis” refer to the recurrence of cancer, i.e., the presence of cancer cells and/or a tumor at a site that is distant from a primary tumor, and are used interchangeably.

As used herein, the term “potency” refers to the ability to develop or sustain a penile erection sufficient to conclude coitus. The term “impotency” refers to the inability to develop or sustain a penile erection sufficient to conclude coitus. In certain embodiments, potency and/or impotency is assessed by interview of a patient by the treating physician.

Linkage disequilibrium (LD) refers to the co-inheritance of alleles (e.g., alternative nucleotides) at two or more different SNP sites at frequencies greater than would be expected from the separate frequencies of occurrence of each allele in a given population. The expected frequency of co-occurrence of two alleles that are inherited independently is the frequency of the first allele multiplied by the frequency of the second allele. Alleles that co-occur at expected frequencies are said to be in “linkage equilibrium.” In contrast, LD refers to any non-random genetic association between allele(s) at two or more different SNP sites, which is generally due to the physical proximity of the two loci along a chromosome. LD can occur when two or more SNP sites are in close physical proximity to each other on a given chromosome and therefore alleles at these SNP sites will tend to remain unseparated for multiple generations with the consequence that a particular nucleotide (allele) at one SNP site will show a non-random association with a particular nucleotide (allele) at a different SNP site located nearby. Hence, genotyping one of the SNP sites will give almost the same information as genotyping the other SNP site that is in LD.

For screening individuals for genetic disorders (e.g. prognostic or risk) purposes, if a particular SNP site is found to be useful for screening a disorder, then the skilled artisan would recognize that other SNP sites which are in LD with this SNP site would also be useful for screening the condition. Accordingly, in certain embodiments, the detection of one or more of the SNPs described herein (“the recited SNP”) also includes detecting a SNP in LD with the recited SNP.

Various degrees of LD can be encountered between two or more SNPs with the result being that some SNPs are more closely associated (i.e., in stronger LD) than others. Furthermore, the physical distance over which LD extends along a chromosome differs between different regions of the genome, and therefore the degree of physical separation between two or more SNP sites necessary for LD to occur can differ between different regions of the genome.

For screening applications, polymorphisms (e.g., SNPs and/or haplotypes) that are not the actual disease-causing (causative) polymorphisms, but are in LD with such causative polymorphisms, are also useful. In such instances, the genotype of the polymorphism(s) that is/are in LD with the causative polymorphism is predictive of the genotype of the causative polymorphism and, consequently, predictive of the phenotype (e.g., disease) that is influenced by the causative SNP(s). Thus, polymorphic markers that are in LD with causative polymorphisms are useful as markers, and are particularly useful when the actual causative polymorphism(s) is/are unknown.

Linkage disequilibrium in the human genome is reviewed in: Wall et al. (2003) NAT REV GENET. 4(8):587-97; Gamer et al. (2003) GENET EPIDEMIOL. 24 (1):57-67; Ardlie et al. (2002) NAT REV GENET. 3(4):299-309 (erratum in (2002) NAT REV GENET 3(7):566); and Remm et al. (2002) CURR OPIN CHEM BIOL. 6(1):24-30.

The screening techniques of the present invention may employ a variety of methodologies to determine whether a test subject has a SNP or a SNP pattern associated with an increased or decreased risk of developing a detectable trait or whether the individual suffers from a detectable trait as a result of a particular polymorphism/mutation, including, for example, methods which enable the analysis of individual chromosomes for haplotyping, family studies, single sperm DNA analysis, or somatic hybrids. The trait analyzed using the diagnostics of the invention may be any detectable trait that is commonly observed in pathologies and disorders.

### SNP Genotyping Methods

The process of determining which specific nucleotide (i.e., allele) is present at each of one or more SNP positions is referred to as SNP genotyping. The present invention provides methods of SNP genotyping in order to determine whether a patient has a particular genotype with respect to the mutations disclosed herein as useful biomarkers in predicting a patient's toxicity response to radiation.

Nucleic acid samples can be genotyped to determine which allele(s) is/are present at any given genetic region (e.g., SNP position) of interest by methods well known in the art. The neighboring sequence can be used to design SNP detection reagents such as oligonucleotide probes, which may optionally be implemented in a kit format. Exemplary SNP genotyping methods are described in Chen et al. (2003) PHARMACOGENOMICS J. 3(2):77-96; Kwok et al. (2003) CURR ISSUES MOL. BIOL. 5(2):43-60; Shi (2002) AM J PHARMACOGENOMICS 2(3):197-205; and Kwok (2001) ANNU REV GENOMICS HUM GENET 2:235-58. Exemplary techniques for high-throughput SNP genotyping are described in Marnellos (2003) CURR OPIN DRUG DISCOV DEVEL. 6(3):317-21. Common SNP genotyping methods include, but are not limited to, quantitative PCR, TaqMan assays, molecular beacon assays, nucleic acid arrays, allele-specific primer extension, allele-specific PCR, arrayed primer extension, homogeneous primer extension assays, primer extension with detection by mass spectrometry, pyrosequencing, multiplex primer extension sorted on genetic arrays, ligation with rolling circle amplification, homogeneous ligation, OLA (U.S. Pat. No. 4,988,167), multiplex ligation reaction sorted on genetic arrays, restriction-fragment length polymorphism, single base extension-tag assays, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, and electrical detection. These methods are well known in the art.

SNP genotyping can include the steps of, for example, collecting a biological sample from a human subject (e.g., sample of tissues, cells, fluids, secretions, etc.), isolating nucleic acids (e.g., genomic DNA, mRNA or both) from the cells of the sample, contacting the nucleic acids with one or more primers which specifically hybridize to a region of the isolated nucleic acid containing a target SNP under conditions such that hybridization and amplification of the target nucleic acid region occurs, and determining the nucleotide present at the SNP position of interest, or, in some assays, detecting the presence or absence of an amplification product (assays can be designed so that hybridization and/or amplification will only occur if a particular SNP allele is present or absent). In some assays, the size of the amplification product is detected and compared to the length of a control sample; for example, deletions and insertions can be detected by a change in size of the amplified product compared to a normal genotype.

A biological sample for determination of the presence or absence of any of the mutations disclosed herein can be any tissue or fluid from the patient that contains nucleic acids. Various embodiments include paraffin imbedded tissue, frozen tissue, surgical fine needle aspirations, and cells of various tissues of the subject, such as blood cells or a cheek swab.

In one embodiment, determining whether a patient is a carrier of a particular germline mutation, or has a particular genotype or zygosity with respect to that mutation, is based on genetically evaluating normal cells (as opposed to tumor cells) from the patient, for example, blood cells or cells from a cheek swab.

In one embodiment, determining whether a patient is a carrier of a particular germline mutation, or has a particular genotype or zygosity with respect to that mutation, includes reviewing a medical record of the patient containing the results of a SNP genotyping test.

### Radiation Treatments

The disclosure relates in part to the discovery of biomarkers that are predictive of a patient's likelihood of developing toxicity to a hypofractionated radiation treatment. A hypofractionated radiation treatment generally provides the same amount of radiation as a traditional radiation treatment, but in a shorter amount of time. Hypofractionated radiation may be more effective and/or more convenient for the patient, but a subset of patients experience toxicity to hypofractionated radiation. The biomarkers described herein can be used to evaluate a patient to determine whether they are likely to develop toxicity to a hypofractionated radiation treatment. If a patient carries certain mutations associated with toxicity, or does not carry certain other mutations that indicate the patient is unlikely to develop toxicity, the patient may be administered a traditional radiation treatment. Likewise, if a patient carries certain mutations that indicate the patient is unlikely to develop toxicity, or does not carry mutations that indicate the patient is likely to develop toxicity, the patient may be administered a hypofractionated radiation treatment.

The disclosure relates in part to the discovery of biomarkers that are predictive of a patient's likelihood of developing toxicity to a stereotactic body radiation treatment (SBRT). An SBRT radiation treatment generally provides the same amount of radiation as a traditional radiation treatment, but in a shorter amount of time. SBRT may be more effective and/or more convenient for the patient, but a subset of patients experience toxicity to hypofractionated radiation. The biomarkers described herein can be used to evaluate a patient to determine whether they are likely to develop toxicity to a SBRT radiation treatment. If a patient carries certain mutations associated with toxicity, or does not carry certain other mutations that indicate the patient is unlikely to develop toxicity, the patient may be administered a traditional radiation treatment. Likewise, if a patient carries certain mutations that indicate the patient is unlikely to develop toxicity, or does not carry mutations that indicate the patient is likely to develop toxicity, the patient may be administered a SBRT radiation treatment.

The disclosure further relates, in part, to the discovery of biomarkers that are predictive of a patient's likelihood of developing toxicity to a standard (conventional) fractionated radiation treatment (CF-RT). The biomarkers described herein can be used to evaluate a patient to determine whether they are likely to develop toxicity to a standard fractionated radiation treatment. If a patient carries certain mutations associated with toxicity, or does not carry certain other mutations that indicate the patient is unlikely to develop toxicity, the patient may be administered a hypofractionated radiation treatment or a cancer treatment that does not include standard fractionated radiation (a different cancer treatment). Likewise, if a patient carries certain mutations that indicate the patient is unlikely to develop toxicity, or does not carry mutations that indicate the patient is likely to develop toxicity, the patient may be administered a standard fractionated radiation treatment.

The disclosure also relates in part to the discovery of biomarkers that are predictive of a patient's likelihood of developing impotency, and likelihood of exhibiting a shortened time to biochemical relapse, distant metastasis and mortality following a cancer treatment (e.g., a standard fractionated radiation treatment as shown in Example 3). The biomarkers described herein can be used to evaluate a patient to determine whether they are likely to experience impotency and/or to experience a shorter time to biochemical relapse, distant metastasis and/or mortality following a cancer treatment. If a patient carries certain mutations associated with impotency, biochemical relapse, distant metastasis and mortality, or does not carry certain other mutations that indicate the patient is unlikely to experience impotency or experience a shorter time to biochemical relapse, distant metastasis and mortality, the patient may be administered a different cancer treatment (e.g., a chemotherapeutic treatment or hypofractionated radiation treatment). In certain embodiments, the different cancer treatment includes brachytherapy, hypofractionated radiation, chemotherapy, androgen deprivation therapy, immunotherapy, high intensity focused ultrasound, cryotherapy, laser ablation, photodynamic therapy, and surgery. Likewise, if a patient carries certain mutations that indicate the patient is unlikely to experience impotency or a shortened time to biochemical relapse, distant metastasis and mortality, or does not carry mutations that indicate the patient is likely to experience impotency or a shorter time to biochemical relapse, distant metastasis and mortality, the patient may be administered the cancer treatment (e.g., a standard fractionation radiation treatment).

A traditional radiation treatment is, in general, administered over a longer period of time than a hypofractionated radiation treatment. In certain embodiments, a traditional radiation treatment comprises administering from about 35 Gy to about 90 Gy radiation over a period of about 3 to about 8 weeks and/or over about 10 to about 30 fractions. In certain embodiments, the dose per fraction is about 2 Gy.

For example, a traditional radiation treatment can comprise administering from about 40 to about 90 Gy radiation, from about 45 to about 90 Gy radiation, from about 50 to about 90 Gy radiation, from about 55 to about 90 Gy radiation, from about 60 to about 90 Gy radiation, from about 65 to about 90 Gy radiation, from about 70 to about 90 Gy radiation, from about 75 to about 90 Gy radiation, from about 80 to about 90 Gy radiation, from about 85 to about 90 Gy radiation, from about 35 to about 85 Gy radiation, from about 40 to about 85 Gy radiation, from about 45 to about 85 Gy radiation, from about 50 to about 85 Gy radiation, from about 55 to about 85 Gy radiation, from about 60 to about 85 Gy radiation, from about 65 to about 85 Gy radiation, from about 70 to about 85 Gy radiation, from about 75 to about 85 Gy radiation, from about 80 to about 85 Gy radiation, from about 35 to about 80 Gy radiation, from about 40 to about 80 Gy radiation, from about 45 to about 80 Gy radiation, from about 50 to about 80 Gy radiation, from about 55 to about 80 Gy radiation, from about 60 to about 80 Gy radiation, from about 65 to about 80 Gy radiation, from about 70 to about 80 Gy radiation, from about 75 to about 80 Gy radiation, from about 35 to about 75 Gy radiation, from about 40 to about 75 Gy radiation, from about 45 to about 75 Gy radiation, from about 50 to about 75 Gy radiation, from about 55 to about 75 Gy radiation, from about 60 to about 75 Gy radiation, from about 65 to about 75 Gy radiation, from about 70 to about 75 Gy radiation, from about 35 to about 70 Gy radiation, from about 40 to about 70 Gy radiation, from about 45 to about 70 Gy radiation, from about 50 to about 70 Gy radiation, from about 55 to about 70 Gy radiation, from about 60 to about 70 Gy radiation, from about 65 to about 70 Gy radiation, from about 35 to about 65 Gy radiation, from about 40 to about 65 Gy radiation, from about 45 to about 65 Gy radiation, from about 50 to about 65 Gy radiation, from about 55 to about 65 Gy radiation, from about 60 to about 65 Gy radiation, from about 35 to about 60 Gy radiation, from about 40 to about 60 Gy radiation, from about 45 to about 60 Gy radiation, from about 50 to about 60 Gy radiation, from about 55 to about 60 Gy radiation, from about 35 to about 55 Gy radiation, from about 40 to about 55 Gy radiation, from about 45 to about 55 Gy radiation, from about 50 to about 55 Gy radiation, from about 35 to about 50 Gy radiation, from about 40 to about 50 Gy radiation, from about 45 to about 50 Gy radiation, from about 35 to about 45 Gy radiation, from about 40 to about 45, or from about 35 to 40 Gy radiation.

The radiation can be administered over a period of from about 3 to about 7 weeks, from about 4 to about 7 weeks, from about 5 to about 7 weeks, from about 6 to about 7 weeks, from about 3 to about 6 weeks, from about 4 to about 6 weeks, from about 5 to about 6 weeks, from about 3 to about 5 weeks, from about 4 to about 5 weeks, or from about 3 to about 4 weeks. In certain embodiments, the radiation is administered over a period of about 3 weeks, 4 weeks, 5 weeks, 6 weeks, or 7 weeks.

The radiation can be administered over about 10 to about 25 fractions, about 10 to about 20 fractions, about 10 to about 15 fractions, about 15 to about 30 fractions, about 15 to about 25 fractions, about 15 to about 20 fractions, about 20 to about 30 fractions, about 20 to about 25 fractions, or about 25 to about 20 fractions. In certain embodiments, the radiation is administered over about 10 fractions, 11 fractions, 12 fractions, 13 fractions, 14 fractions, 15 fractions, 16 fractions, 17 fractions, 18 fractions, 19 fractions, 20 fractions, 21 fractions, 22 fractions, 23 fractions, 24 fractions, 25 fractions, 26 fractions, 27 fractions, 28 fractions, 29 fractions or 30 fractions.

In certain embodiments, a hypofractionated radiation regimen comprises administering from about 20 Gy to about 50 Gy radiation over about 1 week to about 5 weeks, and/or over about 5 to about 15 fractions. In certain embodiments, the dose per fraction is greater than about 2 Gy (e.g., 2, 2%, 3, 3.2, 3.3 or greater).

In certain embodiments, a hypofractionated radiation regimen comprises administering from about 25 to about 50 Gy radiation, from about 35 to about 50 Gy radiation, from about 40 to about 50 Gy radiation, from about 20 to about 45 Gy radiation, from about 25 to about 45 Gy radiation, from about 30 to about 45 Gy radiation, from about 35 to about 45 Gy radiation, from about 40 to about 45 Gy radiation, from about 20 to about 40 Gy radiation, from about 25 to about 40 Gy radiation, from about 30 to about 40 Gy radiation, from about 35 to about 40 Gy radiation, from about 20 to about 35 Gy radiation, from about 25 to about 35 Gy radiation, from about 30 to about 35 Gy radiation, from about 20 to about 30 Gy radiation, from about 25 to about 30 Gy radiation, or from about 20 to about 25 Gy radiation.

In certain embodiments, a hypofractionated radiation regimen is administered over a period of from about 2 to about 5 weeks, about 3 to 5 weeks, about 4 to 5 weeks, about 1 to 4 weeks, about 2 to 4 weeks, about 3 to 4 weeks, about 1 to 3 weeks, about 2 to 3 weeks, or about 1 to 2 weeks. In certain embodiments, the radiation is administered over a period of about 1, about 2, about 3, about 4 or about 5 weeks.

In certain embodiments, a hypofractionated radiation regimen is administered over about 5 to about 15 fractions, about 5 to about 10 fractions, or about 10 to about 15 fractions. In certain embodiments, the radiation is administered over about 5 fractions, about 6 fractions, about 7 fractions, about 8 fractions, about 9 fractions, about 10 fractions, about 11 fractions, about 12 fractions, about 13 fractions, about 14 fractions, or about 15 fractions.

The radiation may be external-beam radiation therapy such as photon beams of x-rays or gamma rays, electron beams, or proton therapy. The radiation may be internal radiation therapy (brachytherapy) where radiation is delivered from a radiation source placed inside or on the body, even inside the tumor tissue. The radiation source may be a radioactive isotope in the form of a seed or pellet implanted in or placed on the patient.

### Cancers

Cancers that may be treated according to the methods of the invention and cancers for which a patient's responsiveness to a treatment therapy can be determined according to the methods of the invention include sarcoma, prostate cancer, adrenal cancer, anal cancer, bile duct cancer, bladder cancer, bone cancer, cancer of the brain or central nervous system, basal cell skin cancer, breast cancer, cervical cancer, colorectal cancer, endometrial cancer, esophageal cancer, eye cancer, gallbladder cancer, gastrointestinal carcinoid tumors, gastrointestinal stromal tumor (GIST), gastric cancer, glioma, glioblastoma, head and neck cancer (including head and neck squamous cell carcinoma), Hodgkin disease, Classical Hodgkin Lymphoma, diffuse large B cell lymphoma, follicular lymphoma, Kaposi sarcoma, kidney cancer, laryngeal and hypopharyngeal cancer, leukemia (including acute myeloid leukemia), liver cancer (including hepatocellular carcinoma), lung cancer (including non-small cell lung cancer and metastatic non-small cell lung cancer), lymphoma, malignant mesothelioma, melanoma (including unresectable or metastatic melanoma), merkel cell carcinoma, metastatic urothelial carcinoma, multiple myeloma, myeloma, myelodysplastic syndrome, nasal cavity and paranasal sinus cancer, nasopharyngeal cancer, neuroendocrine cancer, neuroblastoma, non-Hodgkin lymphoma, oral cavity and oropharyngeal cancer, osteosarcoma, ovarian cancer, pancreatic cancer, penile cancer, pituitary tumors, renal cancer (including renal cell carcinoma), retinoblastoma, rhabdomyosarcoma, salivary gland cancer, squamous cell skin cancer, small intestine cancer, stomach cancer, testicular cancer, thymus cancer, thyroid cancer, uterine cancer, or vaginal cancer.

### EXAMPLES

### Example 1. Biomarkers of Toxicity Resulting from Hypofractionated Radiation

### Patients

Patients with histologically confirmed STS of the extremity or trunk with planned neoadjuvant RT and surgery were eligible. All patients were 18 or older and had Eastern Cooperative Oncology Group (ECOG) performance status 0 to 2. Exclusion criteria included evidence of distant metastases, planned neoadjuvant or adjuvant chemotherapy; prior RT to the area to be irradiated; and active treatment of a second malignancy.

### Study Design and Treatments

Patients were assigned to receive neoadjuvant RT followed by surgery 2 to 6 weeks later. Radiation CT and/or MRI simulation was performed with custom immobilization. The gross, clinical and planning target volumes were defined according to RTOG 06306. A dose of 6 Gy×5 fractions (30 Gy) was delivered to at least 95% of the PTV. Intensity modulated (IMRT), 3D conformal, or electron planning techniques were used. Radiation plans were deemed acceptable if they met dosimetric parameters outlined in TABLE 3. All patients underwent daily image guidance except two patients receiving electron RT. All surgeries were performed by one of four dedicated sarcoma surgeons.

TABLE 3

| Target Volume or Organ at Risk | Dose Constraint |
|---|---|
| PTV | V110(%) ≤10% |
| Skin | V12Gy ≤50% |
| 2 cm longitudinal strip of skin | V12Gy ≤10% |
| Long bones (femur, humerus) | V30Gy ≤50% |
| Femoral or humeral head | V30Gy ≤5 cc, Dmax ≤33 Gy |
| Spinal coord | Dmax <30 Gy |
| Chest wall | V30Gy ≤70 cc |
| Bowel | V32Gy ≤5 cc |
| Liver | V15Gy ≤700 cc |
| Kidney | V10Gy ≤10% |

### Assessments

The primary endpoint of this study was the rate of late grade ≥2 radiation morbidity (fibrosis, lymphedema or joint stiffness) at median two-year follow-up (minimum one year). Fibrosis and joint stiffness were graded based on Radiation Therapy Oncology Group/European Organisation for Research and Treatment of Cancer (RTOG/EORTC) criteria, and lymphedema was graded by Stern's scale. Other secondary endpoints included acute toxicities as assessed by the frequency and severity of adverse events (AEs) using CTCAE v4.0 toxicity criteria, the rate of major wound complications, pathologic treatment effect, and the rate of local and distant recurrences. Major wound complications were defined as per established criteria from prospective clinical trials of extremity soft tissue sarcoma. Pathologic treatment effect was defined as the percentage of surgical tissue with hyalinization or necrosis relative to pre-treatment biopsy. Time-to-event end points were measured from enrollment.

Patients were seen after the completion of radiation and prior to surgery by the radiation oncologist and/or the sarcoma surgeon. Patients were followed closely in the postoperative setting. The patients' status was reviewed 3 months after surgery, and then at least every 6 months thereafter. The patients were followed both clinically and radiographically after treatment with CT or MRI of the primary site and CT of the chest at least every 6 months for the first two years and then at least annually in the third year. Patients with myxoid liposarcoma were also evaluated with CT of the abdomen and pelvis.

A subset of patients were consented to a parallel imaging study under IRB approval from May 2016 to June 2018. Diffusion-weighted images (DWI) were acquired up to four times for each patient using a 0.35T MR-guided radiotherapy machine (ViewRay, MRIdian, Mountain View, Calif.) including before the first fraction of treatment and at least 14 days after RT but prior to surgical resection.

### Statistical Analysis

The study was designed to evaluate the rate of grade ≥2 radiation morbidity (subcutaneous tissue fibrosis, joint stiffness, or edema at 2 years to mirror the initial design of RTOG-06306 with a target absolute improvement of 20% in the rate of grade ≥2 late radiation-associated toxicity compared to the historical neoadjuvant RT arm of the CAN-NCIC-SR2 study from 37% to 17%.

Between May 2016 and May 2018, 52 patients with localized high-risk STS of the extremity or trunk were enrolled. Of these, 50 patients ultimately underwent neoadjuvant RT and surgery (FIG. 2).

The relationship between major wound complications and relevant clinical variables, including tumor size, tumor site, tumor depth, time interval from radiation to surgery, and two radiation dosimetric variables using univariate logistic regression was examined. Dosimetric variables included the maximum radiation dose (Gy) to the skin (minimum 0.5 cc volume), and volume of the skin (cc) receiving 12 Gy. Likelihood ratio test (LRT) was used to assess the significance of categorical variables. Pathologic outcomes were reported with descriptive statistics. Differences in pre- and posttreatment tumor volumes by diffusion-weighted MRI were assessed by paired t-test. Comparisons of average distance traveled and volume of patients between the two years prior to study enrollment and the study period were made using unpaired t-tests.

### Germline Biomarker Analysis

Genomic DNA from blood or saliva was analyzed for single nucleotide polymorphisms (SNPs) disrupting miRNA binding sites, promoter regions or coding sequences as previously identified. Biomarkers in binding sites in genes involved in the immune system and DNA damage response, as well as promoters and coding sequences of miRNAs that regulated key genes known to be critical in the DNA damage or immune response were enriched in the analysis. As a final step in the evaluation for candidate variants to test, variants were limited to those predicted to be found in between 0.5% to 25% of the population, to find biomarkers that are likely to be detected in reasonably small cohorts of patients. This list was reduced to ˜116 variants by fitting the above defined priority parameters for miRNA pathway variants. Panels were run using the Sequenom platform. Each panel was run with internal controls that used Taqman Genotyping as the gold standard. Any biomarker with less than a 90% call rate or more than 1% error found by controls was excluded from further analysis. To insure sufficient marginal variation in the final panel, any biomarker with an observed rate of mutation less than 12.5% in the training sample was excluded from the analysis.

The relationship of this set of 116 SNPs with the incidence of major wound complications was examined. Each SNP was defined as a categorical variable. Lower extremity tumor site was also included as a categorical variable as it was the only clinical variable associated with major wound complications. The association between this panel of potential germline biomarkers and tumor site with wound toxicity was assessed using four classifiers on the set of 50 sarcoma patients, with wound toxicity rate 32%. Trained classifiers, whose hyperparameters were selected to optimize the F1 score with leave-one-out cross-validation (LOOCV), included classification trees (CT) (Breiman et al: Classification and Regression Trees. Boca Raton, Fla., CRC Press, 2017), random forests (RF) (Breiman L: Random Forests. Machine Learning 45:5-32, 2001), boosted trees (BT) (Chen T, Guestrin C: XGBoost: A Scalable Tree Boosting System, KDD. San Francisco, Calif., 2016) and LASSO-regularized logistic regression (LASSO-LR) (Tibshirani R: Regression Shrinkage and Selection via the Lasso. Journal of the Royal Statistical Society Series B (Methodological):267-88, 1996). The CT were tuned on minimum split and minimum observations in any terminal node, RF were tuned on number of trees and variables considered at each split, BT were tuned on the learning parameter eta, tree depth, and the number of rounds, and LASSO-LR models were tuned on the regularization parameter lambda. The subjects with toxicity were up-weighted through oversampling method. The final performance measures, accuracy, specificity, sensitivity, negative predictive value, positive predictive value, area under the curve (AUC), and F1 score were reported using stratified 10-fold cross-validation. The threshold of number of predictors to include in the model was determined as the use of top k predictors allowed with the highest AUC among k=5, 10, 15, . . . , 50. Importance measures via filter method with R package FSelector (Romanski P, Kotthoff L: FSelector: Selecting Attributes, (ed R package version 0.31), 2018) were then used to select top k predictors to train the classifiers and this was determined by mean rank from 1000 sample sets of their respective value. The four important measures are entropy-based information gain between predictors and response, variable importance based on ranger impurity importance, the entropy-based gain ratio between predictors and response and the univariate model score. Via 1000 oversampling sets, the order of significance of these k predictors was determined according to the obtained variable importance measure of mean decrease in the Gini impurity from the trained random forest classifier for its best prediction performance among 4 classifiers. These top k predictors to wound toxicity with their order of significance were reported. CT, RF, BT, and LASSO-LR classifiers were fit in R (version 3.6.0) (R Development Core Team: Stats package (power.prop.test( ) function) in R: A language and environment for statistical computing. Vienna, Austria: R Foundation for Statistical Computing, 2018) with mlr (Bischl et al.: mlr: Machine Learning in R, (ed R package version 2.13), 2018) calling rpart (Therneau T AB, and Ripley B.: rpart: Recursive Partitioning and Regression Trees, (ed R package version 4.1-13), 2018), ranger (Marvin N. Wright S W, Philipp Probst: ranger: A Fast Implementation of Random Forests, (ed R package version 0.11.2), 2019), xgboost (Chen T, He T: xgboost: eXtreme Gradient Boosting, (ed R package version 0.82.1), 2019), and glmnet (Jerome Friedman et al.: glmnet: Lasso and Elastic-Net Regularized Generalized Linear Models, (ed R package version 2.0-16), 2018), respectively, and the tree plot was generated with package reprtree (Banerjee M, Ding Y, Noone A M: Identifying representative trees from ensembles. Stat Med 31:1601-16, 2012).

The final cross validated tuning parameters for the reported classifiers are as follows: minimum split of 5 and minimum observations of 5 in any terminal node for classification trees (CT), three variables considered at each split with 15 trees for random forests (RF), learning parameter eta of 0.367, max depth of 2, and 13 rounds for boosted trees (BT) and regularization parameter lambda equal to 0.001 for LASSO-regularized logistic regression (LASSO-LR). For each classifier, all remaining hyperparameters were assigned their default values as defined through their associated R packages.

### Results

### Patient Characteristics and Accrual

The study enrolled patients across a broad age spectrum, including five patients between age 80 and 90, three of whom had ECOG performance status of 2. With the exception of one tumor, all were intermediate- or high-grade. Tumor size among enrolled patients was heterogeneous, ranging from 1.2 to 28 cm, and twelve patients had tumors greater than 10 cm. Twelve patients (24%) received neoadjuvant RT prior to re-resection for gross (n=9, 18%) or microscopic (n=3, 6%) residual disease. The median time between completion of RT and surgery was 28 days (range 14-55).

### Radiation-Associated Toxicities

The five-day neoadjuvant RT regimen was well-tolerated without grade 3 or higher acute or subacute RT-associated toxicities. The most severe radiation dermatitis was grade 2 and occurred in four patients (8%); other grade 2 toxicities were pain flare (n=3, 6%) and nausea (n=1, 2%). Radiation-associated late toxicities (fibrosis, joint stiffness or lymphedema) as measured by RTOG/EORTC criteria are summarized in TABLE 4.

TABLE 4
Late Toxicities of Five-Day Preoperative Radiation Therapy

| | | |
|---|---|---|
| G1 | G2 | G3 |

Fibrosis

| | | |
|---|---|---|
| 11 (24%) | 5 (11%) | 0 (0%) |

Joint Stiffness

| | | |
|---|---|---|
| 5 (11%) | 5 (11%) | 0 (0%) |

Lymphedema

| | | |
|---|---|---|
| 2 (4%) | 2 (4%) | 0 (0%) |

No grade 3 or higher late toxicities were observed. Overall, 7 of 44 evaluable patients (16%) developed at least one grade 2 or higher radiation-associated late toxicity, which met the primary endpoint. Grade 2 fibrosis (11%) and joint stiffness (11%) were more frequent than grade 2 lymphedema (4%). Of evaluable patients, 34 patients had minimum two years follow-up; five (14.7%) developed grade ≥2 fibrosis, lymphedema and/or joint stiffness. A non-significant trend was observed toward increased grade ≥2 radiation-associated toxicities in patients with tumors larger than the median size of 6.5 cm (p=0.101, χ² test), while no association was observed between RT modality (IMRT, 3D, or electron) and toxicities, though the limited number of patients treated with 3D-conformal and electron RT limits this comparison (data not shown).

### Wound Complications

Major wound complications were observed in 16 of 50 patients (32%). This rate is on par with rates of major wound complications observed in prospective studies of neoadjuvant RT (35% in the neoadjuvant RT arm of the NCIC Phase 3 study; 30.5% in multi-institutional phase 2 study of image guided IMRT, and 36.6% in RTOG 0630; O'Sullivan et al (2002) LANCET 359:2235-41; Wang et al. (2015) J CLIN ONCOL 33:2231-8; O'Sullivan et al. (2013) CANCER 119:1878-84) as well as retrospective analyses. By CTCAE criteria, 12 patients (24%) experienced grade 3 or higher wound complication or wound dehiscence, including three patients who required a reconstruction flap (grade 4 complication). In the 16 patients with major wound complications, 14 have achieved wound closure at a median time to closure of 6.4 months. There were more wound complications in patients with lower extremity tumors (p=0.01; TABLE 5), including five of nine patients with adductor compartment involvement.

TABLE 5
Association of tumor size and tumor site with
wound toxicity using the likelihood ratio test.

| | Resid. | Resid. | | | |
|---|---|---|---|---|---|
| | Df | Dev | Df | Deviance | p-value |
| Tumor Size | | | | | |
| Null Model | 47.00 | 58.98 | NA | NA | NA |
| Model with Tumor Size | 49.00 | 62.69 | −2.00 | −3.71 | 0.16 |
| Tumor site | | | | | |
| Null Model | 47.00 | 52.94 | NA | NA | NA |
| Model with Tumor Site | 49.00 | 62.69 | −2.00 | −9.75 | 0.01 |

Wound complications were not associated with time interval from radiation to surgery, tumor depth, tumor size or either of two parameters for radiation dose to the skin.

Association of Germline Biomarkers with Wound Complications

Given the paucity of clinical factors that predict for wound complications, it was hypothesized that inherent patient radiosensitivity may contribute to the risk of wound complications after neoadjuvant RT. In exploratory analysis, among lower extremity tumor site and a panel of 116 annotated SNPs in miRNA binding sites, tumor site and 19 SNPs were identified as the top 20 predictors for major wound complication rate (TABLE 6).

TABLE 6
Top 20 germline single nucleotide polymorphisms in
miRNA binding sites associated with wound toxicity.

| | Rank [mean decrease | Marginal association |
|---|---|---|
| Predictor name | in the Gini impurity] | p-value |
| Lower extremity site | 1 | 0.024808 |
| miR.99a.promoter | 2 | 0.121972 |
| IL1.B_rs4848306 | 3 | 0.327304 |
| SHC4_rs1062124 | 4 | 0.171285 |
| rs17599026 | 5 | 0.096699 |
| RAC1_rs9374 | 6 | 0.12321 |
| IL24_rs1150258 | 7 | 0.169544 |
| IL1A_rs1800587 | 8 | 0.226869 |
| ERCC1_rs3212948 | 9 | 0.264806 |
| IL.6_rs12700386 | 10 | 0.210617 |
| IL.6_rs2069840 | 11 | 0.239947 |
| CAMK2G_rs2306327 | 12 | 0.184277 |
| BMP2_rs1980499 | 13 | 0.372301 |
| EREG_rs1460008 | 14 | 0.231119 |
| CD274_rs4143815 | 15 | 0.195667 |
| IL17D_rs7787 | 16 | 0.359508 |
| STAT3_rs3744483 | 17 | 0.287639 |
| BMP4_rs17563 | 18 | 0.938445 |
| IRF5_rs10488631 | 19 | 0.201035 |
| UNGC.41.IL1RAP | 20 | 0.169942 |

The prediction performance for four proposed classifiers (classification tree, random forest, boosted tree, and LASSO-LR) using these 20 predictors jointly were fairly similar (TABLE 7), with random forests performing the best, with an accuracy of 0.855, a specificity of 0.792, sensitivity of 0.917, AUC of 0.952 and F1 of 0.868.

TABLE 7
Performance measures for prediction of major wound
toxicity using germline biomarker and tumor site.

| | | specificity | sensitivity | | | | |
|---|---|---|---|---|---|---|---|
| | acc | (TNR) | (TPR) | NPV | PPV | AUC | F1 |
| Classification | 0.692 | 0.650 | 0.733 | 0.750 | 0.757 | 0.757 | 0.696 |
| Tree | | | | | | | |
| Random | 0.855 | 0.792 | 0.917 | 0.922 | 0.850 | 0.952 | 0.868 |
| Forest | | | | | | | |
| Boosted Tree | 0.792 | 0.667 | 0.917 | 0.875 | 0.770 | 0.798 | 0.827 |
| LASSO-LR | 0.780 | 0.667 | 0.892 | 0.917 | 0.767 | 0.839 | 0.790 |

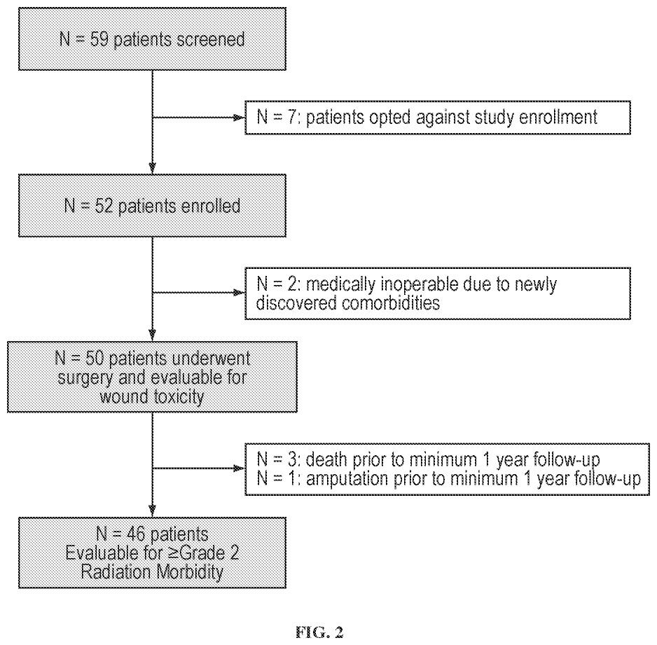

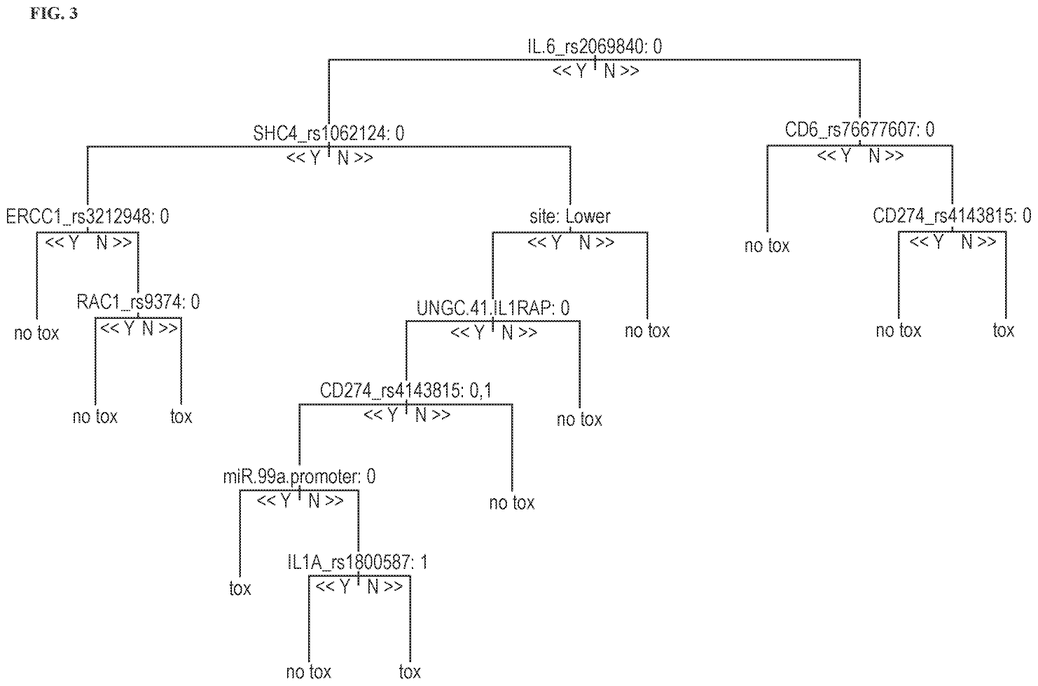

A representative decision tree based on the random forest classifier is shown in FIG. 3. Tree-based classification provides an intuitive and easily interpreted prognostic rule. Each leaf of the tree provides a probability of developing major wound toxicity after neoadjuvant RT and the proportion of the sample falling into each leaf category. The tree provides a method for prognosticating a patient's likelihood of developing toxicity based on a clinical variable (presence of lower extremity tumor) and 9 SNPs—IL6_rs2069840, SHC4_rs1062124, ERCC1_rs3212948, RAC1_rs9374, UNGC.41.IL1RAP, CD274_rs4143815, miR99a promoter, IL1A_rs1800587, and CD6_rs76677607.

As shown in FIG. 3, the presence or absence of a homozygous wild-type (0) marker IL6_rs2069840 is the first variable of predictive value. If a patient is not homozygous wild-type for IL6_rs2069840, by following “N” and travelling right on the tree, the marker CD6_rs76677607 should be considered. If the patient is homozygous wild-type (0) for CD6_rs76677607, by following “Y” and travelling left on the tree, the patient is not predicted to have a toxic response. If the patient is not homozygous wild-type (0) for CD6_rs76677607, by following “N” and travelling right on the tree, the marker CD274_rs4143815 should be considered. If the patient is homozygous wild-type (0) for CD274_rs4143815, by following “Y” and travelling left on the tree, the patient is not predicted to have a toxic response. If the patient is not homozygous wild-type (0) for CD274_rs4143815, by following “N” and travelling right on the tree, the patient is predicted to have a toxic response.

If the patient is homozygous wild-type (0) for the first variable of predictive value, CD6_rs76677607, by following “Y” and travelling left on the tree, the marker SHC4_rs1062124 should be considered. If the patient is homozygous wild-type (0) for SHC4_rs1062124, by following “Y” and travelling left on the tree, the marker ERCC1_rs3212948 should be considered. If the patient is homozygous wild-type (0) for ERCC1_rs3212948, by following “Y” and travelling left on the tree, the patient is not predicted to have a toxic response. If the patient is not homozygous wild-type (0) for ERCC1_rs3212948, by following “N” and travelling right on the tree, the marker RAC1_rs9374 should be considered. If the patient is homozygous wild-type (0) for RAC1_rs9374, by following “Y” and travelling left on the tree, the patient is not predicted to have a toxic response. If the patient is not homozygous wild-type (0) for RAC1_rs9374, by following “N” and travelling right on the tree, the patient is predicted to have a toxic response.

If the patient is not homozygous wild-type (0) for SHC4_rs1062124, by following “N” and travelling right on the tree, the presence of a lower extremity tumor should be considered. If the patient does not have a tumor on a lower extremity, by following “N” and travelling right on the tree, the patient is not predicted to have a toxic response. If the patient does have a tumor on a lower extremity, by following “Y” and travelling left on the tree, the marker UNGC.41.IL1RAP should be considered. If the patient is not homozygous wild-type (0) for UNGC.41.IL1RAP, by following “N” and travelling right on the tree, the patient is not predicted to have a toxic response. If the patient is homozygous wild-type (0) for UNGC.41.IL1RAP, by following “Y” and travelling left on the tree, the marker CD274_rs4143815 should be considered. If the patient is not homozygous wild-type (0) or heterozygous (1) for CD274_rs4143815, by following “N” and travelling right on the tree, the patient is not predicted to have a toxic response. If the patient is homozygous wild-type (0) or heterozygous (1) for CD274_rs4143815, by following “Y” and travelling left on the tree, the marker miR.99a.promoter should be considered. If the patient is homozygous wild-type (0) for miR.99a.promoter, by following “Y” and travelling left on the tree, the patient is predicted to have a toxic response. If the patient is not homozygous wild-type (0) for miR.99a.promoter, by following “N” and travelling right on the tree, the marker IL1A_rs1800587 should be considered. If the patient is heterozygous (1) for IL1A_rs1800587, by following “Y” and travelling left on the tree, the patient is not predicted to have a toxic response. If the patient is not heterozygous (1) for IL1A_rs1800587, by following “N” and travelling right on the tree, the patient is predicted to have a toxic response.

### Discussion

While clinical or dosimetric predictors other than lower extremity tumor location were not associated with wound toxicity, an exploratory analysis of germline SNPs in miRNA binding sites suggests a role for patient-intrinsic biology as a factor in the development of wound complications after neoadjuvant RT. There are indeed germline differences in radiosensitivity of normal tissues between individuals. (See, e.g., Grossberg et al. (2018) JAMA Oncol 4:1751-1757, West et al. (2010) Radiother Oncol 94:117-8, Chang-Claude et al. (2009) Br J Cancer 100:1680-6, Isomura et al. (2008) Clin Cancer Res 14:6683-9, Giotopoulos et al. (2007) Br J Cancer 96:1001-7, Damaraju et al. (2006) Clin Cancer Res 12:2545-54, Andreassen et al. (2006) Int J Radiat Biol 82:577-86, Andreassen et al. (2003) Radiother Oncol 69:127-35, Quarmby et al. (2003) Int J Radiat Biol 79:137-43, Angele et al. (2003) Cancer Res 63:8717-25.) The majority of existing evidence on this topic centers on late skin and tissue response to radiotherapy of the breast.

The above analysis identified a set of 19 germline alterations in microRNA binding sites in genes with roles in immune and DNA damage response that, in combination with lower extremity tumor location, are a predictor of major wound complications. These data are limited by the sample size of the phase 2 study. Nonetheless, these data provide for the identification of patients who are at risk of major wound complications in advance of treatment. For these at-risk patients, more aggressive dosimetric constraints, consideration of adjuvant radiotherapy, alternative surgical approaches or changes in post-surgical wound care may be warranted. An increase in the number of patients treated with neoadjuvant RT and the distance traveled by patients to the high-volume center that coincided with study initiation was observed, suggesting five-day neoadjuvant RT would increase the utilization of neoadjuvant RT and access to care at high-volume sarcoma centers.

### Example 2. Biomarkers Relevant to Distant Failure Status

Fifty (50) sarcoma patients from Example 1 with distant failure status (response variable, 1 when positive, 0 when negative) were studied. One hundred sixteen (116) genotyping data and tumor site, as categorical variable, for each patient were collected.

The association between a panel of potential germline biomarkers and distant failure, disease-free survival, was assessed using four classifiers on a set of 50 sarcoma patients, with distant failure rate 20%, Trained classifiers, whose hyperparameters were selected to optimize the F1 score with leave-one-out cross-validation (LOOCV), included classification trees (Breiman L, Friedman J H, Olshen R A, Stone C J, eds. Classification and regression trees.: Boca Raton, Fla.: CRC Press.; 2017), random forests (Breiman L. Random forests. Machine Learning 2001; 45:5-32, boosted trees (XGBoost: A Scalable Tree Boosting System. 2016. at https://arxiv.org/abs/1603.02754), and LASSO-regularized logistic regression (LASSO-LR) (Tibshirani R. Regression Shrinkage and Selection via the Lasso. Journal of the Royal Statistical Society 1996; Series B (Methodological):267-88).

The Classification Trees were tuned on minimum split and minimum observations in any terminal node, Random Forests were tuned on number of trees and variables considered at each split, Boosted Trees were tuned on the learning parameter eta, tree depth, and the number of rounds, and LASSO-LR models were tuned on the regularization parameter lambda. The subjects with toxicity were up-weighted through oversampling method for training set in each iteration of cross-validation. The final performance measures, accuracy, specificity, sensitivity, negative predictive value, positive predictive value, area under the curve (AUC), and F1 score were reported using LOOCV for 100 over sample sets. The threshold of number of predictors to include in the model was determined as the use of top k predictors allowed highest AUC among k=5, 10, 15, . . . , 50. Importance measures via filter method (Piotr Romanski L. K. (2018). FSelector: Selecting Attributes. R package version 0.31) were used to select top k predictors, as determined by mean rank from 1000 sample sets of their respective value and whose pvalue from a chi-squared test was less or equal to 0.3, to train the classifier. The four important measures are entropy-based information gain between predictors and response, variable importance based on ranger impurity importance, the entropy-based gain ratio between predictors and response and the univariate model score.

Classification Trees, Random Forests, Boosted Trees, and LASSO-LR classifiers were fit in R (version 3.6.0) (R Core Team (2019). R: A language and environment for statistical computing. R Foundation for Statistical Computing, Vienna, Austria. URL http://www.R-project.org/) with mlr (Bischl B, et al. (2018). mlr: Machine Learning in R. R package version 2.13) calling rpart (Therneau T, Atkinson B, and Ripley B. (2018). rpart: Recursive Partitioning and Regression Trees. R package version 4.1-13), ranger (Wright M N, Wager S, and Probst P. (2019). ranger: A Fast Implementation of Random Forests. R package version 0.11.2), xgboost (Chen T, et al. (2019). xgboost: Extreme Gradient Boosting. R package version 0.82.1), glmnet (Friedman J, et al. (2018). glmnet: Lasso and Elastic-Net Regularized Generalized Linear Models. R package version 2.0-16), respectively, and the tree plot was generated with the package reprtree (Banerjee, Mousumi & Ding, Ying & Noone, Anne-Michelle. (2012). Identifying representative trees from ensembles. Statistics in medicine. 31. 1601-16. 10.1002/sim.4492). Via 100 over sampling sets, the order of significance of these k predictors was determined according to the obtained variable importance measure of mean decrease in the Gini impurity from the trained random forest classifier for its best prediction performance of PPV among 4 classifiers.

The classification strategies described are compared in TABLE 8 for predicting distant failure. Sensitivity is intended as the probability of correctly predicting distant failure; specificity is intended as the probability of correctly predicting no distant failure. PPV is intended as the probability of correctly classifying a patient as having distant failure, and NPV is intended as the probability of correctly classifying a patient as not exhibiting distant failure. The F1 Score is an evaluation of test accuracy determined using the PPV and Sensitivity measures.

TABLE 8
Distant Failure

| | | Sen- | Speci- | | | | F1 |
|---|---|---|---|---|---|---|---|
| | Accuracy | sitivity | ficity | NPV | PPV | AUC | score |
| Classification | 0.661 | 0.469 | 0.709 | 0.842 | 0.290 | 0.563 | 0.769 |
| Trees | | | | | | | |
| Random | 0.840 | 0.453 | 0.937 | 0.873 | 0.649 | 0.835 | 0.903 |
| Forests | | | | | | | |
| Boosted | 0.746 | 0.560 | 0.793 | 0.879 | 0.404 | 0.750 | 0.833 |
| Trees | | | | | | | |
| LASSO-LR | 0.848 | 0.671 | 0.893 | 0.916 | 0.616 | 0.890 | 0.904 |

TABLE 9 shows rank order of marginal associations between distant failure and biomarkers. P values as shown are based on independent Pearson's Chi-squared Test for Count Data. As shown, the top predictors of distant failure are TREX1_rs11797, LIN28A_rs9438623, rs2187668, miR.34b.c.promoter_rs4938723, IL.6_rs12700386, CD274_rs4742098, FCGR2A_rs1801274, IL.6_rs2069840, CD274_rs4143815, ERCC4_rs4781562, STAT4_rs7574070, EXO1_rs4150021, HAMP_rs1882694, IL10_rs3024496, KRAS_rs61764370, rs922075, ERCC1_rs11615, SOS1_rs1059313, SPI1_rs2071304, and, SHC4_rs1062124.

TABLE 9

| SNP_name | rank | p-value |
|---|---|---|
| TREX1_rs11797 | 1 | 0.002655 |
| LIN28A_rs9438623 | 2 | 0.022409 |
| rs2187668 | 3 | 0.034549 |
| miR.34b.c.promoter_rs4938723 | 4 | 0.038444 |
| IL.6_rs12700386 | 5 | 0.04198 |
| CD274_rs4742098 | 6 | 0.051623 |
| FCGR2A_rs1801274 | 7 | 0.071158 |
| IL.6_rs2069840 | 8 | 0.095674 |
| CD274_rs4143815 | 9 | 0.122432 |
| ERCC4_rs4781562 | 10 | 0.126526 |
| STAT4_rs7574070 | 11 | 0.128374 |
| EXO1_rs4150021 | 12 | 0.172422 |
| HAMP_rs1882694 | 13 | 0.202298 |
| IL10_rs3024496 | 14 | 0.20374 |
| KRAS_rs61764370 | 15 | 0.209611 |
| rs922075 | 16 | 0.209611 |
| ERCC1_rs11615 | 17 | 0.236306 |
| SOS1_rs1059313 | 18 | 0.253207 |
| SPI1_rs2071304 | 19 | 0.27906 |
| SHC4_rs1062124 | 20 | 0.287184 |

### Example 3. Biomarkers of Toxicity Resulting from Hypofractionated Stereotactic Body Radiation Therapy (SBRT) and Conventionally Fractionated Radiation Treatment (CF-RT)

Given the high effectiveness of definitive treatments for localized prostate cancer, quality of life following treatment is a paramount factor in patient-physician shared decision-making. After definitive radiotherapy, a major quality of life limiting toxicity is late genitourinary (GU) toxicity, which could manifest as increased urinary frequency, retention, pain, and bleeding. The 5-year late grade ≥2 GU toxicity rates following modern radiotherapy ranges from 12-15% with an insidious increase over time. Overall, these rates appear to be similar whether a patient is treated with conventionally-fractionated radiotherapy (CF-RT; 1.8-2.0 Gy per fraction over 39-45 treatment sessions) or stereotactic body radiotherapy (SBRT; >7 Gy per fraction over 5 or fewer sessions). While the overall toxicity rates also are similar, an unanswered question is whether variability in intrinsic radiosensitivity, which should rationally differ based on radiation fraction size delivered, may predict whether individual men are more likely to experience significant toxicity after SBRT versus CF-RT.

The following Example identifies mirSNPs that can help to aid in the prediction of toxicity following CF-RT versus SBRT in a fractionation-dependent manner.

In this study, 93 prostate cancer patients from the University of California, Los Angeles (NCT01059513 [n=63] and NCT02296229 [n=30]) were administered hypofractionated stereotactic body radiation therapy (SBRT) with 40 Gy in 5 fractions (8 Gy per fraction) for a period not exceeding 14 consecutive days. SBRT plans were delivered by volumetric modulated arc therapy, with a planning margin of 5 mm around the prostate, reduced to 3-5 mm posteriorly. Inter- and intrafractional motion management relied on alignment to intraprostatic fiducial markers. Gastrointestinal (GI) and genitourinary (GU) toxicity was assessed.

In addition, gastrointestinal (GI) and genitourinary (GU) toxicity was assessed for prostate cancer patients receiving the conventionally-fractionated radiation treatment (CF-RT; i.e., standard fractionation treatment), using data from the OSLO study. The OSLO cohort dataset is described in, e.g., Hayman et al. (2019) Urol. Oncol. 37(4):289.e19-289.e26 and Lilleby et al. (2013) Prostate 73(10):1038-1047. Briefly, patients in the CF-RT cohort received 74 Gy in 37 fractions to the prostate and 50 Gy in 25 fractions to the pelvic lymph nodes as described previously. The initial portion was delivered with intensity modulated radiotherapy techniques, with expansions on the prostate ranging from 13-15 mm and imaging guidance predominantly consisting of alignment to bony markers.

### Analysis of Germline DNA

Genomic DNA from peripheral blood mononuclear cells, whole blood, or tumor tissue was isolated using standard techniques and analyzed in a Clinical Laboratory Improvement Amendments—certified laboratory as previously described. (Weidhaas et al. (2017) “The KRAS-Variant and Cetuximab Response in Head and Neck Squamous Cell Cancer: A Secondary Analysis of a Randomized Clinical Trial,” JAMA Oncology 3:483-91. Biomarkers were identified from a pool of miRNA-based biomarkers discovered and determined to be functional previously through sequencing and bioinformatic approaches. Chen et al. (2015) “Targeted resequencing of the microRNAome and 3′UTRome reveals functional germline DNA variants with altered prevalence in epithelial ovarian cancer,” Oncogene 34:2125-37. Mutations in DNA damage repair and response genes and immune response genes, in the key gene targets of miRNAs known to be critical in the DNA damage or immune response, and in the promoters of miRNAs known to be important in these responses were prioritized. Therefore, for this analysis, panels were run using the Sequenome platform, an analysis which included approximately 500 single nucleotide polymorphisms or deletions. Each panel was run with internal controls that used Taqman Genotyping as the gold standard. To compare the genetic variation between the CF-RT and SBRT cohorts, we calculated the fixation index for each analyzed mirSNP. Holsinger et al. (2009) “Genetics in geographically structured populations: defining, estimating and interpreting F(ST),” Nat Rev Genet. 10:639-50.

### Variable Selection and Model Fitting

Statistical models and analyses were conducted in R (version 4.0.0). All mirSNPs with variance close to zero (nearZeroVar::caret version 6.0-84)27 or that had an almost perfect correlation (r2≥0.99) were removed. Fisher's exact test was used to test the pairwise independence between each mirSNP and the outcome of experiencing a late grade ≥2 GU toxicity event as scored on the Radiation Therapy Oncology Group (RTOG) scale. (Cox et al. (1995) “Toxicity criteria of the Radiation Therapy Oncology Group (RTOG) and the European Organization for Research and Treatment of Cancer (EORTC),” International journal of radiation oncology, biology, physics. 31:1341-6.

This scoring system was developed in 1985 and grades the severity of radiation-induced reactions from 0 to 5, with grade 2 toxicity being considered moderate. For the initial assessment of pairwise independence, the p-values were only used for model selection and thus no adjustment for multiplicity of testing was performed. Due to the unequal distribution of patients with and without toxicity, up-sampling was used (upSample::caret version 6.0-84) to create balanced populations. Random forest (randomForest::randomForest version 4.6-14), boosted trees (gbm::gbm version 2.1.5), and elastic net (glmnet::glmnet version 3.0-2) models were then generated to predict toxicity in each patient for both the CF-RT and the SBRT cohorts. (Liaw et al. Classification and Regression by Random Forest. R News. 2002; 2:18-22; Greenwell et al. (2019) “Generalized Boosted Regression Models,” R Foundation for Statistical Computation; Kuhn M. caret: Classification and Regression. R Foundation for Statistical Computing; 2019; Friedman et al. (2010) Regularization Paths for Generalized Linear Models via Coordinate Descent,” Journal of Statistical Software 33:1-22.) The p-value threshold for inclusion in the models were 0.3 except for the boosted tree model for the SBRT cohort, which had a p-value threshold of 0.15. Each model was run using various parameters, and model performance was assessed for sensitivity, specificity, negative predictive value (NPV), positive predictive value (PPV), F1 score, and AUC (AUC::cvAUC version 1.1.0), using held-out samples. (LeDell E P, M.; van der Laan, M. cvAUC: Cross-Validated Area Under the ROC Curve Confidence Intervals. R Foundation for Statistical Computing; 2014.) To reduce selection bias and overfitting, these metrics were calculated using leave-one-out-cross validation.

### Analysis of SBRT Set

TABLE 10 shows toxicity events by grade for the 93 prostate cancer patients in the SBRT analysis set.

TABLE 10

| Grade | 0 | 1 | 2 | 3 |
|---|---|---|---|---|
| Acute GI | 91 | 2 | 0 | 0 |
| Acute GU | 81 | 9 | 1 | 2 |
| Long GI | 82 | 6 | 2 | 3 |
| Long GU | 58 | 21 | 11 | 3 |

Toxicity events at or above grade 2 are considered high grade toxicity. No patients experienced acute GI toxicity at or above grade 2, only 3 patients experienced acute GU high grade toxicity. In the long term, 5 patients experienced GI high grade toxicity (grade 2 and 3), and 14 patients experienced long term GU high grade toxicity (grade 2 and 3).

GI and GU toxicities appear uncorrelated within the short and long term. Low grade acute GU toxicity is mildly correlated with long term low grade GU toxicity (X-squared=25.9, p.val=0.04), but no correlation is detected between high grade toxicity in the short and long terms.

### Markers of Long Term GU Toxicity (SBRT)

Long term higher grade toxicity is defined as GU toxicity of grade 2 or higher. The analysis is based on a sample of 93 prostate cancer patients, stratified adaptively using 13 biomarkers.

TABLE 11 shows statistically significant (Fisher test p.val_0.05) marginal association between late term GU toxicity (grade 2 or higher) and genetic mutations (homozygous and heterozygous combined).

TABLE 11

| | No Mutation | Mutation | |
|---|---|---|---|
| mirSNP | (% Tox) | (% Tox) | p-value |
| BMP2_rs1979855 | 6.9 | 28.6 | 0.007 |
| rs1893217 | 8.8 | 32.0 | 0.010 |
| BRCA2_rs15869 | 9.4 | 27.6 | 0.031 |
| ABL1_rs11991 | 20.6 | 3.3 | 0.032 |
| ERCC1_rs11615 | 10.1 | 29.2 | 0.043 |
| RAD23A_rs8240 | 18.7 | 0.0 | 0.064 |
| BATF3_rs6695772 | 9.1 | 23.7 | 0.076 |
| FOXP3_rs2232365 | 20.8 | 7.5 | 0.088 |
| IL19_rs1798 | 10.6 | 25.9 | 0.106 |
| LIN28A_rs9438623 | 0.0 | 17.7 | 0.118 |
| IL18R1_rs11465660 | 12.0 | 27.8 | 0.136 |
| IL2RB_rs228942 | 7.7 | 20.4 | 0.141 |
| CD274_rs1411262 | 9.8 | 21.4 | 0.150 |
| BMP2_rs3178250 | 9.1 | 20.4 | 0.155 |
| SPI1_rs2071304 | 10.0 | 20.9 | 0.159 |
| IL19_rs2243158 | 0.0 | 17.3 | 0.202 |
| rs3024505 | 0.0 | 17.3 | 0.202 |
| TRL4_rs4986790 | 17.3 | 0.0 | 0.202 |
| VEGFA_rs41282644 | 17.1 | 0.0 | 0.206 |
| P2RX7_rs3751143 | 19.0 | 6.7 | 0.213 |
| ERCC1_rs3212948 | 22.6 | 11.3 | 0.218 |
| FANCC_rs9673 | 13.8 | 33.3 | 0.221 |
| CD274_rs2297136 | 8.3 | 19.3 | 0.234 |
| IL6_rs2069840 | 8.3 | 19.3 | 0.234 |
| CD274_rs2282055 | 10.9 | 21.1 | 0.240 |
| CD274_rs822339 | 21.1 | 10.9 | 0.240 |
| HAMP_rs10421768 | 19.2 | 9.8 | 0.252 |
| HAMP_rs1882694 | 20.0 | 11.3 | 0.260 |
| ATM_rs1800057 | 14.0 | 28.6 | 0.283 |
| ILRAP_rs79383051 | 14.0 | 38.6 | 0.283 |
| KIT_rs17084733 | 17.6 | 5.3 | 0.286 |
| rs17599026 | 17.3 | 5.6 | 0.29 |

These results indicate that patients carrying the BMP2_rs1979855 mutation, PTPN2_rs1893217 mutation or BRCA2_rs15869 are more likely to develop toxicity than patients who are homozygous wild type for the respective markers. The results further indicate that patients who are homozygous wild-type for the ABL1_rs11991 or ERCC1_rs11615 SNPs are more likely to develop toxicity than patients who carry the mutation for the respective markers.

Several classification strategies are compared in TABLE 12 for predicting acute and late term genitourinary (GU) toxicity, at or above grade 2. The following parameters were used for the determination of late term toxicity: Random Forest classification p-value threshold of 0.3 and mtry=1; Elastic Net classification p-value threshold of 0.3, α=0.1, and λ=0.0395; and Boosted Trees classification p-value threshold of 0.15, int_depth=1, and num_trees=100. Variable importance is evaluated using a “filter” methodology. Sensitivity is intended as the probability of correctly predicting a toxic response; specificity is intended as the probability of correctly predicting no toxic response. PPV is intended as the probability of correctly classifying a patient as exhibiting a toxic response, and NPV is intended as the probability of correctly classifying a patient as not exhibiting a toxic response. The F1 Score is an evaluation of test accuracy determined using the PPV and Sensitivity measures.

TABLE 12

| Classification | Sensitivity | Specificity | | | F1 |
|---|---|---|---|---|---|
| Methods | (SD) | (SD) | PPV | NPV | Score |
| Random Forest | 0.929 | 0.747 | 0.394 | 0.983 | 0.553 |
| Elastic Net | 0.786 | 0.924 | 0.647 | 0.961 | 0.710 |
| Boosted Trees | 0.714 | 0.848 | 0.455 | 0.944 | 0.556 |

FIG. 4 shows a Variable Importance diagram using a Random Forest classification strategy for late term toxicity. As shown, the top predictors of toxicity at or above grade 2 are LIN28A_rs9438623, FANCC_rs9673, VEGFA_rs41282644, and IL18R1_rs11465660.

FIG. 5 shows a Variable Importance diagram using an Elastic Net classification strategy for late-term toxicity. As shown, the top predictors of toxicity at or above grade 2 are CD274_rs1411262, IL10_rs3024505, IL19_rs1798, BMP2_rs1979855, HAMP_rs10421768, FOXP3_rs2232365, VEGF rs41282644, IL2RB_rs228942, P2RX7_rs3751143, IL19_rs2243158, CD274_rs822339, LIN28A_rs9438623, CD274_rs2282055, and KIT_rs170847733.

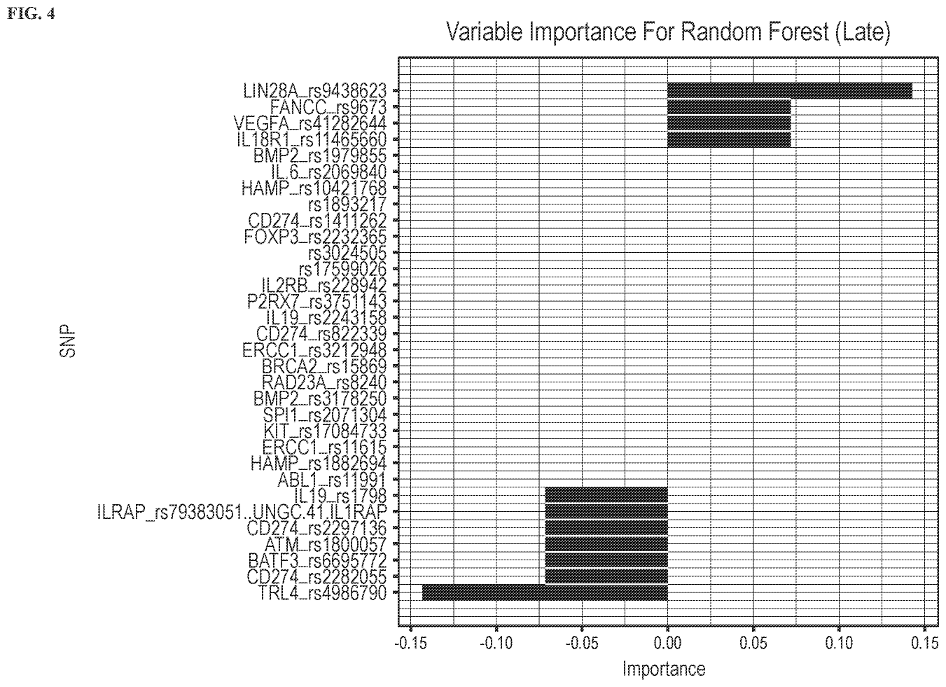

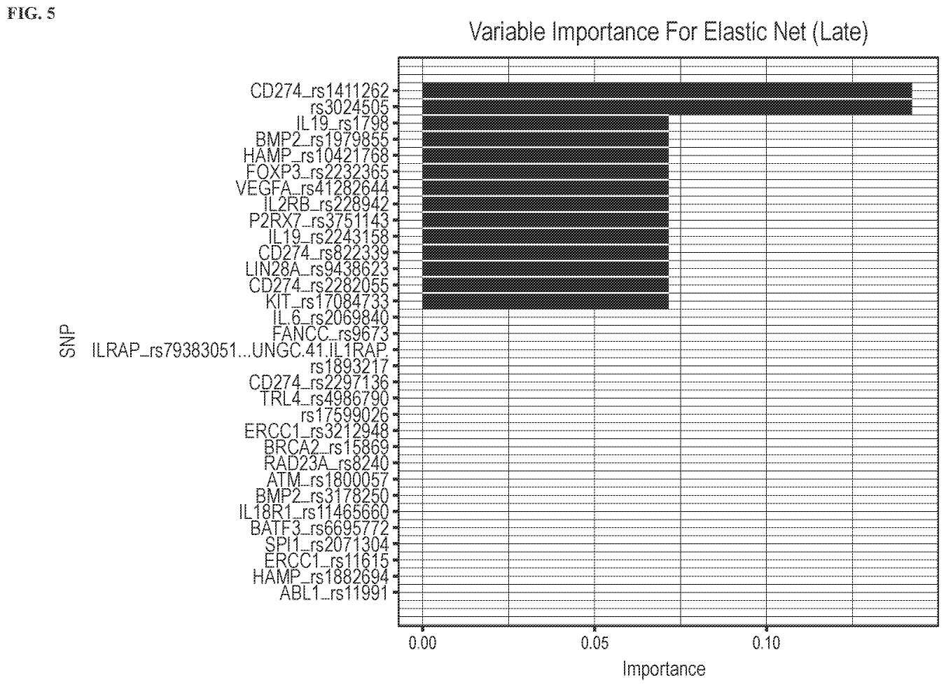

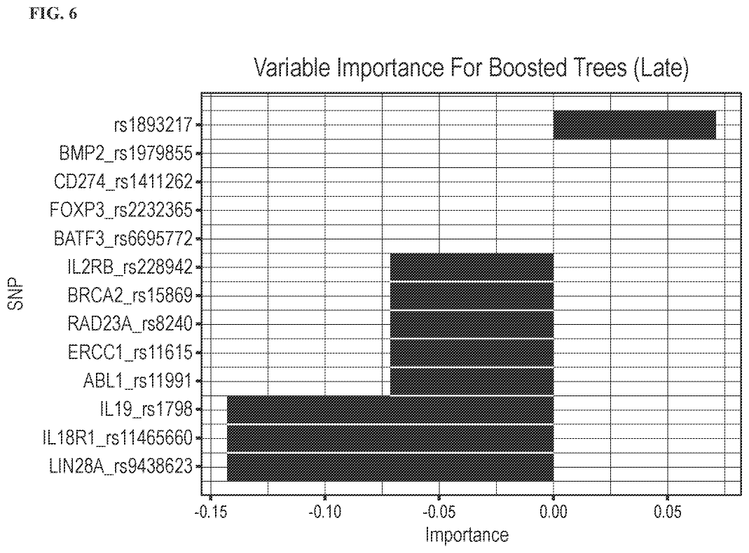

FIG. 6 shows a Variable Importance diagram using a Boosted Trees classification strategy for late-term toxicity. As shown, the top predictor of toxicity at or above grade 2 is rs1893217.

### Analysis of CF-RT Set

TABLE 13 shows descriptive summaries of Acute and Late-term GU Toxicity in the CF-RT data set, as they relate to basic clinical and physiological information. Marginal associations were observed between acute and late term GU toxicity (at or above grade 2) and several clinical and physiological variables. Patients experiencing acute toxicity are, on average, 3 years older (p.val=0.025). Patients experiencing late term toxicity tend to be exposed to longer androgen deprivation therapy (ADT) duration (p.val=0.007).

TABLE 13

| | | | |
|---|---|---|---|
| | \|Grade <2\| | \|Grade ≥2\| | |
| Variable | Mean (SD) | Mean (SD) | p-value |

Acute GU Toxicity

| | | | |
|---|---|---|---|
| Age | 65.18 (6.40) | 68.10 (6.18) | 0.025 |
| Gleason | 4.73 (1.13) | 4.58 (1.20) | 0.530 |
| Initial PSA | 32.21 (25.60) | 31.19 (17.71) | 0.831 |
| T Stage | 6.01 (1.40) | 6.05 (1.45) | 0.890 |
| Duration ADT | 32.89 (20.30) | 29.02 (22.78) | 0.370 |

Late GU Toxicity

| | | | |
|---|---|---|---|
| Age | 65.77 (5.94) | 68.38 (8.58) | 0.126 |
| Gleason | 4.67 (1.12) | 4.75 (1.39) | 0.800 |
| Initial PSA | 30.71 (22.22) | 38.41 (27.84) | 0.222 |
| T Stage | 6.09 (1.35) | 5.71 (1.72) | 0.308 |
| Duration ADT | 29.21 (17.44) | 44.13 (32.91) | 0.007 |

TABLE 14 shows marginal associations between acute toxicity (at or above grade 2) and biomarkers for the CF-RT data set. P values are based on independent Pearson's Chi-squared Test for Count Data. As shown, the top predictors of acute toxicity at or above grade 2 are ATM_rs189037, CD6_rs76677607, IL1A_rs1800587, IL1A_rs17561, BRCA2_rs7334543, ILF3.58_rs118142475, and BIRC5_rs2239680.

TABLE 14
Acute Toxicity

| | No Mutation | Mutation | |
|---|---|---|---|
| SNP | (% Toxicity) | (% Toxicity) | p-value |
| ATM_rs189037 | 61.9 | 27.6 | 0.005 |
| CD6_rs76677607 | 28.6 | 64.7 | 0.010 |
| IL1A_rs1800587 | 45.5 | 22.6 | 0.015 |
| IL1A_rs17561 | 44.6 | 23.1 | 0.025 |
| BRCA2_rs7334543 | 47.5 | 26.5 | 0.036 |
| ILF3.58_rs118142475 | 32.4 | 100.0 | 0.038 |
| BIRC5_rs2239680 | 25.8 | 45.7 | 0.041 |

TABLE 15 shows marginal associations between late toxicity (at or above grade 2) and biomarkers for the CF-RT data set. P values are based on independent Pearson's Chi-squared Test for Count Data. As shown, the top predictors of late toxicity at or above grade 2 are CD6_rs76677607, CD274_rs2297136, BRCA2_rs7334543, NBN_rs1805794, ILI.1_rs4848306, CD274_rs4742098, ERCC4_rs4781562, and MSH2_rs2303428.

TABLE 15
Late Toxicity

| | No Mutation | Mutation | |
|---|---|---|---|
| SNP | (% Toxicity) | (% Toxicity) | p-value |
| CD6_rs76677607 | 9.9 | 47.1 | 0.001 |
| CD274_rs2297136 | 0.0 | 21.2 | 0.005 |
| BRCA2_rs7334543 | 27.5 | 8.8 | 0.014 |
| NBN_rs1805794 | 6.2 | 23.3 | 0.017 |
| ILI.1_rs4848306 | 3.2 | 20.8 | 0.022 |
| CD274_rs4742098 | 21.7 | 5.1 | 0.027 |
| ERCC4_rs4781562 | 7.7 | 23.2 | 0.035 |
| MSH2_rs2303428 | 11.8 | 30.4 | 0.048 |
| rs17599026 | 11.9 | 29.2 | 0.056 |
| XRCC3_rs861539 | 7.0 | 21.5 | 0.058 |
| IL2RA_rs11256497 | 8.0 | 22.4 | 0.062 |
| LIG4_rs3093772 | 19.5 | 3.8 | 0.067 |
| IL17D_rs7787 | 6.1 | 20.0 | 0.087 |
| IL6_rs1800795 | 19.3 | 4.0 | 0.113 |
| CD274_rs4143815 | 20.6 | 8.9 | 0.115 |
| UNGC.96.TGFB2_NA | 14.1 | 33.3 | 0.149 |
| miR.34b.c.promoter_rs4938723 | 22.0 | 11.9 | 0.183 |
| MDM2_rs769412 | 14.3 | 30.0 | 0.191 |
| rs3024505 | 18.7 | 9.1 | 0.261 |
| CETN2_rs8230 | 14.1 | 25.0 | 0.275 |
| BRCA2_rs15869 | 11.3 | 20.0 | 0.292 |
| IL10_rs1800872 | 12.1 | 20.0 | 0.298 |

Several classification strategies are compared in TABLE 16 for predicting acute and late term genitourinary (GU) toxicity, at or above grade 2. All models were fit using variables that had a p-value of 0.3 or less when conducting a chi-squared test for independence. The following parameters were used for the determination of acute term toxicity: Random Forest classification p-value threshold of 0.3 and mtry=1; Elastic Net classification p-value threshold of 0.3, α=0.1, and λ=0.0404; and Boosted Trees classification p-value threshold of 0.15, int_depth=3, and num_trees=100. The following parameters were used for the determination of late term toxicity: Random Forest classification p-value threshold of 0.3 and mtry=3; Elastic Net classification p-value threshold of 0.3, α=0.1, and λ=0.00404; and Boosted Trees classification p-value threshold of 0.3, int_depth=2, and num_trees=150. Variable importance is evaluated using a “filter” methodology. Sensitivity is intended as the probability of correctly predicting a toxic response; specificity is intended as the probability of correctly predicting no toxic response. PPV is intended as the probability of correctly classifying a patient as exhibiting a toxic response, and NPV is intended as the probability of correctly classifying a patient as not exhibiting a toxic response. The F1 Score is an evaluation of test accuracy determined using the PPV and Sensitivity measures.

TABLE 16

| | | | | | |
|---|---|---|---|---|---|
| Classification | Sensitivity | Specificity | | | F1 |
| Methods | (SD) | (SD) | PPV | NPV | Score |

Acute Toxicity

| | | | | | |
|---|---|---|---|---|---|
| Random Forest | 0.784 | 0.690 | 0.569 | 0.860 | 0.659 |
| Elastic Net | 0.622 | 0.803 | 0.622 | 0.803 | 0.622 |
| Boosted Trees | 0.622 | 0.803 | 0.622 | 0.803 | 0.622 |

Late Term Toxicity

| | | | | | |
|---|---|---|---|---|---|
| Random Forest | 0.765 | 0.802 | 0.419 | 0.948 | 0.542 |
| Elastic Net | 0.588 | 0.956 | 0.714 | 0.926 | 0.645 |
| Boosted Trees | 0.647 | 0.967 | 0.786 | 0.936 | 0.710 |

FIG. 7 shows a Variable Importance diagram using a Random Forest classification strategy for late term toxicity. As shown, the top predictors of toxicity at or above grade 2 are CD6_rs76677607, CD274_rs2297136, MSH2_rs2303428, LIG4_rs3093772_P1.P2, miR.34b.c.promoter_rs4938723, rs17599026, BRCA2_rs7334543, UNGC.96.TGFB2_NA, BRCA2_rs15869, CETN2_rs8230, IL10_rs3024505, MDM2_rs769412, IL1.B_rs4848306, IL6_rs1800795, IL2RA_rs11256497, ERCC4_rs4781562, IL17D_rs7787, CD274_rs4742098, NBN_rs1805794, XRCC3_rs861539, IL10_rs1800872, and CD274_rs4143815.

FIG. 8 shows a Variable Importance diagram using an Elastic Net classification strategy for late-term toxicity. As shown, the top predictors of toxicity at or above grade 2 are CD274_rs2297136, miR.34b.c.promoter_rs4938723, LIG4_rs3093772_P1.P2, UNGC.96.TGFB2_NA, IL1.B_rs4848306, ERCC4_rs4781562, BRCA2_rs7334543, NBN_rs1805794, IL6_rs1800795, IL2RA_rs11256497, IL17D_rs7787, XRCC3_rs861539, MSH2_rs2303428, CD6_rs76677607, and BRCA2_rs15869.

FIG. 9 shows a Variable Importance diagram using a Boosted Trees classification strategy for late-term toxicity. As shown, the top predictors of toxicity at or above grade 2 are IL6_rs1800795, BRCA2_rs7334543, ERCC4_rs4781562, IL10_rs3024505, MDM2_rs769412, LIG4_rs3093772, MSH2_rs2303428, IL2RA_rs11256497, CD274_rs4742098, CD274_rs2297136, UNGC.96.TGFB2_NA, CETN2_rs8230, rs17599026, and CD274_rs4143815.

FIG. 10 shows a Variable Importance diagram using a Random Forest classification strategy for acute toxicity. As shown, the top predictors of toxicity at or above grade 2 are IL2RA_rs2476491, IL12A_rs568408, GSK3B_rs2037547, and IL6_rs1800795.

FIG. 11 shows a Variable Importance diagram using an Elastic Net classification strategy for acute GU toxicity. As shown, the top predictors of toxicity at or above grade 2 are BIRC5_rs2239680 and LIG4_rs2232643.

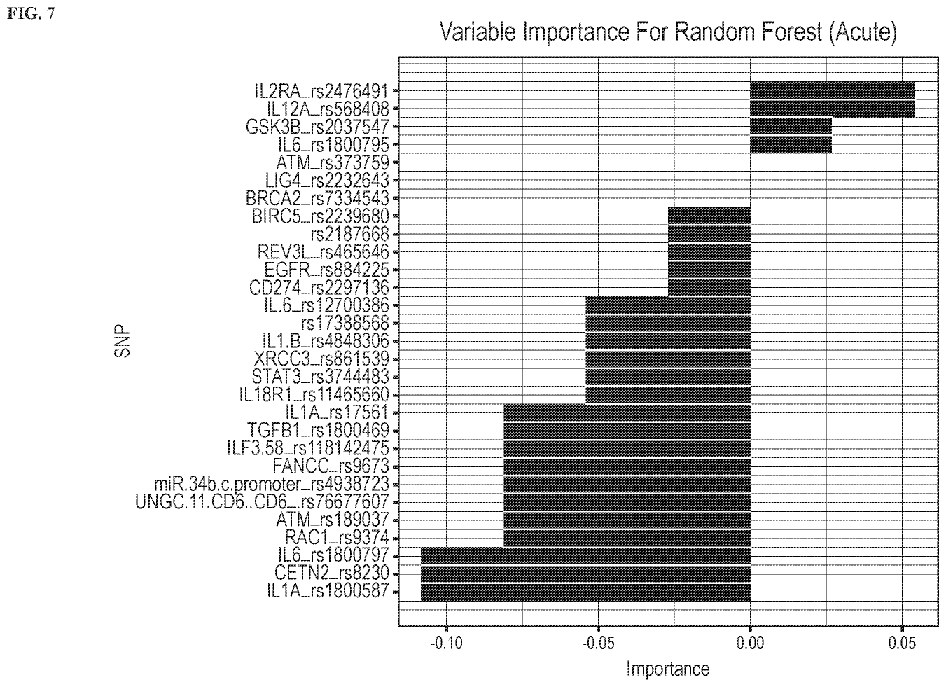

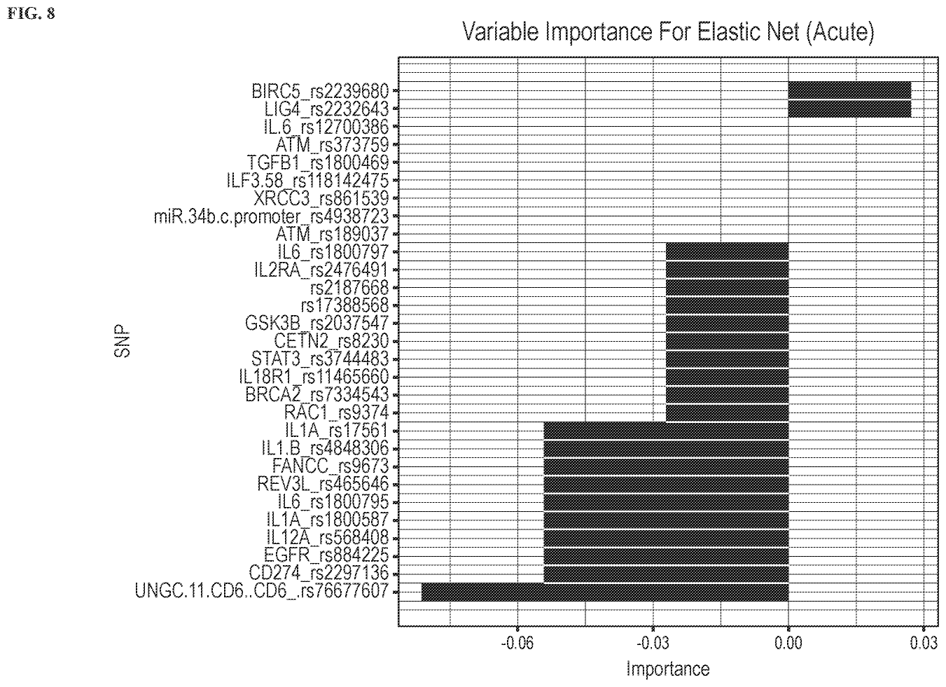

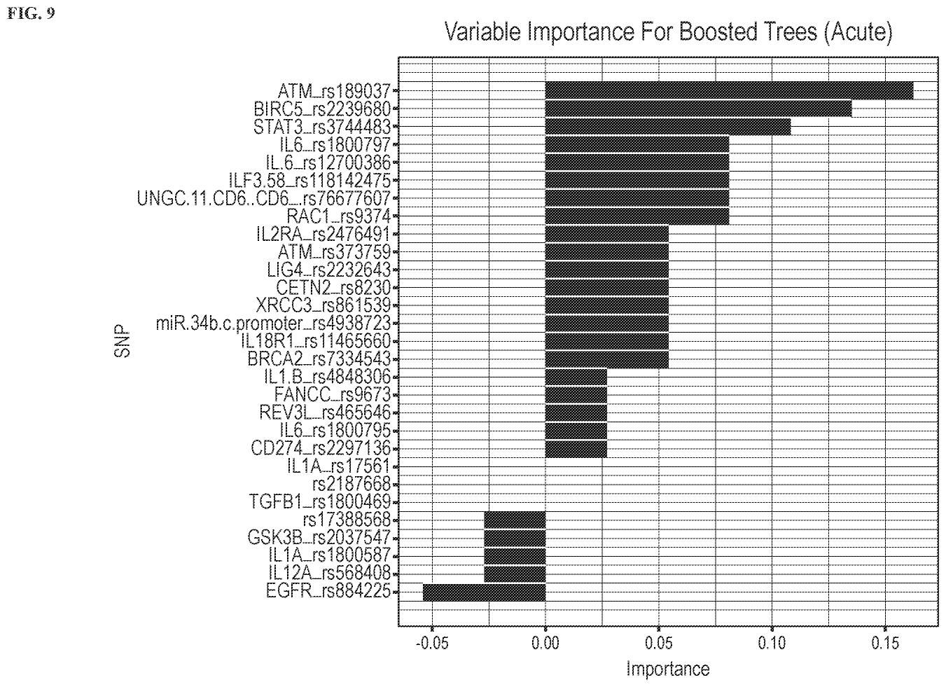

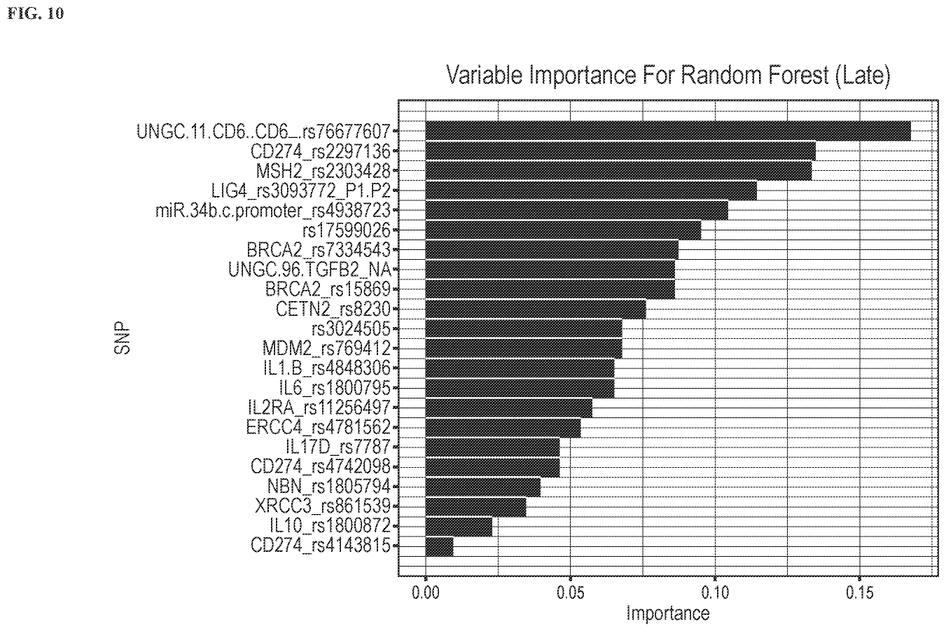

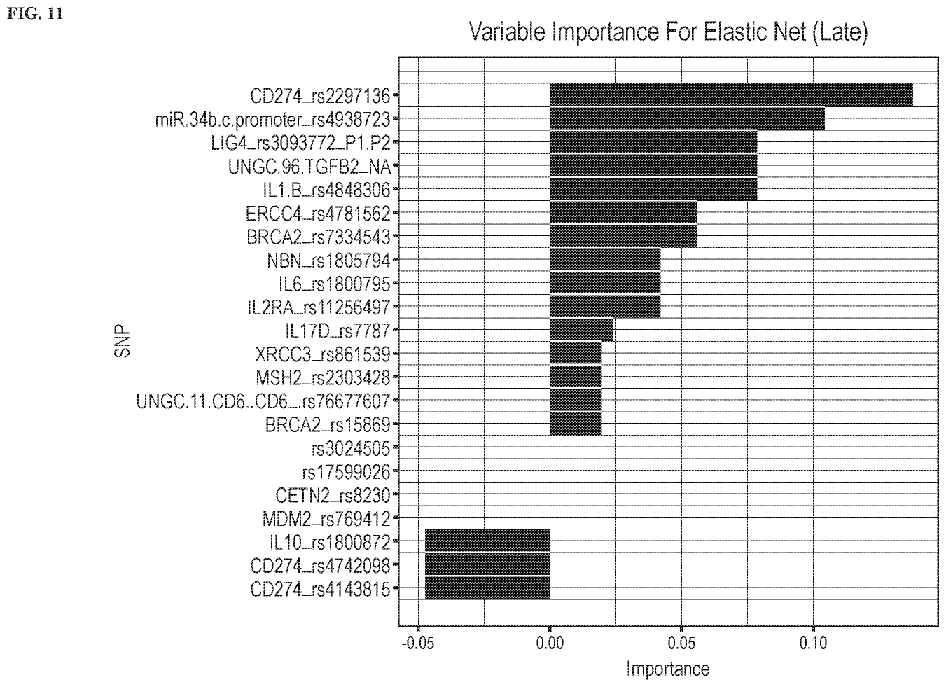

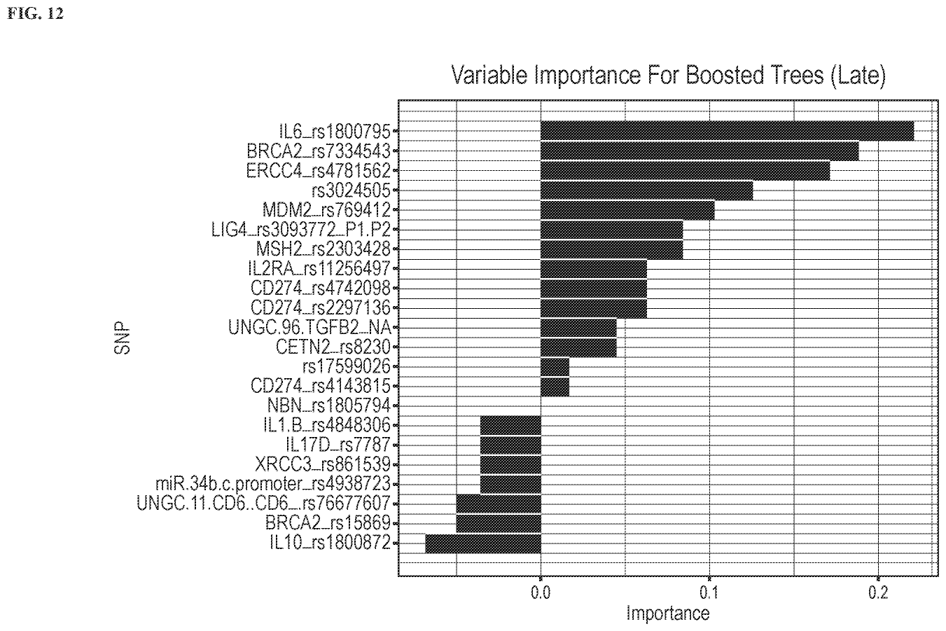

FIG. 12 shows a Variable Importance diagram using a Boosted Trees classification strategy for acute toxicity. As shown, the top predictors of toxicity at or above grade 2 are ATM_rs189037, BIRC5_rs2239680, STAT3_rs3744483, IL6_rs1800797, IL.6_rs12700386, ILF.58_rs11814275, CD6_rs76677607, RAC1_rs9374, IL2RA_rs2476491, ATM_rs373759, LIG4_rs2232643, CETN2_rs8230, XRCC3_rs861539, miR.34b.c.promoter_rs4938723, IL18R1_rs11465660, BRCA2_rs7334543, IL1.B_rs4848306, FANCC_rs9673, REV3L_rs465646, IL6_rs1800795, and CD274_rs2297136.

### SNPs Predictive of Prostate Cancer Mortality, Biochemical Relapse, Distant Metastasis and Potency

SNPs Predictive of Prostate Cancer Mortality, Biochemical Relapse, Distant Metastasis and Potency were also determined.

Survival data was available for the OSLO patients, including time until Prostate Cancer Specific Mortality, time until Distant Metastasis, and time until Biochemical Relapse. Due to the duration of the study, there is right censoring, meaning that the event had not happened in some patients yet. In this analysis, a log-rank test (survival version 2.44-1.1) was conducted to determine a SNPs marginal association with the time until the event of interest. TABLES 17, 18, and 19 contain the unadjusted P-values for the log-rank test for each SNP.

TABLE 17 shows P-values for the Log-Rank test using Prostate Cancer Specific Mortality as the outcome. A significant p-value indicates that the survival curve for a patient with the given gene mutation is significantly different than the survival curve if the patient did not have the gene mutation. This test only considers one gene at a time. As shown, the top predictors of prostate cancer specific mortality are BIRC5_rs2239680, CD6_rs76677607, XRCC4_rs1040363, and SMAD1_rs11724777.

TABLE 17
Prostate Cancer Specific Mortality

| gene | p-value |
|---|---|
| BIRC5_rs2239680 | 0.014 |
| CD6_rs76677607 | 0.028 |
| time | 0.028 |
| XRCC4_rs1040363 | 0.037 |
| SMAD1_rs11724777 | 0.050 |

TABLE 18 shows P-values for the Log-Rank test using Biochemical Relapse (elevated blood levels of Prostate Specific Antigen, PSA) as the outcome. A significant p-value indicates that the survival curve for a patient with the given gene mutation is significantly different than the survival curve if the patient did not have the gene mutation. This test only considers one gene at a time. As shown, the top predictors of biochemical relapse are IL10RB_rs2834167, CD274_rs1411262, CD274_rs822339, SMAD1_rs11724777, IL18R1_rs11465660, and IL16_rs11556218.

TABLE 18
Biochemical Relapse

| gene | p-value |
|---|---|
| IL10RB_rs2834167 | 0.015 |
| CD274_rs1411262 | 0.016 |
| CD274_rs822339 | 0.007 |
| SMAD1_rs11724777 | 0.018 |
| IL18R1_rs11465660 | 0.033 |
| IL16_rs11556218 | 0.050 |

TABLE 19 shows P-values for the Log-Rank test using distant metastasis as the outcome. A significant p-value indicates that the survival curve for a patient with the given gene mutation is significantly different than the survival curve if the patient did not have the gene mutation. This test only considers one gene at a time. As shown, the top predictors of distant metastasis are IL10_rs3024496, FOXP3_rs2280883, BIRC5_rs2239680, IL10_rs3024496_P1.P2, LIG4_rs2232643, RAC1_rs9374, and time.

TABLE 19
Distant Metastasis

| gene | p-value |
|---|---|
| IL10_rs3024496 | 0.008 |
| FOXP3_rs2280883 | 0.013 |
| BIRC5_rs2239680 | 0.014 |
| IL10_rs3024496_P1.P2 | 0.014 |
| LIG4_rs2232643 | 0.017 |
| RAC1_rs9374 | 0.050 |
| time | 0.050 |

Data from the OSLO cohort was also analyzed for potency, using the “Potency column” (Column J). As shown in TABLE 20, the top predictors of potency are HAMP_rs10421768, XRCC1_rs25487, IL.6_rs2069840, XRCC4_rs1040363, and IL19_rs2243158.

TABLE 20
Potency

| | No mutation | Mutation | |
|---|---|---|---|
| Gene | (% impotent) | (% impotent) | p-value |
| HAMP_rs10421768 | 80.0 | 57.1 | 0.016 |
| XRCC1_rs25487 | 63.1 | 83.3 | 0.030 |
| IL.6_rs2069840 | 61.8 | 80.8 | 0.035 |
| XRCC4_rs1040363 | 75.6 | 47.1 | 0.038 |
| IL19_rs2243158 | 75.9 | 54.2 | 0.045 |

Several classification strategies are compared in TABLE 21 for predicting potency. The following parameters were used for the determination of potency: Random Forest classification p-value threshold of 0.2 and mtry=1; Elastic Net classification p-value threshold of 0.2, α=1.0, and λ=0.00395; and Boosted Trees classification p-value threshold of 0.2, int_depth=3, and num_trees=150. PPV is intended as the probability of correctly classifying a patient as exhibiting impotency, and NPV is intended as the probability of correctly classifying a patient as exhibiting potency. The F1 Score is an evaluation of test accuracy determined using the PPV and Sensitivity measures.

TABLE 21
Potency

| Classification | Sensitivity | Specificity | | | F1 |
|---|---|---|---|---|---|
| Methods | (SD) | (SD) | PPV | NPV | Score |
| Random Forest | 0.618 | 0.895 | 0.704 | 0.850 | 0.655 |
| Elastic Net | 0.710 | 0.855 | 0.667 | 0.878 | 0.688 |
| Boosted Trees | 0.742 | 0.882 | 0.719 | 0.893 | 0.730 |

### Example 4: Further Analysis of Biomarkers from SBRT and OSLO Studies

### Methodology

The following analysis was conducted on two cohorts of men (OSLO and SBRT, as described in Example 3), each cohort receiving a different treatment regimen for advanced prostate cancer. The programming language used to analyze the data set was R (Version 3.6.1). The goal of this analysis was to use microRNA-based germline biomarkers to help identify patients that will experience Long-Term (Late) and Acute Genitourinary Toxicity. Toxicity was measured using the RTOG morbidity grading system (Cox et al. (1995) Int J Radiat Oncol Biol Phys 31(5):1341-6, and within the analysis, it was converted to a binary variable of those with low-grade toxicity (RTOG score ≤1) versus patients with high-grade toxicity (RTOG score ≥2). TABLE 22 shows the distribution of the patients based off of type of toxicity and treatment.

No analysis was conducted on Acute Genitourinary Toxicity for SBRT because only 3 patients experienced toxicity.

TABLE 22 shows toxicity grades by type of toxicity. In this study, a toxicity of 2 or higher is considered to be high grade toxicity.

TABLE 22
Toxicity Grades

| Grade | No Toxicity | Toxicity |
|---|---|---|
| Acute GU (OSLO) | 71 | 37 |
| Long-Term GU (OSLO) | 91 | 17 |
| Acute GU (SBRT) | 90 | 3 |
| Long-Term GU (SBRT) | 79 | 14 |

### Variable Selection

Biomarkers with variance close to zero (caret::nearZeroVar version 6.0-84) and biomarkers that had almost perfect correlation r²_{xi, xj}≥0.99) were removed. The Fisher's Exact Test for Count Data was then conducted to test the pairwise independence of the response (Toxicity) and each predictor (microRNA-based germline biomarkers). For each analysis, a p-value threshold was used to determine which predictors would be used in the models. Since the p-values were only used as a selection criteria, they are not adjusted to correct for the multiplicity of testing. Therefore, the p-values obtained should not be used for any inferential purposes. TABLES 23, 24, and 25 contain the selected variables, as well as the percentages of people who are toxic with the mutation and without the mutation.

### Model Fitting

Due to the imbalance in the responses, up-sampling was used (caret::upSample version 6.0-84) to ensure the same number of Toxic patients as Non-Toxic patients. After up-sampling, Random Forest, Boosted Trees, and Elastic Net were fit to predict toxicity in each patient. Each model was run using various model parameters in order to find the optimal parameter. The metrics used for model performance were Sensitivity, Specificity, Negative Predicted Value (NPV), Positive Predicted Value (PPV), and F1 Score (Defined Below). To reduce selection bias and overfitting, each of these metrics were calculated using leave-one-out cross-validation. TABLES 26 and 27 contain the evaluation metrics for SBRT and OSLO patients. TABLES 28 and 29 contain evaluation metrics for predicting patient toxicity in one group while being trained on patients in the other group. The poor performance of the models suggests that the underlying mechanisms that determine Late GU Toxicity are different in each treatment regimen.

### Definitions

Sensitivity: Probability that given you are toxic, you were classified as toxic
Specificity: Probability that given you are not toxic, you were classified as not toxic
PPV: Probability that given you were categorized as toxic, you are toxic
NPV: Probability that given you were categorized as not toxic, you are not toxic
F1 Score: Uses the precision (PPV) and recall (Sensitivity) of the test to evaluate the test accuracy

### Variable Importance

To determine variable importance, the F1 score was used as the main metric. To calculate the variable importance for a single SNP (Sᵢ), the F1 score was calculated using all of the SNPs in the model (F₁*), and then the F1 score was calculated when removing the SNP (Sᵢ) from the model (F^{˜}₁). The F1 scores were all calculated using leave-one-out cross-validation. The variable importance score was then calculated as F₁*−F^{˜}₁. Thus, a simple interpretation of the variable importance score would be the additional F1 score added by including that SNP. This method of calculating variable importance scores allows for negative scores as well. The plots of the variable importance were made using ggplot2 (version 3.2.1).

TABLE 23
Variable Selection - Acute Toxicity (OSLO)

| | No Mutation | Mutation | |
|---|---|---|---|
| SNP | (% Tox) | (% Tox) | p-value |
| ATM_rs189037 | 61.9 | 27.6 | 0.005 |
| CD6_rs76677607 | 28.6 | 64.7 | 0.010 |
| IL1A_rs1800587 | 45.5 | 22.6 | 0.015 |
| IL1A_rs17561 | 44.6 | 23.1 | 0.025 |
| BRCA2_rs7334543 | 47.5 | 26.5 | 0.036 |
| ILF3.58_rs118142475 | 32.4 | 100.0 | 0.038 |
| BIRC5_rs2239680 | 25.8 | 45.7 | 0.041 |
| EGFR_rs884225 | 38.0 | 12.5 | 0.051 |

TABLE 23 shows marginal associations between acute toxicity (at or above grade 2) and genetic mutations. All tests of association are based on Pearson's Chi-squared test.

TABLE 24
Variable Selection - Late Toxicity (OSLO)

| | No Mutation | Mutation | |
|---|---|---|---|
| SNP | (% Tox) | (% Tox) | p-value |
| CD6_rs76677607 | 9.9 | 47.1 | 0.001 |
| CD274_rs2297136 | 0.0 | 21.2 | 0.005 |
| BRCA2_rs7334543 | 27.5 | 21.2 | 0.005 |
| NBN_rs1805794 | 6.2 | 23.3 | 0.017 |
| IL1.B_rs4848306 | 3.2 | 20.8 | 0.022 |
| CD274_rs4742098 | 21.7 | 5.1 | 0.027 |
| ERCC4_rs4781562 | 7.7 | 23.2 | 0.035 |
| MSH2_rs2303428 | 11.8 | 30.4 | 0.048 |

TABLE 24 shows marginal associations between late toxicity (at or above grade 2) and genetic mutations. All tests of association are based on Pearson's Chi-squared test.

TABLE 25
Variable Selection - Late GU Toxicity (SBRT)

| | No Mutation | Mutation | |
|---|---|---|---|
| SNP | (% Tox) | (% Tox) | p-value |
| BMP2_rs1979855 | 6.9 | 28.6 | 0.007 |
| rs1893217 | 8.8 | 32.0 | 0.010 |
| BRCA2_rs15869 | 9.4 | 27.6 | 0.031 |
| ABL1_rs11991 | 20.6 | 3.3 | 0.032 |
| ERCC1_rs11615 | 10.1 | 29.2 | 0.043 |

TABLE 25 shows marginal associations between late toxicity (at or above grade 2) and genetic mutations. All tests of association are based on Pearson's Chi-squared test.

TABLE 26 shows measures of performance for each model for OSLO patients. Parameters were as follows:

Acute Toxicity

Random Forest: P-val thresh: 0.3, mtry=1, nodesize=85, ntree=100

Elastic Net: P-val thresh: 0.3, α=0.1, λ=0.0404

Boosted Trees: P-val thresh: 0.3, int_depth=3, num_trees=100

Late Toxicity

Random Forest: P-val thresh: 0.3, mtry=3, nodesize=85, ntree=100

Elastic Net: P-val thresh: 0.3, α=0.1, λ=0.0404

Boosted Trees: P-val thresh: 0.3, int_depth=2, num_trees=150

TABLE 26

| | | | | | |
|---|---|---|---|---|---|
| Classification | Sensitivity | Specificity | | | F1 |
| Methods | (SD) | (SD) | PPV | NPV | Score |

Acute Toxicity (OSLO)

| | | | | | |
|---|---|---|---|---|---|
| Random Forest | 0.784 | 0.690 | 0.569 | 0.860 | 0.659 |
| Elastic Net | 0.622 | 0.803 | 0.622 | 0.803 | 0.622 |
| Boosted Trees | 0.622 | 0.803 | 0.622 | 0.803 | 0.622 |

Late Term Toxicity (OSLO)

| | | | | | |
|---|---|---|---|---|---|
| Random Forest | 0.765 | 0.802 | 0.419 | 0.948 | 0.542 |
| Elastic Net | 0.588 | 0.956 | 0.714 | 0.926 | 0.645 |
| Boosted Trees | 0.647 | 0.967 | 0.786 | 0.936 | 0.710 |

TABLE 27 shows measures of performance for each model for SBRT patients. Parameters were as follows:

### Late Toxicity

Random Forest: P-val thresh: 0.3, mtry=1, nodesize=85, ntree=100

Elastic Net: P-val thresh: 0.3, α=0.1, λ=0.0395

Boosted Trees: P-val thresh: 0.15, int_depth=1, num_trees=100

TABLE 27
Late Term Toxicity (SBRT)

| Classification | Sensitivity | Specificity | | | F1 |
|---|---|---|---|---|---|
| Methods | (SD) | (SD) | PPV | NPV | Score |
| Random Forest | 0.929 | 0.747 | 0.394 | 0.983 | 0.553 |
| Elastic Net | 0.786 | 0.924 | 0.647 | 0.961 | 0.710 |
| Boosted Trees | 0.714 | 0.848 | 0.455 | 0.944 | 0.556 |

TABLE 28 shows measures of performed created by training a model on the OSLO patients and predicting toxicity in SBRT patients.

TABLE 28
OSLO Model Performance Classifying SBRT

| Classification | Sensitivity | Specificity | | | F1 |
|---|---|---|---|---|---|
| Methods | (SD) | (SD) | PPV | NPV | Score |
| Random Forest | 0.14 | 0.81 | 0.12 | 0.84 | 0.13 |
| Elastic Net | 0.14 | 0.87 | 0.17 | 0.85 | 0.15 |
| Boosted Trees | 0.14 | 0.91 | 0.22 | 0.86 | 0.17 |

TABLE 29 shows measures of performed created by training a model on the SBRT patients and predicting toxicity in OSLO patients.

TABLE 29
SBRT Model Performance Classifying OSLO

| Classification | Sensitivity | Specificity | | | F1 |
|---|---|---|---|---|---|
| Methods | (SD) | (SD) | PPV | NPV | Score |
| Random Forest | 0.27 | 0.65 | 0.29 | 0.63 | 0.28 |
| Elastic Net | 0.27 | 0.87 | 0.53 | 0.70 | 0.36 |
| Boosted Trees | 0.24 | 0.72 | 0.31 | 0.65 | 0.27 |

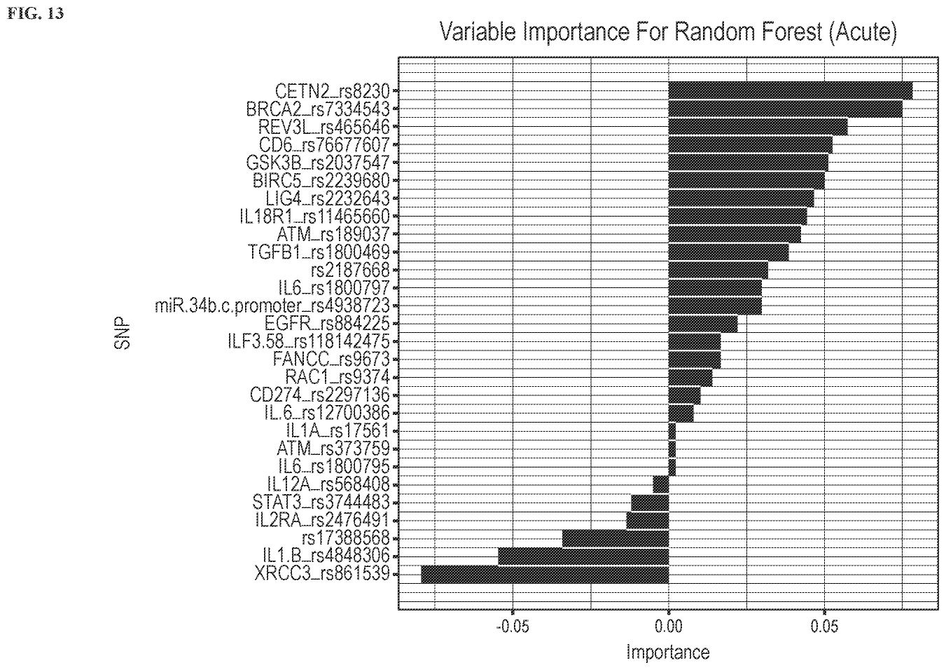

FIG. 13 shows a Variable Importance diagram using a Random Forest classification strategy for acute toxicity in the OSLO cohort. As shown, the top predictors of toxicity at or above grade 2 are CETN2_rs8230, BRCA2_rs73345543, REV3L_rs465646, CD6_rs76677607, GSK3B_rs2037547, BIRC5_rs2239680, LIG4_rs2232643, IL18R1_rs11465660, ATM_rs189037, TGFB1_rs1800469, rs2187668, IL6_rs1800797, miR.34b.c.promoter_rs4938723, EGFR_rs884225, ILF3.58_rs118142475, FANCC_rs9673, RAC1_rs9374, CD274_rs2297136, IL.6_rs12700386, IL1A_rs17561, ATM_rs373759, and IL6_rs1800795.

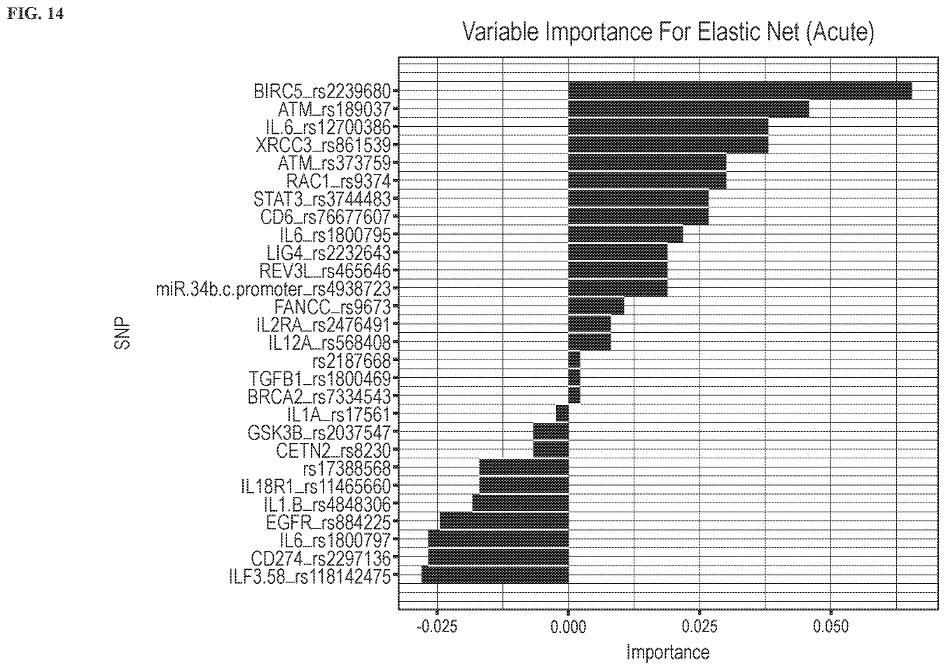

FIG. 14 shows a Variable Importance diagram using an Elastic Net classification strategy for acute toxicity in the OSLO cohort. As shown, the top predictors of toxicity at or above grade 2 are BIRC5_rs2239680, ATM_rs189037, IL.6_rs12700386, XRCC3_rs861539, ATM_rs373759, RAC1_rs9374, STAT3_rs3744483, CD6_rs76677607, IL6_rs1800795, LIG4_rs2232643, REV3L_rs465646, miR.34b.c.promoter_rs4938723, FANCC_rs9673, IL2RA_rs2476491, IL12A_rs568408, rs2187668, TGFB1_rs1800469, and BRCA2_rs73345543.

FIG. 15 shows a Variable Importance diagram using a Boosted Trees classification strategy for acute toxicity in the OSLO cohort. As shown, the top predictors of toxicity at or above grade 2 are FANCC_rs9673, ATM_rs189037, IL2RA_rs2476491, IL6_rs1800795, RAC1_rs9374, IL.6_rs12700386, CD6_rs76677607, BRCA2_rs73345543, rs2187668, ATM_rs373759, TGFB1_rs1800469, XRCC3_rs861539, IL6_rs1800797, rs17388569, STAT3_rs3744483, CETN2_rs8230, REV3L_rs465646, IL1A_rs17561, IL18R1_rs11465660, miR.34b.c.promoter_rs4938723, EGFR_rs884225, BIRC5_rs2239680, ILF3.58_rs118142475, and IL12A_rs568408.

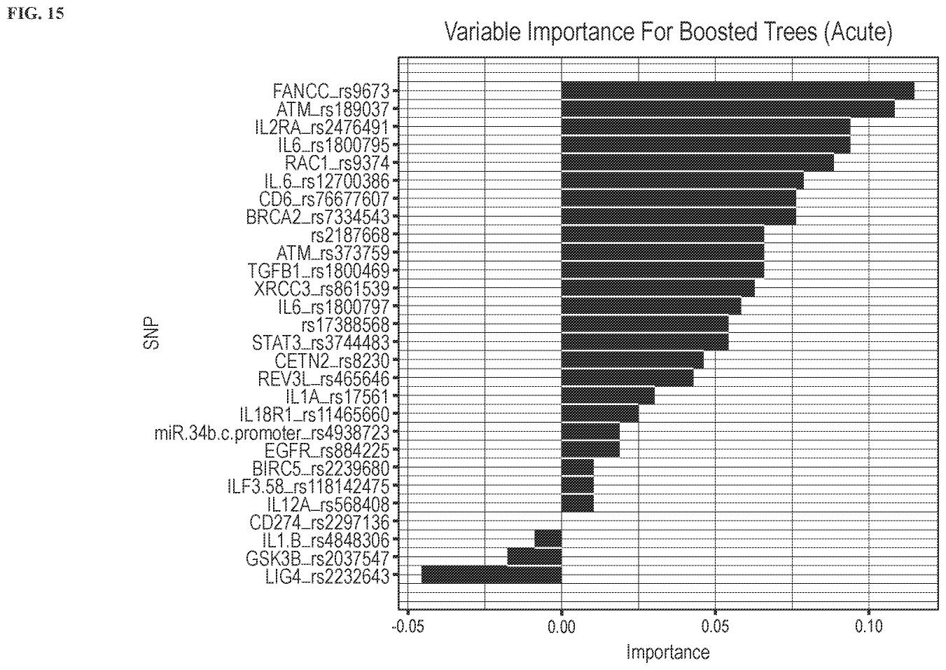

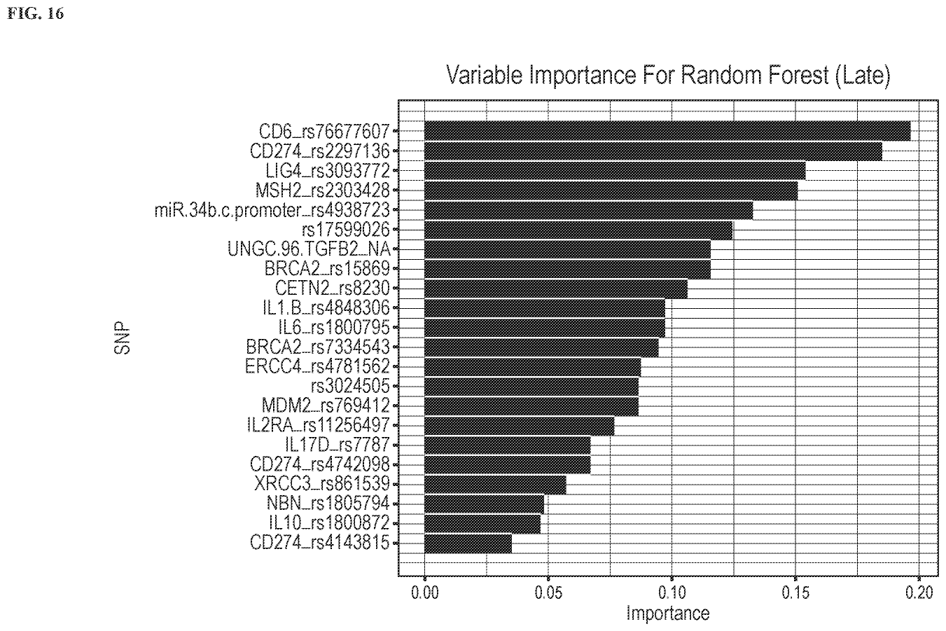

FIG. 16 shows a Variable Importance diagram using a Random Forest classification strategy for late-term toxicity in the OSLO cohort. As shown, the top predictors of toxicity at or above grade 2 are CD6_rs76677607, CD274_rs2297136, LIG4_rs3093772_P1.P2, MSH2_rs2303428, miR.34b.c.promoter_rs4938723, rs17599026, UNGC.96.TGFB2_NA, BRCA2_rs15869, CETN2_rs8230, IL1.B_rs4848306, IL6_rs1800795, BRCA2_rs73345543, ERCC4_rs4781562, IL10_rs3024505, MDM2_rs769412, IL2RA_rs11256497, IL17D_rs7787, CD274_rs4742098, XRCC3_rs861539, NBN_rs1805794, IL10_rs1800872, and CD274_rs4143815.

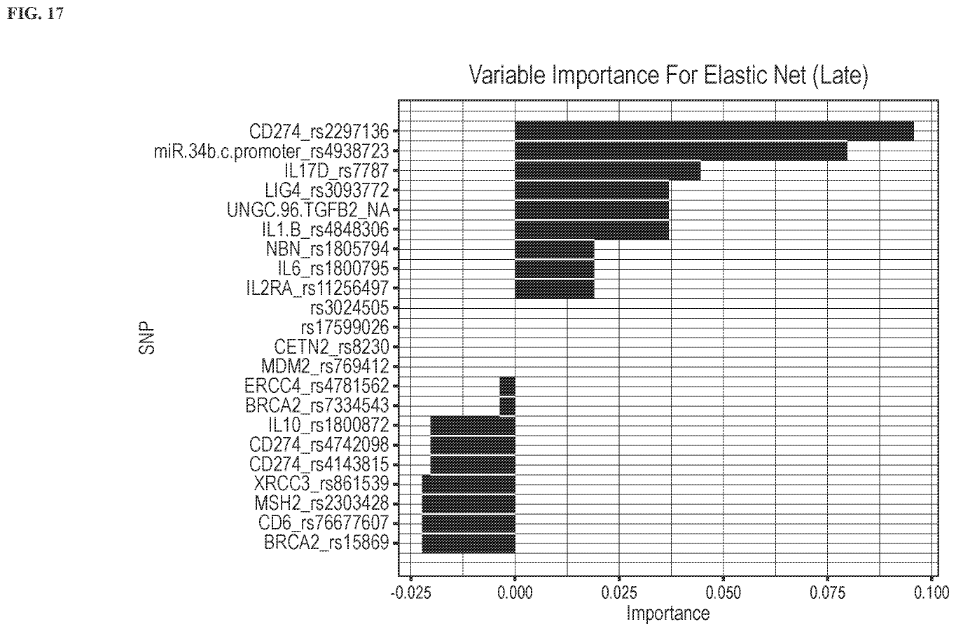

FIG. 17 shows a Variable Importance diagram using an Elastic Net classification strategy for late-term toxicity in the OSLO cohort. As shown, the top predictors of toxicity at or above grade 2 are CD274_rs2297136, miR.34b.c.promoter_rs4938723, IL17D_rs7787, LIG4_rs3093772_P1.P2, UNGC.96.TGFB2_NA, IL1.B_rs4848306, NBN_rs1805794, IL6_rs1800795, and IL2RA_rs11256497.

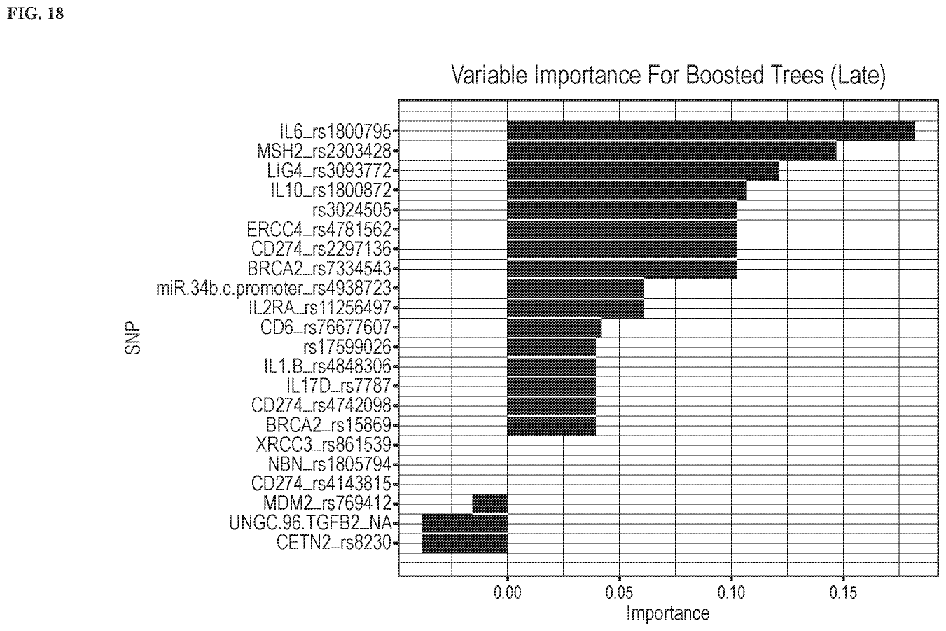

FIG. 18 shows a Variable Importance diagram using a Boosted Trees classification strategy for late-term toxicity in the OSLO cohort. As shown, the top predictors of toxicity at or above grade 2 are IL6_rs1800795, MSH2_rs2303428, LIG4_rs3093772_P1.P2, IL10_rs1800872, IL10_rs3024505, ERCC4_rs4781562, CD274_rs2297136, BRCA2_rs73345543, miR.34b.c.promoter_rs4938723, IL2RA_rs11256497, CD6_rs76677607, rs17599026, IL1.B_rs4848306, IL17D_rs7787, CD274_rs4742098, and BRCA2_rs15869.

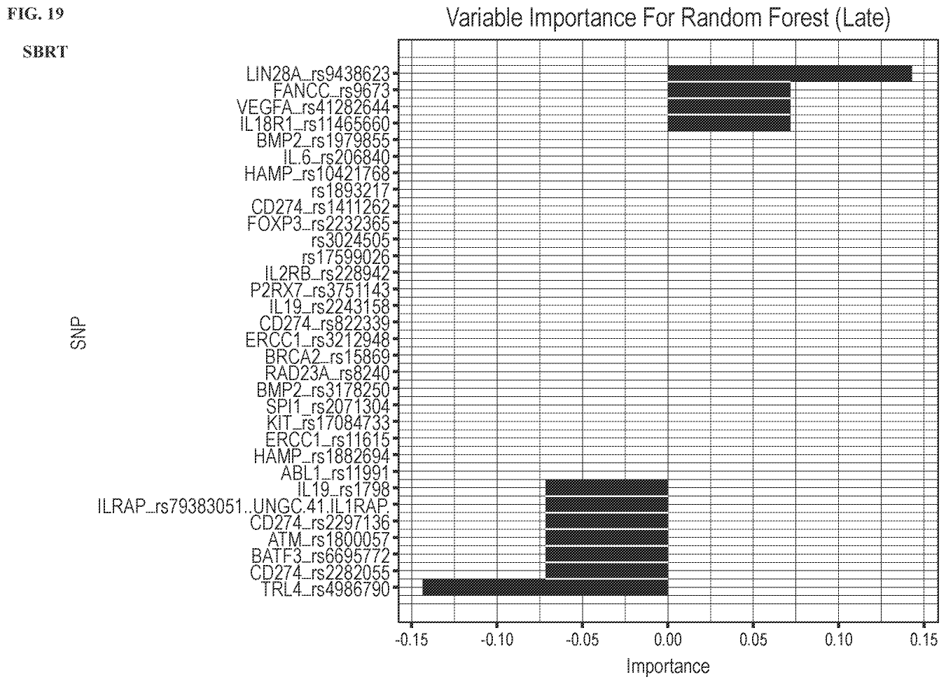

FIG. 19 shows a Variable Importance diagram using a Random Forest classification strategy for late-term toxicity in the SBRT cohort. As shown, the top predictors of toxicity at or above grade 2 are LIN28A_rs9438623, FANCC_rs9673, VEGFA_rs41282644, and IL18R1_rs11465660.

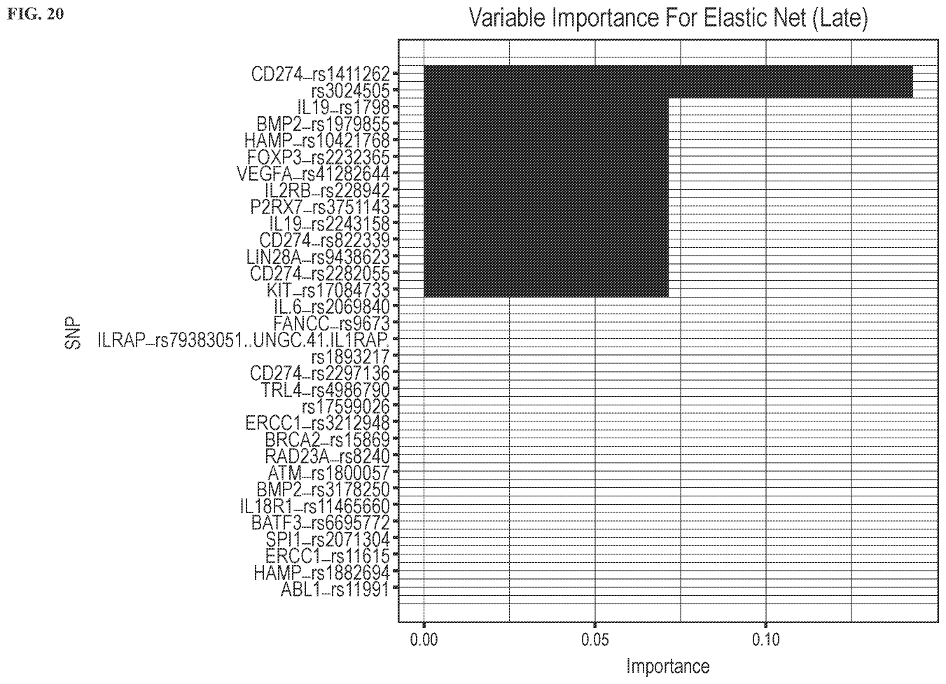

FIG. 20 shows a Variable Importance diagram using an Elastic Net classification strategy for late-term toxicity in the SBRT cohort. As shown, the top predictors of toxicity at or above grade 2 are CD274_rs1411262, IL10_rs3024505, IL19_rs1798, BMP2_rs1979855, HAMP_rs10421768, FOXP3_rs2232365, VEGFA_rs41282644, IL2RB_rs228942, P2RX7_rs3751143, IL19_rs2243158, CD274_rs822339, LIN28A_rs9438623, CD274_rs2282055, and KIT_rs17084733.

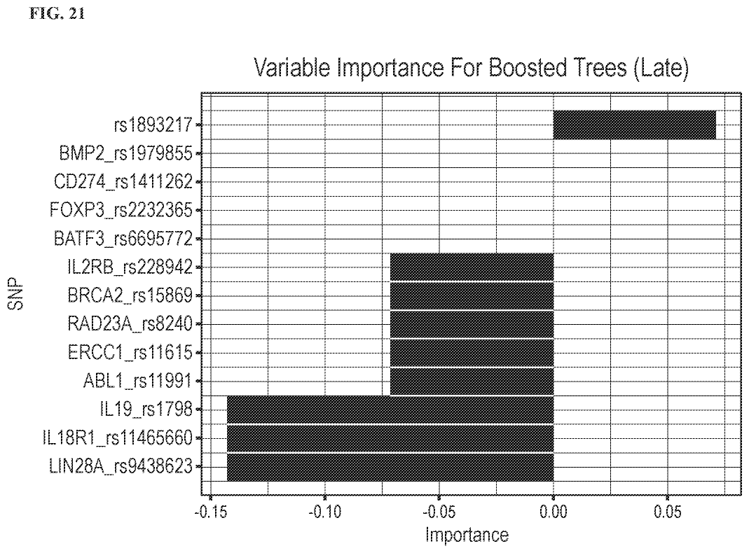

FIG. 21 shows a Variable Importance diagram using a Boosted Trees classification strategy for late-term toxicity in the SBRT cohort. As shown, the top predictor of toxicity at or above grade 2 is rs1893217.

Further analysis of mortality, biochemical relapse, and distant metastases was conducted as follows. In this analysis, a log-rank test (survival version 2.44-1.1) was conducted to determine a SNPs marginal association with the time until the event of interest. The log-rank test compares the estimated hazard functions of patient with the mutation of a SNP to those without a mutation at each observed event time. The hazard ratio for each SNP was estimated using a Cox proportional hazards regression model (coxph::survival version 2.44-1.1). Due to the duration of the study, right censoring occurred, meaning that the event had not happened in some patients yet. TABLES 30, 31, 32, and 33 contain the unadjusted P-values for the log-rank test, as well as the hazard ratio for each SNP.

TABLE 30
Prostate Cancer Specific Mortality

| gene | p-value | hazard ratio |
|---|---|---|
| BIRC5_rs2239680 | 0.014 | 0.187 |
| CD6_rs76677607 | 0.028 | 3.451 |
| XRCC4_rs1040363 | 0.037 | 2.978 |
| SMAD1_rs11724777 | 0.050 | 4.003 |

TABLE 31
Biochemical Relapse

| gene | p-value | hazard ratio |
|---|---|---|
| RAC1_rs9374 | 0.015 | 1.563 |
| CD274_rs2297136 | 0.016 | 0.605 |
| CD6_rs76677607 | 0.007 | 1.201 |
| IL2RA_rs11256497 | 0.033 | 0.849 |
| STAT3_rs3744483 | 0.018 | 0.985 |
| IL17D_rs7787 | 0.050 | 0.870 |

TABLE 32
Distant Metastasis

| gene | p-value | hazard ratio |
|---|---|---|
| IL10_rs3024496 | 0.008 | 0.393 |
| FOXP3_rs2280883 | 0.013 | 0.390 |
| BIRC5_rs2239680 | 0.014 | 0.380 |
| IL10_rs3024496_P1.P2 | 0.014 | 0.418 |
| LIG4_rs2232643 | 0.017 | 8.007 |
| RAC1_rs9374 | 0.050 | 2.017 |

TABLE 33
Potency

| gene | p-value | hazard ratio |
|---|---|---|
| LIG4_rs3093772 | 0.033 | — |

### Example 5: Further Analysis of Biomarkers from SBRT and OSLO Studies

Further analysis of mortality, biochemical relapse, and distant metastases was conducted as follows. In this analysis, a log-rank test (survival version 2.44-1.1) was conducted to determine a SNPs marginal association with the time until the event of interest. The log-rank test compares the estimated hazard functions of patient with the mutation of a SNP to those without a mutation at each observed event time. The hazard ratio for each SNP was estimated using a Cox proportional hazards regression model (coxph::survival version 2.44-1.1). Due to the duration of the study, right censoring occurred, meaning that the event had not happened in some patients yet. In this analysis, we adjusted for the Age, Gleason grade, T stage, and the initial PSA of the patient. TABLES 34, 35, 36, and 37 contain the P-values and the hazard ratio estimated by the Cox regression for each SNP.

TABLE 34
Prostate Cancer Specific Mortality

| gene | p-value | hazard ratio |
|---|---|---|
| BIRC5_rs2239680 | 0.020 | 0.152 |
| SMAD1_rs11724777 | 0.025 | 10.635 |
| IL12A_rs568408 | 0.041 | 3.327 |
| IL13_rs568408 | 0.044 | 3.998 |

TABLE 35
Biochemical Relapse

| gene | p-value | hazard ratio |
|---|---|---|
| BMP2_rs3178250 | 0.007 | 2.297 |
| SMAD1_rs11724777 | 0.016 | 2.329 |
| IL10RB_rs2834167 | 0.020 | 0.486 |
| CD274_rs822339 | 0.021 | 2.041 |
| CD274_rs2282055 | 0.026 | 1.984 |
| IL12A_rs568408 | 0.032 | 1.978 |
| PARP1_rs8679 | 0.032 | 1.986 |
| CD274_rs1411262 | 0.033 | 1.936 |
| BMP2_rs1979855 | 0.039 | 1.876 |
| P2RX7_rs3751143 | 0.042 | 1.943 |

TABLE 36
Distant Metastasis

| gene | p-value | hazard ratio |
|---|---|---|
| IL8_rs4073 | 0.016 | 0.373 |
| IL10_rs3024496_P1 | 0.016 | 0.394 |
| BIRC5_rs2239680 | 0.018 | 0.376 |
| RAC1_rs9374 | 0.021 | 2.464 |
| IL10_rs3024496_P1.P2 | 0.021 | 0.411 |
| BMP2_rs3178250 | 0.038 | 2.168 |

TABLE 37 provides Marginal Association for each predictor and Potency. To find the p-value for marginal association between Testosterone level, Age, Time on ADT treatment, and Time since ending ADT treatment and Potency, we created a simple logistic regression and evaluated the Wald statistic. We used the same three models used in modeling Toxicity, and used Leave-one-out Cross-validation to obtain our measures of performance. Variable Importance was also obtained in the same fashion that it was obtained in modeling Toxicity.

The tests for marginal association between the SNPS and Potency are based on Pearson's Chi-squared test. The test for marginal association between Testosterone, Age, Time since ending ADT treatment, and Time on ADT treatment and Potency were conducted using a Wald statistic.

TABLE 37
Potency

| gene | p-value | hazard ratio |
|---|---|---|
| HAMP_rs10421768 | 0.008 | — |

Potency (OSLO)

| Classification | Sensitivity | Specificity | | | F1 | |
|---|---|---|---|---|---|---|
| Methods | (SD) | (SD) | PPV | NPV | Score | AUC |
| Random Forest | 0.840 | 0.794 | 0.618 | 0.926 | 0.712 | 0.817 |
| Elastic Net | 0.680 | 0.810 | 0.586 | 0.864 | 0.630 | 0.741 |
| Boosted Trees | 0.640 | 0.857 | 0.640 | 0.857 | 0.640 | 0.749 |

Potency

Random Forest: P-val thresh: 0.3, mtry=1, nodesize=85, ntree=100

Elastic Net: P-val thresh: 0.2, α=0.1, λ=0.00395

Boosted Trees: P-val thresh: 0.2, int_depth=2, num_trees=50

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A method of treating cancer comprising administering a radiation treatment to a patient identified as carrying or not carrying one or more mutations selected from:
a) a G nucleotide at a position corresponding to position 101 of SEQ ID NO: 53 (BMP2_rs1979855);
b) a C nucleotide at a position corresponding to position 101 of SEQ ID NO: 1 (miR.99a.promoter);
c) an A nucleotide at a position corresponding to position 101 of SEQ ID NO: 3 rs4848306);
d) a G nucleotide at a position corresponding to position 101 of SEQ ID NO: 5 (SHC4_rs1062124);
e) a T nucleotide at a position corresponding to position 101 of SEQ ID NO: 7 (rs17599026);
f) an A nucleotide at a position corresponding to position 101 of SEQ ID NO: 9 (RAC1_rs9374);
g) a C nucleotide at a position corresponding to position 101 of SEQ ID NO: 11 (IL24_rs1150258);
h) an A or C nucleotide at a position corresponding to position 101 of SEQ ID NO: 13 (IL1A_rs1800587);
i) a C nucleotide at a position corresponding to position 101 of SEQ ID NO: 15 (ERCC1_rs3212948);
j) a G nucleotide at a position corresponding to position 101 of SEQ ID NO: 17 (IL.6_rs12700386);
k) a G nucleotide at a position corresponding to position 101 of SEQ ID NO: 19 (IL.6_rs2069840);
l) a T nucleotide at a position corresponding to position 101 of SEQ ID NO: 21 (CAMK2G_rs2306327);
m) a T nucleotide at a position corresponding to position 101 of SEQ ID NO: 23 (BMP2_rs1980499);
n) a C nucleotide at a position corresponding to position 101 of SEQ ID NO: 25 (EREG_rs1460008);
o) a C nucleotide at a position corresponding to position 101 of SEQ ID NO: 27 (CD274_rs4143815);
p) an A nucleotide at a position corresponding to position 101 of SEQ ID NO: 29 (IL17D_rs7787);
q) a C nucleotide at a position corresponding to position 101 of SEQ ID NO: 31 (STAT3/rs3744483);
r) a C nucleotide at a position corresponding to position 101 of SEQ ID NO: 33 (BMP4_rs17563)
s) a C nucleotide at a position corresponding to position 101 of SEQ ID NO: 35 (IRF5_rs10488631);
t) a T nucleotide at a position corresponding to position 101 of SEQ ID NO: 37 (UNGC.41.IL1RAP);
u) a G nucleotide at a position corresponding to position 101 of SEQ ID NO: 39 (IL19_rs1798);
v) an A nucleotide at a position corresponding to position 101 of SEQ ID NO: 41 (RAD23A_rs8240);
w) a C nucleotide at a position corresponding to position 101 of SEQ ID NO: 43 (BRCA2_rs15869);
x) a G nucleotide at a position corresponding to position 101 of SEQ ID NO: 45 (CD274_rs2282055);
y) a C nucleotide at a position corresponding to position 101 of SEQ ID NO: 47 (BMP2_rs3178250);
z) a T nucleotide at a position corresponding to position 101 of SEQ ID NO: 49 (CD274_rs1411262);
aa) an A nucleotide at a position corresponding to position 101 of SEQ ID NO: 51 (CD274_rs2297136);
bb) a G nucleotide at a position corresponding to position 101 of SEQ ID NO: 55 (PTPN2_rs1893217);
cc) a G nucleotide at a position corresponding to position 101 of SEQ ID NO: 57 (CD274_rs822339);
dd) a G nucleotide at a position corresponding to position 101 of SEQ ID NO: 59 (BATF3_rs6695772);
ee) an A nucleotide at a position corresponding to position 101 of SEQ ID NO: 61 (IL18R1_rs11465660);
ff) a G nucleotide at a position corresponding to position 101 of SEQ ID NO: 63 (ERCC1_rs11615);
gg) a C nucleotide at a position corresponding to position 101 of SEQ ID NO: 65 (ABL1_rs11991);
hh) an A nucleotide at a position corresponding to position 101 of SEQ ID NO: 67 (ATM_rs189037);
ii) an A nucleotide at a position corresponding to position 101 of SEQ ID NO: 69 (CD6_rs76677607);
jj) an A nucleotide at a position corresponding to position 101 of SEQ ID NO: 71 (IL1A_rs17561);
kk) a G nucleotide at a position corresponding to position 101 of SEQ ID NO: 73 (BRCA2_rs7334543);
ll) a T nucleotide at a position corresponding to position 101 of SEQ ID NO: 75 (ILF3.58_rs118142475);
mm) a C nucleotide at a position corresponding to position 101 of SEQ ID NO: 77 (BIRC5_rs2239680);
nn) a G nucleotide at a position corresponding to position 101 of SEQ ID NO: 79 (NBN_rs1805794);
oo) a T nucleotide at a position corresponding to position 101 of SEQ ID NO: 81 (TREX_rs11797);
pp) a G nucleotide at a position corresponding to position 101 of SEQ ID NO: 83 (CD274_rs4742098);
qq) a T nucleotide at a position corresponding to position 101 of SEQ ID NO: 85 (ERCC4_rs4781562);
rr) an A, C or G nucleotide at a position corresponding to position 101 of SEQ ID NO: 87 (MSH2_rs2303428);
ss) a C nucleotide at a position corresponding to position 101 of SEQ ID NO: 89 (XRCC4_rs1040363);
tt) a G nucleotide at a position corresponding to position 101 of SEQ ID NO: 91 (IL10RB_rs2834167);
uu) an A nucleotide at a position corresponding to position 101 of SEQ ID NO: 93 (SMAD1_rs11724777);
vv) a G nucleotide at a position corresponding to position 101 of SEQ ID NO: 95 (HAMP_rs10421768);
ww) a C nucleotide at a position corresponding to position 101 of SEQ ID NO: 97 (XRCC1_rs25487);
xx) a G nucleotide at a position corresponding to position 101 of SEQ ID NO: 99 (IL19_rs2243158);
yy) a C nucleotide at a position corresponding to position 101 of SEQ ID NO: 101 (FOXP3_rs2280883);
zz) a G nucleotide at a position corresponding to position 101 of SEQ ID NO: 103 (IL10_rs3024496_P1.P2);
aaa) a C nucleotide at a position corresponding to position 101 of SEQ ID NO: 105 (LIG4_rs2232643);
bbb) a T nucleotide at a position corresponding to position 101 of SEQ ID NO: 107 (IL2RA_rs2476491);
ccc) an A nucleotide at a position corresponding to position 101 of SEQ ID NO: 109 (IL12A_rs568408);
ddd) a C nucleotide at a position corresponding to position 101 of SEQ ID NO: 111 (GSK3B_rs2037547);
eee) an A nucleotide at a position corresponding to position 101 of SEQ ID NO: 113 (ATM_rs189037);
fff) a G nucleotide at a position corresponding to position 101 of SEQ ID NO: 115 (IL6_rs1800797);
ggg) an A nucleotide at a position corresponding to position 101 of SEQ ID NO: 117 (RAC1_rs9374);
hhh) a C or G nucleotide at a position corresponding to position 101 of SEQ ID NO: 119 (FCGR2A_rs1801274);
iii) a T nucleotide at a position corresponding to position 101 of SEQ ID NO: 121 (ATM_rs373759);
jjj) an A nucleotide at a position corresponding to position 101 of SEQ ID NO: 123 (CETN2_rs8230);
kkk) an A nucleotide at a position corresponding to position 101 of SEQ ID NO: 125 (XRCC3_rs861539);
lll) a C nucleotide at a position corresponding to position 101 of SEQ ID NO: 127 (miR.34b.c.promoter_rs4938723);
mmm) a G nucleotide at a position corresponding to position 101 of SEQ ID NO: 129 (LIN28A_rs9438623);
nnn) a G or C nucleotide at a position corresponding to position 101 of SEQ ID NO: 131 (FANCC_rs9673);
ooo) an A nucleotide at a position corresponding to position 101 of SEQ ID NO: 133 (REV3L_rs465646);
ppp) a G nucleotide at a position corresponding to position 101 of SEQ ID NO: 135 (IL6_rs1800795);
qqq) a C nucleotide at a position corresponding to position 101 of SEQ ID NO: 137 (SPI1_rs2071304);
rrr) a G nucleotide at a position corresponding to position 101 of SEQ ID NO: 139 (IL10_rs3024496);
sss) a deletion of a T nucleotide in the sequence ATTTTG of SEQ ID NO: 142 (wild-type sequence) to give a corresponding sequence of ATTTG as in SEQ ID NO: 141 (mutant) (EXO1_rs4150021);
ttt) an A nucleotide at a position corresponding to position 101 of SEQ ID NO: 143 (FOXP3_rs2232365);
uuu) a C nucleotide at a position corresponding to position 101 of SEQ ID NO: 145 (HAMP_rs1882694);
vvv) a G nucleotide at a position corresponding to position 101 of SEQ ID NO: 147 (IL10_rs1800872);
www) a G nucleotide at a position corresponding to position 101 of SEQ ID NO: 149 (IL16_rs11556218);
xxx) an A nucleotide at a position corresponding to position 101 of SEQ ID NO: 151 (IL2RA_rs11256497);
yyy) a T nucleotide at a position corresponding to position 101 of SEQ ID NO: 153 (IL2RB_rs228942);
zzz) an A nucleotide at a position corresponding to position 101 of SEQ ID NO: 155 (KIT_rs17084733);
aaaa) a C nucleotide at a position corresponding to position 101 of SEQ ID NO: 157 (KRAS_rs61764370);
bbbb) a C nucleotide at a position corresponding to position 101 of SEQ ID NO: 159 (LIG4_rs3093772_P1.P2);
cccc) a G nucleotide at a position corresponding to position 101 of SEQ ID NO: 161 (MDM2_rs769412);
dddd) a C or G nucleotide at a position corresponding to position 101 of SEQ ID NO: 163 (P2RX7_rs3751143);
eeee) a T nucleotide at a position corresponding to position 101 of SEQ ID NO: 165 (rs2187668);
ffff) an A nucleotide at a position corresponding to position 101 of SEQ ID NO: 167 (IL10_rs3024505);
gggg) a G nucleotide at a position corresponding to position 101 of SEQ ID NO: 169 (rs922075);
hhhh) a T nucleotide at a position corresponding to position 101 of SEQ ID NO: 171 (SOS1_rs1059313);
iiii) a C nucleotide at a position corresponding to position 101 of SEQ ID NO: 173 (STAT4_rs7574070);
jjjj) an A nucleotide at a position corresponding to position 101 of SEQ ID NO: 175 (UNGC.96.TGFB2_NA);
kkkk) an A nucleotide at a position corresponding to position 101 of SEQ ID NO: 177 (VEGFA_rs41282644);
llll) an A nucleotide at a position corresponding to position 101 of SEQ ID NO: 179 (IL1A_rs17561);
mmmm) a G nucleotide at a position corresponding to position 101 of SEQ ID NO: 181 (TGFB1_rs1800469);
nnnn) a C nucleotide at a position corresponding to position 101 of SEQ ID NO: 183 (EGFR_rs884225);
oooo) a G nucleotide at a position corresponding to position 101 of SEQ ID NO: 185 (rs17388569);
pppp) a G nucleotide at a position corresponding to position 101 of SEQ ID NO: 187 (TRL4_rs4986790);
qqqq) a G nucleotide at a position corresponding to position 101 of SEQ ID NO: 189 (ATM_rs1800057); and
rrrr) a T nucleotide at a position corresponding to position 101 of SEQ ID NO: 191 (ILRAP_rs79383051).

2. The method of claim 1, wherein the radiation treatment comprises hypofractionated or traditional radiation, and the patient is identified as carrying or not carrying one or more mutations selected from:
a) a C nucleotide at a position corresponding to position 101 of SEQ ID NO: 1 (miR.99a.promoter);
b) an A nucleotide at a position corresponding to position 101 of SEQ ID NO: 3 (IL1.B_rs4848306);
c) a G nucleotide at a position corresponding to position 101 of SEQ ID NO: 5 (SHC4_rs1062124);
d) a T nucleotide at a position corresponding to position 101 of SEQ ID NO: 7 (rs17599026);
e) an A nucleotide at a position corresponding to position 101 of SEQ ID NO: 9 (RAC1_rs9374);
f) a C nucleotide at a position corresponding to position 101 of SEQ ID NO: 11 (IL24_rs1150258);
g) an A nucleotide at a position corresponding to position 101 of SEQ ID NO: 13 (IL1A_rs1800587);
h) a C nucleotide at a position corresponding to position 101 of SEQ ID NO: 15 (ERCC1_rs3212948);
i) a G nucleotide at a position corresponding to position 101 of SEQ ID NO: 17 (IL.6_rs12700386);
j) a G nucleotide at a position corresponding to position 101 of SEQ ID NO: 19 (IL.6_rs2069840);
k) a T nucleotide at a position corresponding to position 101 of SEQ ID NO: 21 (CAMK2G_rs2306327);
l) a T nucleotide at a position corresponding to position 101 of SEQ ID NO: 23 (BMP2_rs1980499);
m) a C nucleotide at a position corresponding to position 101 of SEQ ID NO: 25 (EREG_rs1460008);
n) a C nucleotide at a position corresponding to position 101 of SEQ ID NO: 27 (CD274_rs4143815);
o) an A nucleotide at a position corresponding to position 101 of SEQ ID NO: 29 (IL17D_rs7787);
p) a C nucleotide at a position corresponding to position 101 of SEQ ID NO: 31 (STAT3/rs3744483);
q) a C nucleotide at a position corresponding to position 101 of SEQ ID NO: 33 (BMP4_rs17563)
r) a C nucleotide at a position corresponding to position 101 of SEQ ID NO: 35 (IRF5_rs10488631);
s) a T nucleotide at a position corresponding to position 101 of SEQ ID NO: 37 (UNGC.41.IL1RAP).

3. The method of claim 2, wherein the radiation treatment comprises hypofractionated radiation, and the patient is identified as carrying or not carrying one or more mutations selected from:
a) not carrying or carrying only one copy of a C nucleotide at a position corresponding to position 101 of SEQ ID NO: 1 (miR.99a.promoter);
b) not carrying or carrying only one copy of an A nucleotide at a position corresponding to position 101 of SEQ ID NO: 3 (IL1.B_rs4848306);
c) not carrying or carrying only one copy of a G nucleotide at a position corresponding to position 101 of SEQ ID NO: 5 (SHC4_rs1062124);
d) carrying a T nucleotide at a position corresponding to position 101 of SEQ ID NO: 7 (rs17599026);
e) not carrying or carrying only one copy of an A nucleotide at a position corresponding to position 101 of SEQ ID NO: 9 (RAC1_rs9374);
f) not carrying a C nucleotide at a position corresponding to position 101 of SEQ ID NO: 11 (IL24_rs1150258);
g) not carrying or carrying only one copy of an A nucleotide at a position corresponding to position 101 of SEQ ID NO: 13 (IL1A_rs1800587);
h) not carrying a C nucleotide at a position corresponding to position 101 of SEQ ID NO: 15 (ERCC1_rs3212948);
i) not carrying or carrying only one copy of a G nucleotide at a position corresponding to position 101 of SEQ ID NO: 17 (IL.6_rs12700386);
j) not carrying or carrying only one copy of a G nucleotide at a position corresponding to position 101 of SEQ ID NO: 19 (IL.6_rs2069840);
k) not carrying or carrying only one copy of a T nucleotide at a position corresponding to position 101 of SEQ ID NO: 21 (CAMK2G_rs2306327);
l) not carrying or carrying only one copy of a T nucleotide at a position corresponding to position 101 of SEQ ID NO: 23 (BMP2_rs1980499);
m) not carrying or carrying only one copy of a C nucleotide at a position corresponding to position 101 of SEQ ID NO: 25 (EREG_rs1460008);
n) not carrying or carrying only one copy of a C nucleotide at a position corresponding to position 101 of SEQ ID NO: 27 (CD274_rs4143815);
o) not carrying or carrying only one copy of an A nucleotide at a position corresponding to position 101 of SEQ ID NO: 29 (IL17D_rs7787);
p) not carrying a C nucleotide at a position corresponding to position 101 of SEQ ID NO: 31 (STAT3/rs3744483), or carrying only one copy of the C nucleotide at a position corresponding to position 101 of SEQ ID NO: 31 (STAT3/rs3744483);
q) not carrying or carrying only one copy of a C nucleotide at a position corresponding to position 101 of SEQ ID NO: 33 (BMP4_rs17563)
r) not carrying a C nucleotide at a position corresponding to position 101 of SEQ ID NO: 35 (IRF5_rs10488631); or
s) not carrying or carrying only one copy of a T nucleotide at a position corresponding to position 101 of SEQ ID NO: 37 (UNGC.41.IL1RAP).

4. The method of claim 2, wherein the radiation treatment comprises conventionally fractionated (traditional) radiation, and the patient is identified as carrying or not carrying one or more mutations selected from:
a) carrying a C nucleotide at a position corresponding to position 101 of SEQ ID NO: 1 (miR.99a.promoter);
b) carrying an A nucleotide at a position corresponding to position 101 of SEQ ID NO: 3 (IL1.B_rs4848306);
c) carrying a G nucleotide at a position corresponding to position 101 of SEQ ID NO: 5 (SHC4_rs1062124);
d) not carrying a T nucleotide at a position corresponding to position 101 of SEQ ID NO: 7 (rs17599026);
e) carrying an A nucleotide at a position corresponding to position 101 of SEQ ID NO: 9 (RAC1_rs9374);
f) carrying a C nucleotide at a position corresponding to position 101 of SEQ ID NO: 11 (IL24_rs1150258);
g) carrying an A nucleotide at a position corresponding to position 101 of SEQ ID NO: 13 (IL1A_rs1800587);
h) carrying a C nucleotide at a position corresponding to position 101 of SEQ ID NO: 15 (ERCC1_rs3212948);
i) carrying a G nucleotide at a position corresponding to position 101 of SEQ ID NO: 17 (IL.6_rs12700386);
j) carrying a G nucleotide at a position corresponding to position 101 of SEQ ID NO: 19 (IL.6_rs2069840);
k) carrying a T nucleotide at a position corresponding to position 101 of SEQ ID NO: 21 (CAMK2G_rs2306327);
l) carrying a T nucleotide at a position corresponding to position 101 of SEQ ID NO: 23 (BMP2_rs1980499);
m) carrying a C nucleotide at a position corresponding to position 101 of SEQ ID NO: 25 (EREG_rs1460008);
n) carrying a C nucleotide at a position corresponding to position 101 of SEQ ID NO: 27 (CD274_rs4143815);
o) carrying an A nucleotide at a position corresponding to position 101 of SEQ ID NO: 29 (IL17D_rs7787);
p) carrying a C nucleotide at a position corresponding to position 101 of SEQ ID NO: 31 (STAT3/rs3744483), or carrying only one copy of the C nucleotide at a position corresponding to position 101 of SEQ ID NO: 31 (STAT3/rs3744483);
q) carrying a C nucleotide at a position corresponding to position 101 of SEQ ID NO: 33 (BMP4_rs17563)
r) carrying a C nucleotide at a position corresponding to position 101 of SEQ ID NO: 35 (IRF5_rs10488631); or
s) carrying a T nucleotide at a position corresponding to position 101 of SEQ ID NO: 37 (UNGC.41.IL1RAP).

5. The method of any one of claims 1-4, wherein the cancer is selected from adrenal cancer, anal cancer, bile duct cancer, bladder cancer, bone cancer, brain/CNS, basal cell skin cancer, breast cancer, cervical cancer, colorectal cancer, endometrial cancer, esophageal cancer, eye cancer, gallbladder cancer, gastrointestinal carcinoid tumors, gastrointestinal stromal tumor (GIST), gastric cancer, glioma, glioblastoma, head and neck cancer, Hodgkin disease, Kaposi sarcoma, kidney cancer, laryngeal and hypopharyngeal cancer, leukemia, liver cancer, lymphoma, malignant mesothelioma, merkel cell carcinoma, metastatic urothelial carcinoma, multiple myeloma, myeloma, myelodysplastic syndrome, nasal cavity and paranasal sinus cancer, nasopharyngeal cancer, neuroendocrine cancer, neuroblastoma, non-Hodgkin lymphoma, oral cavity and oropharyngeal cancer, osteosarcoma, ovarian cancer, pancreatic cancer, penile cancer, pituitary tumors, prostate cancer, renal cancer, retinoblastoma, rhabdomyosarcoma, salivary gland cancer, sarcoma, squamous cell skin cancer, small intestine cancer, stomach cancer, testicular cancer, thymus cancer, thyroid cancer, uterine cancer, or vaginal cancer.

6. The method of claim 5, wherein the cancer is sarcoma.

7. The method of claim 6, wherein the method is a reduced toxicity method, and the toxicity is wound toxicity.

8. The method of claim 7, wherein the patient has a tumor on a lower extremity.

9. The method of claim 1, wherein the radiation treatment comprises hypofractionated radiation (e.g., stereotactic body radiation) or conventionally fractionated (traditional) radiation, and the patient is identified as carrying or not carrying one or more mutations selected from:
a) a G nucleotide at position 101 of SEQ ID NO: 53 (BMP2_rs1979855);
b) a G nucleotide at position 101 of SEQ ID NO: 55 (PTPN2_rs1893217);
c) a C nucleotide at position 101 of SEQ ID NO: 43 (BRCA2_rs15869);
d) a C nucleotide at position 101 of SEQ ID NO: 65 (ABL1_rs11991);
e) a G nucleotide at position 101 of SEQ ID NO: 63 (ERCC1_rs11615);
f) an A nucleotide at position 101 of SEQ ID NO: 41 (RAD23A_rs8240);
g) a G nucleotide at position 101 of SEQ ID NO: 59 (BATF3_rs6695772);
h) a C nucleotide at position 101 of SEQ ID NO: 143 (FOXP3_rs2232365);
i) a G nucleotide at position 101 of SEQ ID NO: 39 (IL19_rs1798);
j) a G or T nucleotide at position 101 of SEQ ID NO: 129 (LIN28A_rs9438623);
k) an A nucleotide at position 101 of SEQ ID NO: 61 (IL18R1_rs11465660);
l) a T nucleotide at position 101 of SEQ ID NO: 153 (IL2RB_rs228942);
m) a T nucleotide at position 101 of SEQ ID NO: 49 (CD274_rs1411262);
n) a C nucleotide at position 101 of SEQ ID NO: 47 (BMP2_rs3178250);
o) a C nucleotide at position 101 of SEQ ID NO: 137 (SPI1_rs2071304);
p) a G nucleotide at position 101 of SEQ ID NO: 99 (IL19_rs2243158);
q) an A nucleotide at position 101 of SEQ ID NO: 167 (IL10_rs3024505);
r) a G nucleotide at position 101 of SEQ ID NO: 187 (TRL4_rs4986790);
s) an A nucleotide at position 101 of SEQ ID NO: 177 (VEGFA_rs41282644);
t) a G or C nucleotide at position 101 of SEQ ID NO: 163 (P2RX7_rs3751143);
u) a C nucleotide at position 101 of SEQ ID NO: 15 (ERCC1_rs3212948);
v) a G or C nucleotide at position 101 of SEQ ID NO: 131 (FANCC_rs9673);
w) a G nucleotide at position 101 of SEQ ID NO: 51 (CD274_rs2297136);
x) a G nucleotide at position 101 of SEQ ID NO: 19 (IL6_rs2069840);
y) a G nucleotide at position 101 of SEQ ID NO: 45 (CD274_rs2282055);
z) a G nucleotide at position 101 of SEQ ID NO: 57 (CD274_rs822339);
aa) a G nucleotide at position 101 of SEQ ID NO: 95 (HAMP_rs10421768);
bb) a C nucleotide at position 101 of SEQ ID NO: 145 (HAMP_rs1882694);
cc) a G nucleotide at position 101 of SEQ ID NO: 189 (ATM_rs1800057);
dd) a C nucleotide at position 101 of SEQ ID NO: 191 (ILRAP_rs79383051);
ee) an A nucleotide at position 101 of SEQ ID NO: 155 (KIT_rs17084733);
ff) a T nucleotide at position 101 of SEQ ID NO: 7 (rs17599026);
gg) an A nucleotide at position 101 of SEQ ID NO: 67 (ATM_rs189037);
hh) a G nucleotide at position 101 of SEQ ID NO: 69 (CD6_rs76677607);
ii) an A nucleotide at position 101 of SEQ ID NO: 13 (IL1A_rs1800587);
jj) an A nucleotide at position 101 of SEQ ID NO: 179 (IL1A_rs17561);
kk) a G nucleotide at position 101 of SEQ ID NO: 73 (BRCA2_rs7334543);
ll) a T nucleotide at position 101 of SEQ ID NO: 75 (ILF3.58_rs118142475);
mm) a C nucleotide at position 101 of SEQ ID NO: 77 (BIRC5_rs2239680);
nn) a T nucleotide at position 101 of SEQ ID NO: 107 (IL2RA_rs2476491);
oo) an A nucleotide at position 101 of SEQ ID NO: 109 (IL12A_rs568408);
pp) a C nucleotide at position 101 of SEQ ID NO: 111 (GSK3B_rs2037547);
qq) a G nucleotide at position 101 of SEQ ID NO: 135 (IL6_rs1800795);
rr) a T nucleotide at position 101 of SEQ ID NO: 105 (LIG4_rs2232643);
ss) a C nucleotide at position 101 of SEQ ID NO: 31 (STAT3_rs3744483);
tt) a G nucleotide at position 101 of SEQ ID NO: 115 (IL6_rs1800797);
uu) a G nucleotide at position 101 of SEQ ID NO: 17 (IL.6_rs12700386);
vv) an A nucleotide at position 101 of SEQ ID NO: 9 (RAC1_rs9374);
ww) a T nucleotide at position 101 of SEQ ID NO: 121 (ATM_rs373759);
xx) an A nucleotide at position 101 of SEQ ID NO: 123 (CETN2_rs8230);
yy) an A nucleotide at position 101 of SEQ ID NO: 126 (XRCC3_rs861539);
zz) a C nucleotide at position 101 of SEQ ID NO: 127 (miR.34b.c.promoter_rs4938723);
aaa) an A nucleotide at position 101 of SEQ ID NO: 3 (IL1.B_rs4848306);
bbb) an A nucleotide at position 101 of SEQ ID NO: 133 (REV3L_rs465646);
ccc) a C nucleotide at position 101 of SEQ ID NO: 183 (EGFR_rs884225);
ddd) a G nucleotide at position 101 of SEQ ID NO: 181 (TGFB1_rs1800469);
eee) a T nucleotide at position 101 of SEQ ID NO: 165 (rs2187668);
fff) a G nucleotide at position 101 of SEQ ID NO: 185 (rs17388569);
ggg) a G nucleotide at position 101 of SEQ ID NO: 79 (NBN_rs1805794);
hhh) a G nucleotide at position 101 of SEQ ID NO: 83 (CD274_rs4742098);
iii) a G nucleotide at position 101 of SEQ ID NO: 85 (ERCC4_rs4781562);
jjj) an A, C, or G nucleotide at position 101 of SEQ ID N: 87 (MSH2_rs2303428);
kkk) an A nucleotide at position 101 of SEQ ID NO: 151 (IL2RA_rs11256497);
lll) a C nucleotide at position 101 of SEQ ID NO: 159 (LIG4_rs3093772_P1.P2);
mmm) an A nucleotide at position 101 of SEQ ID NO: 29 (IL17D_rs7787);
nnn) a C nucleotide at position 101 of SEQ ID NO: (CD274_rs4143815);
ooo) an A nucleotide at position 101 of SEQ ID NO: 175 (UNGC.96.TGFB2_NA);
ppp) a G nucleotide at position 101 of SEQ ID NO: 161 (MDM2_rs769412); and
qqq) a G nucleotide at position 101 of SEQ ID NO: 147 (IL10_rs1800872).

10. The method of claim 9, wherein the radiation treatment comprises hypofractionated radiation, and the patient is identified as carrying or not carrying one or more mutations selected from:
a) not carrying or carrying only one copy of a G nucleotide at position 101 of SEQ ID NO: 53 (BMP2_rs1979855);
b) not carrying or carrying only one copy of a G nucleotide at position 101 of SEQ ID NO: 55 (PTPN2_rs1893217);
c) not carrying or carrying only one copy of a C nucleotide at position 101 of SEQ ID NO: 43 (BRCA2_rs15869);
d) carrying a C nucleotide at position 101 of SEQ ID NO: 65 (ABL1_rs11991);
e) not carrying or carrying only one copy of a G nucleotide at position 101 of SEQ ID NO: 63 (ERCC1_rs11615);
f) not carrying or carrying only one copy of an A nucleotide at position 101 of SEQ ID NO: 41 (RAD23A_rs8240);
g) carrying a G nucleotide at position 101 of SEQ ID NO: 59 (BATF3_rs6695772);
h) carrying a C nucleotide at position 101 of SEQ ID NO: 143 (FOXP3_rs2232365);
i) not carrying or carrying only one copy of a G nucleotide at position 101 of SEQ ID NO: 39 (IL19_rs1798);
j) not carrying a G or T nucleotide at position 101 of SEQ ID NO: 129 (LIN28A_rs9438623);
k) not carrying or carrying only one copy of an A nucleotide at position 101 of SEQ ID NO: 61 (IL18R1_rs11465660);
l) not carrying a T nucleotide at position 101 of SEQ ID NO: 153 (IL2RB_rs228942);
m) not carrying or carrying only one copy of a T nucleotide at position 101 of SEQ ID NO: 49 (CD274_rs1411262);
n) not carrying or carrying only one copy of a C nucleotide at position 101 of SEQ ID NO: 47 (BMP2_rs3178250);
o) not carrying a C nucleotide at position 101 of SEQ ID NO: 137 (SPIT_rs2071304);
p) not carrying a G nucleotide at position 101 of SEQ ID NO: 99 (IL19_rs2243158);
q) not carrying an A nucleotide at position 101 of SEQ ID NO: 167 (IL10_rs3024505);
r) carrying a G nucleotide at position 101 of SEQ ID NO: 187 (TRL4_rs4986790);
s) carrying an A nucleotide at position 101 of SEQ ID NO: 177 (VEGFA_rs41282644);
t) carrying a G or C nucleotide at position 101 of SEQ ID NO: 163 (P2RX7_rs3751143);
u) not carrying a C nucleotide at position 101 of SEQ ID NO: 15 (ERCC1_rs3212948);
v) not carrying a G or C nucleotide at position 101 of SEQ ID NO: 131 (FANCC_rs9673);
w) not carrying a G nucleotide at position 101 of SEQ ID NO: 51 (CD274_rs2297136);
x) not carrying or carrying only one copy of a G nucleotide at position 101 of SEQ ID NO: 19 (IL6_rs2069840);
y) not carrying or carrying only one copy of a G nucleotide at position 101 of SEQ ID NO: 45 (CD274_rs2282055);
z) carrying a G nucleotide at position 101 of SEQ ID NO: 57 (CD274_rs822339);
aa) carrying a G nucleotide at position 101 of SEQ ID NO: 95 (HAMP_rs10421768);
bb) carrying a C nucleotide at position 101 of SEQ ID NO: 145 (HAMP_rs1882694);
cc) not carrying a G nucleotide at position 101 of SEQ ID NO: 189 (ATM_rs1800057);
dd) not carrying a C nucleotide at position 101 of SEQ ID NO: 191 (ILRAP_rs79383051);
ee) carrying an A nucleotide at position 101 of SEQ ID NO: 155 (KIT_rs17084733); and
ff) carrying a T nucleotide at position 101 of SEQ ID NO: 7 (rs17599026).

11. The method of claim 9 or 10, wherein the cancer is selected from adrenal cancer, anal cancer, bile duct cancer, bladder cancer, bone cancer, brain/CNS, basal cell skin cancer, breast cancer, cervical cancer, colorectal cancer, endometrial cancer, esophageal cancer, eye cancer, gallbladder cancer, gastrointestinal carcinoid tumors, gastrointestinal stromal tumor (GIST), gastric cancer, glioma, glioblastoma, head and neck cancer, Hodgkin disease, Kaposi sarcoma, kidney cancer, laryngeal and hypopharyngeal cancer, leukemia, liver cancer, lymphoma, malignant mesothelioma, merkel cell carcinoma, metastatic urothelial carcinoma, multiple myeloma, myeloma, myelodysplastic syndrome, nasal cavity and paranasal sinus cancer, nasopharyngeal cancer, neuroendocrine cancer, neuroblastoma, non-Hodgkin lymphoma, oral cavity and oropharyngeal cancer, osteosarcoma, ovarian cancer, pancreatic cancer, penile cancer, pituitary tumors, prostate cancer, renal cancer, retinoblastoma, rhabdomyosarcoma, salivary gland cancer, sarcoma, squamous cell skin cancer, small intestine cancer, stomach cancer, testicular cancer, thymus cancer, thyroid cancer, uterine cancer, or vaginal cancer.

12. The method of claim 10 or 11, wherein the cancer is prostate cancer.

13. The method any one of claims 10-12, wherein the hypofractionated radiation regimen comprises administering from about 20 Gy to about 35 Gy radiation over about 5 to about 8 fractions.

14. The method of any one of claims 10-13, wherein the hypofractionated radiation regimen comprises administering stereotactic body radiation therapy.

15. The method of claim 9, wherein the radiation treatment comprises conventionally fractionated (traditional) radiation, and the patient is identified as carrying or not carrying one or more mutations selected from:
a) carrying two copies of a G nucleotide at position 101 of SEQ ID NO: 53 (BMP2_rs1979855);
b) carrying two copies of a G nucleotide at position 101 of SEQ ID NO: 55 (PTPN2_rs1893217);
c) carrying two copies of a C nucleotide at position 101 of SEQ ID NO: 43 (BRCA2_rs15869);
d) not carrying a C nucleotide at position 101 of SEQ ID NO: 65 (ABL1_rs11991);
e) carrying two copies of a G nucleotide at position 101 of SEQ ID NO: 63 (ERCC1_rs11615);
f) carrying two copies of an A nucleotide at position 101 of SEQ ID NO: 41 (RAD23A_rs8240);
g) not carrying a G nucleotide at position 101 of SEQ ID NO: 59 (BATF3_rs6695772);
h) not carrying a C nucleotide at position 101 of SEQ ID NO: 143 (FOXP3_rs2232365);
i) carrying two copies of a G nucleotide at position 101 of SEQ ID NO: 39 (IL19_rs1798);
j) carrying a G or T nucleotide at position 101 of SEQ ID NO: 129 (LIN28A_rs9438623);
k) carrying two copies of an A nucleotide at position 101 of SEQ ID NO: 61 (IL18R1_rs11465660);
l) carrying a T nucleotide at position 101 of SEQ ID NO: 153 (IL2RB_rs228942);
m) carrying two copies of a T nucleotide at position 101 of SEQ ID NO: 49 (CD274_rs1411262);
n) carrying two copies of a C nucleotide at position 101 of SEQ ID NO: 47 (BMP2_rs3178250);
o) carrying a C nucleotide at position 101 of SEQ ID NO: 137 (SPIT_rs2071304);
p) carrying a G nucleotide at position 101 of SEQ ID NO: 99 (IL19_rs2243158);
q) carrying an A nucleotide at position 101 of SEQ ID NO: 167 (IL10_rs3024505);
r) not carrying a G nucleotide at position 101 of SEQ ID NO: 187 (TRL4_rs4986790);
s) not carrying an A nucleotide at position 101 of SEQ ID NO: 177 (VEGFA_rs41282644);
t) not carrying a G or C nucleotide at position 101 of SEQ ID NO: 163 (P2RX7_rs3751143);
u) carrying a C nucleotide at position 101 of SEQ ID NO: 15 (ERCC1_rs3212948);
v) carrying a G or C nucleotide at position 101 of SEQ ID NO: 131 (FANCC_rs9673);
w) carrying a G nucleotide at position 101 of SEQ ID NO: 51 (CD274_rs2297136);
x) carrying two copies of a G nucleotide at position 101 of SEQ ID NO: 19 (IL6_rs2069840);
y) carrying two copies of a G nucleotide at position 101 of SEQ ID NO: 45 (CD274_rs2282055);
z) not carrying a G nucleotide at position 101 of SEQ ID NO: 57 (CD274_rs822339);
aa) not carrying a G nucleotide at position 101 of SEQ ID NO: 95 (HAMP_rs10421768);
bb) not carrying a C nucleotide at position 101 of SEQ ID NO: 145 (HAMP_rs1882694);
cc) carrying a G nucleotide at position 101 of SEQ ID NO: 189 (ATM_rs1800057);
dd) carrying a C nucleotide at position 101 of SEQ ID NO: 191 (ILRAP_rs79383051);
ee) not carrying an A nucleotide at position 101 of SEQ ID NO: 155 (KIT_rs17084733); and
ff) not carrying a T nucleotide at position 101 of SEQ ID NO: 7 (rs17599026).

16. The method of claim 9, wherein the patient is treated with conventionally fractionated (traditional) radiation, and the patient is identified as carrying or not carrying one or more mutations selected from:
a) carrying an A nucleotide at position 101 of SEQ ID NO: 67 (ATM_rs189037);
b) not carrying a G nucleotide at position 101 of SEQ ID NO: 69 (CD6_rs76677607);
c) not carrying or carrying only one copy of an A nucleotide at position 101 of SEQ ID NO: 13 (IL1A_rs1800587);
d) carrying an A nucleotide at position 101 of SEQ ID NO: 179 (IL1A_rs17561);
e) carrying a G nucleotide at position 101 of SEQ ID NO: 73 (BRCA2_rs7334543);
f) not carrying a T nucleotide at position 101 of SEQ ID NO: 75 (ILF3.58_rs118142475);
g) carrying a C nucleotide at position 101 of SEQ ID NO: 77 (BIRC5_rs2239680);
h) carrying a T nucleotide at position 101 of SEQ ID NO: 107 (IL2RA_rs2476491);
i) carrying an A nucleotide at position 101 of SEQ ID NO: 109 (IL12A_rs568408);
j) not carrying a C nucleotide at position 101 of SEQ ID NO: 111 (GSK3B_rs2037547);
k) carrying a G nucleotide at position 101 of SEQ ID NO: 135 (IL6_rs1800795);
l) not carrying a T nucleotide at position 101 of SEQ ID NO: 105 (LIG4_rs2232643);
m) not carrying or carrying only one copy of a C nucleotide at position 101 of SEQ ID NO: 31 (STAT3_rs3744483);
n) carrying a G nucleotide at position 101 of SEQ ID NO: 115 (IL6_rs1800797);
o) not carrying or carrying only one copy of a G nucleotide at position 101 of SEQ ID NO:17 (IL.6_rs12700386);
p) not carrying or carrying only one copy of an A nucleotide at position 101 of SEQ ID NO: 9 (RAC1_rs9374);
q) not carrying a T nucleotide at position 101 of SEQ ID NO: 121 (ATM_rs373759);
r) not carrying an A nucleotide at position 101 of SEQ ID NO: 123 (CETN2_rs8230);
s) not carrying an A nucleotide at position 101 of SEQ ID NO: 126 (XRCC3_rs861539);
t) carrying a C nucleotide at position 101 of SEQ ID NO: 127 (miR.34b.c.promoter_rs4938723);
u) not carrying or carrying only one copy of an A nucleotide at position 101 of SEQ ID NO: 61 (IL18R1_rs11465660);
v) not carrying or carrying only one copy of an A nucleotide at position 101 of SEQ ID NO: 3 (IL1.B_rs4848306);
w) not carrying a G or C nucleotide at position 101 of SEQ ID NO: 131 (FANCC_rs9673);
x) carrying an A nucleotide at position 101 of SEQ ID NO: 133 (REV3L_rs465646);
y) not carrying a G nucleotide at position 101 of SEQ ID NO: 51 (CD274_rs2297136);
z) carrying a C nucleotide at position 101 of SEQ ID NO: 183 (EGFR_rs884225);
aa) not carrying a G nucleotide at position 101 of SEQ ID NO: 181 (TGFB1_rs1800469);
bb) carrying a T nucleotide at position 101 of SEQ ID NO: 165 (rs2187668);
cc) carrying a G nucleotide at position 101 of SEQ ID NO: 185 (rs17388569);
dd) not carrying a G nucleotide at position 101 of SEQ ID NO: 79 (NBN_rs1805794);
ee) carrying a G nucleotide at position 101 of SEQ ID NO: 83 (CD274_rs4742098);
ff) carrying a G nucleotide at position 101 of SEQ ID NO: 85 (ERCC4_rs4781562);
gg) not carrying an A, C, or G nucleotide at position 101 of SEQ ID N: 87 (MSH2_rs2303428);
hh) carrying a T nucleotide at position 101 of SEQ ID NO: 7 (rs17599026);
ii) not carrying an A nucleotide at position 101 of SEQ ID NO: 151 (IL2RA_rs11256497);
jj) carrying a C nucleotide at position 101 of SEQ ID NO: 159 (LIG4_rs3093772_P1.P2);
kk) not carrying or carrying only one copy of an A nucleotide at position 101 of SEQ ID NO: 29 (IL17D_rs7787);
ll) not carrying or carrying only one copy of a C nucleotide at position 101 of SEQ ID NO: (CD274_rs4143815);
mm) not carrying an A nucleotide at position 101 of SEQ ID NO: 175 (UNGC.96.TGFB2_NA);
nn) not carrying a G nucleotide at position 101 of SEQ ID NO: 161 (MDM2_rs769412);
oo) not carrying an A nucleotide at position 101 of SEQ ID NO: 167 (IL10_rs3024505);
pp) not carrying or carrying only one copy of a C nucleotide at position 101 of SEQ ID NO: 43 (BRCA2_rs15869);
qq) not carrying a G nucleotide at position 101 of SEQ ID NO: 147 (IL10_rs1800872);
rr) not carrying or carrying only one copy of a G nucleotide at position 101 of SEQ ID NO: 53 (BMP2_rs1979855);
ss) not carrying or carrying only one copy of a G nucleotide at position 101 of SEQ ID NO: 55 (PTPN2_rs1893217);
tt) carrying a C nucleotide at position 101 of SEQ ID NO: 65 (ABL1_rs11991); and
uu) not carrying or carrying only one copy of a G nucleotide at position 101 of SEQ ID NO: 63 (ERCC1_rs11615).

17. The method of claim 15 or 16, wherein the cancer is selected from adrenal cancer, anal cancer, bile duct cancer, bladder cancer, bone cancer, brain/CNS, basal cell skin cancer, breast cancer, cervical cancer, colorectal cancer, endometrial cancer, esophageal cancer, eye cancer, gallbladder cancer, gastrointestinal carcinoid tumors, gastrointestinal stromal tumor (GIST), gastric cancer, glioma, glioblastoma, head and neck cancer, Hodgkin disease, Kaposi sarcoma, kidney cancer, laryngeal and hypopharyngeal cancer, leukemia, liver cancer, lymphoma, malignant mesothelioma, merkel cell carcinoma, metastatic urothelial carcinoma, multiple myeloma, myeloma, myelodysplastic syndrome, nasal cavity and paranasal sinus cancer, nasopharyngeal cancer, neuroendocrine cancer, neuroblastoma, non-Hodgkin lymphoma, oral cavity and oropharyngeal cancer, osteosarcoma, ovarian cancer, pancreatic cancer, penile cancer, pituitary tumors, prostate cancer, renal cancer, retinoblastoma, rhabdomyosarcoma, salivary gland cancer, sarcoma, squamous cell skin cancer, small intestine cancer, stomach cancer, testicular cancer, thymus cancer, thyroid cancer, uterine cancer, or vaginal cancer.

18. The method of claim 17, wherein the cancer is prostate cancer.

19. The method of any one of claims 15-18, wherein the method is a reduced toxicity method and the toxicity is acute or long-term toxicity.

20. The method of claim 19, wherein the toxicity is GI and/or GU toxicity.

21. The method of any one of claims 15-20, wherein the method results in less toxicity than a hypofractionated radiation regimen.

22. The method of any one of claims 15-21, wherein the traditional radiation regimen comprises administering from about 40 Gy to about 60 Gy radiation over a period of about 5 to about 6 weeks.

23. The method of any one of claims 15-21, wherein the traditional radiation regimen comprises administering from about 40 Gy to about 60 Gy radiation over about 15 to about 30 fractions.

24. The method of any one of claims 15-23, wherein the radiation therapy is external beam radiation therapy.

25. A method of treating cancer comprising administering a radiation treatment to a cancer patient identified as carrying or not carrying one or more mutations in a SNP selected from: TREX1_rs11797, LIN28A_rs9438623, miR.34b.c.promoter_rs4938723, FCGR2A_rs1801274, CD274_rs4742098, IL.6_rs12700386, SPI1_rs2071304, ERCC4_rs4781562, IL10_rs3024496, IL.6_rs2069840, rs2187668, CD274_rs4143815, KRAS_rs61764370, SHC4_rs1062124, HAMP_rs1882694, rs922075, ERCC1_rs11615, EXO1_rs4150021, STAT4_rs7574070, and SOS1_rs1059313; wherein the presence or the absence of the SNP is associated with a reduced risk of distant failure in response to radiation therapy.

26. The method of claim 25, wherein the patient is identified as carrying or not carrying one or more mutations in a SNP selected from:
a) carrying a T nucleotide at position 101 of SEQ ID NO: 81 (TREX1_rs11797);
b) carrying a G or T nucleotide at position 101 of SEQ ID NO: 129 (LIN28A_rs9438623);
c) carrying or not carrying a C nucleotide at position 101 of SEQ ID NO: 127 (miR.34b.c.promoter_rs4938723);
d) carrying a C or G nucleotide at position 101 of SEQ ID NO: 119 (FCGR2A_rs1801274);
e) carrying a G nucleotide at position 101 of SEQ ID NO: 83 (CD274_rs4742098);
f) not carrying a G nucleotide at position 101 of SEQ ID NO: 17 (IL.6_rs12700386);
g) not carrying a C nucleotide at position 101 of SEQ ID NO: 137 (SPI1_rs2071304);
h) carrying a G nucleotide at position 101 of SEQ ID NO: 85 (ERCC4_rs4781562);
i) carrying a G nucleotide at position 101 of SEQ ID NO: 103 (IL10_rs3024496_P1.P2);
j) carrying a G nucleotide at position 101 of SEQ ID NO: 139 (IL10_rs3024496);
k) not carrying or carrying only one copy of a G nucleotide at position 101 of SEQ ID NO: 19 (IL.6_rs2069840);
l) not carrying a T nucleotide at position 101 of SEQ ID NO: 165 (rs2187668);
m) not carrying a C nucleotide at position 101 of SEQ ID NO: 27 (CD274_rs4143815);
n) carrying a C nucleotide at position 101 of SEQ ID NO: 157 (KRAS_rs61764370);
o) carrying a G nucleotide at position 101 of SEQ ID NO: 5 (SHC4_rs1062124);
p) carrying a G nucleotide at position 101 of SEQ ID NO: 169 (rs922075);
q) carrying a G nucleotide at position 101 of SEQ ID NO: 63 (ERCC1_rs11615);
r) not carrying the deletion of a T nucleotide at [ATTTTG] of SEQ ID NO: 142 (EXO1_rs4150021);
s) carrying a C nucleotide at position 101 of SEQ ID NO: 173 (STAT4_rs7574070); and
t) carrying a T nucleotide at position 101 of SEQ ID NO: 171 (SOS1_rs1059313).

27. The method of claim 25 or 26, wherein the radiation therapy is hypofractionated radiation.

28. The method of any one of claims 25-27, wherein the cancer is sarcoma.

29. A method of treating cancer in a cancer patient comprising administering a radiation treatment and/or a different cancer treatment to a patient identified as carrying or not carrying one or more mutations in a SNP selected from: BIRC5_rs2239680, SMAD1_rs11724777, IL12A_rs568408, and IL13_rs20541, wherein the presence or the absence of the SNP is associated with an increased risk of mortality or a reduced risk of mortality.

30. The method of claim 29, wherein if the patient is identified as carrying or not carrying one or more mutations in a SNP associated with a reduced risk of mortality and selected from:
a) not carrying a C nucleotide at position 101 of SEQ ID NO: 77 (BIRC5_rs2239680);
b) carrying an A nucleotide at position 101 of SEQ ID NO: 93 (SMAD1_rs11724777);
c) carrying an A nucleotide at position 101 of SEQ ID NO: 109 (IL12A_rs568408); and
d) carrying a T nucleotide at position 101 of SEQ ID NO: 193 (IL13_rs20541), the patient is administered a radiation treatment.

31. The method of claim 29 or 30, wherein the radiation therapy is conventionally fractionated (traditional) radiation.

32. The method of claim 29, wherein if the patient is identified as carrying or not carrying one or more mutations in a SNP associated with an increased risk of mortality and selected from:
a) carrying a C nucleotide at position 101 of SEQ ID NO: 77 (BIRC5_rs2239680);
b) not carrying an A nucleotide at position 101 of SEQ ID NO: 93 (SMAD1_rs11724777);
c) not carrying an A nucleotide at position 101 of SEQ ID NO: 109 (IL12A_rs568408); and
d) not carrying a T nucleotide at position 101 of SEQ ID NO: 193 (IL13_rs20541);
the patient is administered a radiation treatment and an additional cancer treatment, or a different cancer treatment.

33. The method of claim 29 or 30, wherein the radiation therapy is conventionally fractionated (traditional) radiation.

34. The method of claim 32, wherein the additional treatment or the different treatment is hypofractionated radiation, brachytherapy, chemotherapy, androgen deprivation therapy, immunotherapy, high intensity focused ultrasound, cryotherapy, laser ablation, photodynamic therapy, or surgery.

35. The method of any one of claims 29-34, wherein the cancer is prostate cancer.

36. A method of treating cancer in a cancer patient comprising administering a radiation treatment and/or a different cancer treatment to a patient identified as carrying or not carrying one or more mutations in a SNP selected from: BMP2_rs3178250, SMAD1_rs11724777, IL10RB_rs2834167, CD274_rs822339, CD274_rs2282055, IL12A_rs568408, PARP1_rs8679, CD274_rs1411262, BMP2_rs1979855, and P2RX7_rs3751143, wherein the presence or the absence of the SNP is associated with an increased risk of (decreased time to) biochemical relapse or is associated with a reduced risk of biochemical relapse following radiation therapy.

37. The method of claim 36, wherein if the patient is identified as carrying or not carrying one or more mutations in a SNP associated with increased risk of biochemical relapse and selected from:
a) carrying a C nucleotide at position 101 of SEQ ID NO: 47 (BMP2_rs3178250);
b) not carrying a G nucleotide at position 101 of SEQ ID NO: 91 (IL10RB_rs2834167);
c) not carrying a G nucleotide at position 101 of SEQ ID NO: 57 (CD274_rs822339);
d) carrying a G nucleotide at position 101 of SEQ ID NO: 45 (CD274_rs2282055);
e) carrying an A nucleotide at position 101 of SEQ ID NO: 109 (IL12A_rs568408);
f) carrying a G nucleotide at position 101 of SEQ ID NO: 195 (PARP1_rs8679);
g) carrying a T nucleotide at position 101 of SEQ ID NO: 49 (CD274_rs1411262);
h) carrying a G nucleotide at position 101 of SEQ ID NO: 53 (BMP2_rs1979855); and
i) carrying a C nucleotide at position 101 of SEQ ID NO: 163 (P2RX7_rs3751143);
the patient is treated with a treatment in addition to radiation therapy, or with a different treatment.

38. The method of claim 36, wherein if the patient is identified as carrying or not carrying one or more mutations in a SNP associated with decreased risk of biochemical relapse and selected from:
a) not carrying a C nucleotide at position 101 of SEQ ID NO: 47 (BMP2_rs3178250);
b) carrying a G nucleotide at position 101 of SEQ ID NO: 91 (IL10RB_rs2834167);
c) carrying a G nucleotide at position 101 of SEQ ID NO: 57 (CD274_rs822339);
d) not carrying a G nucleotide at position 101 of SEQ ID NO: 45 (CD274_rs2282055);
e) not carrying an A nucleotide at position 101 of SEQ ID NO: 109 (IL12A_rs568408);
f) not carrying a G nucleotide at position 101 of SEQ ID NO: 195 (PARP1_rs8679);
g) not carrying a T nucleotide at position 101 of SEQ ID NO: 49 (CD274_rs1411262);
h) not carrying a G nucleotide at position 101 of SEQ ID NO: 53 (BMP2_rs1979855); and
i) carrying a C nucleotide at position 101 of SEQ ID NO: 163 (P2RX7_rs3751143);
the patient is treated with radiation therapy.

39. The method of any one of claims 36-38, wherein the radiation therapy is conventionally fractionated (traditional) radiation.

40. The method of claim 37, wherein the additional treatment or the different treatment is hypofractionated radiation, brachytherapy, chemotherapy, androgen deprivation therapy, immunotherapy, high intensity focused ultrasound, cryotherapy, laser ablation, photodynamic therapy, or surgery.

41. The method of any one of claims 36-40, wherein the cancer is prostate cancer.

42. A method of treating cancer in a cancer patient comprising administering a radiation treatment and/or a different cancer treatment to a patient identified as carrying or not carrying one or more mutations in a SNP selected from: IL8_rs4073, IL10_rs3024496_P1, BIRC5_rs2239680, RAC1_rs9374, IL10_rs3024496_P1.P2, BMP2_rs3178250, and NBN_rs1805794, wherein the presence or the absence of the SNP is associated with an increased risk of distant metastasis or is associated with a decreased risk of distant metastasis.

43. The method of claim 42, wherein if the patient is identified as carrying or not carrying one or more mutations in a SNP associated with increased risk of distant metastasis and selected from:
a) carrying a T nucleotide at position 101 of SEQ ID NO: 197 (IL8_rs4073);
b) not carrying a G or C nucleotide at position 101 of SEQ ID NO: 103 (IL10_rs3024496_P1);
c) not carrying a C nucleotide occurs at position 101 of SEQ ID NO: 77 (BIRC5_rs2239680);
d) carrying an A nucleotide at position 101 of SEQ ID NO: 9 (RAC1_rs9374);
e) not carrying a G nucleotide occurs at position 101 of SEQ ID NO: 103 (IL10_rs3024496_P1.P2);
f) carrying a G nucleotide at position 101 of SEQ ID NO: 53 (BMP2_rs1979855); and
g) carrying a G nucleotide at position 101 of SEQ ID NO: 79 (NBN_rs1805794);
the patient is treated with a treatment in addition to radiation therapy, or with a different treatment.

44. The method of claim 42, wherein if the patient is identified as carrying or not carrying one or more mutations in a SNP associated with decreased risk of distant metastasis and selected from:
a) not carrying a T nucleotide at position 101 of SEQ ID NO: 197 (IL8_rs4073);
b) carrying a G or C nucleotide at position 101 of SEQ ID NO: 103 (IL10_rs3024496_P1);
c) carrying a C nucleotide occurs at position 101 of SEQ ID NO: 77 (BIRC5_rs2239680);
d) not carrying an A nucleotide at position 101 of SEQ ID NO: 9 (RAC1_rs9374);
e) carrying a G nucleotide occurs at position 101 of SEQ ID NO: 103 (IL10_rs3024496_P1.P2);
f) not carrying a G nucleotide at position 101 of SEQ ID NO: 53 (BMP2_rs1979855); and
g) not carrying a G nucleotide at position 101 of SEQ ID NO: 79 (NBN_rs1805794);
the patient is treated with a radiation treatment.

45. The method of any one of claims 42-44, wherein the radiation therapy is conventionally fractionated (traditional) radiation.

46. The method of claim 43, wherein additional treatment or the different treatment is hypofractionated radiation, brachytherapy, chemotherapy, androgen deprivation therapy, immunotherapy, high intensity focused ultrasound, cryotherapy, laser ablation, photodynamic therapy, or surgery.

47. The method of any one of claims 42-46, wherein the cancer is prostate cancer.

48. A method of treating prostate cancer in a patient comprising administering a radiation treatment to a patient identified as carrying or not carrying one or more mutations in a SNP selected from: HAMP_rs10421768, XRCC1_rs25487, IL.6_rs2069840, XRCC4_rs1040363, and IL19_rs2243158, wherein the presence or the absence of the SNP is associated with a reduced risk of impotency.

49. The method of claim 48, wherein the patient is identified as carrying or not carrying one or more mutations in a SNP selected from:
a) carrying a G nucleotide at position 101 of SEQ ID NO: 95 (HAMP_rs10421768);
b) not carrying a C nucleotide at position 101 of SEQ ID NO: 97 (XRCC1_rs25487);
c) not carrying a G nucleotide at position 101 of SEQ ID NO: 19 (IL.6_rs2069840);
d) carrying a C nucleotide at position 101 of SEQ ID NO: 89 (XRCC4_rs1040363); and
e) not carrying a G nucleotide at position 101 of SEQ ID NO: 99 (IL19_rs2243158).

50. The method of claim 48 or 49, wherein the radiation therapy is conventionally fractionated (traditional) radiation.

51. A method for determining the toxicity of a radiation treatment in a cancer patient comprising determining whether the patient carries one or more mutations selected from:
a) a G nucleotide at a position corresponding to position 101 of SEQ ID NO: 53 (BMP2_rs1979855);
b) a C nucleotide at a position corresponding to position 101 of SEQ ID NO: 1 (miR.99a.promoter);
c) an A nucleotide at a position corresponding to position 101 of SEQ ID NO: 3 rs4848306);
d) a G nucleotide at a position corresponding to position 101 of SEQ ID NO: 5 (SHC4_rs1062124);
e) a T nucleotide at a position corresponding to position 101 of SEQ ID NO: 7 (rs17599026);
f) an A nucleotide at a position corresponding to position 101 of SEQ ID NO: 9 (RAC1_rs9374);
g) an A, C, or G nucleotide at a position corresponding to position 101 of SEQ ID NO: 11 (IL24_rs1150258);
h) an A nucleotide at a position corresponding to position 101 of SEQ ID NO: 13 (IL1A_rs1800587);
i) a C nucleotide at a position corresponding to position 101 of SEQ ID NO: 15 (ERCC1_rs3212948);
j) a G nucleotide at a position corresponding to position 101 of SEQ ID NO: 17 (IL.6_rs12700386);
k) a G nucleotide at a position corresponding to position 101 of SEQ ID NO: 19 (IL.6_rs2069840);
l) a T nucleotide at a position corresponding to position 101 of SEQ ID NO: 21 (CAMK2G_rs2306327);
m) a T nucleotide at a position corresponding to position 101 of SEQ ID NO: 23 (BMP2_rs1980499);
n) a C nucleotide at a position corresponding to position 101 of SEQ ID NO: 25 (EREG_rs1460008);
o) a C nucleotide at a position corresponding to position 101 of SEQ ID NO: 27 (CD274_rs4143815);
p) an A nucleotide at a position corresponding to position 101 of SEQ ID NO: 29 (IL17D_rs7787);
q) a C nucleotide at a position corresponding to position 101 of SEQ ID NO: 31 (STAT3/rs3744483);
r) a C nucleotide at a position corresponding to position 101 of SEQ ID NO: 33 (BMP4_rs17563)
s) a C nucleotide at a position corresponding to position 101 of SEQ ID NO: 35 (IRF5_rs10488631);
t) a T nucleotide at a position corresponding to position 101 of SEQ ID NO: 37 (UNGC.41.IL1RAP);
u) a G nucleotide at a position corresponding to position 101 of SEQ ID NO: 39 (IL19_rs1798);
v) an A nucleotide at a position corresponding to position 101 of SEQ ID NO: 41 (RAD23A_rs8240);
w) a C nucleotide at a position corresponding to position 101 of SEQ ID NO: 43 (BRCA2_rs15869);
x) a G nucleotide at a position corresponding to position 101 of SEQ ID NO: 45 (CD274_rs2282055);
y) a C nucleotide at a position corresponding to position 101 of SEQ ID NO: 47 (BMP2_rs3178250);
z) a T nucleotide at a position corresponding to position 101 of SEQ ID NO: 49 (CD274_rs1411262);
aa) an A nucleotide at a position corresponding to position 101 of SEQ ID NO: 51 (CD274_rs2297136);
bb) a G nucleotide at a position corresponding to position 101 of SEQ ID NO: 55 (PTPN2_rs1893217);
cc) a G nucleotide at a position corresponding to position 101 of SEQ ID NO: 57 (CD274_rs822339);
dd) a G nucleotide at a position corresponding to position 101 of SEQ ID NO: 59 (BATF3_rs6695772);
ee) an A nucleotide at a position corresponding to position 101 of SEQ ID NO: 61 (IL18R1_rs11465660);
ff) a G nucleotide at a position corresponding to position 101 of SEQ ID NO: 63 (ERCC1_rs11615);
gg) a C nucleotide at a position corresponding to position 101 of SEQ ID NO: 65 (ABL1_rs11991);
hh) an A nucleotide at a position corresponding to position 101 of SEQ ID NO: 67 (ATM_rs189037);
ii) an A nucleotide at a position corresponding to position 101 of SEQ ID NO: 69 (CD6_rs76677607);
jj) an A nucleotide at a position corresponding to position 101 of SEQ ID NO: 71 (IL1A_rs17561);
kk) a G nucleotide at a position corresponding to position 101 of SEQ ID NO: 73 (BRCA2_rs7334543);
ll) a T nucleotide at a position corresponding to position 101 of SEQ ID NO: 75 (ILF3.58_rs118142475);
mm) a C nucleotide at a position corresponding to position 101 of SEQ ID NO: 77 (BIRC5_rs2239680);
nn) a G nucleotide at a position corresponding to position 101 of SEQ ID NO: 79 (NBN_rs1805794);
oo) a T nucleotide at a position corresponding to position 101 of SEQ ID NO: 81 (TREX_rs11797);
pp) a G nucleotide at a position corresponding to position 101 of SEQ ID NO: 83 (CD274_rs4742098);
qq) a T nucleotide at a position corresponding to position 101 of SEQ ID NO: 85 (ERCC4_rs4781562);
rr) an A, C or G nucleotide at a position corresponding to position 101 of SEQ ID NO: 87 (MSH2_rs2303428);
ss) a C nucleotide at a position corresponding to position 101 of SEQ ID NO: 89 (XRCC4_rs1040363);
tt) a G nucleotide at a position corresponding to position 101 of SEQ ID NO: 91 (IL10RB_rs2834167);
uu) an A nucleotide at a position corresponding to position 101 of SEQ ID NO: 93 (SMAD1_rs11724777);
vv) a G nucleotide at a position corresponding to position 101 of SEQ ID NO: 95 (HAMP_rs10421768);
ww) a C nucleotide at a position corresponding to position 101 of SEQ ID NO: 97 (XRCC1_rs25487);
xx) a G nucleotide at a position corresponding to position 101 of SEQ ID NO: 99 (IL19_rs2243158);
yy) a C nucleotide at a position corresponding to position 101 of SEQ ID NO: 101 (FOXP3_rs2280883);
zz) a G nucleotide at a position corresponding to position 101 of SEQ ID NO: 103 (IL10_rs3024496_P1.P2);
aaa) a C nucleotide at a position corresponding to position 101 of SEQ ID NO: 105 (LIG4_rs2232643);
bbb) a T nucleotide at a position corresponding to position 101 of SEQ ID NO: 107 (IL2RA_rs2476491);
ccc) an A nucleotide at a position corresponding to position 101 of SEQ ID NO: 109 (IL12A_rs568408);
ddd) a C nucleotide at a position corresponding to position 101 of SEQ ID NO: 111 (GSK3B_rs2037547);
eee) an A nucleotide at a position corresponding to position 101 of SEQ ID NO: 113 (ATM_rs189037);
fff) a G nucleotide at a position corresponding to position 101 of SEQ ID NO: 115 (IL6_rs1800797);
ggg) an A nucleotide at a position corresponding to position 101 of SEQ ID NO: 117 (RAC1_rs9374);
hhh) a C or G nucleotide at a position corresponding to position 101 of SEQ ID NO: 119 (FCGR2A_rs1801274);
iii) a T nucleotide at a position corresponding to position 101 of SEQ ID NO: 121 (ATM_rs373759);
jjj) an A nucleotide at a position corresponding to position 101 of SEQ ID NO: 123 (CETN2_rs8230);
kkk) an A nucleotide at a position corresponding to position 101 of SEQ ID NO: 125 (XRCC3_rs861539);
lll) a C nucleotide at a position corresponding to position 101 of SEQ ID NO: 127 (miR.34b.c.promoter_rs4938723);
mmm) a G nucleotide at a position corresponding to position 101 of SEQ ID NO: 129 (LIN28A_rs9438623);
nnn) a G or C nucleotide at a position corresponding to position 101 of SEQ ID NO: 131 (FANCC_rs9673);
ooo) an A nucleotide at a position corresponding to position 101 of SEQ ID NO: 133 (REV3L_rs465646);
ppp) a G nucleotide at a position corresponding to position 101 of SEQ ID NO: 135 (IL6_rs1800795);
qqq) a C nucleotide at a position corresponding to position 101 of SEQ ID NO: 137 (SPI1_rs2071304);
rrr) a G nucleotide at a position corresponding to position 101 of SEQ ID NO: 139 (IL10_rs3024496);
sss) a deletion of a T nucleotide in the sequence ATTTTG of SEQ ID NO: 142 (wild-type sequence) to give a corresponding sequence of ATTTG as in SEQ ID NO: 141 (mutant) (EXO1_rs4150021);
ttt) an A nucleotide at a position corresponding to position 101 of SEQ ID NO: 143 (FOXP3_rs2232365);
uuu) a C nucleotide at a position corresponding to position 101 of SEQ ID NO: 145 (HAMP_rs1882694);
vvv) a G nucleotide at a position corresponding to position 101 of SEQ ID NO: 147 (IL10_rs1800872);
www) a G nucleotide at a position corresponding to position 101 of SEQ ID NO: 149 (IL16_rs11556218);
xxx) an A nucleotide at a position corresponding to position 101 of SEQ ID NO: 151 (IL2RA_rs11256497);
yyy) a T nucleotide at a position corresponding to position 101 of SEQ ID NO: 153 (IL2RB_rs228942);
zzz) an A nucleotide at a position corresponding to position 101 of SEQ ID NO: 155 (KIT_rs17084733);
aaaa) a C nucleotide at a position corresponding to position 101 of SEQ ID NO: 157 (KRAS_rs61764370);
bbbb) a C nucleotide at a position corresponding to position 101 of SEQ ID NO: 159 (LIG4_rs3093772_P1.P2);
cccc) a G nucleotide at a position corresponding to position 101 of SEQ ID NO: 161 (MDM2_rs769412);
dddd) a C or G nucleotide at a position corresponding to position 101 of SEQ ID NO: 163 (P2RX7_rs3751143);
eeee) a T nucleotide at a position corresponding to position 101 of SEQ ID NO: 165 (rs2187668);
ffff) an A nucleotide at a position corresponding to position 101 of SEQ ID NO: 167 (IL10_rs3024505);
gggg) a G nucleotide at a position corresponding to position 101 of SEQ ID NO: 169 (rs922075);
hhhh) a T nucleotide at a position corresponding to position 101 of SEQ ID NO: 171 (SOS1_rs1059313);
iiii) a C nucleotide at a position corresponding to position 101 of SEQ ID NO: 173 (STAT4_rs7574070);
jjjj) an A nucleotide at a position corresponding to position 101 of SEQ ID NO: 175 (UNGC.96.TGFB2_NA);
kkkk) an A nucleotide at a position corresponding to position 101 of SEQ ID NO: 177 (VEGFA_rs41282644);
llll) an A nucleotide at a position corresponding to position 101 of SEQ ID NO: 179 (IL1A_rs17561);
mmmm) a G nucleotide at a position corresponding to position 101 of SEQ ID NO: 181 (TGFB1_rs1800469);
nnnn) a C nucleotide at a position corresponding to position 101 of SEQ ID NO: 183 (EGFR_rs884225); and
oooo) a G nucleotide at a position corresponding to position 101 of SEQ ID NO: 185 (rs17388569).

52. The method of claim 51, the method comprising determining whether the patient carries one or more mutations selected from:
a) a C nucleotide at a position corresponding to position 101 of SEQ ID NO: 1 (miR.99a.promoter);
b) an A nucleotide at a position corresponding to position 101 of SEQ ID NO: 3 rs4848306);
c) a G nucleotide at a position corresponding to position 101 of SEQ ID NO: 5 (SHC4_rs1062124);
d) a T nucleotide at a position corresponding to position 101 of SEQ ID NO: 7 (rs17599026);
e) an A nucleotide at a position corresponding to position 101 of SEQ ID NO: 9 (RAC1_rs9374);
f) a C nucleotide at a position corresponding to position 101 of SEQ ID NO: 11 (IL24_rs1150258);
g) an A nucleotide at a position corresponding to position 101 of SEQ ID NO: 13 (IL1A_rs1800587);
h) a C nucleotide at a position corresponding to position 101 of SEQ ID NO: 15 (ERCC1_rs3212948);
i) a G nucleotide at a position corresponding to position 101 of SEQ ID NO: 17 (IL.6_rs12700386);
j) a G nucleotide at a position corresponding to position 101 of SEQ ID NO: 19 (IL.6_rs2069840);
k) a T nucleotide at a position corresponding to position 101 of SEQ ID NO: 21 (CAMK2G_rs2306327);
l) a T nucleotide at a position corresponding to position 101 of SEQ ID NO: 23 (BMP2_rs1980499);
m) a C nucleotide at a position corresponding to position 101 of SEQ ID NO: 25 (EREG_rs1460008);
n) a C nucleotide at a position corresponding to position 101 of SEQ ID NO: 27 (CD274_rs4143815);
o) an A nucleotide at a position corresponding to position 101 of SEQ ID NO: 29 (IL17D_rs7787);
p) a C nucleotide at a position corresponding to position 101 of SEQ ID NO: 31 (STAT3/rs3744483);
q) a C nucleotide at a position corresponding to position 101 of SEQ ID NO: 33 (BMP4_rs17563)
r) a C nucleotide at a position corresponding to position 101 of SEQ ID NO: 35 (IRF5_rs10488631); or
s) a T nucleotide at a position corresponding to position 101 of SEQ ID NO: 37 (UNGC.41.IL1RAP).

53. The method of claim 51 or 52, wherein the patient has a decreased likelihood of having a toxic response to the radiation treatment if the patient is carrying or not carrying one or more mutations selected from:
a) not carrying or carrying only one copy of a C nucleotide at a position corresponding to position 101 of SEQ ID NO: 1 (miR.99a.promoter);
b) not carrying or carrying only one copy of an A nucleotide at a position corresponding to position 101 of SEQ ID NO: 3 (IL1.B_rs4848306);
c) not carrying or carrying only one copy of a G nucleotide at a position corresponding to position 101 of SEQ ID NO: 5 (SHC4_rs1062124);
d) carrying a T nucleotide at a position corresponding to position 101 of SEQ ID NO: 7 (rs17599026);
e) not carrying or carrying only one copy of an A nucleotide at a position corresponding to position 101 of SEQ ID NO: 9 (RAC1_rs9374);
f) not carrying a C nucleotide at a position corresponding to position 101 of SEQ ID NO: 11 (IL24_rs1150258);
g) not carrying or carrying only one copy of an A nucleotide at a position corresponding to position 101 of SEQ ID NO: 13 (IL1A_rs1800587);
h) not carrying a C nucleotide at a position corresponding to position 101 of SEQ ID NO: 15 (ERCC1_rs3212948);
i) not carrying or carrying only one copy of a G nucleotide at a position corresponding to position 101 of SEQ ID NO: 17 (IL.6_rs12700386);
j) not carrying or carrying only one copy of a G nucleotide at a position corresponding to position 101 of SEQ ID NO: 19 (IL.6_rs2069840);
k) not carrying or carrying only one copy of a T nucleotide at a position corresponding to position 101 of SEQ ID NO: 21 (CAMK2G_rs2306327);
l) not carrying or carrying only one copy of a T nucleotide at a position corresponding to position 101 of SEQ ID NO: 23 (BMP2_rs1980499);
m) not carrying or carrying only one copy of a C nucleotide at a position corresponding to position 101 of SEQ ID NO: 25 (EREG_rs1460008);
n) not carrying or carrying only one copy of a C nucleotide at a position corresponding to position 101 of SEQ ID NO: 27 (CD274_rs4143815);
o) not carrying or carrying only one copy of an A nucleotide at a position corresponding to position 101 of SEQ ID NO: 29 (IL17D_rs7787);
p) not carrying or carrying only one copy of a C nucleotide at a position corresponding to position 101 of SEQ ID NO: 31 (STAT3/rs3744483);
q) not carrying or carrying only one copy of a C nucleotide at a position corresponding to position 101 of SEQ ID NO: 33 (BMP4_rs17563)
r) not carrying a C nucleotide at a position corresponding to position 101 of SEQ ID NO: 35 (IRF5_rs10488631); or
s) not carrying or carrying only one copy of a T nucleotide at a position corresponding to position 101 of SEQ ID NO: 37 (UNGC.41.IL1RAP).

54. The method of claim 51 or 52, wherein the patient has an increased likelihood of having a toxic response to the radiation treatment if the patient carries or does not carry one or more mutations selected from:
a) carrying a C nucleotide at a position corresponding to position 101 of SEQ ID NO: 1 (miR.99a.promoter);
b) carrying an A nucleotide at a position corresponding to position 101 of SEQ ID NO: 3 (IL1.B_rs4848306);
c) carrying a G nucleotide at a position corresponding to position 101 of SEQ ID NO: 5 (SHC4_rs1062124);
d) not carrying a T nucleotide at a position corresponding to position 101 of SEQ ID NO: 7 (rs17599026);
e) carrying an A nucleotide at a position corresponding to position 101 of SEQ ID NO: 9 (RAC1_rs9374);
f) carrying a C nucleotide at a position corresponding to position 101 of SEQ ID NO: 11 (IL24_rs1150258);
g) carrying an A nucleotide at a position corresponding to position 101 of SEQ ID NO: 13 (IL1A_rs1800587);
h) carrying a C nucleotide at a position corresponding to position 101 of SEQ ID NO: 15 (ERCC1_rs3212948);
i) carrying a G nucleotide at a position corresponding to position 101 of SEQ ID NO: 17 (IL.6_rs12700386);
j) carrying a G nucleotide at a position corresponding to position 101 of SEQ ID NO: 19 (IL.6_rs2069840);
k) carrying a T nucleotide at a position corresponding to position 101 of SEQ ID NO: 21 (CAMK2G_rs2306327);
l) carrying a T nucleotide at a position corresponding to position 101 of SEQ ID NO: 23 (BMP2_rs1980499);
m) carrying a C nucleotide at a position corresponding to position 101 of SEQ ID NO: 25 (EREG_rs1460008);
n) carrying a C nucleotide at a position corresponding to position 101 of SEQ ID NO: 27 (CD274_rs4143815);
o) carrying an A nucleotide at a position corresponding to position 101 of SEQ ID NO: 29 (IL17D_rs7787);
p) carrying two copies of a C nucleotide at a position corresponding to position 101 of SEQ ID NO: 31 (STAT3/rs3744483);
q) carrying a C nucleotide at a position corresponding to position 101 of SEQ ID NO: 33 (BMP4_rs17563)
r) carrying a C nucleotide at a position corresponding to position 101 of SEQ ID NO: 35 (IRF5_rs10488631); or
s) carrying a T nucleotide at a position corresponding to position 101 of SEQ ID NO: 37 (UNGC.41.IL1RAP).

55. The method of any one of claims 51-54, wherein the cancer is selected from adrenal cancer, anal cancer, bile duct cancer, bladder cancer, bone cancer, brain/CNS, basal cell skin cancer, breast cancer, cervical cancer, colorectal cancer, endometrial cancer, esophageal cancer, eye cancer, gallbladder cancer, gastrointestinal carcinoid tumors, gastrointestinal stromal tumor (GIST), gastric cancer, glioma, glioblastoma, head and neck cancer, Hodgkin disease, Kaposi sarcoma, kidney cancer, laryngeal and hypopharyngeal cancer, leukemia, liver cancer, lymphoma, malignant mesothelioma, merkel cell carcinoma, metastatic urothelial carcinoma, multiple myeloma, myeloma, myelodysplastic syndrome, nasal cavity and paranasal sinus cancer, nasopharyngeal cancer, neuroendocrine cancer, neuroblastoma, non-Hodgkin lymphoma, oral cavity and oropharyngeal cancer, osteosarcoma, ovarian cancer, pancreatic cancer, penile cancer, pituitary tumors, prostate cancer, renal cancer, retinoblastoma, rhabdomyosarcoma, salivary gland cancer, sarcoma, squamous cell skin cancer, small intestine cancer, stomach cancer, testicular cancer, thymus cancer, thyroid cancer, uterine cancer, or vaginal cancer.

56. The method of claim 55, wherein the cancer is sarcoma.

57. The method of any one of claims 51-56, wherein the radiation treatment comprises hypofractionated radiation.

58. The method of any one of claims 51-57, wherein the toxicity comprises wound toxicity.

59. The method of any one of claims 51-58, wherein the patient has a tumor on a lower extremity.

60. The method of claim 51, the method comprising determining whether the patient carries one or more mutations selected from:
a) a G nucleotide at position 101 of SEQ ID NO: 53 (BMP2_rs1979855);
b) a G nucleotide at position 101 of SEQ ID NO: 55 (PTPN2_rs1893217);
c) a C nucleotide at position 101 of SEQ ID NO: 43 (BRCA2_rs15869);
d) a C nucleotide at position 101 of SEQ ID NO: 65 (ABL1_rs11991);
e) a G nucleotide at position 101 of SEQ ID NO: 63 (ERCC1_rs11615);
f) an A nucleotide at position 101 of SEQ ID NO: 41 (RAD23A_rs8240);
g) a G nucleotide at position 101 of SEQ ID NO: 59 (BATF3_rs6695772);
h) a C nucleotide at position 101 of SEQ ID NO: 143 (FOXP3_rs2232365);
i) a G nucleotide at position 101 of SEQ ID NO: 39 (IL19_rs1798);
j) a G or T nucleotide at position 101 of SEQ ID NO: 129 (LIN28A_rs9438623);
k) an A nucleotide at position 101 of SEQ ID NO: 61 (IL18R1_rs11465660);
l) a T nucleotide at position 101 of SEQ ID NO: 153 (IL2RB_rs228942);
m) a T nucleotide at position 101 of SEQ ID NO: 49 (CD274_rs1411262);
n) a C nucleotide at position 101 of SEQ ID NO: 47 (BMP2_rs3178250);
o) a C nucleotide at position 101 of SEQ ID NO: 137 (SPI1_rs2071304);
p) a G nucleotide at position 101 of SEQ ID NO: 99 (IL19_rs2243158);
q) an A nucleotide at position 101 of SEQ ID NO: 167 (IL10_rs3024505);
r) a G nucleotide at position 101 of SEQ ID NO: 187 (TRL4_rs4986790);
s) an A nucleotide at position 101 of SEQ ID NO: 177 (VEGFA_rs41282644);
t) a G or C nucleotide at position 101 of SEQ ID NO: 163 (P2RX7_rs3751143);
u) a C nucleotide at position 101 of SEQ ID NO: 15 (ERCC1_rs3212948);
v) a G or C nucleotide at position 101 of SEQ ID NO: 131 (FANCC_rs9673);
w) a G nucleotide at position 101 of SEQ ID NO: 51 (CD274_rs2297136);
x) a G nucleotide at position 101 of SEQ ID NO: 19 (IL6_rs2069840);
y) a G nucleotide at position 101 of SEQ ID NO: 45 (CD274_rs2282055);
z) a G nucleotide at position 101 of SEQ ID NO: 57 (CD274_rs822339);
aa) a G nucleotide at position 101 of SEQ ID NO: 95 (HAMP_rs10421768);
bb) a C nucleotide at position 101 of SEQ ID NO: 145 (HAMP_rs1882694);
cc) a G nucleotide at position 101 of SEQ ID NO: 189 (ATM_rs1800057);
dd) a C nucleotide at position 101 of SEQ ID NO: 191 (ILRAP_rs79383051);
ee) an A nucleotide at position 101 of SEQ ID NO: 155 (KIT_rs17084733);
ff) a T nucleotide at position 101 of SEQ ID NO: 7 (rs17599026);
gg) an A nucleotide at position 101 of SEQ ID NO: 67 (ATM_rs189037);
hh) a G nucleotide at position 101 of SEQ ID NO: 69 (CD6_rs76677607);
ii) an A nucleotide at position 101 of SEQ ID NO: 13 (IL1A_rs1800587);
jj) an A nucleotide at position 101 of SEQ ID NO: 179 (IL1A_rs17561);
kk) a G nucleotide at position 101 of SEQ ID NO: 73 (BRCA2_rs7334543);
ll) a T nucleotide at position 101 of SEQ ID NO: 75 (ILF3.58_rs118142475);
mm) a C nucleotide at position 101 of SEQ ID NO: 77 (BIRC5_rs2239680);
nn) a T nucleotide at position 101 of SEQ ID NO: 107 (IL2RA_rs2476491);
oo) an A nucleotide at position 101 of SEQ ID NO: 109 (IL12A_rs568408);
pp) a C nucleotide at position 101 of SEQ ID NO: 111 (GSK3B_rs2037547);
qq) a G nucleotide at position 101 of SEQ ID NO: 135 (IL6_rs1800795);
rr) a T nucleotide at position 101 of SEQ ID NO: 105 (LIG4_rs2232643);
ss) a C nucleotide at position 101 of SEQ ID NO: 31 (STAT3_rs3744483);
tt) a G nucleotide at position 101 of SEQ ID NO: 115 (IL6_rs1800797);
uu) a G nucleotide at position 101 of SEQ ID NO: 17 (IL.6_rs12700386);
vv) an A nucleotide at position 101 of SEQ ID NO: 9 (RAC1_rs9374);
ww) a T nucleotide at position 101 of SEQ ID NO: 121 (ATM_rs373759);
xx) an A nucleotide at position 101 of SEQ ID NO: 123 (CETN2_rs8230);
yy) an A nucleotide at position 101 of SEQ ID NO: 126 (XRCC3_rs861539);
zz) a C nucleotide at position 101 of SEQ ID NO: 127 (miR.34b.c.promoter_rs4938723);
aaa) an A nucleotide at position 101 of SEQ ID NO: 3 (IL1.B_rs4848306);
bbb) an A nucleotide at position 101 of SEQ ID NO: 133 (REV3L_rs465646);
ccc) a C nucleotide at position 101 of SEQ ID NO: 183 (EGFR_rs884225);
ddd) a G nucleotide at position 101 of SEQ ID NO: 181 (TGFB1_rs1800469);
eee) a T nucleotide at position 101 of SEQ ID NO: 165 (rs2187668);
fff) a G nucleotide at position 101 of SEQ ID NO: 185 (rs17388569);
ggg) a G nucleotide at position 101 of SEQ ID NO: 79 (NBN_rs1805794);
hhh) a G nucleotide at position 101 of SEQ ID NO: 83 (CD274_rs4742098);
iii) a G nucleotide at position 101 of SEQ ID NO: 85 (ERCC4_rs4781562);
jjj) an A, C, or G nucleotide at position 101 of SEQ ID N: 87 (MSH2_rs2303428);
kkk) an A nucleotide at position 101 of SEQ ID NO: 151 (IL2RA_rs11256497);
lll) a C nucleotide at position 101 of SEQ ID NO: 159 (LIG4_rs3093772_P1.P2);
mmm) an A nucleotide at position 101 of SEQ ID NO: 29 (IL17D_rs7787);
nnn) a C nucleotide at position 101 of SEQ ID NO: (CD274_rs4143815);
ooo) an A nucleotide at position 101 of SEQ ID NO: 175 (UNGC.96.TGFB2_NA);
ppp) a G nucleotide at position 101 of SEQ ID NO: 161 (MDM2_rs769412); and
qqq) a G nucleotide at position 101 of SEQ ID NO: 147 (IL10_rs1800872).

61. The method of claim 60, wherein the patient has a decreased likelihood of having a toxic response if the patient carries or does not carry one or more mutations selected from:
a) not carrying or carrying only one copy of a G nucleotide at position 101 of SEQ ID NO: 53 (BMP2_rs1979855);
b) not carrying or carrying only one copy of a G nucleotide at position 101 of SEQ ID NO: 55 (PTPN2_rs1893217);
c) not carrying or carrying only one copy of a C nucleotide at position 101 of SEQ ID NO: 43 (BRCA2_rs15869);
d) carrying a C nucleotide at position 101 of SEQ ID NO: 65 (ABL1_rs11991);
e) not carrying or carrying only one copy of a G nucleotide at position 101 of SEQ ID NO: 63 (ERCC1_rs11615);
f) not carrying or carrying only one copy of an A nucleotide at position 101 of SEQ ID NO: 41 (RAD23A_rs8240);
g) carrying a G nucleotide at position 101 of SEQ ID NO: 59 (BATF3_rs6695772);
h) carrying a C nucleotide at position 101 of SEQ ID NO: 143 (FOXP3_rs2232365);
i) not carrying or carrying only one copy of a G nucleotide at position 101 of SEQ ID NO: 39 (IL19_rs1798);
j) not carrying a G or T nucleotide at position 101 of SEQ ID NO: 129 (LIN28A_rs9438623);
k) not carrying or carrying only one copy of an A nucleotide at position 101 of SEQ ID NO: 61 (IL18R1_rs11465660);
l) not carrying a T nucleotide at position 101 of SEQ ID NO: 153 (IL2RB_rs228942);
m) not carrying or carrying only one copy of a T nucleotide at position 101 of SEQ ID NO: 49 (CD274_rs1411262);
n) not carrying or carrying only one copy of a C nucleotide at position 101 of SEQ ID NO: 47 (BMP2_rs3178250);
o) not carrying a C nucleotide at position 101 of SEQ ID NO: 137 (SPIT_rs2071304);
p) not carrying a G nucleotide at position 101 of SEQ ID NO: 99 (IL19_rs2243158);
q) not carrying an A nucleotide at position 101 of SEQ ID NO: 167 (IL10_rs3024505);
r) carrying a G nucleotide at position 101 of SEQ ID NO: 187 (TRL4_rs4986790);
s) carrying an A nucleotide at position 101 of SEQ ID NO: 177 (VEGFA_rs41282644);
t) carrying a G or C nucleotide at position 101 of SEQ ID NO: 163 (P2RX7_rs3751143);
u) not carrying a C nucleotide at position 101 of SEQ ID NO: 15 (ERCC1_rs3212948);
v) not carrying a G or C nucleotide at position 101 of SEQ ID NO: 131 (FANCC_rs9673);
w) not carrying a G nucleotide at position 101 of SEQ ID NO: 51 (CD274_rs2297136);
x) not carrying or carrying only one copy of a G nucleotide at position 101 of SEQ ID NO: 19 (IL6_rs2069840);
y) not carrying or carrying only one copy of a G nucleotide at position 101 of SEQ ID NO: 45 (CD274_rs2282055);
z) carrying a G nucleotide at position 101 of SEQ ID NO: 57 (CD274_rs822339);
aa) carrying a G nucleotide at position 101 of SEQ ID NO: 95 (HAMP_rs10421768);
bb) carrying a C nucleotide at position 101 of SEQ ID NO: 145 (HAMP_rs1882694);
cc) not carrying a G nucleotide at position 101 of SEQ ID NO: 189 (ATM_rs1800057);
dd) not carrying a C nucleotide at position 101 of SEQ ID NO: 191 (ILRAP_rs79383051);
ee) carrying an A nucleotide at position 101 of SEQ ID NO: 155 (KIT_rs17084733); and
ff) carrying a T nucleotide at position 101 of SEQ ID NO: 7 (rs17599026).

62. The method of claim 60, wherein the patient has an increased likelihood of having a toxic response if the patient carries or does not carry one or more mutations selected from:
a) carrying two copies of a G nucleotide at position 101 of SEQ ID NO: 53 (BMP2_rs1979855);
b) carrying two copies of a G nucleotide at position 101 of SEQ ID NO: 55 (PTPN2_rs1893217);
c) carrying two copies of a C nucleotide at position 101 of SEQ ID NO: 43 (BRCA2_rs15869);
d) not carrying a C nucleotide at position 101 of SEQ ID NO: 65 (ABL1_rs11991);
e) carrying two copies of a G nucleotide at position 101 of SEQ ID NO: 63 (ERCC1_rs11615);
f) carrying two copies of an A nucleotide at position 101 of SEQ ID NO: 41 (RAD23A_rs8240);
g) not carrying a G nucleotide at position 101 of SEQ ID NO: 59 (BATF3_rs6695772);
h) not carrying a C nucleotide at position 101 of SEQ ID NO: 143 (FOXP3_rs2232365);
i) carrying two copies of a G nucleotide at position 101 of SEQ ID NO: 39 (IL19_rs1798);
j) carrying a G or T nucleotide at position 101 of SEQ ID NO: 129 (LIN28A_rs9438623);
k) carrying two copies of an A nucleotide at position 101 of SEQ ID NO: 61 (IL18R1_rs11465660);
l) carrying a T nucleotide at position 101 of SEQ ID NO: 153 (IL2RB_rs228942);
m) carrying two copies of a T nucleotide at position 101 of SEQ ID NO: 49 (CD274_rs1411262);
n) carrying two copies of a C nucleotide at position 101 of SEQ ID NO: 47 (BMP2_rs3178250);
o) carrying a C nucleotide at position 101 of SEQ ID NO: 137 (SPIT_rs2071304);
p) carrying a G nucleotide at position 101 of SEQ ID NO: 99 (IL19_rs2243158);
q) carrying an A nucleotide at position 101 of SEQ ID NO: 167 (IL10_rs3024505);
r) not carrying a G nucleotide at position 101 of SEQ ID NO: 187 (TRL4_rs4986790);
s) not carrying an A nucleotide at position 101 of SEQ ID NO: 177 (VEGFA_rs41282644);
t) not carrying a G or C nucleotide at position 101 of SEQ ID NO: 163 (P2RX7_rs3751143);
u) carrying a C nucleotide at position 101 of SEQ ID NO: 15 (ERCC1_rs3212948);
v) carrying a G or C nucleotide at position 101 of SEQ ID NO: 131 (FANCC_rs9673);
w) carrying a G nucleotide at position 101 of SEQ ID NO: 51 (CD274_rs2297136);
x) carrying two copies of a G nucleotide at position 101 of SEQ ID NO: 19 (IL6_rs2069840);
y) carrying two copies of a G nucleotide at position 101 of SEQ ID NO: 45 (CD274_rs2282055);
z) not carrying a G nucleotide at position 101 of SEQ ID NO: 57 (CD274_rs822339);
aa) not carrying a G nucleotide at position 101 of SEQ ID NO: 95 (HAMP_rs10421768);
bb) not carrying a C nucleotide at position 101 of SEQ ID NO: 145 (HAMP_rs1882694);
cc) carrying a G nucleotide at position 101 of SEQ ID NO: 189 (ATM_rs1800057);
dd) carrying a C nucleotide at position 101 of SEQ ID NO: 191 (ILRAP_rs79383051);
ee) not carrying an A nucleotide at position 101 of SEQ ID NO: 155 (KIT_rs17084733); and
ff) not carrying a T nucleotide at position 101 of SEQ ID NO: 7 (rs17599026).

63. The method of any one of claims 60-62, wherein the radiation treatment is a hypofractionated radiation regimen.

64. The method of claim 63, wherein the hypofractionated radiation regimen comprises administering from about 20 Gy to about 35 Gy radiation over about 5 to about 8 fractions.

65. The method of claim 63 or 64, wherein the hypofractionated radiation regimen comprises administering stereotactic body radiation therapy.

66. The method of any one of claims 60-65, wherein the cancer is selected from adrenal cancer, anal cancer, bile duct cancer, bladder cancer, bone cancer, brain/CNS, basal cell skin cancer, breast cancer, cervical cancer, colorectal cancer, endometrial cancer, esophageal cancer, eye cancer, gallbladder cancer, gastrointestinal carcinoid tumors, gastrointestinal stromal tumor (GIST), gastric cancer, glioma, glioblastoma, head and neck cancer, Hodgkin disease, Kaposi sarcoma, kidney cancer, laryngeal and hypopharyngeal cancer, leukemia, liver cancer, lymphoma, malignant mesothelioma, merkel cell carcinoma, metastatic urothelial carcinoma, multiple myeloma, myeloma, myelodysplastic syndrome, nasal cavity and paranasal sinus cancer, nasopharyngeal cancer, neuroendocrine cancer, neuroblastoma, non-Hodgkin lymphoma, oral cavity and oropharyngeal cancer, osteosarcoma, ovarian cancer, pancreatic cancer, penile cancer, pituitary tumors, prostate cancer, renal cancer, retinoblastoma, rhabdomyosarcoma, salivary gland cancer, sarcoma, squamous cell skin cancer, small intestine cancer, stomach cancer, testicular cancer, thymus cancer, thyroid cancer, uterine cancer, or vaginal cancer.

67. The method of claim 65, wherein the cancer is prostate cancer.

68. The method of claim 60, wherein the patient has an decreased likelihood of having a toxic response if the patient carries or does not carry one or more mutations selected from:
a) carrying an A nucleotide at position 101 of SEQ ID NO: 67 (ATM_rs189037);
b) not carrying a G nucleotide at position 101 of SEQ ID NO: 69 (CD6_rs76677607);
c) not carrying or carrying only one copy of an A nucleotide at position 101 of SEQ ID NO: 13 (IL1A_rs1800587);
d) carrying an A nucleotide at position 101 of SEQ ID NO: 179 (IL1A_rs17561);
e) carrying a G nucleotide at position 101 of SEQ ID NO: 73 (BRCA2_rs7334543);
f) not carrying a T nucleotide at position 101 of SEQ ID NO: 75 (ILF3.58_rs118142475);
g) carrying a C nucleotide at position 101 of SEQ ID NO: 77 (BIRC5_rs2239680);
h) carrying a T nucleotide at position 101 of SEQ ID NO: 107 (IL2RA_rs2476491);
i) carrying an A nucleotide at position 101 of SEQ ID NO: 109 (IL12A_rs568408);
j) not carrying a C nucleotide at position 101 of SEQ ID NO: 111 (GSK3B_rs2037547);
k) carrying a G nucleotide at position 101 of SEQ ID NO: 135 (IL6_rs1800795);
l) not carrying a T nucleotide at position 101 of SEQ ID NO: 105 (LIG4_rs2232643);
m) not carrying or carrying only one copy of a C nucleotide at position 101 of SEQ ID NO: 31 (STAT3_rs3744483);
n) carrying a G nucleotide at position 101 of SEQ ID NO: 115 (IL6_rs1800797);
o) not carrying or carrying only one copy of a G nucleotide at position 101 of SEQ ID NO: 17 (IL.6_rs12700386);
p) not carrying or carrying only one copy of an A nucleotide at position 101 of SEQ ID NO: 9 (RAC1_rs9374);
q) not carrying a T nucleotide at position 101 of SEQ ID NO: 121 (ATM_rs373759);
r) not carrying an A nucleotide at position 101 of SEQ ID NO: 123 (CETN2_rs8230);
s) not carrying an A nucleotide at position 101 of SEQ ID NO: 126 (XRCC3_rs861539);
t) carrying a C nucleotide at position 101 of SEQ ID NO: 127 (miR.34b.c.promoter_rs4938723);
u) not carrying or carrying only one copy of an A nucleotide at position 101 of SEQ ID NO: 61 (IL18R1_rs11465660);
v) not carrying or carrying only one copy of an A nucleotide at position 101 of SEQ ID NO: 3 (IL1.B_rs4848306);
w) not carrying a G or C nucleotide at position 101 of SEQ ID NO: 131 (FANCC_rs9673);
x) carrying an A nucleotide at position 101 of SEQ ID NO: 133 (REV3L_rs465646);
y) not carrying a G nucleotide at position 101 of SEQ ID NO: 51 (CD274_rs2297136);
z) carrying a C nucleotide at position 101 of SEQ ID NO: 183 (EGFR_rs884225);
aa) not carrying a G nucleotide at position 101 of SEQ ID NO: 181 (TGFB1_rs1800469);
bb) carrying a T nucleotide at position 101 of SEQ ID NO: 165 (rs2187668);
cc) carrying a G nucleotide at position 101 of SEQ ID NO: 185 (rs17388569);
dd) not carrying a G nucleotide at position 101 of SEQ ID NO: 79 (NBN_rs1805794);
ee) carrying a G nucleotide at position 101 of SEQ ID NO: 83 (CD274_rs4742098);
ff) carrying a G nucleotide at position 101 of SEQ ID NO: 85 (ERCC4_rs4781562);
gg) not carrying an A, C, or G nucleotide at position 101 of SEQ ID N: 87 (MSH2_rs2303428);
hh) carrying a T nucleotide at position 101 of SEQ ID NO: 7 (rs17599026);
ii) not carrying an A nucleotide at position 101 of SEQ ID NO: 151 (IL2RA_rs11256497);
jj) carrying a C nucleotide at position 101 of SEQ ID NO: 159 (LIG4_rs3093772_P1.P2);
kk) not carrying or carrying only one copy of an A nucleotide at position 101 of SEQ ID NO: 29 (IL17D_rs7787);
ll) not carrying or carrying only one copy of a C nucleotide at position 101 of SEQ ID NO: (CD274_rs4143815);
mm) not carrying an A nucleotide at position 101 of SEQ ID NO: 175 (UNGC.96.TGFB2_NA);
nn) not carrying a G nucleotide at position 101 of SEQ ID NO: 161 (MDM2_rs769412);
oo) not carrying an A nucleotide at position 101 of SEQ ID NO: 167 (IL10_rs3024505);
pp) not carrying or carrying only one copy of a C nucleotide at position 101 of SEQ ID NO: 43 (BRCA2_rs15869);
qq) not carrying a G nucleotide at position 101 of SEQ ID NO: 147 (IL10_rs1800872);
rr) not carrying or carrying only one copy of a G nucleotide at position 101 of SEQ ID NO: 53 (BMP2_rs1979855);
ss) not carrying or carrying only one copy of a G nucleotide at position 101 of SEQ ID NO: 55 (PTPN2_rs1893217);
tt) carrying a C nucleotide at position 101 of SEQ ID NO: 65 (ABL1_rs11991); and
uu) not carrying or carrying only one copy of a G nucleotide at position 101 of SEQ ID NO: 63 (ERCC1_rs11615).

69. The method of claim 60, wherein the patient has an increased likelihood of having a toxic response if the patient carries or does not carry one or more mutations selected from:
a) not carrying an A nucleotide at position 101 of SEQ ID NO: 67 (ATM_rs189037);
b) carrying a G nucleotide at position 101 of SEQ ID NO: 69 (CD6_rs76677607);
c) carrying two copies of an A nucleotide at position 101 of SEQ ID NO: 13 (IL1A_rs1800587);
d) not carrying an A nucleotide at position 101 of SEQ ID NO: 179 (IL1A_rs17561);
e) not carrying a G nucleotide at position 101 of SEQ ID NO: 73 (BRCA2_rs7334543);
f) carrying a T nucleotide at position 101 of SEQ ID NO: 75 (ILF3.58_rs118142475);
g) not carrying a C nucleotide at position 101 of SEQ ID NO: 77 (BIRC5_rs2239680);
h) not carrying a T nucleotide at position 101 of SEQ ID NO: 107 (IL2RA_rs2476491);
i) not carrying an A nucleotide at position 101 of SEQ ID NO: 109 (IL12A_rs568408);
j) carrying a C nucleotide at position 101 of SEQ ID NO: 111 (GSK3B_rs2037547);
k) not carrying a G nucleotide at position 101 of SEQ ID NO: 135 (IL6_rs1800795);
l) carrying a T nucleotide at position 101 of SEQ ID NO: 105 (LIG4_rs2232643);
m) carrying two copies of a C nucleotide at position 101 of SEQ ID NO: 31 (STAT3_rs3744483);
n) not carrying a G nucleotide at position 101 of SEQ ID NO: 115 (IL6_rs1800797);
o) carrying two copies of a G nucleotide at position 101 of SEQ ID NO: 17 (IL.6_rs12700386);
p) carrying two copies of an A nucleotide at position 101 of SEQ ID NO: 9 (RAC1_rs9374);
q) carrying a T nucleotide at position 101 of SEQ ID NO: 121 (ATM_rs373759);
r) carrying an A nucleotide at position 101 of SEQ ID NO: 123 (CETN2_rs8230);
s) carrying an A nucleotide at position 101 of SEQ ID NO: 126 (XRCC3_rs861539);
t) not carrying a C nucleotide at position 101 of SEQ ID NO: 127 (miR.34b.c.promoter_rs4938723);
u) carrying two copies of an A nucleotide at position 101 of SEQ ID NO: 61 (IL18R1_rs11465660);
v) carrying two copies of an A nucleotide at position 101 of SEQ ID NO: 3 rs4848306);
w) carrying a G or C nucleotide at position 101 of SEQ ID NO: 131 (FANCC_rs9673);
x) not carrying an A nucleotide at position 101 of SEQ ID NO: 133 (REV3L_rs465646);
y) carrying a G nucleotide at position 101 of SEQ ID NO: 51 (CD274_rs2297136);
z) not carrying a C nucleotide at position 101 of SEQ ID NO: 183 (EGFR_rs884225);
aa) carrying a G nucleotide at position 101 of SEQ ID NO: 181 (TGFB1_rs1800469);
bb) not carrying a T nucleotide at position 101 of SEQ ID NO: 165 (rs2187668);
cc) not carrying a G nucleotide at position 101 of SEQ ID NO: 185 (rs17388569);
dd) carrying a G nucleotide at position 101 of SEQ ID NO: 79 (NBN_rs1805794);
ee) not carrying a G nucleotide at position 101 of SEQ ID NO: 83 (CD274_rs4742098);
ff) not carrying a G nucleotide at position 101 of SEQ ID NO: 85 (ERCC4_rs4781562);
gg) carrying an A, C, or G nucleotide at position 101 of SEQ ID N: 87 (MSH2_rs2303428);
hh) not carrying a T nucleotide at position 101 of SEQ ID NO: 7 (rs17599026);
ii) carrying an A nucleotide at position 101 of SEQ ID NO: 151 (IL2RA_rs11256497);
jj) not carrying a C nucleotide at position 101 of SEQ ID NO: 159 (LIG4_rs3093772_P1.P2);
kk) carrying two copies of an A nucleotide at position 101 of SEQ ID NO: 29 (IL17D_rs7787);
ll) carrying two copies of a C nucleotide at position 101 of SEQ ID NO: (CD274_rs4143815);
mm) carrying an A nucleotide at position 101 of SEQ ID NO: 175 (UNGC.96.TGFB2_NA);
nn) carrying a G nucleotide at position 101 of SEQ ID NO: 161 (MDM2_rs769412);
oo) carrying an A nucleotide at position 101 of SEQ ID NO: 167 (IL10_rs3024505);
pp) carrying two copies of a C nucleotide at position 101 of SEQ ID NO: 43 (BRCA2_rs15869);
qq) carrying a G nucleotide at position 101 of SEQ ID NO: 147 (IL10_rs1800872);
rr) carrying two copies of a G nucleotide at position 101 of SEQ ID NO: 53 (BMP2_rs1979855);
ss) carrying two copies of a G nucleotide at position 101 of SEQ ID NO: 55 (PTPN2_rs1893217);
tt) not carrying a C nucleotide at position 101 of SEQ ID NO: 65 (ABL1_rs11991); and
uu) carrying two copies of a G nucleotide at position 101 of SEQ ID NO: 63 (ERCC1_rs11615).

70. The method of claim 68 or 69, wherein the cancer is selected from adrenal cancer, anal cancer, bile duct cancer, bladder cancer, bone cancer, brain/CNS, basal cell skin cancer, breast cancer, cervical cancer, colorectal cancer, endometrial cancer, esophageal cancer, eye cancer, gallbladder cancer, gastrointestinal carcinoid tumors, gastrointestinal stromal tumor (GIST), gastric cancer, glioma, glioblastoma, head and neck cancer, Hodgkin disease, Kaposi sarcoma, kidney cancer, laryngeal and hypopharyngeal cancer, leukemia, liver cancer, lymphoma, malignant mesothelioma, merkel cell carcinoma, metastatic urothelial carcinoma, multiple myeloma, myeloma, myelodysplastic syndrome, nasal cavity and paranasal sinus cancer, nasopharyngeal cancer, neuroendocrine cancer, neuroblastoma, non-Hodgkin lymphoma, oral cavity and oropharyngeal cancer, osteosarcoma, ovarian cancer, pancreatic cancer, penile cancer, pituitary tumors, prostate cancer, renal cancer, retinoblastoma, rhabdomyosarcoma, salivary gland cancer, sarcoma, squamous cell skin cancer, small intestine cancer, stomach cancer, testicular cancer, thymus cancer, thyroid cancer, uterine cancer, or vaginal cancer.

71. The method of claim 70, wherein the cancer is prostate cancer.

72. The method of any one of claims 68-71, wherein the toxicity is acute or long-term toxicity.

73. The method of claim 68, wherein the toxicity is GI and/or GU toxicity.

74. The method of any one of claims 68-72, wherein the radiation treatment is a conventionally fractionated (traditional) radiation regimen.

75. The method of claim 74, wherein the traditional radiation regimen comprises administering from about 40 Gy to about 60 Gy radiation over a period of about 5 to about 6 weeks.

76. The method of claim 74, wherein the traditional radiation regimen comprises administering from about 40 Gy to about 60 Gy radiation over about 15 to about 30 fractions.

77. The method of any of claims 68-76, wherein the radiation treatment is external beam radiation therapy.

78. A method for determining whether a cancer patient has a decreased likelihood distant failure in response to radiation, the method comprising determining whether the patient carries one or more mutations selected from: TREX1_rs11797, LIN28A_rs9438623, miR.34b.c.promoter_rs4938723, FCGR2A_rs1801274, CD274_rs4742098, IL.6_rs12700386, SPI1_rs2071304, ERCC4_rs4781562, IL10_rs3024496, IL.6_rs2069840, rs2187668, CD274_rs4143815, KRAS_rs61764370, SHC4_rs1062124, HAMP_rs1882694, rs922075, ERCC1_rs11615, EXO1_rs4150021, STAT4_rs7574070, and SOS1_rs1059313.

79. The method of claim 78, wherein the method comprises determining whether a patient is carrying or not carrying one or more mutations in a SNP selected from:
a) carrying a T nucleotide at position 101 of SEQ ID NO: 81 (TREX1_rs11797);
b) carrying a G or T nucleotide at position 101 of SEQ ID NO: 129 (LIN28A_rs9438623);
c) carrying or not carrying a C nucleotide at position 101 of SEQ ID NO: 127 (miR.34b.c.promoter_rs4938723);
d) carrying a C or G nucleotide at position 101 of SEQ ID NO: 119 (FCGR2A_rs1801274);
e) carrying a G nucleotide at position 101 of SEQ ID NO: 83 (CD274_rs4742098);
f) not carrying a G nucleotide at position 101 of SEQ ID NO: 17 (IL.6_rs12700386);
g) not carrying a C nucleotide at position 101 of SEQ ID NO: 137 (SPI1_rs2071304);
h) carrying a G nucleotide at position 101 of SEQ ID NO: 85 (ERCC4_rs4781562);
i) carrying a G nucleotide at position 101 of SEQ ID NO: 103 (IL10_rs3024496_P1.P2);
j) carrying a G nucleotide at position 101 of SEQ ID NO: 139 (IL10_rs3024496);
k) not carrying or carrying only one copy of a G nucleotide at position 101 of SEQ ID NO: 19 (IL.6_rs2069840);
l) not carrying a T nucleotide at position 101 of SEQ ID NO: 165 (rs2187668);
m) not carrying a C nucleotide at position 101 of SEQ ID NO: 27 (CD274_rs4143815);
n) carrying a C nucleotide at position 101 of SEQ ID NO: 157 (KRAS_rs61764370);
o) carrying a G nucleotide at position 101 of SEQ ID NO: 5 (SHC4_rs1062124);
p) carrying a G nucleotide at position 101 of SEQ ID NO: 169 (rs922075);
q) carrying a G nucleotide at position 101 of SEQ ID NO: 63 (ERCC1_rs11615);
r) not carrying the deletion of a T nucleotide at [ATTTTG] of SEQ ID NO: 142 (EXO1_rs4150021);
s) carrying a C nucleotide at position 101 of SEQ ID NO: 173 (STAT4_rs7574070); and
t) carrying a T nucleotide at position 101 of SEQ ID NO: 171 (SOS1_rs1059313).

80. The method of claim 78 or 79, wherein the radiation therapy is conventionally fractionated (traditional) radiation.

81. The method of any one of claims 78-80, wherein the cancer is prostate cancer.

82. A method for determining whether a cancer patient has an increased risk of mortality following a radiation treatment, the method comprising determining whether the patient carries one or more mutations selected from: BIRC5_rs2239680, SMAD1_rs11724777, IL12A_rs568408, and IL13_rs20541, wherein the presence or the absence of the SNP is not associated with an increased risk of mortality.

83. The method of claim 82, wherein the patient is identified as carrying or not carrying one or more mutations in a SNP selected from:
a) not carrying a C nucleotide at position 101 of SEQ ID NO: 77 (BIRC5_rs2239680);
b) carrying an A nucleotide at position 101 of SEQ ID NO: 93 (SMAD1_rs11724777);
c) carrying an A nucleotide at position 101 of SEQ ID NO: 109 (IL12A_rs568408); and
d) carrying a T nucleotide at position 101 of SEQ ID NO: 193 (IL13_rs20541).

84. The method of claim 82 or 83, wherein the radiation treatment is conventionally fractionated (traditional) radiation.

85. The method of any one of claims 82-84, wherein the cancer is prostate cancer.

86. A method for determining whether a cancer patient has an increased risk of biochemical relapse following a radiation treatment, the method comprising determining whether the patient carries one or more mutations selected from: BMP2_rs3178250, SMAD1_rs11724777, IL10RB_rs2834167, CD274_rs822339, CD274_rs2282055, IL12A_rs568408, PARP1_rs8679, CD274_rs1411262, BMP2_rs1979855, and P2RX7_rs3751143.

87. The method of claim 86, wherein the patient has a decreased risk of biochemical relapse following a radiation treatment if the patient is identified as carrying or not carrying one or more mutations in a SNP selected from:
a) carrying a C nucleotide at position 101 of SEQ ID NO: 47 (BMP2_rs3178250);
b) not carrying a G nucleotide at position 101 of SEQ ID NO: 91 (IL10RB_rs2834167);
c) not carrying a G nucleotide at position 101 of SEQ ID NO: 57 (CD274_rs822339);
d) carrying a G nucleotide at position 101 of SEQ ID NO: 45 (CD274_rs2282055);
e) carrying an A nucleotide at position 101 of SEQ ID NO: 109 (IL12A_rs568408);
f) carrying a G nucleotide at position 101 of SEQ ID NO: 195 (PARP1_rs8679);
g) carrying a T nucleotide at position 101 of SEQ ID NO: 49 (CD274_rs1411262);
h) carrying a G nucleotide at position 101 of SEQ ID NO: 53 (BMP2_rs1979855); and
i) carrying a C nucleotide at position 101 of SEQ ID NO: 163 (P2RX7_rs3751143).

88. The method of claim 86 or 87, wherein the radiation therapy is conventionally fractionated (traditional) radiation.

89. The method of any one of claims 86-88, wherein the cancer is prostate cancer.

90. A method for determining whether a cancer patient has a decreased risk of distant metastasis following a radiation treatment, the method comprising determining whether the patient is carrying or not carrying one or more mutations in a SNP selected from: IL10_rs3024496, FOXP3_rs2280883, BIRC5_rs2239680, IL10_rs3024496_P1.P2, LIG4_rs2232643, and RAC1_rs9374, wherein the presence or the absence of the SNP is not associated with distant metastasis or is associated with a reduced risk of distant metastasis.

91. The method of claim 90, wherein the patient has a decreased risk of distant metastasis following a radiation treatment if the patient is identified as carrying or not carrying one or more mutations in a SNP selected from:
a) carrying a G nucleotide at position 101 of SEQ ID NO: 103 (IL10_rs3024496);
b) carrying a C nucleotide occurs at position 101 of SEQ ID NO: 101 (FOXP3_rs2280883);
c) carrying a C nucleotide occurs at position 101 of SEQ ID NO: 77 (BIRC5_rs2239680);
d) carrying a G nucleotide occurs at position 101 of SEQ ID NO: 103 IL10_rs3024496_P1.P2;
e) not carrying a T nucleotide at position 101 of SEQ ID NO: 105 (LIG4_rs2232643); and
f) not carrying an A nucleotide at position 101 of SEQ ID NO: 9 (RAC1_rs9374).

92. The method of claim 90 or 91, wherein the radiation therapy is conventionally fractionated (traditional) radiation.

93. The method of any one of claims 90-92, wherein the cancer is prostate cancer.

94. A method for determining whether a prostate cancer patient has a decreased risk of impotency following treatment with radiation, the method comprising determining whether the patient is carrying or not carrying one or more mutations in a SNP selected from: HAMP_rs10421768, XRCC1_rs25487, IL.6_rs2069840, XRCC4_rs1040363, and IL19_rs2243158.

95. The method of claim 94, wherein the patient is identified as carrying or not carrying one or more mutations in a SNP selected from:
a) carrying a G nucleotide at position 101 of SEQ ID NO: 95 (HAMP_rs10421768);
b) not carrying a C nucleotide at position 101 of SEQ ID NO: 97 (XRCC1_rs25487);
c) not carrying a G nucleotide at position 101 of SEQ ID NO: 19 (IL.6_rs2069840);
d) carrying a C nucleotide at position 101 of SEQ ID NO: 89 (XRCC4_rs1040363); and
e) not carrying a G nucleotide at position 101 of SEQ ID NO: 99 (IL19_rs2243158).

96. The method of claim 94 or 95, wherein the radiation treatment is conventionally fractionated (traditional) radiation.

97. A method for determining the toxicity of a radiation treatment in a cancer patient, wherein the is patient identified as carrying or not carrying a G nucleotide at a position corresponding to position 101 of SEQ ID NO: 19 (IL.6_rs2069840).

98. The method of claim 97, wherein the patient is further identified as carrying or not carrying an A nucleotide at a position corresponding to position 101 of SEQ ID NO: 69 (CD6_rs76677607).

99. The method of claim 98, wherein the patient is further identified as carrying or not carrying a C nucleotide at a position corresponding to position 101 of SEQ ID NO: 27 (CD274_rs4143815).

100. The method of claim 99, wherein the patient is further identified as carrying or not carrying a G nucleotide at a position corresponding to position 101 of SEQ ID NO: 5 (SHC4_rs1062124).

101. The method of claim 99, wherein the patient is further identified as carrying or not carrying a C nucleotide at a position corresponding to position 101 of SEQ ID NO: 15 (ERCC1_rs3212948).

102. The method of any one of claims 98-101, wherein the patient is further identified as carrying or not carrying an A nucleotide at a position corresponding to position 101 of SEQ ID NO: 117 (RAC1_rs9374).

103. The method of claim 98, wherein the method further comprises determining whether the patient has a lower extremity tumor.

104. The method of claim 103, wherein the patient is further identified as carrying or not carrying a T nucleotide at a position corresponding to position 101 of SEQ ID NO: 37 (UNGC.41.IL1RAP).

105. The method of claim 104, wherein the patient is further identified as carrying or not carrying a C nucleotide at a position corresponding to position 101 of SEQ ID NO: 27 (CD274_rs4143815).

106. The method of claim 105, wherein the patient is further identified as carrying or not carrying a C nucleotide at a position corresponding to position 101 of SEQ ID NO: 1 (miR.99a.promoter).

107. The method of claim 106, wherein the patient is further identified as carrying or not carrying an A nucleotide at a position corresponding to position 101 of SEQ ID NO: 13 (IL1A_rs1800587).

108. The method of any one of claims 97-107, wherein the method further comprises administering a reduced toxicity radiation treatment to the patient.

109. The method of any one of claims 97-108, wherein the cancer is selected from adrenal cancer, anal cancer, bile duct cancer, bladder cancer, bone cancer, brain/CNS, basal cell skin cancer, breast cancer, cervical cancer, colorectal cancer, endometrial cancer, esophageal cancer, eye cancer, gallbladder cancer, gastrointestinal carcinoid tumors, gastrointestinal stromal tumor (GIST), gastric cancer, glioma, glioblastoma, head and neck cancer, Hodgkin disease, Kaposi sarcoma, kidney cancer, laryngeal and hypopharyngeal cancer, leukemia, liver cancer, lymphoma, malignant mesothelioma, merkel cell carcinoma, metastatic urothelial carcinoma, multiple myeloma, myeloma, myelodysplastic syndrome, nasal cavity and paranasal sinus cancer, nasopharyngeal cancer, neuroendocrine cancer, neuroblastoma, non-Hodgkin lymphoma, oral cavity and oropharyngeal cancer, osteosarcoma, ovarian cancer, pancreatic cancer, penile cancer, pituitary tumors, prostate cancer, renal cancer, retinoblastoma, rhabdomyosarcoma, salivary gland cancer, sarcoma, squamous cell skin cancer, small intestine cancer, stomach cancer, testicular cancer, thymus cancer, thyroid cancer, uterine cancer, or vaginal cancer.

110. The method of claim 109, wherein the cancer is sarcoma.

111. The method of any one of claims 97-110, wherein the toxicity is wound toxicity.

112. The method of any one of claims 108-111, wherein the radiation treatment comprises a hypofractionated radiation regimen.

113. The method of claim 112, wherein the hypofractionated radiation regimen comprises administering from about 20 Gy to about 35 Gy radiation over about 5 to about 8 fractions.

114. The method of claim 112 or 113, wherein the hypofractionated radiation regimen comprises administering stereotactic body radiation therapy.

115. The method of any one of claims 108-114, wherein the radiation treatment comprises a conventionally fractionated (traditional) radiation regimen.

116. The method of claim 115, wherein the traditional radiation regimen comprises administering from about 40 Gy to about 60 Gy radiation over a period of about 5 to about 6 weeks.

117. The method of claim 115, wherein the traditional radiation regimen comprises administering from about 40 Gy to about 60 Gy radiation over about 15 to about 30 fractions.
